(19) United States
(12) Patent Application Publication
(10) Pub. No.: US 2024/0368153 A1
(43) Pub. Date: Nov. 7, 2024

(54) Aminopyridine-based MTA-Cooperative PRMT5 Inhibitors

(71) Applicant: Mirati Therapeutics, Inc., San Diego, CA (US)

(72) Inventors: Christopher Ronald Smith, San Diego, CA (US); Svitlana Kulyk, Redwood City, CA (US); Matthew Arnold Marx, San Diego, CA (US); John David Lawson, Carlsbad, CA (US); Anthony Ivetac, San Diego, CA (US); Xiaolun Wang, San Diego, CA (US); Aaron Craig Burns, San Diego, CA (US)

(21) Appl. No.: 18/572,110

(22) PCT Filed: Jun. 29, 2022
(86) PCT No.: PCT/US2022/035508

§ 371 Date: Dec. 19, 2023

Related U.S. Application Data
(60) Provisional application No. 63/218,090, filed on Jul. 2, 2021.

(51) Int. Cl. C07D 471/04; A61K 31/4365; A61K 31/437; A61K 31/4375; A61K 31/498; A61K 31/506; A61K 31/551; C07D 471/14; C07D 495/04

(52) U.S. Cl. CPC C07D 471/04; A61K 31/4365; A61K 31/437; A61K 31/4375; A61K 31/498; A61K 31/506; A61K 31/551; C07D 471/14; C07D 495/04

(57) ABSTRACT

The present invention relates to compounds that inhibit Protein Arginine N-Methyl Transferase 5 (PRMT5) activity. In particular, the present invention relates to compounds, pharmaceutical compositions and methods of use, such as methods of treating cancer using the compounds and pharmaceutical compositions of the present invention.

### CROSS-REFERENCE TO RELATED APPLICATIONS

This application claims the benefit of priority of U.S. Provisional Application No. 63/218,090, filed Jul. 2, 2021, the disclosure of which is hereby incorporated by reference in its entirety.

### FIELD OF THE INVENTION

The present invention relates to compounds that are MTA-cooperative inhibitors of Protein Arginine N-Methyl Transferase 5 (PRMT5). In particular, the present invention relates to compounds, pharmaceutical compositions comprising the compounds and methods for use therefor.

### BACKGROUND OF THE INVENTION

Protein Arginine N-Methyl Transferase (PRMT5) is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recognition and orientation and is also required for PRMT5-catalyzed histone 2A and histone 4 methyltransferase activity (e.g., see Ho et al., (2013) PLOS ONE 8(8): 10.1371/annotation/e6b5348e-9052-44ab-8f06-90d01dc88fc2).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139(39):13754-13760. doi: 10.1021/jacs.7b05803. Epub 2017 Sep. 20).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

### SUMMARY OF THE INVENTION

Thus, we realized that MTA-cooperative inhibition of PRMT5 activity in MTAP deleted cancers will provide therapeutic benefit for a wide range of cancers. The compounds of the present invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

There is a need to develop new MTA-cooperative PRMT5 inhibitors that are capable of inhibiting PRMT5 activity in the presence of elevated MTA concentrations, particularly in MTAP-deficient cells.

In one aspect of the invention, compounds are provided that are represented by Formula (I): A compound of Formula I:

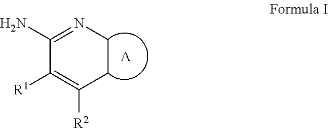

wherein:
A is selected from

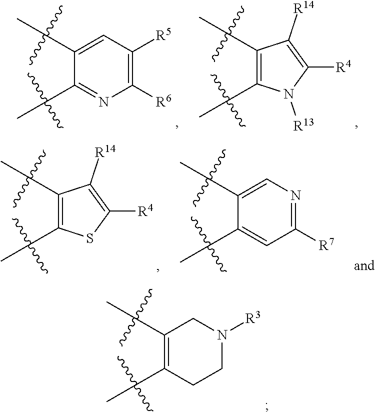

R¹ is hydrogen, F, Br, —C1-C2 alkyl, —C3-C4 cycloalkyl or —CF₃;
R² is hydrogen or C1-C2 alkyl;
R³ is hydrogen, pyrazolyl optionally substituted with C1-C3 alkyl or phenyl optionally substituted with cyano, or pyridine optionally substituted with —O-phenyl;
R⁴ is
   hydrogen,
   —C(O)—O—(C1-C2 alkyl),
   -L⁴-NH—C(O)-phenyl where phenyl is optionally substituted with one or more fluoro,
   -L⁴-NH—C(O)-pyrimidine, imidazole or triazole where the imidazole and triazole are optionally substituted with bromo,
   -L⁴-(CO)N(R¹⁰)(R¹¹) where R¹⁰ is pyridyl(C₁-C₆, alkyl) where the pyridyl is optionally substituted with halogen (preferably bromo) or trifluoromethyl and R¹¹ is pyridyl(C₁-C₆ alkyl), pyrimidinyl(C₁-C₆ alkyl) or 5,6,7,8-tetrahydroquinoxalinyl,
   -L⁴-1,3-dioxoisoindolin-2-yl(C₀-C₂ alkyl) where the alkyl is optionally substituted with cyano,
   -L⁴-1-oxo-3,4-dihydroisoquinolin-2-yl(C₀-C₂ alkyl) where the alkyl is optionally substituted with cyano,
   -L⁴-1-oxoisoquinolin-2-yl(C₀-C₂ alkyl) where the alkyl is optionally substituted with cyano,
   -L⁴-2,4-dioxoimidazolidin-1-yl,
   -L⁴-NH—C(O)C₁-C₂ alkyl)(R¹²) where R¹² is hydrogen, C₁-C₂ alkyl, or naphthyl optionally substituted with cyano, or
   -L⁴-3-(C₁-C₃ alkyl)-2,4-dioxoimidazolidin-1-yl,
where L⁴ is absent or C₁-C₂-alkyl, and provided that when R⁴ is hydrogen, at least one of R¹ and R² is not hydrogen; R⁵ is
   hydrogen,
   -L⁵-phenyl optionally substituted with one or more substituents selected from fluoro, chloro, cyano and C1-C2 alkyl,
   -L⁵-pyrimidine optionally substituted with one or more substituents selected from hydroxy and —NH-cyclopropyl,
   -L⁵-pyridine,
   -L⁵-pyradazine,
   -L⁵-isoxazole,
   -L⁵-thiazole,
   -L⁵-1,3-dioxoisoindolin-2-yl,
   -L⁵-(CO)N(R¹⁶)(R¹⁷) where R¹⁶ is pyridyl(C₁-C₆ alkyl) where the pyridyl is optionally substituted with halogen or trifluoromethyl and R¹⁷ is pyridyl(C₁-C₆ alkyl), pyrimidinyl(C₁-C₆ alkyl) or 5,6,7,8-tetrahydroquinoxalinyl,
   -L⁵-NH—C(O)(C₁-C₂ alkyl)(R¹⁸) where R¹⁸ is hydrogen, C₁-C₂ alkyl, or naphthyl optionally substituted with cyano, or
   -L5-1-methyl-pyrazole or bromo,
   where L⁵ is absent, —CH₂—NH—C(O)—, C1-C2 alkylene optionally substituted with cyano, —O— or —CH₂O—CH₂—;
R⁶ is hydrogen, -L⁶-phenyl optionally substituted with fluoro, -L⁶-pyridine, -L⁶-isothiazole, -L⁶-thiazole, -L⁶-1-methyl-pyrazole, —NH—(C1-C2 alkyl) or —NH—(C3-C4 cycloalkyl), where L⁶ is absent or C1-C1-alkylene, and provided that at least one of R⁵ and R⁶ is not hydrogen;
R⁷ is C1-C2 alkyl, chloro, 1-methyl-pyrazole or phenyl optionally substituted with one or more substituents selected from fluoro, chloro, cyano and C1-C2 alkyl;
R¹³ is hydrogen, C2-C3 acyl, C1-C2 alkyl or C3-C6 cycloalkyl; or
R¹³ and R¹⁴ together with the atoms to which they are attached form a 5-7 membered ring containing one nitrogen atom, and wherein the ring is optionally substituted with one or more of oxo and C2-C3 acyl; and
R¹⁴ is hydrogen, cyano or C₂-C₃ acyl; or a pharmaceutically acceptable salt thereof.

In another aspect of the invention, intermediates are provided that are useful for the preparation of compounds of Formula I.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting PRMT5 activity in a in a cell, comprising contacting the cell with a compound of Formula I. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula I or a pharmaceutically acceptable salt thereof, or a pharmaceutical composition thereof as defined herein. In one embodiment, the cell is an MTAP-deficient cell.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method for treating cancer in a patient in need thereof, the method comprising (a) determining that the cancer is associated with MTAP double deletion (e.g., an MTAP-associated cancer); and (b) administering to the patient a therapeutically effective amount of a compound of Formula I or a pharmaceutically acceptable salt thereof, or a pharmaceutical composition thereof.

### DETAILED DESCRIPTION OF THE INVENTION

The present invention relates to MTA-cooperative PRMT5 inhibitors. In particular, the present invention relates to compounds that inhibit PRMT5 activity in the presence of bound MTA, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

As used herein, “PRMT5” refers to a mammalian Protein Arginine N-Methyl Transferase 5 (PRMT5) enzyme.

As used herein, a “PRMT5 inhibitor” or “MTA-cooperative PRMT5 inhibitor” refers to compounds of the present invention that are represented by Formula (I) as described herein. These compounds are capable of negatively modulating or inhibiting all or a portion of the enzymatic activity of the PRMT5 in the presence of bound MTA in vitro or in vivo, or in cells expressing elevated levels of MTA.

As used herein, “MTAP” refers to a mammalian methylthioadenosine phosphorylase (MTAP) enzyme.

An “MTAP-associated disease or disorder” as used herein refers to diseases or disorders associated with or mediated by or having a loss of MTAP activity resulting in sensitizing the disorder to selective inhibition of PRMT5 activity. A non-limiting example of an MTAP-associated disease or disorder is an MTAP-associated cancer.

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC1-C6 alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NR^{x}, wherein R^{x} is hydrogen or C1-C3 alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C1-C6)alkyl(C6-C10)aryl, including, without limitation, benzyl, phenethyl, and naphthylmethyl. For example, an arC1-C3alkyl is an aryl group covalently linked to a C1-C3 alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, THFyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via a linker (e.g., an alkylene linker).

As used herein, the term “heteroaryl” refers to a group having 5 to 14 ring atoms, preferably 5, 6, 10, 13 or 14 ring atoms; having 6, 10, or 14 n electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom.

Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaralkyl” or “L-heteroarylalkyl” group comprises a heteroaryl group covalently linked to another group via a linker (e.g., an alkylene linker). Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

As used herein, “an effective amount” of a compound is an amount that is sufficient to negatively modulate or inhibit the activity of PRMT5 enzyme.

As used herein, a “therapeutically effective amount” of a compound is an amount that is sufficient to ameliorate or in some manner reduce a symptom or stop or reverse progression of a condition, or negatively modulate or inhibit the activity of PRMT5. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect of the invention, compounds are provided that are represented by Formula I:

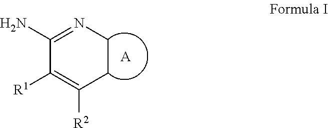

wherein:
A is selected from

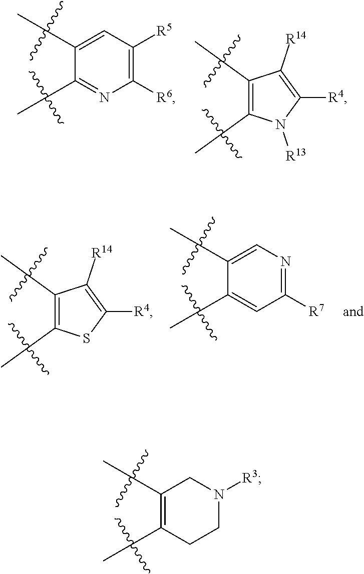

R¹ is hydrogen, F, Br, —C1-C2 alkyl, —C3-C4 cycloalkyl or —CF₃;
R² is hydrogen or C1-C2 alkyl;
R³ is hydrogen, pyrazolyl optionally substituted with C₁-C₃ alkyl or phenyl optionally substituted with cyano, or pyridine optionally substituted with —O-phenyl;
R⁴ is
   hydrogen,
   —C(O)—O—(C1-C2 alkyl),
   -L⁴-NH—C(O)-phenyl where phenyl is optionally substituted with one or more fluoro,
   -L⁴-NH—C(O)-pyrimidine, imidazole or triazole where the imidazole and triazole are optionally substituted with bromo,
   -L⁴-(CO)N(R¹⁰)(R¹¹) where R¹⁰ is pyridyl(C₁-C₆ alkyl) where the pyridyl is optionally substituted with halogen (preferably bromo) or trifluoromethyl and R¹¹ is pyridyl(C₁-C₆ alkyl), pyrimidinyl(C₁-C₆ alkyl) or 5,6,7,8-tetrahydroquinoxalinyl,
   -L⁴-1,3-dioxoisoindolin-2-yl(C₀-C₂ alkyl) where the alkyl is optionally substituted with cyano,
   -L⁴-1-oxo-3,4-dihydroisoquinolin-2-yl(C₀-C₂ alkyl) where the alkyl is optionally substituted with cyano,
   -L⁴-1-oxoisoquinolin-2-yl(C₀-C₂ alkyl) where the alkyl is optionally substituted with cyano,
   -L⁴-2,4-dioxoimidazolidin-1-yl,
   -L⁴-NH—C(O)C₁-C₂ alkyl)(R¹²) where R¹² is hydrogen, C₁-C₂ alkyl, or naphthyl optionally substituted with cyano, or
   -L⁴-3-(C₁-C₃ alkyl)-2,4-dioxoimidazolidin-1-yl,
where L⁴ is absent or C₁-C₂-alkyl, and provided that when R⁴ is hydrogen, at least one of R¹ and
R² is not hydrogen; R⁵ is
   hydrogen,
   -L⁵-phenyl optionally substituted with one or more substituents selected from fluoro, chloro, cyano and C₁-C₂ alkyl,
   -L⁵-pyrimidine optionally substituted with one or more substituents selected from hydroxy and —NH-cyclopropyl,
   -L⁵-pyridine,
   -L⁵-pyradazine,
   -L⁵-isoxazole,
   -L⁵-thiazole,
   -L⁵-1,3-dioxoisoindolin-2-yl,
   -L⁵-(CO)N(R¹⁶)(R¹⁷) where R¹⁶ is pyridyl(C₁-C₆ alkyl) where the pyridyl is optionally substituted with halogen or trifluoromethyl and R¹⁷ is pyridyl(C₁-C₆ alkyl), pyrimidinyl(C₁-C₆ alkyl) or 5,6,7,8-tetrahydroquinoxalinyl,
   -L⁵-NH—C(O)(C₁-C₂ alkyl)(R¹⁸) where R¹⁸ is hydrogen, C₁-C₂ alkyl, or naphthyl optionally substituted with cyano, or
   -L⁵-1-methyl-pyrazole or bromo,
   where L⁵ is absent, —CH₂—NH—C(O)—, C₁-C₂ alkylene optionally substituted with cyano, —O— or —CH₂OCH₂—;
R⁶ is hydrogen, -L⁶-phenyl optionally substituted with fluoro, -L⁶-pyridine, -L⁶-isothiazole, -L⁶-thiazole, -L⁶-1-methyl-pyrazole, —NH—(C1-C2 alkyl) or —NH—(C3-C4 cycloalkyl), where L⁶ is absent or C1-C1-alkylene, and provided that at least one of R⁵ and R⁶ is not hydrogen;
R⁷ is C1-C2 alkyl, chloro, 1-methyl-pyrazole or phenyl optionally substituted with one or more substituents selected from fluoro, chloro, cyano and C1-C2 alkyl;
R¹³ is hydrogen, C₂-C₃ acyl, C₁-C₂ alkyl or C₃-C₆ cycloalkyl; or
R¹³ and R¹⁴ together with the atoms to which they are attached form a 5-7 membered ring containing one nitrogen atom, and wherein the ring is optionally substituted with one or more of oxo and C₂-C₃ acyl; and
R¹⁴ is hydrogen, cyano or C₂-C₃ acyl; or a pharmaceutically acceptable salt thereof.

In one embodiment, the compound of Formula I is provided wherein R¹ is hydrogen.

In one embodiment, the compound of Formula I is provided wherein R¹ is Br, methyl, ethyl or cyclopropyl.

In one embodiment, the compound of Formula I is provided wherein R² is methyl.

In one embodiment, the compound of Formula I is provided wherein A is

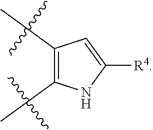

In one embodiment, the compound of Formula I is provided wherein A is

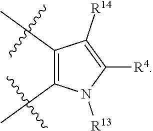

In one embodiment, the compound of Formula I is provided wherein A is

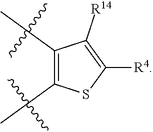

In one embodiment, the compound of Formula I is provided wherein R⁴ is hydrogen.

In one embodiment, the compound of Formula I is provided wherein A is:

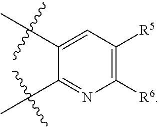

In one embodiment, the compound of Formula I is provided wherein L⁵ is absent.

In one embodiment, the compound of Formula I is provided wherein L⁵ is methylene.

In one embodiment, the compound of Formula I is provided wherein L⁵ is —O—.

In one embodiment, the compound of Formula I is provided wherein R⁵ is -phenyl optionally substituted with one or more substituents selected from fluoro, chloro, cyano and C1-C2 alkyl, or R⁵ is -pyrimidine optionally substituted with one or more substituents selected from hydroxy and —NH-cyclopropyl.

In one embodiment, the compound of Formula I is provided wherein L⁵ is —CH₂—NH—C(O)—.

In one embodiment, the compound of Formula I is provided wherein R⁵ is hydrogen and R⁵ is not hydrogen.

In one embodiment, the compound of Formula I is provided wherein R⁵ is not hydrogen and R⁶ is hydrogen.

In one embodiment, the compound of Formula I is provided wherein A is

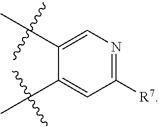

In one embodiment, the compound of Formula I is provided wherein R⁷ is C1-C2 alkyl, chloro, 1-methyl-pyrazole or phenyl.

In one embodiment, the compound of Formula I is provided wherein R⁷ is phenyl optionally substituted with one or more substituents selected from fluoro, chloro, cyano and C1-C2 alkyl.

In one embodiment, the compound of Formula I is provided wherein A is

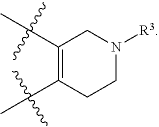

In one embodiment, the compound of Formula I is:

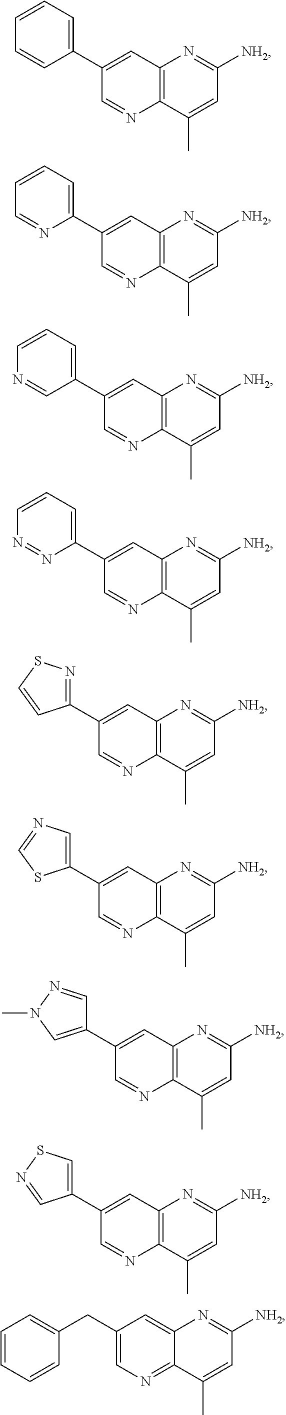

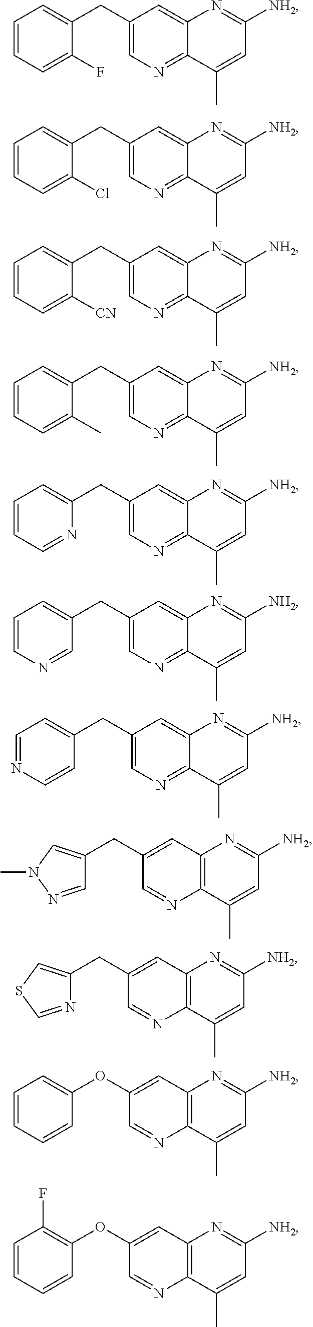

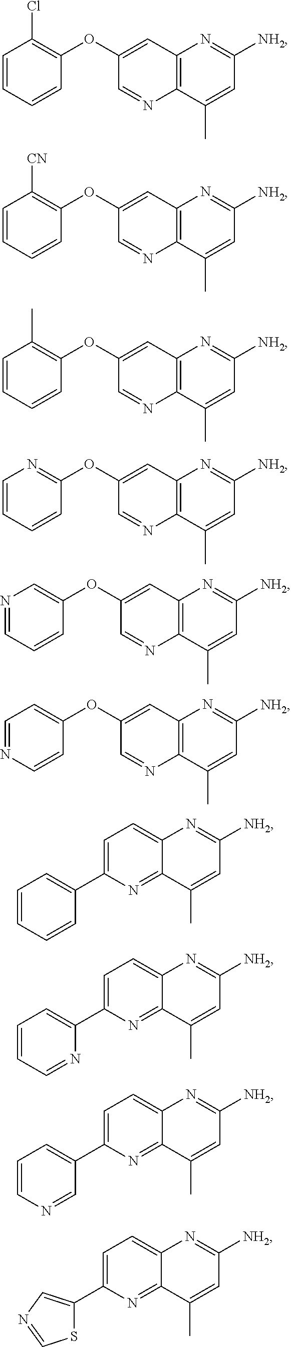

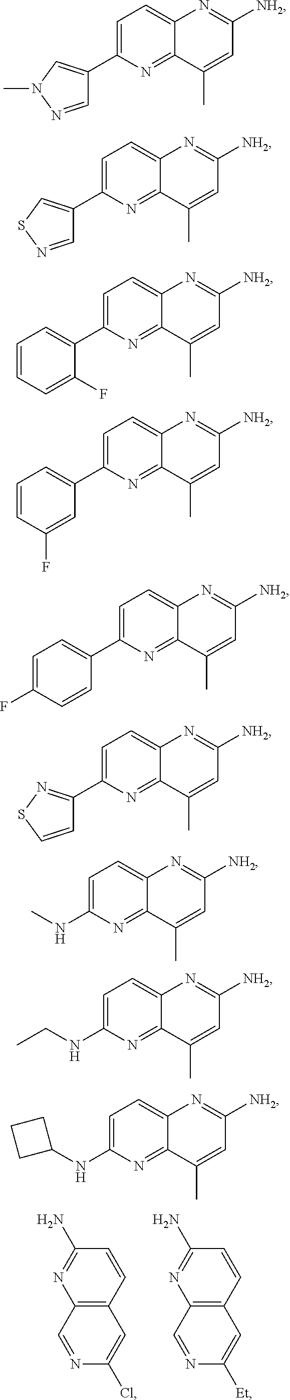

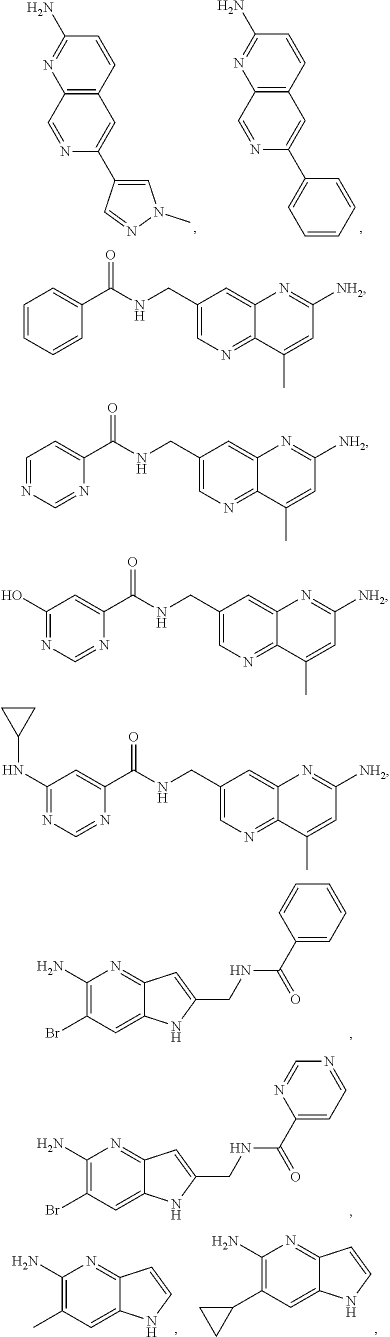

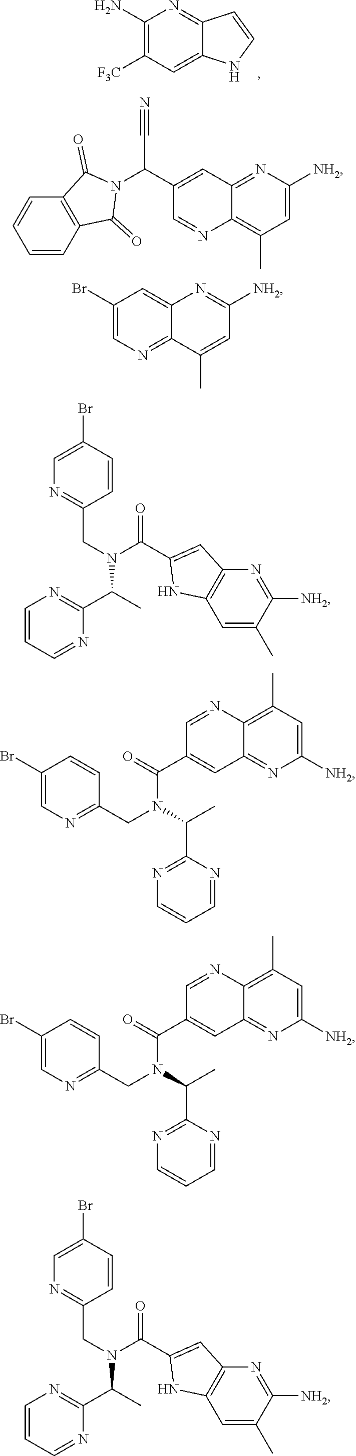

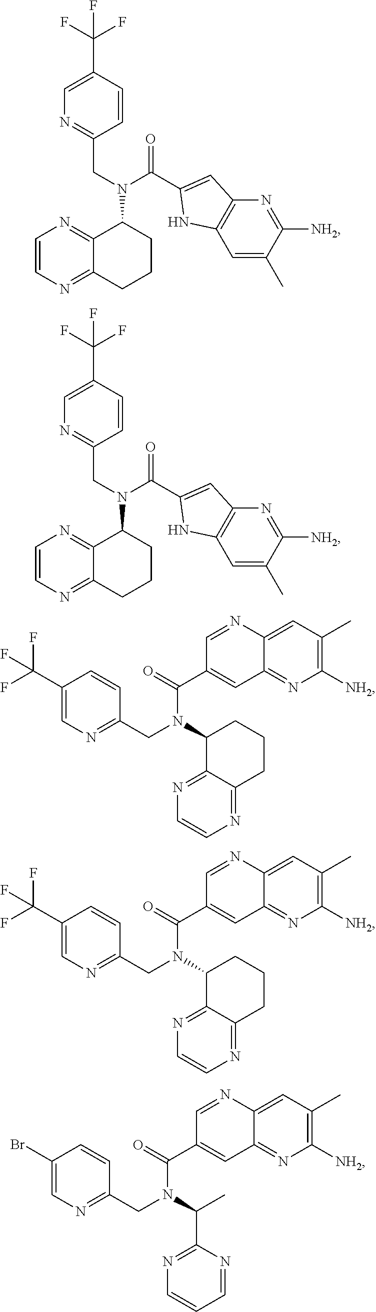

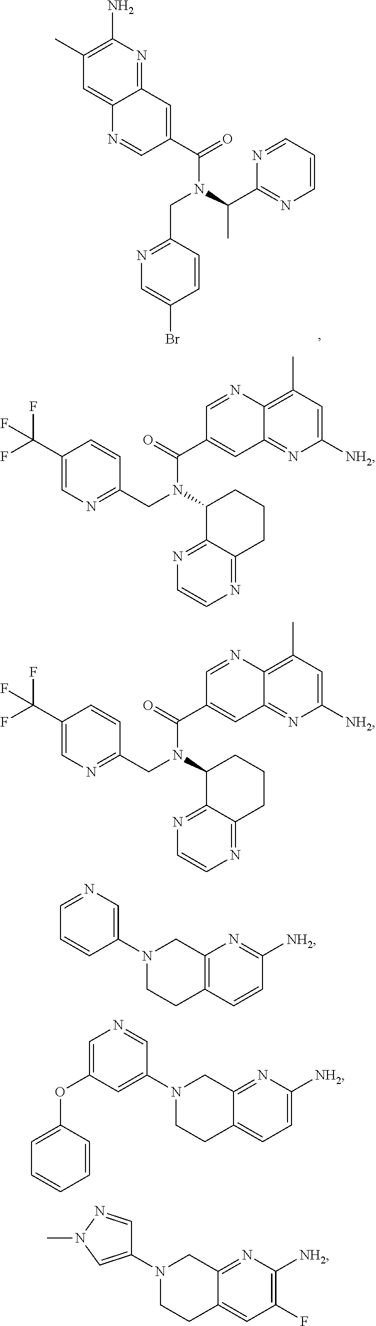

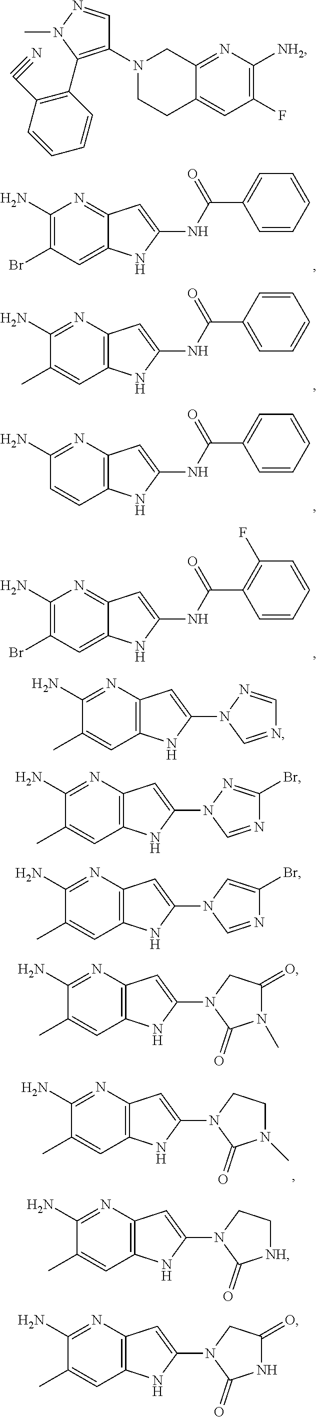

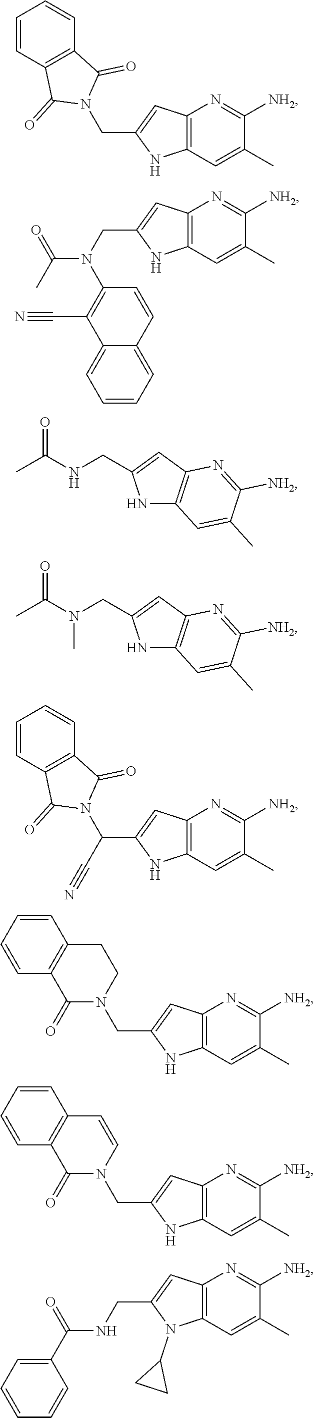

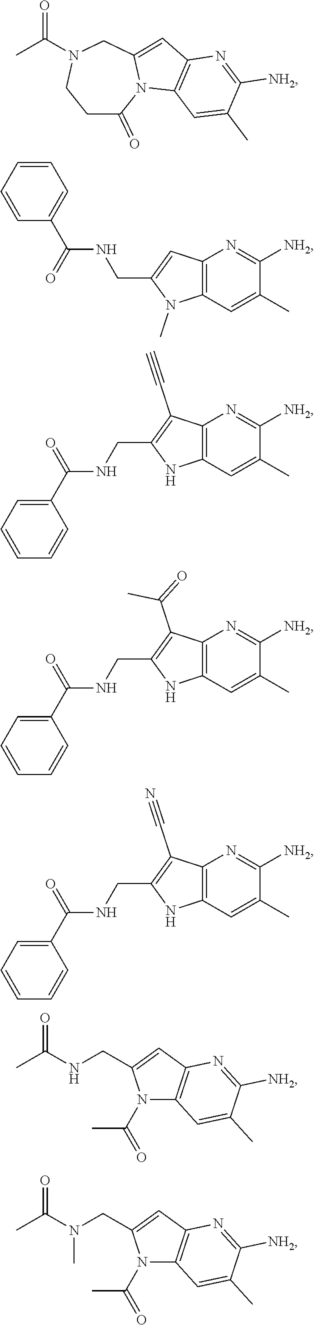

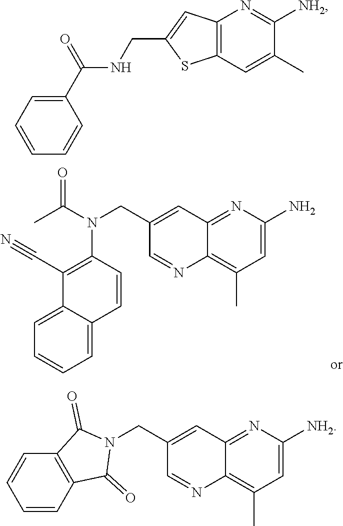

or a pharmaceutically acceptable salt of the foregoing compounds.

### Pharmaceutical Compositions

The compounds of Formula I may be formulated into pharmaceutical compositions.

In another aspect, the invention provides pharmaceutical compositions comprising a PRMT5 inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In yet another aspect, the invention provides for methods for inhibiting PRMT5 activity in a cell, comprising contacting the cell in which inhibition of PRMT5 activity is desired in vitro with an effective amount of a compound of Formula I, pharmaceutically acceptable salts thereof or pharmaceutical compositions containing the compound or pharmaceutically acceptable salt thereof. In one embodiment, the cell is an MTAP-deficient cell.

The compositions and methods provided herein are particularly deemed useful for inhibiting PRMT5 activity in a cell in vivo. In one embodiment, a cell in which inhibition of PRMT5 activity is desired is contacted in vivo with a therapeutically effective amount of a compound of Formula I, or a pharmaceutically acceptable salt thereof, to negatively modulate the activity of PRMT5. In other embodiments, a therapeutically effective amount of pharmaceutically acceptable salt or pharmaceutical compositions containing the compound of Formula I may be used. In one embodiment, the cell is an MTAP-deficient cell. In one embodiment, the negatively modulating the activity of PRMT5 occurs in the presence of bound MTA.

By negatively modulating the activity of PRMT5, particularly in cases for cells that lack MTAP activity, the methods are designed to inhibit PRMT5 activity to block cellular proliferation. The cells may be contacted in a single dose or multiple doses in accordance with a particular treatment regimen to affect the desired negative modulation of PRMT5. The degree PRMT5 inhibition may be monitored in vitro against the enzyme in the presence and absence of MTA and in the cell using well known methods, including those described in Example B below, to assess the effectiveness of treatment and dosages.

In another aspect, methods of treating cancer comprising administering to a patient having cancer a therapeutically effective amount of a compound of Formula I, pharmaceutically acceptable salts thereof or pharmaceutical compositions comprising the compound or pharmaceutically acceptable salts thereof are provided. In one embodiment, the cancer is an MTAP-associated cancer.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone; osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is an MTAP-associated cancer selected from hepatocellular carcinoma, breast cancer, skin cancer, bladder cancer, liver cancer, pancreatic cancer, and head and neck cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### General Reaction Scheme, Intermediates and Examples

### General Reaction Schemes

The compounds of the present invention may be prepared using commercially available reagents and intermediates in the synthetic methods and reaction schemes described herein, or may be prepared using other reagents and conventional methods well known to those skilled in the art.

For instance, compounds of Formula (I) of the present invention may be prepared according to General Reaction Schemes I-VII.

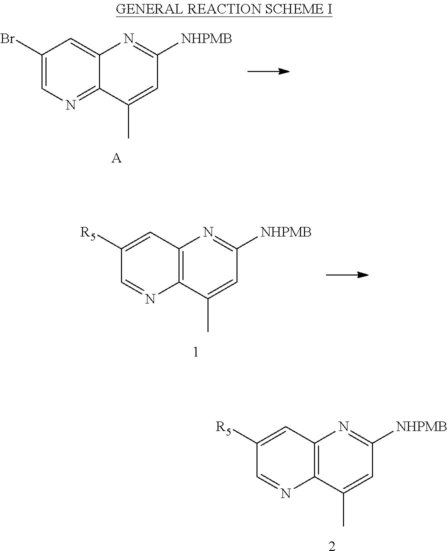

Compound 2 of Formula (I) wherein R¹ is Hydrogen, R² is methyl and A is

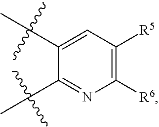

R⁶ is hydrogen and R⁵ is substituted aryl, heteroaryl may be prepared according to General Reaction Scheme I. Intermediate A is subjected to palladium catalyzed cross coupling conditions, such as the Stille coupling or the Suzuki coupling with aryl/heteroaryl metal reactants, for example with the corresponding aryl/heteroaryl-tributyltin or aryl/heteroaryl boronic acids/esters to provide substituted naphthyridine 1 which is then transformed into compound 2 via an acid-catalyzed protecting group removal.

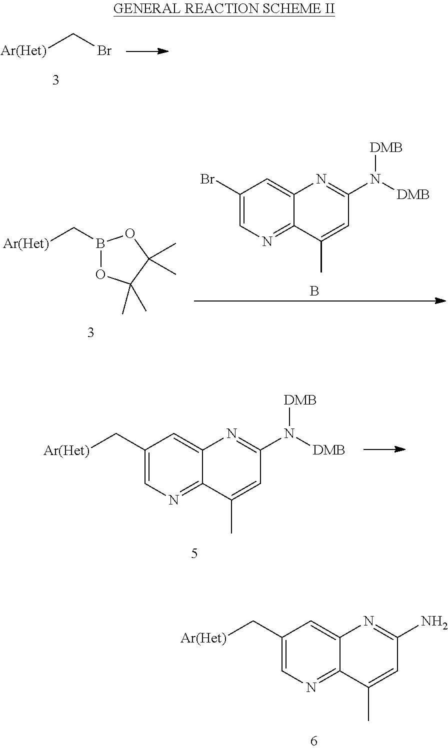

Compound 6 of Formula (I) wherein R1 is Hydrogen, R2 is methyl and A is

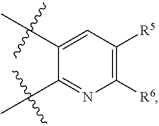

R⁶ is hydrogen and R⁵ is CH₂-aryl or heteroaryl may be prepared according to General Reaction Scheme H. Commercially available bromide 3 is initially transformed into boronate 4 and then is subjected to palladium catalyzed cross coupling conditions, such as the Suzuki coupling with intermediate bromide B to yield intermediate 5 that in finally treated with acid such as TFA to remove protecting groups and afford 6.

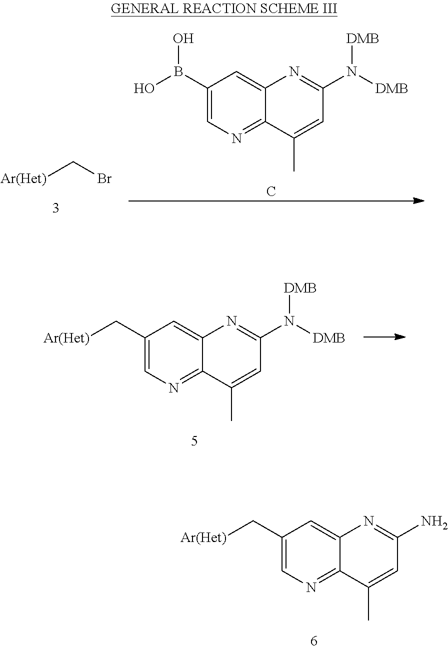

Compound 6 of Formula (I) wherein R¹ is Hydrogen, R² is methyl and A is

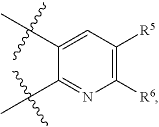

R⁶ is hydrogen and R⁵ is CH₂-aryl or heteroaryl may be prepared according to General Reaction Scheme III. Commercially available halide 3 is subjected to palladium catalyzed cross coupling conditions, such as the Suzuki coupling with intermediate boronic acid derivative C to yield intermediate 5 that is further treated with acid such as TFA to remove protecting groups and afford 6.

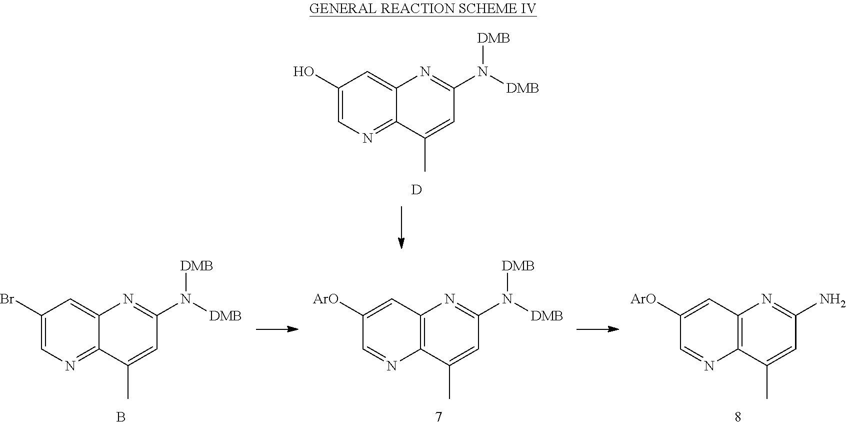

Compound 8 of Formula (I) wherein R¹ is Hydrogen, R² is methyl and A is

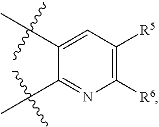

R⁶ is hydrogen and R⁵ is O-aryl or heteroaryl may be prepared according to General Reaction Scheme IV. Ether intermediate 7 was prepared from either via Copper-catalyzed reaction between bromide B and a phenol or starting from phenol D via SN_{Ar} reaction or metal-catalyzed coupling. Followed by a typical acid-mediated deprotection reaction compound 8 was obtained.

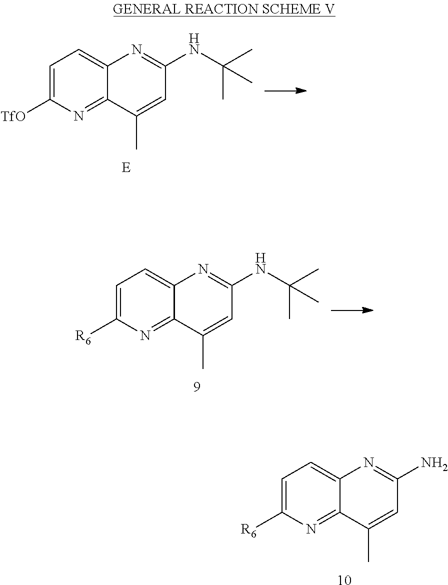

Compound 10 of Formula (I) wherein R¹ is Hydrogen, R² is methyl and A is

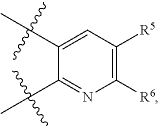

R⁵ is hydrogen and R⁶ is aryl, heteroaryl, NH-alkyl may be prepared according to General Reaction Scheme V. Triflate E was subjected to palladium or copper catalyzed cross coupling conditions, such as the Suzuki, Stille, Buchwald or Ullmann coupling with aryl and heteroaryl boronic acids or organostannanes or amines. The resulting intermediate product 9 was converted to final compound 10 via an acid-mediated deprotection step.

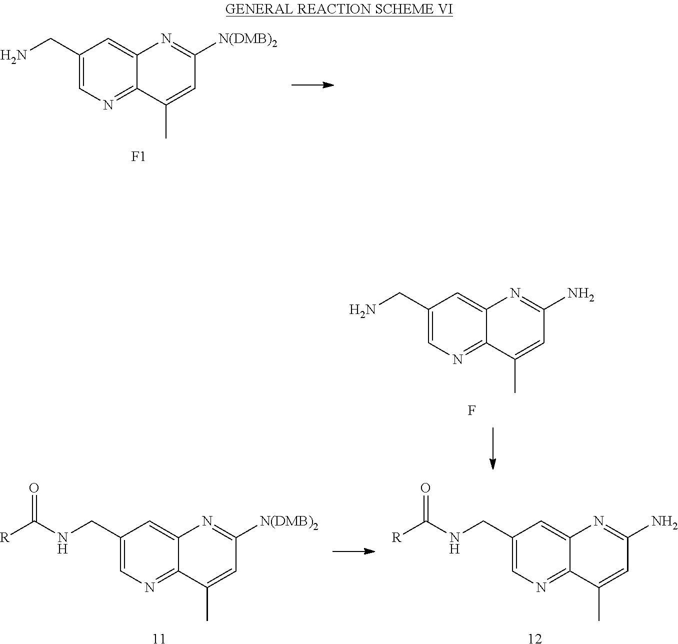

Compound 10 of Formula (I) wherein R¹ is Hydrogen, R² is methyl and A is

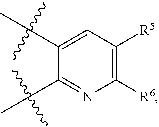

R⁶ is hydrogen and R⁵ is CH₂—NH—CO-alkyl, aryl or heteroaryl may be prepared according to General Reaction Scheme VI. Benzyl amine F was reacted with carboxylic acid using typical amid coupling reagents such as T3P and HATU to produce amide 12. Alternatively, benzyl amine F1 was subjected to similar coupling with carboxylic acid to produce intermediate 11 that was consequently transformed into final product 12 upon treatment with acid such as TFA that led to removal of protecting groups.

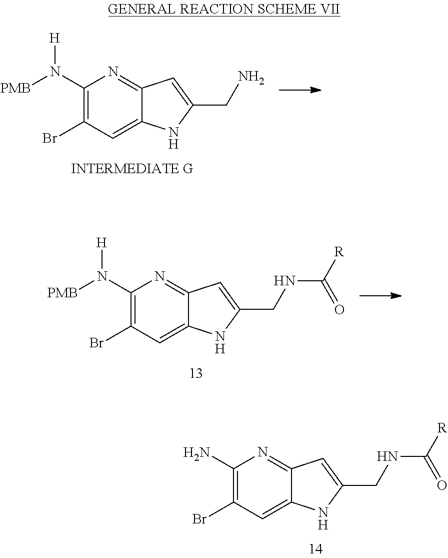

Compound 10 of Formula (I) wherein R¹ is Bromine, R² is Hydrogen and A is

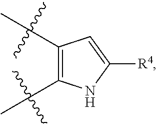

R⁴ is CH₂—NH—CO-alkyl, aryl or heteroaryl may be prepared according to General Reaction Scheme VII. Benzyl amine G was subjected to a coupling with a carboxylic acid using typical amide coupling reagents such as T3P and HATU to produce intermediate amide 13 that was consequently transformed into final product 14 upon treatment with acid such as TFA that led to removal of protecting groups.

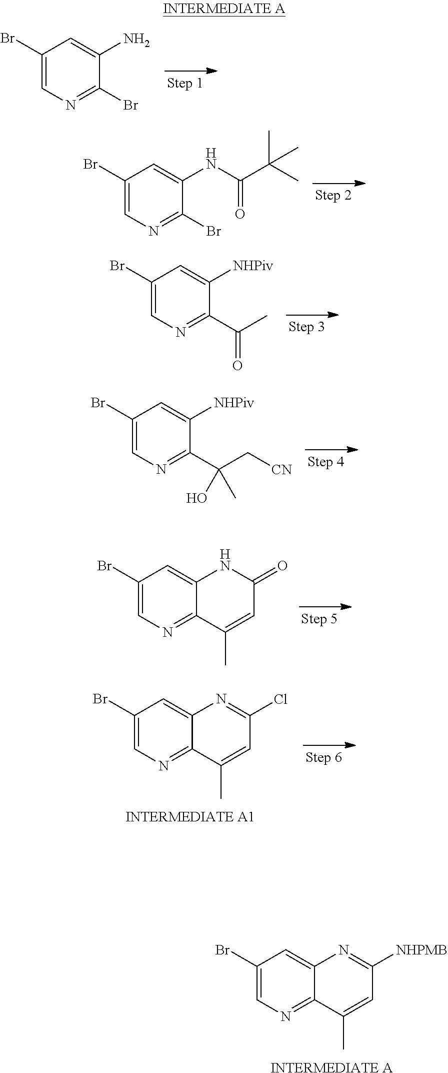

Step 1: To a solution of 2,5-dibromopyridin-3-amine (25.0 g, 99.2 mmol, 1.00 eq.) in pyridine (250 mL) was added 2,2-dimethylpropanoyl chloride (18.0 g, 149 mmol, 18.3 mL, 1.50 eq.) at 0° C. The mixture was stirred at 20° C. for 0.5 hr. The reaction mixture was diluted with water (200 mL) and extracted with ethyl acetate (200 mL×3). Combined organic phase was washed with brine (100 mL), dried over sodium sulfate, filtered and concentrated to give a residue. The residue was purified by column chromatography (SiO₂, Ethyl acetate/Petroleum ether 2-5%) to give N-(2,5-dibromo-3-pyridyl)-2,2-dimethyl-propanamide (35.0 g, crude) as a yellow solid. ¹H NMR (400 MHz, CDCl₃) δ=8.95 (d, J=2.4 Hz, 1H), 8.14 (d, J=2.4 Hz, 1H), 8.01 (br s, 1H), 1.35 (s, 9H).

Step 2: A mixture of N-(2,5-dibromo-3-pyridyl)-2,2-dimethyl-propanamide (33.0 g, 98.2 mmol, 1.00 eq.), tri-butyl(1-ethoxyvinyl)stannane (28.4 g, 78.6 mmol, 26.5 mL, 0.80 eq.) and Pd(PPh₃)₄ (11.4 g, 9.82 mmol, 0.10 eq.) in toluene (1.32 L) was degassed and stirred at 80° C. for 12 hours under nitrogen atmosphere. The reaction mixture was concentrated under reduced pressure to give a residue. The residue was purified by column chromatography (SiO₂, Ethyl acetate/Petroleum ether 2-5%) to give N-(2,5-dibromo-3-pyridyl)-2,2-dimethyl-propanamide (16.0 g, 47.6 mmol, 48.5% yield) as a white solid. ¹H NMR (400 MHz, CDCl₃) δ=9.19 (br s, 1H), 9.03-8.99 (m, 1H), 8.34-8.27 (m, 1H), 5.06 (dd, J=2.8, 5.2 Hz, 1H), 4.63-4.55 (m, 1H), 4.13-4.00 (m, 2H), 1.52-1.44 (m, 3H), 1.32-1.27 (m, 9H). A mixture of N-[5-bromo-2-(1-ethoxyvinyl)-3-pyridyl]-2,2-dimethyl-propanamide (9.50 g, 29.0 mmol, 1.00 eq.) in hydrochloric acid/dioxane (4 M, 38.0 mL, 5.24 eq.) was stirred at 20° C. for 10 minutes. The reaction mixture was concentrated under reduced pressure to give a residue. The residue was diluted with water (50.0 mL) and extracted with ethyl acetate (50.0 mL×3). Combined organic phase was washed with saturated sodium bicarbonate aqueous solution (50.0 mL×2), brine (50.0 mL), dried over sodium sulfate, filtered and concentrated under reduced pressure to give a residue. The residue was purified by column chromatography (SiO₂, Ethyl acetate/Petroleum ether 2-5%) to give N-(2-acetyl-5-bromo-3-pyridyl)-2,2-dimethyl-propanamide (6.90 g, 17.4 mmol, 59.8% yield) as a white solid. ¹H NMR (400 MHz, CDCl₃) δ=11.82 (br s, 1H), 9.44 (d, J=2.0 Hz, 1H), 8.39 (d, J=2.0 Hz, 1H), 2.77 (s, 3H), 1.36 (s, 9H).

Step 3: To a solution of acetonitrile (1.84 g, 44.9 mmol, 2.36 mL, 2.10 eq.) in tetrahydrofuran (45.0 mL) was added lithium diisopropylamide (2 M, 22.5 mL, 2.10 eq.) drop wise at −78° C. After stirring for 0.5 hour, a solution of N-(2-acetyl-5-bromo-3-pyridyl)-2,2-dimethyl-propanamide (6.40 g, 21.4 mmol, 1.00 eq.) in tetrahydrofuran (20.0 mL) was added to the reaction mixture. The reaction mixture was stirred at −78° C. for 30 minutes. The reaction mixture was quenched with water (50.0 mL) and extracted with dichloromethane (50.0 mL×3). Combined organic phase was washed with brine (50.0 mL), dried over sodium sulfate, filtered and concentrated to give N-[5-bromo-2-(2-cyano-1-hydroxy-1-methyl-ethyl)-3-pyridyl]-2,2-dimethyl-propanamide (8.10 g, crude) as a brown oil, which was used in the next step directly without further purification. ¹H NMR (400 MHz, CDCl₃) δ=10.11 (br s, 1H), 8.98 (d, J=2.0 Hz, 1H), 8.23 (d, J=2.0 Hz, 1H), 4.46 (br s, 1H), 3.31-3.21 (d, J=16.4 Hz, 1H), 3.17-3.06 (d, J=16.4 Hz, 1H), 1.68 (s, 3H), 1.30 (s, 9H)

Step 4: A solution of N-[5-bromo-2-(2-cyano-1-hydroxy-1-methyl-ethyl)-3-pyridyl]-2,2-dimethyl-propanamide (0.95 g, 2.79 mmol, 1.00 eq.) in hydrochloric acid (3 M, 3.80 mL, 4.08 eq.) was micro waved at 160° C. for 5 minutes. The resulting mixture was basified to pH=9 with saturated sodium bicarbonate (10.0 mL). The precipitate formed was filtered and washed with water to give 7-bromo-4-methyl-1H-1,5-naphthyridin-2-one (5.00 g, 20.9 mmol, 93.6% yield) as a brown solid. ¹H NMR (400 MHz, DMSO-d₆) δ=11.75 (br s, 1H), 8.55 (d, J=2.0 Hz, 1H), 7.82 (d, J=2.0 Hz, 1H), 6.68 (d, J=0.8 Hz, 1H), 2.43 (d, J=0.8 Hz, 3H).

Step 5: A mixture of 7-bromo-4-methyl-1H-1,5-naphthyridin-2-one (2.50 g, 10.5 mmol, 1.00 eq.) and phosphorus oxychloride (41.3 g, 269 mmol, 25.0 mL, 25.7 eq.) was stirred at 120° C. for 3 hours. The reaction mixture was concentrated under reduced pressure to give a residue. The residue was diluted with ethyl acetate (50.0 mL) and quenched with ice water (50.0 mL). The aqueous phase was separated and extracted with ethyl acetate (50.0 mL×3). Combined organic phase was washed with saturated sodium bicarbonate aqueous solution (50.0 mL), brine (50.0 mL), dried over sodium sulfate, filtered and concentrated to give 7-bromo-2-chloro-4-methyl-1,5-naphthyridine, Intermediate A (5.00 g, crude) as a brown solid which was used in the next step directly without further purification. ¹H NMR (400 MHz, DMSO-d₆) δ=9.08 (d, J=2.4 Hz, 1H), 8.66 (d, J=2.4 Hz, 11H), 7.79 (d, J=1.2 Hz, 1H), 2.72 (d, J=1.2 Hz, 3H).

Step 6: To a solution of 7-bromo-2-chloro-4-methyl-1,5-naphthyridine (0.80 g, 3.11 mmol, 1.00 eq.) in dimethylsulfoxide (8.00 mL) was added potassium fluoride (541 mg, 9.32 mmol, 218 μL, 3.00 eq.) and (4-methoxyphenyl)methanamine (852 mg, 6.21 mmol, 804 μL, 2 eq.). The mixture was stirred at 130° C. for 2 hours. The reaction mixture was diluted with water (10.0 mL) and extracted with ethyl acetate (10.0 mL×3). Combined organic phase was washed with brine (10.0×2 mL), dried over sodium sulfate, filtered and concentrated to give 7-bromo-N-[(4-methoxyphenyl)methyl]-4-methyl-1,5-naphthyridin-2-amine, Intermediate A (1.30 g, crude) as a yellow oil which was used in the next step directly without further purification. LCMS [M+1]: 358.1

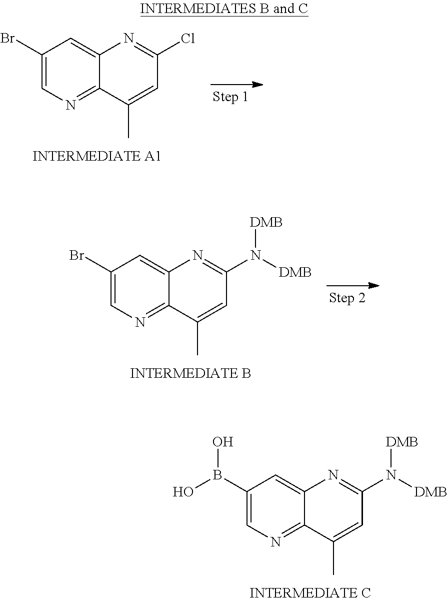

Step 1: A mixture of Intermediate A1 (1.00 g, 3.88 mmol, 1.00 eq.), 1-(2,4-dimethoxyphenyl)-N-[(2,4-dimethoxyphenyl)methyl]methanamine (2.46 g, 7.77 mmol, 2.00 eq.), potassium fluoride (677 mg, 11.7 mmol, 273 μL, 3.00 eq.) in dimethyl sulfoxide (10.0 mL) was degassed and stirred at 130° C. for 12 hours under nitrogen atmosphere. The mixture was diluted with brine (10.0 mL), extracted with ethyl acetate (20.0 mL-2), the organic phase was dried over sodium sulfate, filtered, concentrated in vacuo. The residue was purified by column chromatography (SiO₂, ethyl acetate/petroleum ether 5-30%) to give 7-bromo-N,N-bis[(2,4-dimethoxyphenyl)methyl]-4-methyl-1,5-naphthyridin-2-amine, Intermediate B (1.70 g, 3.08 mmol, 79.4% yield) as a white solid. LCMS [M+1]: 540.0. ¹H NMR (400 MHz, CDCl₃) δ=8.54 (d, J=2.0 Hz, 1H), 8.12 (d, J=2.4 Hz, 1H), 7.10 (br d, J=7.2 Hz, 2H), 6.85 (d, J=0.8 Hz, 1H), 6.48 (d, J=2.4 Hz, 2H), 6.41 (d, J=2.4 Hz, 1H), 6.39 (d, J=2.0 Hz, 1H), 4.84 (br s, 4H), 3.79 (d, J=2.4 Hz, 12H), 2.58 (s, 3H), 1.60 (s, 1H).

Step 2: A mixture of Intermediate B (300 mg, 557 μmol, 1.00 eq.), 4,4,5,5-tetramethyl-2-(4,4,5,5-tetramethyl-1,3,2-dioxaborolan-2-yl)-1,3,2-dioxaborolane (212 mg, 836 μmol, 1.50 eq.), potassium acetate (164 mg, 1.67 mmol, 3.00 eq.) and Pd(dppf)Z-DCM (45.5 mg, 55.7 μmol, 0.10 eq) in dioxane (5.00 mL) was degassed stirred at 110° C. for 3 hr under nitrogen atmosphere. The reaction mixture was filtered and concentrated under vacuum to give a residue. The residue was washed with methyl alcohol (20.0 mL) and filtered to giver [6-[bis[(2,4-dimethoxyphenyl)methyl]amino]-8-methyl-1,5-naphthyridin-3-yl]boronic acid, Intermediate C (450 mg, crude) as a black solid. The material was used directly for the next step without further purification. LCMS [M+1]: 503.9

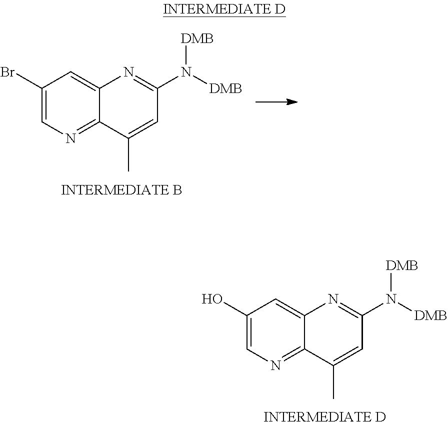

A mixture of Intermediate B (200 mg, 371 μmol, 1.00 eq), Pd₂(dba)₃ (34.0 mg, 37.1 μmol, 0.100 eq), t-BuXphos (31.6 mg, 74.3 μmol, 0.200 eq) and potassium hydroxide (208 mg, 3.71 mmol, 10.0 eq) in dioxane (1.50 mL) and water (1.50 mL) was degassed and stirred at 100° C. for 12 hours under nitrogen atmosphere. The pH of the reaction mixture was adjusted to pH 7 with HCl (1.00 N). Then the mixture was poured into water (30.0 mL) and extracted with ethyl acetate (15.0 mL×3). The combined organic layer was dried over sodium sulfate, filtered and concentrated under reduced pressure to give a residue. The residue was purified by column chromatography (SiO₂, ethyl acetate/petroleum ether 10-50%) to give 6-[bis[(2,4-dimethoxyphenyl)methyl]amino]-8-methyl-1,5-naphthyridin-3-ol, Intermediate D (170 mg, 354 μmol, 95.0% yield) as a yellow solid. ¹H NMR (400 MHz, CDCl₃) δ=7.85-7.77 (m, 1H), 7.10-7.01 (m, 3H), 6.65 (s, 1H), 6.41-6.33 (m, 4H), 4.75 (s, 4H), 3.68 (d, J=9.2 Hz, 12H), 2.58 (s, 3H).

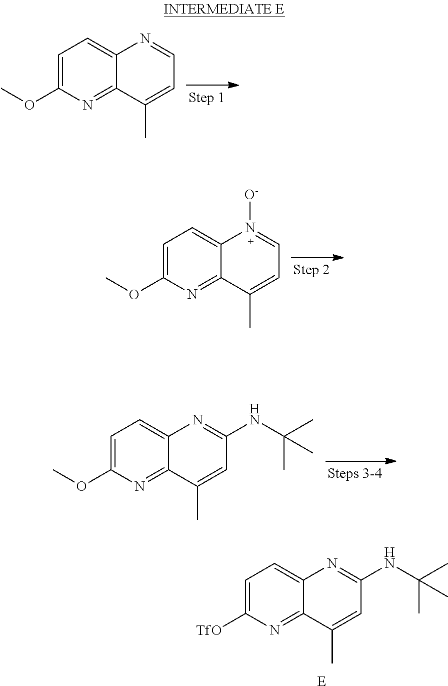

Step 1: To a solution of 2-methoxy-8-methyl-1,5-naphthyridine (prepared following the procedure from US2003/212084) (1.20 g, 6.89 mmol, 1.00 eq.) in dichloromethane (10.0 mL) was added m-CPBA (1.40 g, 6.89 mmol, 85.0% purity, 1.00 eq.) and the mixture was stirred at 0° C. for 1 hour. Then another portion of m-CPBA (1.40 g, 6.89 mmol, 85.0% purity, 1.00 eq.) was added and the mixture was stirred at 15° C. for 3 hours. The reaction mixture was filtered and concentrated under reduced pressure to give a residue. The residue was purified by column chromatography (SiO₂, ethyl acetate/petroleum ether 30-100% followed by methyl alcohol/dichloromethane 10%) to give 6-methoxy-4-methyl-1-oxido-1,5-naphthyridin-1-ium (883 mg, 4.64 mmol, 67.4% yield) as a red solid. ¹H NMR (400 MHz, CDCl₃) δ=8.87 (d, J=9.2 Hz, 1H), 8.35 (d, J=6.0 Hz, 1H), 7.29 (d, J=6.0 Hz, 1H), 7.13 (d, J=9.2 Hz, 1H), 4.10 (s, 3H), 2.66 (s, 3H).

Step 2: In a sealed tube, to a solution of 6-methoxy-4-methyl-1-oxido-1,5-naphthyridin-1-ium (882 mg, 4.64 mmol, 1.00 eq.) and 2-methylpropan-2-amine (509 mg, 6.96 mmol, 731 μL, 1.50 eq.) in dichloroethane (15.0 mL) was added triethylamine (1.64 g, 16.2 mmol, 2.26 mL, 3.50 eq.) and BOP (3.24 g, 6.96 mmol, 1.50 eq.). The mixture was stirred at 100° C. for 12 hours. The reaction mixture was diluted with water (150 mL) and extracted with dichloromethane (100 mL×3). The combined organic layers were washed with brine (100 mL), dried, filtered and concentrated under reduced pressure to give a residue. The residue was purified by column chromatography (SiO₂, ethyl acetate/petroleum ether 10-50%) to give N-tert-butyl-6-methoxy-4-methyl-1,5-naphthyridin-2-amine (635 mg, 2.59 mmol, 55.8% yield) as a yellow solid. ¹H NMR (400 MHz, CDCl₃) δ=7.83 (d, J=8.8 Hz, 1H), 6.94 (d, J=8.8 Hz, 1H), 6.65 (s, 1H), 4.01 (s, 3H), 1.51 (s, 9H).

Step 4: To a solution of N-tert-butyl-6-methoxy-4-methyl-1,5-naphthyridin-2-amine (850 mg, 3.46 mmol, 1.00 eq.) in acetonitrile (10.0 mL) was added trimethylchlorosilane (1.13 g, 10.4 mmol, 1.32 mL, 3.00 eq.) and sodium iodide (1.56 g, 10.4 mmol, 3.00 eq.). The mixture was stirred at 80° C. for 2.5 hours. The pH of the reaction mixture was adjusted to pH 8 with ammonium hydroxide and the resulting was concentrated under reduced pressure to give a residue. The residue was purified by column chromatography (SiO₂, ethyl acetate/petroleum ether 20-100% followed by methyl alcohol/dichloromethane 10%) to give 6-(tert-butylamino)-8-methyl-1,5-naphthyridin-2-ol (543 mg, 2.35 mmol, 67.8% yield) as a yellow solid. ¹H NMR (400 MHz, CDCl₃) δ=7.83 (d, J=8.8 Hz, 1H), 6.94 (d, J=8.8 Hz, 1H), 6.65 (s, 1H), 4.01 (s, 3H), 1.51 (s, 9H).

Step 5: To a solution of 6-(tert-butylamino)-8-methyl-1,5-naphthyridin-2-ol (433 mg, 1.87 mmol, 1.00 eq.), 4A MS (1.00 g), triethylamine (568 mg, 5.62 mmol, 782 μL, 3.00 eq.) and DMAP (22.9 mg, 187 μmol, 0.10 eq.) in dichloromethane (1.00 mL) was added trifluoro mesylate anhydride (581 mg, 2.06 mmol, 340 μL, 1.10 eq.) dropwise at 0° C. The mixture was stirred at 0° C. for 1 hour. The reaction mixture was concentrated under reduced pressure to give a residue. The residue was purified by column chromatography (SiO₂, ethyl acetate/petroleum ether 30-100%) to give [6-(tert-butylamino)-8-methyl-1,5-naphthyridin-2-yl]trifluoromethanesulfonate, Intermediate E (627 mg, 1.73 mmol, 92.2% yield) as a brownish solid. ¹H NMR (400 MHz, CDCl₃) δ=8.04 (d, J=8.8 Hz, 1H), 7.23 (d, J=8.8 Hz, 1H), 6.66 (d, J=1.2 Hz, 1H), 2.54 (d, J=1.2 Hz, 3H), 1.53 (s, 9H).

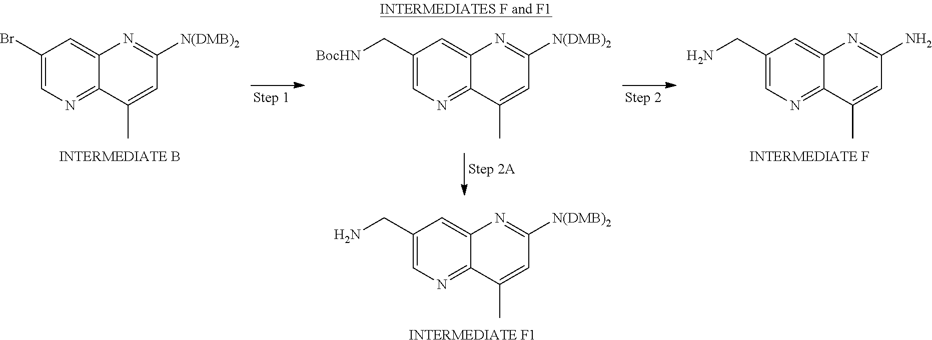

Step 1: A mixture of Intermediate B (1.70 g, 3.16 mmol, 1.00 eq.), Potassium [[(tert-Butoxycarbonyl)amino]methyl]trifluoroborate (1.50 g, 6.31 mmol, 2.00 eq.), cataCXium® A Pd G3 (230 mg, 316 μmol, 0.10 eq.), sodium carbonate (1.00 g, 9.47 mmol, 3.0 eq.) in a mixed solvent of water (15.0 mL) and dioxane (75.0 mL) was degassed and stirred at 100° C. for 12 hours under nitrogen atmosphere. The mixture was concentrated under reduced pressure to give a residue. The crude product was triturated with petroleum ether/ethyl acetate 10:1 (50.0 mL) at 20° C. for 0.5 hour to give tert-butyl N-[[6-[bis[(2,4-dimethoxyphenyl)methyl]amino]-8-methyl-1,5-naphthyridin-3-yl]methyl]carbamate (1.60 g, 2.72 mmol, 86.1% yield) as a white solid. LCMS [M+1]⁺=589.3

Step 2: A mixture of tert-butyl N-[[6-[bis[(2,4-dimethoxyphenyl)methyl]amino]-8-methyl-1,5-naphthyridin-3-yl]methyl]carbamate (1.50 g, 2.55 mmol, 1.00 eq.) in trifluoroacetic acid (7.70 g, 67.5 mmol, 5.00 mL, 26.5 eq.) was stirred at 70° C. for 1 hour. The mixture was concentrated under reduced pressure to give a residue and diluted with water (10.0 mL). The mixture was extracted with ethyl acetate (20.0 mL×2). Then the aqueous phase was basified with sodium bicarbonate to pH 9 and exacted with ethyl acetate (100 mL×10). The combined organic phase was washed with brine (30.0 mL-2), dried over anhydrous sodium sulfate, filtered and concentrated under reduced pressure to give a 7-(aminomethyl)-4-methyl-1,5-naphthyridin-2-amine, Intermediate F (280 mg, 1.49 mmol, 58.4% yield) as a yellow solid. The residue was used for the next step without further purification. LCMS [M+1]⁺=189.1. ¹H NMR (400 MHz, CD₃OD-d₄) δ=8.55 (d, J=2.0 Hz, 1H), 7.85 (d, J=2.0 Hz, 1H), 6.92 (d, J=1.0 Hz, 1H), 4.07 (s, 2H), 2.62 (d, J=0.8 Hz, 3H).

Step 2A: To a solution of tert-butyl N-[[6-[bis[(2,4-dimethoxyphenyl)methyl]amino]-8-methyl-1,5-naphthyridin-3-yl]methyl]carbamate (780 mg, 1.32 mmol, 1.00 eq.) in dichloromethane (10.0 mL) was added zinc bromide (895 mg, 3.97 mmol, 199 μL, 3.00 eq.). The mixture was stirred at 25° C. for 12 hours. The reaction mixture was concentrated under reduced pressure to remove dichloromethane. The residue was diluted with water (20.0 mL) and extracted with ethyl acetate (20.0 mL×3). The combined organic layers were washed with brine (50.0 mL), dried over anhydrous sodium sulfate, filtered and concentrated under reduced pressure to give a residue. The crude product was triturated with ethyl acetate (10 mL) at 15° C. for 30 minutes to give 7-(aminomethyl)-N,N-bis[(2,4-dimethoxyphenyl)methyl]-4-methyl-1,5-naphthyridin-2-amine, Intermediate F1 (480 mg, 982 μmol, 74.2% yield) as a yellow solid. LCMS [M+1]⁺=489.1.

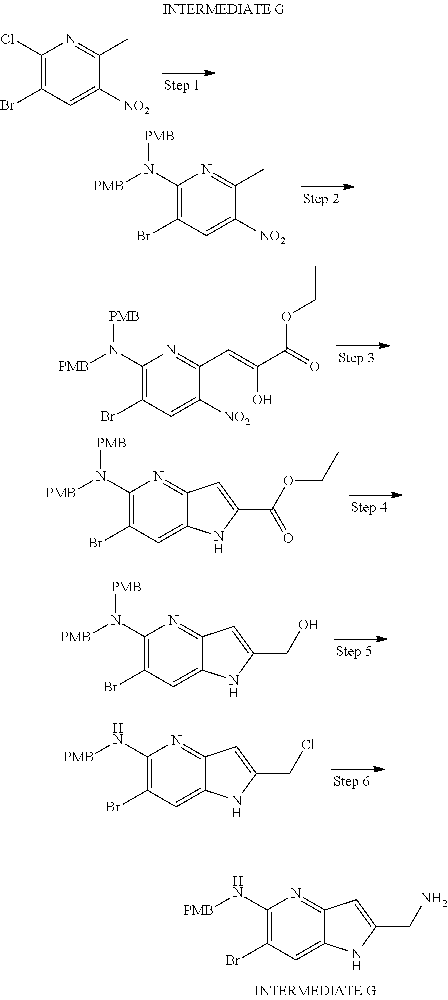

Step 1: To a solution of 3-bromo-2-chloro-6-methyl-5-nitro-pyridine (19.0 g, 75.5 mmol, 1.00 eq.) and 1-(4-methoxyphenyl)-N-[(4-methoxyphenyl)methyl]methanamine (23.3 g, 90.6 mmol, 1.20 eq.) in tetrahydrofuran (190 mL) was added sodium carbonate (9.61 g, 90.6 mmol, 1.20 eq). The mixture was stirred at 75° C. for 16 h. The mixture was concentrated, diluted with ethyl acetate (500 mL), washed with water (200.0 mL), dried over sodium sulfate, filtered and concentrated. The residue was purified by silica gel chromatography (Ethyl acetate/Petroleum ether 0-50%) to afford 3-bromo-N,N-bis[(4-methoxyphenyl)methyl]-6-methyl-5-nitro-pyridin-2-amine (33.0 g, 68.4 mmol, 90.6% yield) as a yellow solid. LCMS [EST, M+1]⁺=472.0. ¹HNMR (400 MHz, CDCl₃) δ=8.53 (s, 1H), 7.20 (d, J=8.8 Hz, 4H), 6.94-6.80 (m, 4H), 4.75 (s, 4H), 3.82 (s, 6H), 2.76 (s, 3H).

Step 2: To a mixture of 3-bromo-N,N-bis[(4-methoxyphenyl)methyl]-6-methyl-5-nitro-pyridin-2-amine (10.0 g, 21.1 mmol, 1.00 eq.) and diethyl oxalate (9.20 g, 63.5 mmol, 8.60 mL, 3.00 eq.) was added DABCO (3.87 g, 25.4 mmol, 3.83 mL, 1.20 eq.). The mixture was stirred at 30° C. for 16 h. The mixture was diluted with ethyl acetate (500 mL). Acetic acid (4.00 mL) was added. The resulting solution was washed with water (300 mL), dried over sodium sulfate, filtered and concentrated. The residue was purified by silica gel chromatography (Ethyl acetate/Petroleum ether 0-50%) to afford ethyl (Z)-3-[6-[bis[(4-methoxyphenyl)methyl]amino]-5-bromo-3-nitro-2-pyridyl]-2-hydroxy-prop-2-enoate (7.20 g, 10.6 mmol, 50.5% yield, 85.0% purity) as a yellow solid. LCMS [ESI, M+1]⁺=572.2. ¹HNMR (400 MHz, CDCl₃) δ=8.70-8.63 (m, 1H), 7.53 (s, 1H), 7.07-6.98 (m, 4H), 6.88-6.83 (m, 4H), 4.64-4.54 (m, 4H), 4.38 (q, J=7.2 Hz, 2H), 3.81 (s, 6H), 1.43-1.30 (m, 3H).

Step 3: To a solution of ethyl (Z)-3-[6-[bis[(4-methoxyphenyl)methyl]amino]-5-bromo-3-nitro-2-pyridyl]-2-hydroxy-prop-2-enoate (5.10 g, 8.91 mmol, 1.00 eq.) in tetrahydrofuran (15.0 mL), ethanol (90.0 mL) and water (10.0 mL) was added ammonium chloride (571 mg, 10.6 mmol, 1.20 eq) and iron powder (1.99 g, 35.6 mmol, 4.00 eq.) at 25° C. The mixture was stirred at 60° C. for 6 hours. The mixture was diluted with dichloromethane (600 mL) and water (600 mL), stirred for 10 min, filtered and the organic layer was separated, dried over sodium sulfate, filtered and concentrated. The residue was purified by silica gel chromatography (Ethyl acetate/Petroleum ether 0-80%) to afford ethyl 5-[bis [(4-methoxyphenyl) methyl] amino]-6-bromo-1H-pyrrolo [3, 2-b] pyridine-2-carboxylate (2.10 g, 3.88 mmol, 43.6% yield, 97.0% purity) as a yellow solid. LCMS [ESI, M+1]⁺=526.1. ¹HNMR (400 MHz, CDCl₃) δ=8.92 (brs, 1H), 7.94 (s, 1H), 7.32 (d, J=8.4 Hz, 4H), 7.20 (d, J=1.2 Hz, 1H), 6.89-6.78 (m, 4H), 4.44 (q, J=7.2 Hz, 2H), 4.36 (s, 4H), 3.78 (s, 6H), 1.43 (t, J=7.2 Hz, 3H).

Step 4: To a solution of ethyl 5-[bis[(4-methoxyphenyl)methyl]amino]-6-bromo-1H-pyrrolo[3,2-b]pyridine-2-carboxylate (900 mg, 1.72 mmol, 1.00 eq.) in tetrahydrofuran (20.0 mL) was added lithium aluminum hydride (195 mg, 5.15 mmol, 3.00 eq.) at 0° C. The mixture was stirred at 25° C. for 0.5 hour. The mixture was diluted with tetrahydrofuran (100 mL). Sodium sulfate decahydrate (5.00 g) was added. The resulting solution was stirred for 0.5 hour. The resulting solution was filtered and concentrated to afford [5-[bis[(4-methoxyphenyl)methyl]amino]-6-bromo-1H-pyrrolo[3,2-b]pyridin-2-yl]methanol (827 mg, crude) as a brown solid. LCMS [ESI, M+1]⁺=484.2. ¹HNMR (400 MHz, CDCl₃) δ=8.53 (br s, 1H), 7.66-7.58 (m, 1H), 7.22 (br d, J=8.4 Hz, 4H), 6.91 (s, 1H), 6.71 (d, J=8.4 Hz, 4H), 4.62 (s, 2H), 4.20 (s, 4H), 3.66 (s, 6H).

Step 5: To a solution of [5-[bis[(4-methoxyphenyl)methyl]amino]-6-bromo-1H-pyrrolo[3,2-b]pyridin-2-yl]methanol (827 mg, 1.71 mmol, 1.00 eq.) in dichloromethane (20.0 mL) was added thionyl chloride (1.02 g, 8.57 mmol, 621 μL, 5.00 eq.) and dimethylformamide (125 mg, 1.71 mmol, 131 μL, 1.00 eq.) at 0° C. The mixture was stirred at 25° C. for 0.5 hour. The mixture was concentrated to afford 6-bromo-2-(chloromethyl)-N-[(4-methoxyphenyl)methyl]-1H-pyrrolo [3,2-b] pyridin-5-amine (652 mg, 1.71 mmol, 99.9% yield) as a black solid.

Step 6: Ammonia gas was passed through ethanol (20.0 mL) at 0° C. for 10 min. 6-bromo-2-(chloromethyl)-N-[(4-methoxyphenyl)methyl]-1H-pyrrolo[3,2-b]pyridin-5-amine (652 mg, 1.71 mmol, 1.00 eq.) in methanol (15.0 mL) was added to the ammonia solution. The mixture was stirred at 25° C. for 16 hours. The mixture was concentrated. The residue was purified by silica gel chromatography (Methanol/Dichloromethane 0-50%, 5‰ ammonium hydroxide) to afford 2-(aminomethyl)-6-bromo-N-[(4-methoxyphenyl) methyl]-1H-pyrrolo [3, 2-b] pyridin-5-amine, Intermediate G (75.0 mg, 159 μmol, 9.33% yield) as a brown solid. LCMS [ESI, M+1]⁺=363.1.

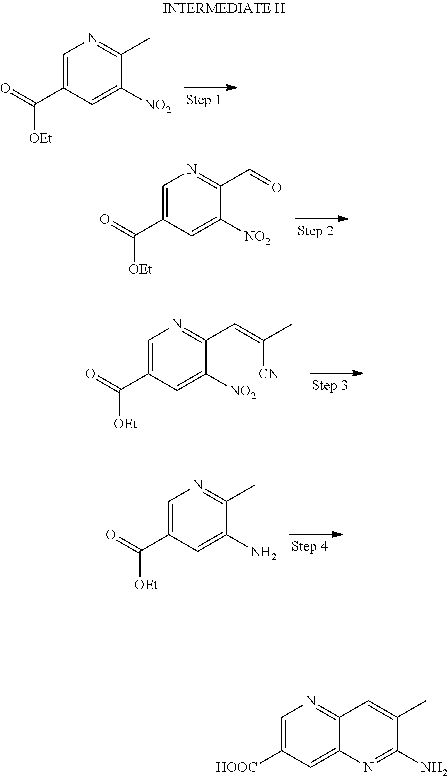 [SMILES: CCOC(=O)c1cnc(C)c([N+](=O)[O-])c1 ; CCOC(=O)c1cnc(C=O)c([N+](=O)[O-])c1 ; CCOC(=O)c1cnc(/C=C(/C)C#N)c([N+](=O)[O-])c1 ; CCOC(=O)c1cnc(C)c(N)c1 ; Cc1cc2ncc(C(=O)O)cc2nc1N]

Step 1: To a solution of ethyl 6-methyl-5-nitro-pyridine-3-carboxylate (0.50 g, 1.00 eq.) in dioxane (3.00 mL) was added selenium dioxide (396 mg, 1.50 eq.). The mixture was stirred at 100° C. for 4 hours. The reaction mixture was filtered and the filtrate was concentrated under reduced pressure to give a residue. The residue was dissolved in ethyl acetate (10.0 mL) and filtered. The filtrate was concentrated under reduced pressure to afford ethyl 6-formyl-5-nitronicotinate (500 mg, crude) as a yellow oil. ¹H NMR (400 MHz, CDCl₃) δ=10.32 (s, 1H), 9.51 (d, J=1.6 Hz, 1H), 8.81 (d, J=1.6 Hz, 1H), 4.52 (q, J=7.2 Hz, 2H), 1.47 (t, J=7.2 Hz, 3H).

Step 2: To a solution of diethyl (1-cyanoethyl)phosphonate (221 mg, 1.30 eq.) in tetrahydrofuran (3.00 mL) was added potassium tert-butoxide (130 mg, 1.30 eq.) at 0° C. The mixture was stirred at 0° C. for 10 mins. Then ethyl 6-formyl-5-nitronicotinate (200 mg, 1.00 eq.) was added to the mixture, and the mixture was stirred at 60° C. for 3 hours. The reaction solution was cooled to 25° C., and then to the reaction solution was added saturated aqueous sodium bicarbonate solution (15.0 mL), and the mixture was extracted with ethyl acetate (2×15.0 mL). The organic layers were combined, washed with brine (15.0 mL), dried over anhydrous sodium sulfate, and then concentrated under reduced pressure. The residue was purified by flash silica gel chromatography (0-25%, ethyl acetate/petroleum ether) to afford (Z)-ethyl 6-(2-cyanoprop-1-en-1-yl)-5-nitronicotinate (160 mg, 68.0% yield) as a yellow oil. ¹H NMR (400 MHz, CDCl₃) δ=9.52-9.40 (m, 1H), 8.96-8.89 (m, 1H), 7.59 (d, J=1.6 Hz, 1H), 4.54-4.46 (m, 2H), 1.48-1.44 (m, 3H).

Step 3: To a solution of (Z)-ethyl 6-(2-cyanoprop-1-en-1-yl)-5-nitronicotinate (140 mg, 1.00 eq.) in ethanol (1.40 mL) and water (0.20 mL) was added iron powder (120 mg, 4.00 eq.) and ammonium chloride (115 mg, 4.00 eq.). The mixture was stirred at 60° C. for 6 hours. The reaction mixture was diluted with ethyl acetate (15.0 mL) and filtered through a pad of celite. The filtrate was washed with brine (10.0 mL) and concentrated under reduced pressure to afford ethyl 6-amino-7-methyl-1,5-naphthyridine-3-carboxylate (80.0 mg, crude) as a yellow solid. ¹H NMR (400 MHz, CDCl₃) δ=9.23 (d, J=1.6 Hz, 1H), 8.60 (d, J=1.2 Hz, 1H), 8.01 (s, 1H), 5.23 (br s, 2H), 4.47 (q, J=7.2 Hz, 2H), 2.41 (d, J=0.8 Hz, 3H), 1.46 (t, J=7.2 Hz, 3H).

Step 4: To a solution of ethyl 6-amino-7-methyl-1,5-naphthyridine-3-carboxylate (80.0 mg, 1.00eq.) in methanol (1.00 mL) was added 2 M lithium hydroxide aqueous solution (692 μL, 4.00 eq.). The mixture was stirred at 40° C. for 1 hour. The reaction mixture was concentrated under reduced pressure to remove most of methanol. The residue was diluted with was water (2.00 mL) and the pH was adjusted to 3 with 1 N hydrochloric acid. The precipitate was collected through filtration and dried in vacuo to afford 6-amino-7-methyl-1,5-naphthyridine-3-carboxylic acid, Intermediate H (48.0 mg, crude) as a white solid. ¹H NMR (400 MHz, DMSO-dh) δ=8.92 (s, 1H), 8.23 (s, 1H), 7.88 (s, 1H), 6.78 (br s, 1H), 2.29 (s, 3H)

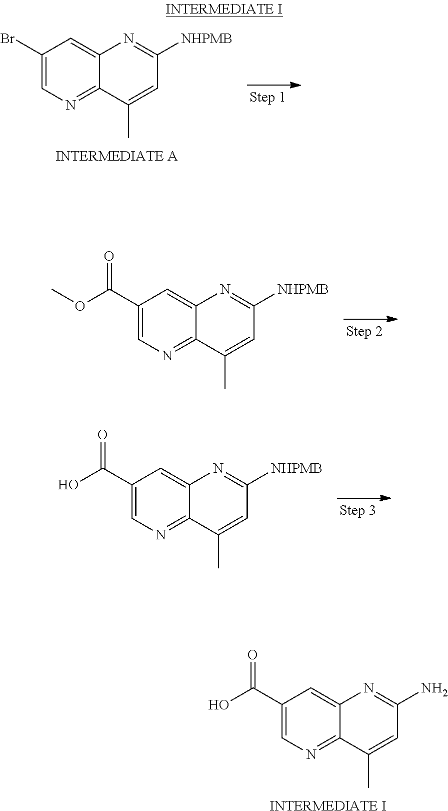

Step 1: To a mixture of Intermediate A (300 mg, 1.00 eq.) in N,N-dimethylformamide (1.80 mL) and methanol (1.80 mL) was added Pd(OAc)₂ (18.8 mg, 0.10 eq.), DPPF (46.4 mg, 0.10 eq.) and triethylamine (254 mg, 350 μL, 3.00 eq.). The mixture was stirred at 75° C. under CO (50.0 Psi) for 10 hours. The mixture was cooled to 25° C. Then the mixture was filtered, the filter cake was washed with methanol (30.0 mL) and the filtrate was concentrated under reduced pressure to give a residue. The residue was purified by column chromatography (SiO₂, Petroleum ether/Ethyl acetate 7:1 to 4:1) to afford methyl 6-((4-methoxybenzyl)amino)-8-methyl-1,5-naphthyridine-3-carboxylate (240 mg, 84.9% yield) as a yellow solid. LCMS [M+1]⁺=338.2. ¹H NMR (400 MHz, DMSO-d₆) δ=8.93 (d, J=2.0 Hz, 1H), 8.24 (d, J=2.0 Hz, 1H), 7.34-7.30 (m, 2H), 7.02 (d, J=0.9 Hz, 1H), 6.91-6.86 (m, 2H), 4.56 (d, J=5.6 Hz, 2H), 3.92 (s, 3H), 3.72 (s, 3H), 3.32 (s, 1H), 2.55 (d, J=0.8 Hz, 3H).

Step 2: To a solution of methyl 6-((4-methoxybenzyl)amino)-8-methyl-1,5-naphthyridine-3-carboxylate (240 mg, 1.00 eq.) in methanol (2.50 mL) and water (2.50 mL) was added LiOH·H₂O (149 mg, 5.00 eq.). The mixture was stirred at 40° C. for 12 hours. The reaction mixture was concentrated under reduced pressure to remove methanol. The pH value of the residue was adjusted to 3-5 with 1M hydrochloric acid, and lots of solids were precipitated. The solid was collected by filtration. The filter cake was washed with water and dried in vacuo to afford 6-((4-methoxybenzyl)amino)-8-methyl-1,5-naphthyridine-3-carboxylic acid (230 mg, crude) as a yellow solid, which was used into next step directly without further purification. LCMS [M+1]⁺=324.2. ¹H NMR (400 MHz, DMSO-d₆) δ=13.87-13.33 (m, 1H), 8.99 (br s, 1H), 8.63-8.22 (m, 1H), 7.36 (br d, J=8.2 Hz, 2H), 7.16-7.07 (m, 1H), 6.91 (br d, J=8.4 Hz, 2H), 4.64 (br s, 2H), 3.73 (s, 3H), 2.57 (s, 3H).

Step 3: A solution of 6-((4-methoxybenzyl)amino)-8-methyl-1,5-naphthyridine-3-carboxylic acid (100 mg, 1.00 eq.) in TFA (2.00 mL) was stirred at 60° C. for 12 hours. The reaction mixture was concentrated under reduced pressure to remove TFA. The pH value of the residue was adjusted to 7-9 with sat. aq. sodium bicarbonate, and lots of solids were precipitated. The solid was collected by filtration. The filter cake was washed with water and dried in vacuo to afford 6-amino-8-methyl-1,5-naphthyridine-3-carboxylic acid (66.0 mg, 66.0% yield) as a yellow solid. LCMS [M+1]⁺=204.1

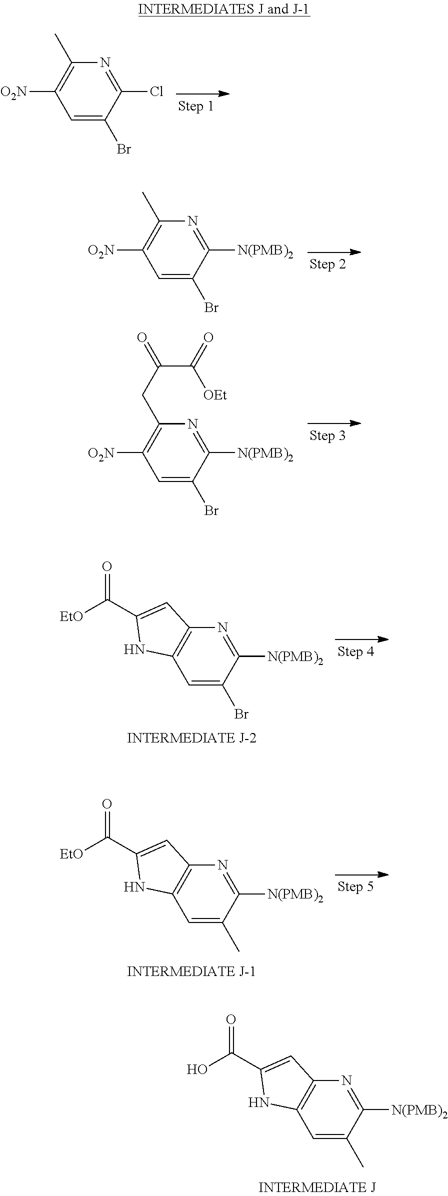

Step 1: To a solution of 3-bromo-2-chloro-6-methyl-5-nitro-pyridine (10 g, 39.7 mmol 1 eq.) in THF (50 mL) was added 1-(4-methoxyphenyl)-N-[(4-methoxyphenyl)methyl]methanamine (12.3 g, 47.7 mmol 1.2 eq.) and Na₂CO₃ (5.06 g, 47.7 mmol 1.2 eq.). The mixture was stirred at 75° C. for 16 hours. The reaction mixture was partitioned between ethyl acetate (30 mL) and water (20 mL). The organic phase was separated, and concentrated under reduced pressure to give a residue. The residue was purified by column chromatography (SiO₂, petroleum ether/ethyl acetate 50:1 to 10:1) to afford 3-bromo-N,N-bis[(4-methoxyphenyl)methyl]-6-methyl-5-nitro-pyridin-2-amine (15.6 g, 33.3 mmol 83.1% yield) as a yellow solid, LCMS [M+1]⁺=474.0.

Step 2: To a solution of 3-bromo-N,N-bis[(4-methoxyphenyl)methyl]-6-methyl-5-nitro-pyridin-2-amine (15.6 g, 33.0 mmol, 1 eq.) was added DBU (6.03 g, 5.97 mL, 1.2 eq.), diethyl oxalate (14.48 g, 13.53 mL, 3 eq.). The mixture was stirred at 40° C. for 16 hours. The reaction mixture was quenched with EtOH (20 ml), and partitioned between ethyl acetate (50 mL) and water (20 mL). The organic phase was separated, and concentrated under reduced pressure to give a residue. The residue was purified by column chromatography (SiO₂, petroleum ether/ethyl acetate 10:1 to 3:1) to afford ethyl 3-[6-[bis[(4-methoxyphenyl)methyl]amino]-5-bromo-3-nitro-2-pyridyl]-2-oxo-propanoate (6.8 g, 11.9 mmol 36% yield) as a yellow solid.

Step 3: To a solution of ethyl 3-[6-[bis[(4-methoxyphenyl)methyl]amino]-5-bromo-3-nitro-2-pyridyl]-2-oxo-propanoate (6.8 g, 11.9 mmol 1.0 eq.) in acetic acid (20 mL) was added Iron (2.65 g, 47.5 mmol 4.0 eq.). The mixture was stirred at 25° C. for 16 hours. The reaction mixture was partitioned between ethyl acetate (100 mL) and water (50 mL). The organic phase was separated, concentrated and purified by column chromatography (SiO₂, petroleum ether/ethyl acetate 10:1 to 4:1) to afford ethyl 5-[bis[(4-methoxyphenyl)methyl]amino]-6-bromo-1H-pyrrolo[3,2-b]pyridine-2-carboxylate, Intermediate J-2 (5 g, 9.5 mmol 80.3% yield) as a yellow solid, LCMS [M+1]⁺=524.1.

Step 4: A mixture of ethyl 5-[bis[(4-methoxyphenyl)methyl]amino]-6-bromo-1H-pyrrolo[3,2-b]pyridine-2-carboxylate (3.00 g, 3.8 mmol 1.0 eq.), methylboronic acid (3.42 g, 19.1 mmol 10.0 eq.), Pd(dppf)Cl₂ (745 mg, 381.3 μmmol 0.2 eq.) and K₂CO₃ (2.37 g, 11.4 mmol 3.0 eq.) in dioxane (30 mL) was degassed stirred at 100° C. for 12 hours under N₂ atmosphere. The reaction mixture was concentrated and purified by column chromatography (SiO₂, petroleum ether/ethyl acetate 10:1 to 3:1) to afford ethyl 5-[bis[(4-methoxyphenyl)methyl]amino]-6-methyl-1H-pyrrolo[3,2-b]pyridine-2-carboxylate, Intermediate J-1 as a yellow solid (1.7 g, 64% yield), LCMS [M+1]⁺=460.3.

Step 5: To a solution of Intermediate J-1 (1.7 g, 3.7 mmol 1 eq.) in methanol (10 mL) was added sodium hydroxide (443 mg, 11.1 mmol 3 eq.) and H₂O (10 mL). The mixture was stirred at 60° C. for 2 hours. The reaction mixture was concentrated and purified by column chromatography (SiO₂, petroleum ether/ethyl acetate 1:1 to 0:1) to afford 5-[bis[(4-methoxyphenyl)methyl]amino]-6-methyl-1H-pyrrolo[3,2-b]pyridine-2-carboxylic acid, Intermediate J (800 mg, 1.85 mmol 50% yield), as a yellow solid LCMS [M+1]⁺=432.2.

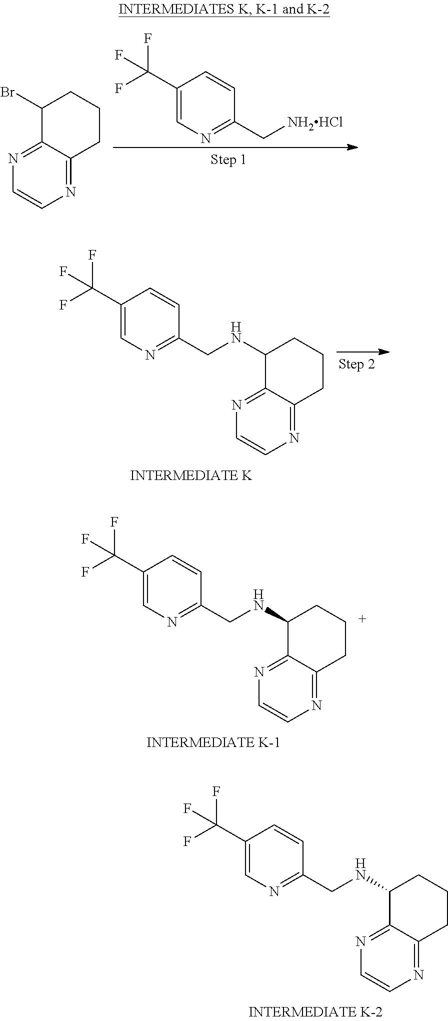

Step 1: A mixture of 5-bromo-5,6,7,8-tetrahydroquinoxaline (625 mg, 1.00 eq.), [5-(trifluoromethyl)-2-pyridyl]methanamine hydrochloride (748 mg, 3.00 mmol, 1.02 eq.) and potassium carbonate (1.62 g, 11.7 mmol, 4.00 eq.) in dimethyl formamide (15.0 mL) was degassed and stirred at 40° C. for 2 hours under nitrogen atmosphere. The mixture was extracted with ethyl acetate (30.0 mL×3), washed with brine, dried over sodium sulfate, filtered and concentrated under reduced pressure to give a residue. The residue was purified by prep-HPLC (neutral condition) to give N-[[5-(trifluoromethyl)-2-pyridyl]methyl]-5,6,7,8-tetrahydroquinoxalin-5-amine, Intermediate K (150 mg, 48.0% yield) as a yellow oil. LCMS (ESI, M+1): m/z=309.0. ¹H NMR (400 MHz, CDCl3) δ=8.85 (s, 1H), 8.39 (s, 2H), 7.90 (dd, J=2.0, 8.4 Hz, 1H), 7.57 (d, J=8.4 Hz, 1H), 4.19 (s, 2H), 3.96 (br t, J=6.4 Hz, 1H), 3.04-2.97 (m, 2H), 2.26-2.11 (m, 2H), 1.90-1.81 (m, 2H)

Step 2: Racemic N-[[5-(trifluoromethyl)-2-pyridyl]methyl]-5,6,7,8-tetrahydroquinoxalin-5-amine (150 mg) was separated by SFC (condition: column: DAICEL CHIRALPAK IC (250 mm*30 mm, 5 um); mobile phase: [ACN/IPA(0.1% NH₃H₂O)]; B %: 20%-20%, 6 min) to give peak 1 (5S)—N-[[5-(trifluoromethyl)-2-pyridyl]methyl]-5,6,7,8-tetrahydroquinoxalin-5-amine, Intermediate K-1 (50.0 g, 28.2% yield, 84.6% purity) as a white solid. LCMS (ESI, M+1); m/z=309.0. ¹H NMR (400 MHz, CDCl₃) δ=8.85 (s, 1H), 8.39 (s, 2H), 7.90 (dd, J=1.8, 8.2 Hz, 1H), 7.57 (d, J=8.2 Hz, 1H), 4.19 (s, 2H), 3.99-3.92 (m, 1H), 3.04-2.95 (m, 2H), 2.25-2.12 (m, 2H), 1.93-1.79 (m, 2H) and peak 2 (5R)—N-[[5-(trifluoromethyl)-2-pyridyl]methyl]-5,6,7,8-tetrahydroquinoxalin-5-amine, Intermediate K-2 ((50 mg, 26.7% yield) as a white solid. LCMS (ESI, M+1): m/z=309.0. ¹H NMR (400 MHz, CDCl₃) δ=8.85 (s, 1H), 8.39 (s, 2H), 7.90 (dd, J=1.7, 8.1 Hz, 1H), 7.58 (d, J=8.3 Hz, 1H), 4.20 (s, 2H), 3.96 (br t, J=6.4 Hz, 1H), 3.06-2.97 (m, 2H), 2.26-2.11 (m, 2H), 1.93-1.80 (m, 2H).

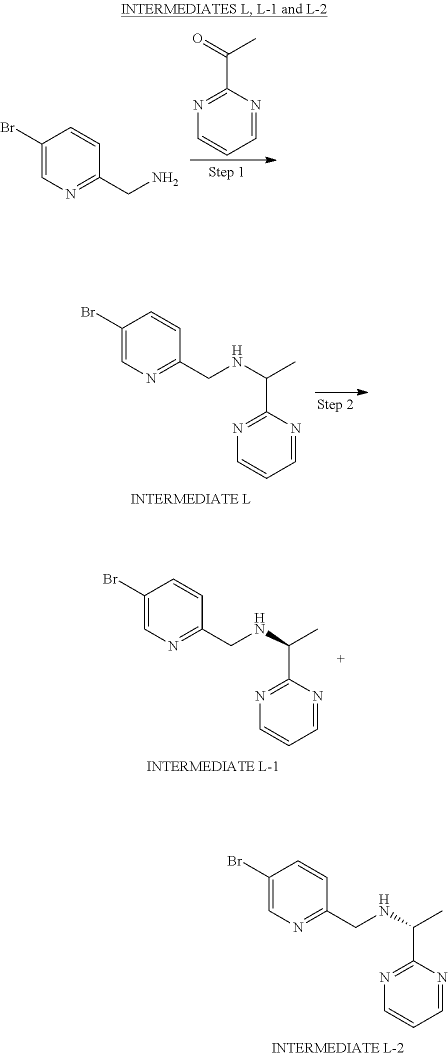

Step 1: To a solution of (5-bromo-2-pyridyl) methanamine (1.60 g, 8.55 mmol, 1.0 equiv) and 1-pyrimidin-2-ylethanone (1.25 g, 10.3 mmol, 1.2 equiv) in dichloromethane (20 mL) was added potassium acetate (1.26 g, 12.8 mmol, 1.5 equiv). The mixture was stirred at 25° C. for 0.5 hour. Then NaBH(OAc)₃ (2.72 g, 12.8 mmol, 1.5 equiv) was added and the mixture was stirred at 25° C. for 1.5 hours. The mixture was diluted with water (50 mL) and the pH was adjusted to ˜4 with HCl (aq., 1.0 M). The mixture was washed with dichloromethane (50 mL×2). Then the aqueous phase was basified with sodium hydroxide (aq., 10%) to pH 9 and exacted with dichloromethane (100 mL×3). The combined organic layers were washed with brine (30 mL×3), dried over anhydrous sodium sulfate, filtered and concentrated under reduced pressure to give N-[(5-bromo-2-pyridyl)methyl]-1-pyrimidin-2-yl-ethanamine, Intermediate L (2.12 g, 7.23 mmol, 84.5% yield) as a yellow oil.

Step 2: Racemic N-[(5-bromo-2-pyridyl)methyl]-1-pyrimidin-2-yl-ethanamine was separated by prep-SFC (column: DAICEL CHIRALPAK AD(250 mm*30 mm, 10 um);mobile phase: [0.1% NH3H2O ETOH];B %: 40%-40%,4 min) to give peak 1 (S)—N-((5-bromopyridin-2-yl)methyl)-1-(pyrimidin-2-yl)ethan-1-amine, Intermediate L-1, LCMS (ESI, M+1): m/z=293.1 and peak 2 (R)—N-((5-bromopyridin-2-yl)methyl)-1-(pyrimidin-2-yl)ethan-1-amine, Intermediate L-2, LCMS (ESI, M+1): m/z=293.1

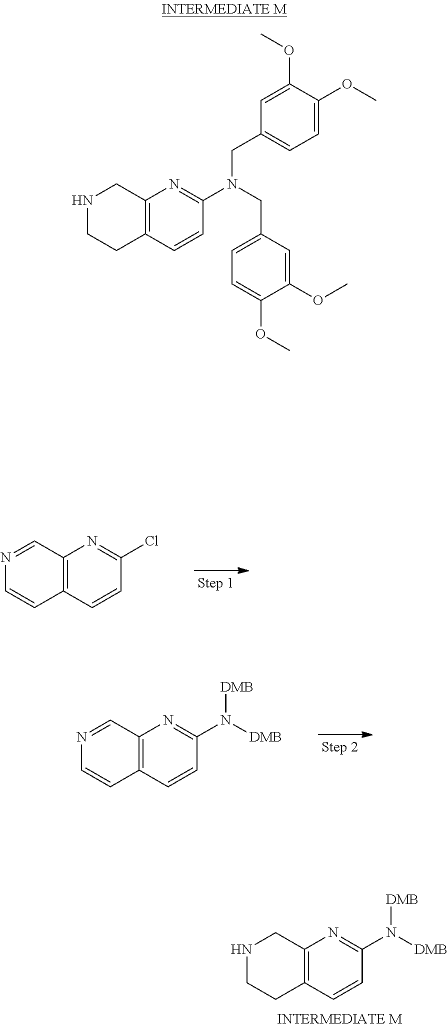

Step 1: To a solution of 2-chloro-1,7-naphthyridine (200 mg, 1.22 mmol, 1.00 eq.) in DMSO (5 mL) was added KF (84.7 mg, 1.46 mmol, 34.2 μL, 1.20 eq.) and 1-(2,4-dimethoxyphenyl)-N-[(2,4-dimethoxyphenyl)methyl]methanamine (463 mg, 1.46 mmol, 1.20 eq.). The mixture was stirred at 130° C. for 12 hours. The reaction mixture was diluted with water (100 mL) and extracted with ethyl acetate (60 mL×3). The combined organic layers were washed with brine (100 mL), dried over anhydrous sodium sulfate, filtered and concentrated under reduced pressure to give a residue. The residue was purified by column chromatography (SiO₂, Petroleum ether/Ethyl acetate 2:1 to 0:1) to give N,N-bis[(2,4-dimethoxyphenyl)methyl]-1,7-naphthyridin-2-amine (357 mg, 801 μmol, 65.9% yield) as a yellow oil. LCMS (ESI, M+1): m/z=446.3 Step 2: To a solution of N,N-bis[(2,4-dimethoxyphenyl)methyl]-1,7-naphthyridin-2-amine (300 mg, 673 μmol, 1.00 eq.) in MeOH (20 mL) was added PtO2 (152 mg, 673 μmol, 1.00 eq.) and HOAc (40.4 mg, 673 μmol, 38.5 μL, 1.0 eq.) under N₂ atmosphere. The suspension was degassed under vacuum and purged with H2 several times. The mixture was stirred under H2 (15 psi) at 25° C. for 12 hours. The reaction mixture was filtered and concentrated under reduced pressure to give a residue. The residue was purified by column chromatography (SiO₂, Petroleum ether/Ethyl acetate 1:1 to 0:1 followed by DCM/MeOH 10:1) to give N,N-bis[(2,4-dimethoxyphenyl)methyl]-5,6,7,8-tetrahydro-1,7-naphthyridin-2-amine, Intermediate M (193 mg, 429 μmol, 63.7% yield) as a yellow solid. LCMS (ESI, M+1); m/z=450.3. ¹H NMR (400 MHz, CHLOROFORM-d) δ=7.09-7.00 (m, 4H), 6.45 (d, J=2.4 Hz, 2H), 6.39 (dd, J=2.4, 8.4 Hz, 2H), 4.68 (s, 4H), 4.09 (s, 2H), 3.79 (s, 6H), 3.77 (s, 6H), 3.31 (br t, J=6.0 Hz, 2H), 2.85 (br t, J=5.6 Hz, 2H).

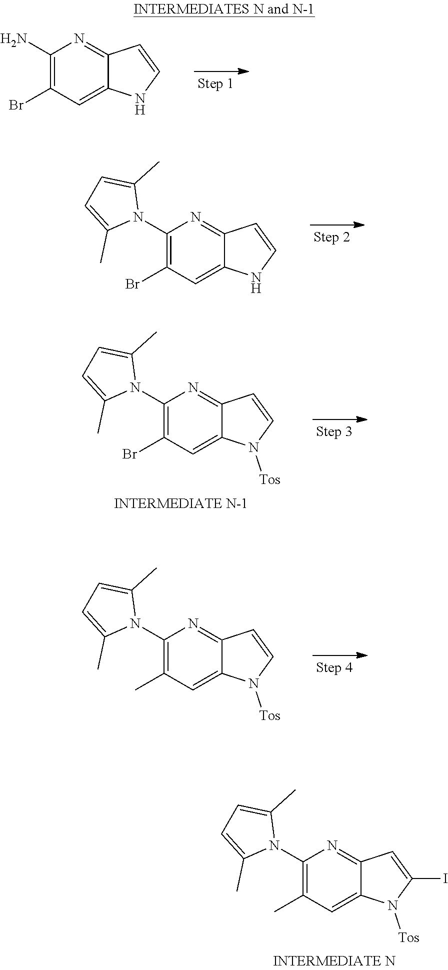

Step 1: A mixture of 6-bromo-1H-pyrrolo [3,2-b]pyridin-5-amine (8.74 g, 41.2 mmol, 1.00 eq.), hexane-2,5-dione (23.5 g, 206 mmol, 24.1 mL, 5.00 eq.) and p-Toluenesulfonic acid monohydrate (470 mg, 2.47 mmol, 0.06 eq.) in toluene (500 mL) was stirred at 140° C. for 16 hours in a flask equipped with a Dean Stark distillation trap. The mixture was concentrated. The residue was diluted with ethyl acetate (300 mL) and water (200 mL), stirred for 5 min and filtered. The organic layer was separated, dried over sodium sulfate, filtered and concentrated. The residue was purified by silica gel chromatography with Petroleum ether/Ethyl acetate (0-80% of Ethyl acetate). The eluent was concentrated, diluted with petroleum ether (100 mL) and ethyl acetate (20.0 mL), filtered and the filter cake was dried in vacuum to afford 6-bromo-5-(2,5-dimethylpyrrol-1-yl)-1H-pyrrolo[3,2-b]pyridine (5.00 g, 15.5 mmol, 37.6% yield) as a blue solid. ¹HNMR (400 MHz, DMSO-d₆) δ=11.69 (br s, 1H), 8.26 (s, 1H), 7.81 (t, J=2.8 Hz, 1H), 6.63 (br s, 1H), 5.77 (s, 2H), 1.87 (s, 6H).

Step 2: To a solution of 6-bromo-5-(2, 5-dimethylpyrrol-1-yl)-1H-pyrrolo[3,2-b]pyridine (5.00 g, 17.2 mmol, 1.00 eq.) in dichloromethane (50.0 mL) was added tetrabutylammonium hydrogen sulfate (292 mg, 861 μmol, 0.05 eq), sodium hydroxide (2.76 g, 68.9 mmol, 4.00 eq.) and 4-methylbenzenesulfonyl chloride (6.57 g, 34.4 mmol, 2.00 eq.). The mixture was stirred at 25° C. for 3 hours. The mixture was diluted with water (100 mL), extracted with dichloromethane (100 mL), dried over sodium sulfate, filtered and concentrated. The residue was purified by silica gel chromatography with Petroleum ether/Ethyl acetate (0-50% of Ethyl acetate) to afford 6-bromo-5-(2,5-dimethylpyrrol-1-yl)-1-(p-tolylsulfonyl)pyrrolo[3,2-b]pyridine, Intermediate N-1 (5.80 g, 12.9 mmol, 74.9% yield) as a white solid. LCMS [ESI, M+1]⁺=446.1. ¹HNMR (400 MHz, CDCl₃) δ=8.63 (s, 1H), 8.09-7.75 (m, 3H), 7.37 (d, J=8.4 Hz, 2H), 6.86 (d, J=3.6 Hz, 1H), 5.92 (s, 2H), 2.44 (s, 3H), 1.96 (s, 6H).

Step 3: To a solution of 6-bromo-5-(2,5-dimethylpyrrol-1-yl)-1-(p-tolylsulfonyl)pyrrolo[3,2-b]pyridine (4.80 g, 10.8 mmol, 1.00 eq.) and methylboronic acid (3.23 g, 54.0 mmol, 5.00 eq.) in dioxane (50.0 mL) and water (10.0 mL) was added potassium carbonate (2.99 g, 21.6 mmol, 2.00 eq.) and [1,1′-bis(diphenylphosphino)ferrocene]dichloropalladium(II) (441 mg, 540 μmol, 0.05 eq.). The mixture was stirred at 100° C. for 16 hours. The resulting solution was diluted with water (200 mL), extracted with ethyl acetate (200 mL), dried over sodium sulfate, filtered and concentrated. The residue was purified by silica gel chromatography with Petroleum ether/Ethyl acetate (0-50% of Ethyl acetate) to afford 5-(2,5-dimethylpyrrol-1-yl)-6-methyl-1-(p-tolylsulfonyl)pyrrolo[3,2-b]pyridine (4.50 g, crude) as a white solid. LCMS [ESI, M+1]⁺=380.2.

Step 4: To a solution of 5-(2,5-dimethylpyrrol-1-yl)-6-methyl-1-(p-tolylsulfonyl)pyrrolo[3,2-b]pyridine (1.00 g, 2.64 mmol, 1.00 eq.) in tetrahydrofuran (15.0 mL) was added diisopropylamino lithium (2.00 M, 2.64 mL, 2.00 eq.) and N,N,N′,N′-tetramethylethane-1,2-diamine (367 mg, 3.16 mmol, 477 μL 1.20 eq.) at −65° C. dropwise. The mixture was stirred at −65° C. for 1 hour. A solution of iodine (1.34 g, 5.27 mmol, 1.06 mL, 2.00 eq.) in tetrahydrofuran (5.00 mL) was added dropwise at −65° C. The mixture was stirred at −65° C. for 1 hour and stirred at 25° C. for 1 hour. The mixture was quenched with saturated ammonium chloride (100 mL), extracted with ethyl acetate (100 mL), dried over sodium sulfate, filtered and concentrated. The residue was purified by silica gel chromatography with Petroleum ether/Ethyl acetate (0-50% of Ethyl acetate) to afford 5-(2,5-dimethylpyrrol-1-yl)-2-iodo-6-methyl-1-(p-tolylsulfonyl)pyrrolo[3,2-b]pyridine, Intermediate N (1.00 g, 1.81 mmol, 68.7% yield) as an off-white solid. LCMS [EST, M+1]⁺=506.1. ¹HNMR (400 MHz, CDCl₃) δ=8.51 (s, 1H), 7.87 (d, J=8.4 Hz, 2H), 7.33 (d, J=8.0 Hz, 2H), 7.18 (s, 1H), 5.91 (s, 2H), 2.43 (s, 3H), 2.14 (s, 3H), 1.92 (s, 6H).

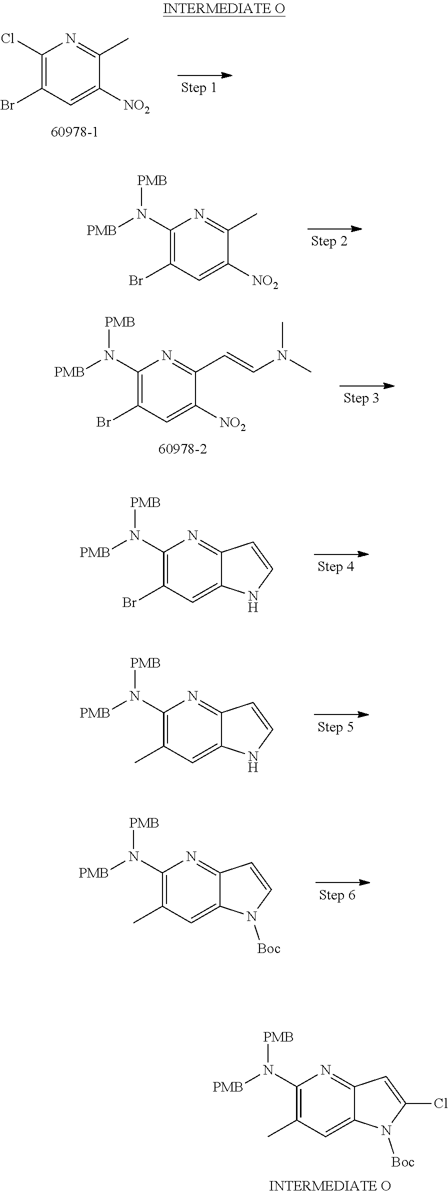

Step 1: To a solution of 3-bromo-2-chloro-6-methyl-5-nitro-pyridine (25.0 g, 99.4 mmol, 1.0 equiv) and 1-(4-methoxyphenyl)-N-[(4-methoxyphenyl)methyl]methanamine (28.1 g, 109.4 mmol, 1.1 equiv) in tetrahydrofuran (250 mL) was added sodium carbonate (11.6 g, 109.4 mmol, 1.1 equiv). The mixture was stirred at 70° C. for 14 hours. The reaction mixture was diluted with water (300.0 mL) and stirred for 5 minutes, the aqueous phase was extracted with ethyl acetate (300.0 mL×3), the combined organic phase was washed with brine (100.0 mL), dried, filtered and concentrated to give the residue, which was purified by silica gel column chromatography (0-30% Ethyl acetate/Petroleum) to give 3-bromo-N,N-bis(4-methoxybenzyl)-6-methyl-5-nitropyridin-2-amine (36.2 g, 76.6 mmol, 77.1% yield) as a yellow oil. LCMS (ESI, M+1). m/z=472.0. ¹H NMR δ=8.57-8.49 (m, 1H), 7.20 (d, J=8.4 Hz, 4H), 6.87 (d, J=8.8 Hz, 4H), 4.75 (s, 4H), 3.82 (s, 6H), 2.76 (s, 3H).

Step 2: To a solution of 3-bromo-N,N-bis[(4-methoxyphenyl)methyl]-6-methyl-5-nitro-pyridin-2-amine (31.2 g, 66.1 mmol, 1.0 equiv) in DMF (300 mL) was added 1,1-dimethoxy-N,N-dimethyl-methanamine (27.6 g, 231.2 mmol, 30.7 mL, 3.5 equiv). The mixture was stirred at 120° C. for 1 hours. The reaction mixture was diluted with water (300.0 mL) and stirred for 5 minutes, the aqueous phase was extracted with ethyl acetate (300.0 mL×3), the combined organic phase was washed with brine (800 mL), dried, filtered and concentrated to give 3-bromo-6-[(E)-2-(dimethylamino)vinyl]-N,N-bis[(4-methoxyphenyl)methyl]-5-nitro-pyridin-2-amine (34.4 g, crude) as a red oil. LCMS (ESI, M+1); m/z=529.1.

Step 3: A mixture of 3-bromo-6-[(E)-2-(dimethylamino)vinyl]-N,N-bis[(4-methoxyphenyl)methyl]-5-nitro-pyridin-2-amine (11.0 g, 20.9 mmol, 1.0 equiv), Pt/C (1.09 g, 2.09 mmol, 50% purity, 0.1 equiv) in MeOH (10 mL) was degassed and purged with H2 for 3 times, and then the mixture was stirred at 25° C. for 12 hours under H2 atmosphere. The reaction mixture was filtered and the filtrate was concentrated under reduced pressure to give a residue, which was purified by reversed-phase HPLC to give 6-bromo-N,N-bis[(4-methoxyphenyl)methyl]-1H-pyrrolo[3,2-b]pyridin-5-amine (17.4 g, 38.1 mmol, 91.3% yield) as a dark brown solid. LCMS (ESI, M+1): m/z=452.0.

Step 4: A mixture of 6-bromo-N,N-bis[(4-methoxyphenyl)methyl]-1H-pyrrolo[3,2-b]pyridin-5-amine (8.00 g, 17.7 mmol, 1.00 equiv), Catacxium® A Pd-G3 (644 mg, 884 μmol, 0.05 equiv), potassium carbonate (14.7 g, 106 mmol, 6.00 equiv), methylboronic acid (8.47 g, 141 mmol, 8.00 equiv) in water (8.00 mL) and dioxane (80.0 mL) was degassed and stirred at 100° C. for 5 hours under nitrogen atmosphere. The reaction mixture was diluted with water (20.0 mL), the aqueous phase was extracted with ethyl acetate (20.0 mL×3), the combined organic phase was washed with brine (10.0 mL), dried, filtered and concentrated to give a residue, which was purified by silica gel column chromatography (0-40% Ethyl acetate/Petroleum) to give N,N-bis[(4-methoxyphenyl)methyl]-6-methyl-1H-pyrrolo[3,2-b]pyridin-5-amine (5.88 g, 15.0 mmol, 85.0% yield) as a yellow solid. LCMS (ESI, M+1): m/z=388.2. ¹H NMR (400 MHz, CDCl₃) δ=8.27-8.08 (m, 1H), 7.30 (s, 1H), 7.14 (d, J=8.8 Hz, 5H), 6.70 (d, J=8.4 Hz, 4H), 6.54-6.46 (m, 1H), 4.13 (s, 4H), 3.67 (s, 6H), 2.37 (s, 3H).

Step 5: To a solution of N,N-bis[(4-methoxyphenyl)methyl]-6-methyl-1H-pyrrolo[3,2-b]pyridin-5-amine (5.88 g, 15.2 mmol, 1 equiv) in dichloromethane (60 mL) was added Boc₂O (6.62 g, 30.4 mmol, 6.97 mL, 2.00 equiv), DMAP (556 mg, 4.55 mmol, 0.30 equiv) and triethylamine (3.07 g, 30.4 mmol, 4.22 mL, 2.00 equiv) at 0° C., the mixture was stirred at 25° C. for 12 hours. The reaction mixture was diluted with water (100.0 mL), the aqueous phase was extracted with ethyl acetate (150 mL×3), the combined organic phase was washed with brine (50.0 mL), dried, filtered and concentrated to give the residue. The residue was purified by silica gel column chromatography (˜15% Ethyl acetate/Petroleum ether) to give tert-butyl 5-[bis[(4-methoxyphenyl)methyl]amino]-6-methyl-pyrrolo[3,2-b]pyridine-1-carboxylate (6.7 g, 13.3 mmol, 87.8% yield) as a yellow oil. LCMS (ESI, M+1): m/z=488.2. ¹H NMR (400 MHz, CDCl₃) δ=8.22-8.08 (m, 1H), 7.69-7.60 (m, 1H), 7.20 (d, J=8.8 Hz, 4H), 6.79 (d, J=8.4 Hz, 4H), 6.66-6.60 (m, 1H), 4.22 (s, 4H), 3.77 (s, 6H), 2.49 (s, 3H), 1.67 (s, 9H).

Step 6: To a solution of tert-butyl 5-[bis[(4-methoxyphenyl)methyl]amino]-6-methyl-pyrrolo[3,2-b]pyridine-1-carboxylate (2.1 g, 4.31 mmol, 1.00 equiv) in tetrahydrofuran (20.0 mL) was added n-BuLi (2.5 M, 6.89 mL, 4.00 equiv) at −65° C. The mixture was stirred at −65° C. for 2 hours, then to the mixture was added 1,1,1,2,2,2-hexachloroethane (2.04 g, 8.61 mmol, 975 μL 2.00 equiv) at −65° C. Then the mixture was stirred at 25° C. for 12 hours. The reaction mixture was diluted with water (20.0 mL), the aqueous phase was extracted with ethyl acetate (20.0 mL>×3). The combined organic phase was washed with brine (10.0 mL), dried, filtered and concentrated to give the residue. The residue was purified by silica gel column chromatography (0-10% Ethyl acetate/Petroleum ether) to give tert-butyl 5-[bis[(4-methoxyphenyl)methyl]amino]-2-chloro-6-methyl-pyrrolo[3,2-b]pyridine-1-carboxylate, Intermediate O (680 mg, 1.30 mmol, 30.2% yield) as a yellow oil. ¹H NMR (400 MHz, CDCl₃) δ=8.14-8.05 (m, 1H), 7.19 (d, J=8.4 Hz, 4H), 6.79 (d, J=8.8 Hz, 4H), 6.67-6.60 (m, 1H), 4.20 (s, 4H), 3.77 (s, 6H), 2.48 (s, 3H), 1.68 (s, 9H).

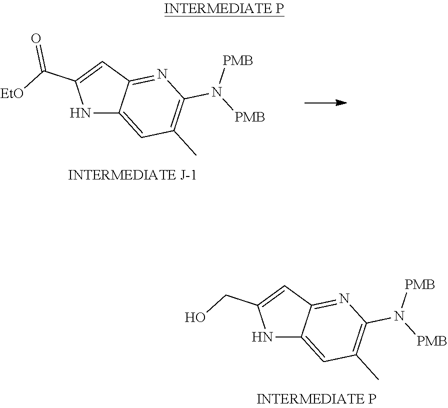

To solution of ethyl Intermediate J-1 (1.70 g, 3.70 mmol, 1.00 eq.) in tetrahydrofuran (20.0 mL) was added lithium aluminum hydride (421 mg, 11.1 mmol, 3.00 eq.) portion wise at 0° C. under nitrogen atmosphere. The resulting mixture was stirred at 0° C. for 0.5 hour and then warmed to 25° C. and stirred for 1 hour. The mixture was quenched with sodium sulfate decahydrate at 0-5° C. under nitrogen atmosphere. The mixture was filtered and the filter cake was washed with tetrahydrofuran (10.0 mL-3). The combined organic layers were concentrated. The residue was triturated with petroleum ether/ethyl acetate 10:1 (50.0 mL) at 25° C. for 1 hour. Then the mixture was filtered and the filter cake was concentrated to obtain [5-[bis[(4-methoxyphenyl)methyl]amino]-6-methyl-1H-pyrrolo[3,2-b]pyridine-2-yl] methanol, Intermediate P (1.20 g, 2.87 mmol, 77.7% yield) as off-white solid. LCMS [ESI, M+1]: 418.3. ¹H NMR (400 MHz, CD₃OD-d₆) δ=7.49 (s, 1H), 7.14 (d, J=8.4 Hz, 4H), 6.77 (d, J=8.4 Hz, 4H), 6.38 (s, 1H), 4.72 (s, 2H), 4.12 (s, 4H), 3.73 (s, 6H), 2.40 (s, 3H)

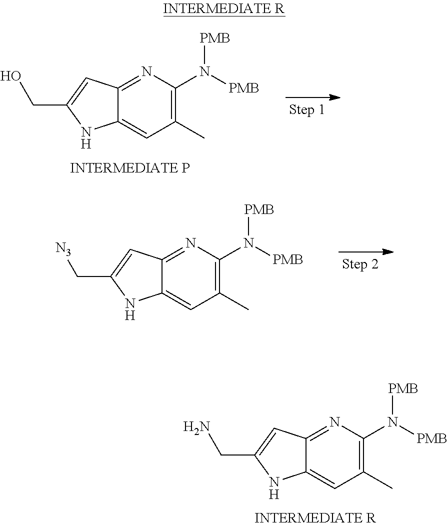

Step 1: To a solution of Intermediate P (50.0 mg, 120 μmol, 1.00 eq.) and diphenyl phosphoryl azide (98.9 mg, 359 μmol, 77.9 μL 3.00 eq.) in dichloromethane (2.50 mL) was added a solution of 1,8-diazabicyclo[5.4.0]undec-7-ene (54.7 mg, 359 μmol, 54.2 μL 3.00 eq.) in dichloromethane (0.50 mL) dropwised at 0° C. The mixture was stirred at 25° C. for 2 hours. The mixture was diluted with water (50.0 mL) and extracted with dichloromethane (10.0 mL×2). The organic layer was dried over sodium sulfate, filtered and concentrated to 5 mL. The mixture was diluted with tetrahydrofuran (10.0 mL) and concentrated to 3 mL to afford 2-(azidomethyl)-N,N-bis(4-methoxybenzyl)-6-methyl-1H-pyrrolo[3,2-b]pyridin-5-amine (crude solution) as a light yellow liquid.

Step 2: To a solution of 2-(azidomethyl)-N,N-bis(4-methoxybenzyl)-6-methyl-1H-pyrrolo[3,2-b]pyridin-5-amine (50.0 mg, 113 μmol, 1.00 eq.) in tetrahydrofuran (2.5 mL) and water (0.80 mL) was added triphenylphosphine (88.9 mg, 339 μmol, 3.00 eq.). The mixture was stirred at 20° C. for 3 hours. The mixture was concentrated to afford 2-(aminomethyl)-N,N-bis(4-methoxybenzyl)-6-methyl-1H-pyrrolo[3,2-b]pyridin-5-amine, Intermediate R (40.0 mg, crude) as a light yellow oil.

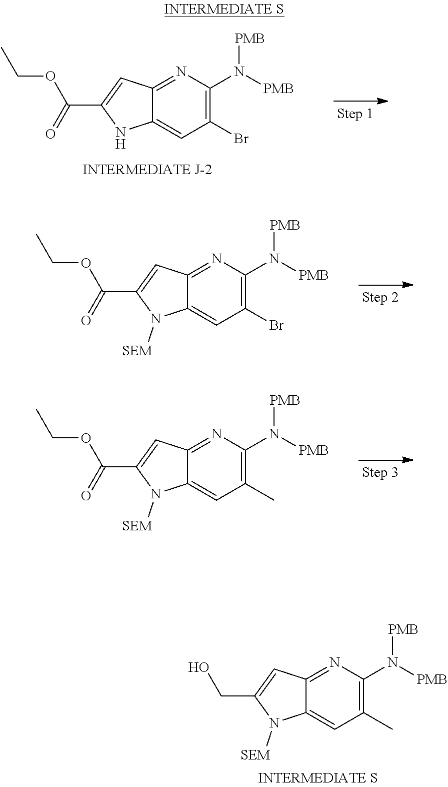

Step 1: To a solution of Intermediate J-2 (3.50 g, 6.67 mmol, 1.00 eq.) in dimethyl formamide (70.0 mL) was added sodium hydride (320 mg, 8.01 mmol, 60.0% purity, 1.20 eq.) at 0° C. under nitrogen atmosphere. The mixture was stirred at 0° C. for 0.5 hours. Then (2-(chloromethoxy)ethyl)trimethylsilane (1.67 g, 10.0 mmol, 1.77 mL, 1.50 eq.) was added to the above reaction mixture. The resulting reaction mixture was stirred at 20° C. for 1 hour. The mixture was quenched with water (500 mL) carefully at 0° C. and extracted with ethyl acetate (100 mL×3). The organic layer was washed with brine (200 mL), dried over sodium sulfate, filtered and concentrated. The residue was purified by flash silica gel chromatography (5-209% Ethyl acetate/Petroleum ether) to afford ethyl 5-(bis(4-methoxybenzyl)amino)-6-bromo-1-((2-(trimethylsilyl)ethoxy)methyl)-1H-pyrrolo[3,2-b]pyridine-2-carboxylate (4.20 g, 6.34 mmol, 95.00% yield) as a light yellow oil. LCMS [ESI, M+1]⁺=656.2. ¹HNMR (400 MHz, CDCl₃) δ=8.14-8.09 (m, 1H), 7.29 (d, J=8.4 Hz, 5H), 6.81 (d, J=8.4 Hz, 4H), 5.93 (s, 2H), 4.42-4.32 (m, 6H), 3.77 (s, 6H), 3.57-3.51 (m, 2H), 1.41 (t, J=7.2 Hz, 3H), -0.02-0.11 (m, 9H).

Step 2: To a solution of ethyl 5-(bis(4-methoxybenzyl)amino)-6-bromo-1-((2-(trimethylsilyl)ethoxy)methyl)-1H-pyrrolo[3,2-b]pyridine-2-carboxylate (2.00 g, 3.05 mmol, 1.00 eq.) and 2,4,6-trimethyl-1,3,5,2,4,6-trioxatriborinane (1.92 g, 7.64 mmol, 2.14 mL, 50.0% purity, 2.50 eq.) in dioxane (50.0 mL) was added potassium carbonate (1.27 g, 9.16 mmol, 3.00 eq.) and [1,1′-bis(diphenylphosphino)ferrocene]dichloropalladium(II) (224 mg, 306 μmol, 0.10 eq). The mixture was stirred at 100° C. for 16 hours under nitrogen atmosphere. The mixture was diluted with water (200 mL) and extracted with ethyl acetate (80.0 mL 2). The organic layer was washed with brine (100 mL), dried over sodium sulfate, filtered and concentrated. The residue was purified by flash silica gel chromatography (5-10% Ethyl acetate/Petroleum ether) to afford ethyl 5-(bis(4-methoxybenzyl)amino)-6-methyl-1-((2-(trimethylsilyl)ethoxy) methyl)-1H-pyrrolo[3,2-b]pyridine-2-carboxylate (1.70 g, 2.58 mmol, 84.6% yield) as a light yellow oil. LCMS [ESI, M+1]⁺=590.4. ¹HNMR (400 MHz, CDCl₃) δ=7.63 (br s, 1H), 7.39-7.32 (m, 1H), 7.22 (br d, J=8.4 Hz, 4H), 6.82-6.77 (m, 4H), 5.95 (s, 2H), 4.38 (q, J=7.2 Hz, 2H), 4.23 (br s, 3H), 3.77 (s, 6H), 3.58-3.49 (m, 2H), 2.53 (s, 3H), 1.40 (t, J=7.2 Hz, 3H), -0.07 (s, 9H).

Step 3: To a solution of ethyl 5-(bis(4-methoxybenzyl)amino)-6-methyl-1-((2-(trimethylsilyl)ethoxy)methyl)-1H-pyrrolo[3,2-b]pyridine-2-carboxylate (2.90 g, 4.92 mmol, 1.00 eq.) in ethyl alcohol (50.0 mL) was added lithium chloride (1.67 g, 39.3 mmol, 806 μL 8.00 eq.) and sodium borohydride (1.49 g, 39.3 mmol, 8.00 eq.) at 0° C. Then the mixture was stirred at 20° C. for 2 hours. The mixture was stirred at 40° C. for another 16 hours. The mixture was quenched by saturated ammonium chloride (300 mL) and water (200 mL) at 0° C. The mixture was extracted with ethyl acetate (100 mL×3). The organic layer was washed with brine (300 mL), dried over sodium sulfate, filtered and concentrated The residue was purified by flash silica gel chromatography (5˜30% Ethyl acetate/Petroleum ether) to afford (5-(bis(4-methoxybenzyl)amino)-6-methyl-1-((2-(trimethylsilyl)ethoxy)methyl)-1H-pyrrolo[3,2-b]pyridin-2-yl)methanol (2.50 g, 4.56 mmol, 92.8% yield) as a light yellow oil. ¹H NMR (400 MHz, CDCl₃) δ=7.52 (br s, 1H), 7.23 (d, J=8.4 Hz, 4H), 6.80-6.76 (m, 4H), 6.72-6.60 (m, 1H), 5.52 (s, 2H), 4.83 (d, J=6.0 Hz, 2H), 4.24 (br s, 4H), 3.77 (s, 7H), 3.57-3.49 (m, 2H), 2.49 (s, 3H), -0.05 (s, 9H).

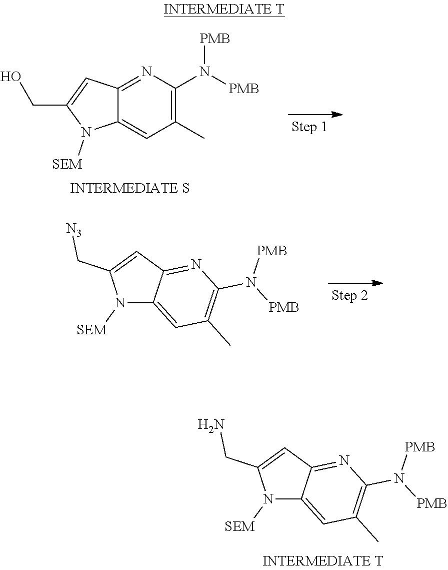

Step 1: To a solution of Intermediate S (500 mg, 913 μmol, 1.00 eq.) and diphenyl phosphoryl azide (754 mg, 2.74 mmol, 593 μL 3.00 eq.) in dichloromethane (15.0 mL) was added a solution of 1,8-diazabicyclo[5.4.0]undec-7-ene (417 mg, 2.74 mmol, 413 μL 3.00 eq.) in dichloromethane (2.00 mL) dropwised at 0° C. The mixture was stirred at 25° C. for 3 hours. The mixture was diluted with tetrahydrofuran (30.0 mL) and concentrated to 5.00 mL to afford 2-(azidomethyl)-N,N-bis(4-methoxybenzyl)-6-methyl-1-((2-(trimethylsilyl)ethoxy)methyl)-1H-pyrrolo[3,2-b]pyridin-5-amine (520 mg, crude solution) as a light yellow liquid.

Step 2: To a solution of 2-(azidomethyl)-N,N-bis(4-methoxybenzyl)-6-methyl-1-((2-(trimethylsilyl)ethoxy)methyl)-1H-pyrrolo[3,2-b]pyridin-5-amine (520 mg, 908 μmol, 1.00 eq.) in tetrahydrofuran (10.0 mL) and water (3.50 mL) was added triphenylphosphine (714 mg, 2.72 mmol, 3.00 eq.). The mixture was stirred at 40° C. for 2 hours. The mixture was concentrated. The residue was purified by flash silica gel chromatography (5˜10% Methanol/dichloromethane) to afford 2-(aminomethyl)-N,N-bis(4-methoxybenzyl)-6-methyl-1-((2-(trimethylsilyl)ethoxy)methyl)-1H-pyrrolo[3,2-b]pyridin-5-amine, Intermediate T (450 mg, crude) as a light yellow oil. LCMS [ESI, M+1]⁺=547.3.

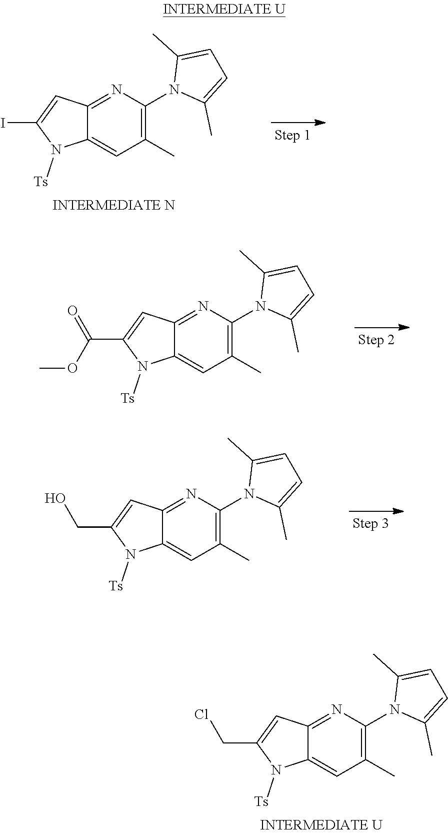 [SMILES: Cc1ccc(S(=O)(=O)n2c(I)cc3nc(-n4c(C)ccc4C)c(C)cc32)cc1 ; COC(=O)c1cc2nc(-n3c(C)ccc3C)c(C)cc2n1S(=O)(=O)c1ccc(C)cc1 ; Cc1ccc(S(=O)(=O)n2c(CO)cc3nc(-n4c(C)ccc4C)c(C)cc32)cc1 ; Cc1ccc(S(=O)(=O)n2c(CCl)cc3nc(-n4c(C)ccc4C)c(C)cc32)cc1]

Step 1: To a solution of Intermediate N (1.00 g, 1.98 mmol, 1.00 eq.) in dimethylformamide (5.00 mL) were added [1,1-bis(diphenylphosphino)ferrocene]dichloropalladium(II) (144 mg, 197 μmol, 0.01 eq.), triethylamine (400 mg, 3.96 mmol, 550 μL 2.00 eq.) and methanol (5.00 mL) under nitrogen atmosphere. The suspension was degassed and purged with nitrogen for 3 times. The mixture was stirred under carbon monoxide (50 psi) at 80° C. for 12 hours. After being cooled to room temperature, the mixture was concentrated under vacuum to get a residue. The residue was diluted with ethyl acetate (20.0 mL) and water (20.0 mL). The layers were separated and the aqueous phase was extracted with ethyl acetate (3-20.0 mL). The organic layer was dried over anhydrous sodium sulfate, filtered and concentrated under vacuum. The residue was purified by column chromatography on silica gel (petroleum ether/ethyl acetate 10:1 to 8:1) to give methyl 5-(2,5-dimethylpyrrol-1-yl)-6-methyl-1-(p-tolylsulfonyl)pyrrolo[3,2-b] pyridine-2-carboxylate (800 mg, 1.70. mmol, 85.9% yield) as a yellow solid. ¹H NMR (400 MHz, CDCl₃-d) 5=8.41 (s, 1H), 8.04 (d, J=8.4 Hz, 2H), 7.38 (d, J=8.4 Hz, 2H), 7.30 (s, 1H), 5.91 (s, 2H), 3.95 (s, 3H), 2.45 (s, 3H), 2.15 (s, 3H), 1.92 (s, 6H).

Step 2: To a solution of methyl 5-(2,5-dimethylpyrrol-1-yl)-6-methyl-1-(p-tolylsulfonyl)pyrrolo[3,2-b]pyridine-2-carboxylate (1.60 g, 3.66 mmol, 1.00 eq.) in tetrahydrofuran (20.0 mL) was added Lithium aluminum hydride (181 mg, 4.76 mmol, 1.30 eq.) at 0° C. for 8 hours. The reaction was quenched with saturated ammonium chloride (10.0 mL). The mixture was extracted with ethyl acetate (3×10.0 mL). The combined organic extracts were washed with brine (15.0 mL) and then dried over anhydrous sodium sulfate, and it was concentrated under vacuum. The residue was purified by column chromatography (SiO₂, petroleum ether/ethyl acetate 8:1) to afford [5-(2,5-dimethylpyrrol-1-yl)-6-methyl-1-(p-tolylsulfonyl)pyrrolo[3,2-b]pyridin-2-yl]methanol (510 mg, 1.17 mmol, 32.1% yield) as a light yellow solid. ¹H NMR (400 MHz, CDCl₃-d) δ=8.31 (s, 1H), 7.79 (d, J=8.4 Hz, 2H), 7.31 (d, J=8.0 Hz, 2H), 6.82 (s, 1H), 5.89 (s, 2H), 4.95 (s, 2H), 2.41 (s, 3H), 2.10 (s, 3H), 1.90 (s, 6H).

Step 3: To a solution of [5-(2,5-dimethylpyrrol-1-yl)-6-methyl-1-(p-tolylsulfonyl)pyrrolo[3,2-b]pyridin-2-yl]methanol (310 mg, 757 μmol, 1.00 eq.) in dichloromethane (20.0 mL) were added trifluoroacetic acid (76.6 mg, 757 μmol, 105 μL 1.00 eq.) and 4-dimethylaminopyridine (92.5 mg, 757 μmol, 1.00 eq.), 4-methylbenzenesulfonyl chloride (216 mg, 1.14 mmol, 1.50 eq.) at 0° C. The mixture was stirred at 20° C. for 2 hours. The reaction mixture was purified by column chromatography (SiO₂, Petroleum ether/Ethyl acetate 6:1) to get 2-(chloromethyl)-5-(2, 5-dimethylpyrrol-1-yl)-6-methyl-1-(p-tolylsulfonyl)pyrrolo[3,2-b]pyridine, Intermediate U (200 mg, 382 μmol, 50.4% yield) as a red solid. ¹H NMR (400 MHz, CDCl₃-d) δ=8.35 (s, 1H), 7.84 (d, J=8.4 Hz, 2H), 7.32 (d, J=8.4 Hz, 2H), 6.97 (s, 1H), 5.90 (s, 2H), 5.07 (s, 2H), 2.42 (s, 3H), 2.12 (s, 3H), 1.91 (s, 6H).

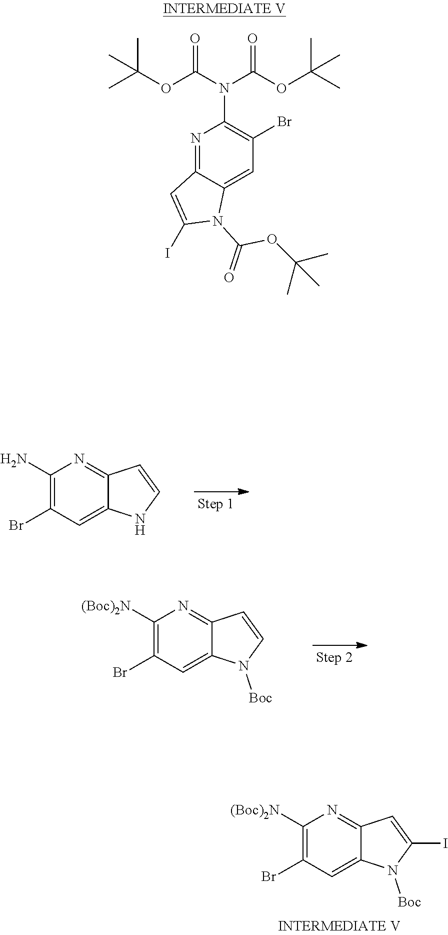

Step 1: To a solution of 6-bromo-1H-pyrrolo[3,2-b]pyridin-5-amine (1.6 g, 7.55 mmol, 1.00 eq.) in DCM (50 mL) was added DMAP (184 mg, 1.51 mmol, 0.20 eq.) and Boc₂O (4.94 g, 22.6 mmol, 5.20 mL, 3.0 eq.). The mixture was stirred at 25° C. for 12 hours. The reaction mixture was concentrated under reduced pressure to give a residue. The residue was purified by column chromatography (SiO₂, Petroleum ether/Ethyl acetate 10:1 to 3:1) to give tert-butyl 5-[bis(tert-butoxycarbonyl)amino]-6-bromo-pyrrolo[3,2-b]pyridine-1-carboxylate (2.80 g, 5.46 mmol, 72.4% yield) as a yellow solid. LCMS (ESI, M+1); m/z=512.2. ¹H NMR (400 MHz, DMSO-d₆) δ=8.58 (s, 1H), 8.06 (d, J=4.0 Hz, 1H), 6.87 (d, J=3.2 Hz, 1H), 1.64 (s, 9H), 1.34 (s, 18H) Step 2: n-BuLi (2.5 M, 491 μL, 2.1 eq.) was added a solution of N-isopropylpropan-2-amine (142.19 mg, 1.41 mmol, 198.59 μL, 2.4 eq.) in THF (15 mL) at −70° C., the mixture was stirred at −70° C. for 0.5 hour. Then tert-butyl 5-[bis(tert-butoxycarbonyl)amino]-6-bromo-pyrrolo[3,2-b]pyridine-1-carboxylate (300 mg, 585.49 μmol, 1 eq) and N,N,N′,N′-tetramethylethane-1,2-diamine (102 mg, 878 μmol, 132 μL, 1.50 eq) in THE (1 mL) was added and stirred at −70° C. and the resulting was stirred for 0.5 hour. A solution of iodine (193 mg, 761 μmol, 153 μL, 1.30 eq) in THE (1 mL) was added to the mixture and the resulting was stirred at −70° C. for 0.5 hour. The reaction mixture was diluted with water (80 mL) and extracted with ethyl acetate (50.0 mL×3). The combined organic layers were washed with brine (100 mL), dried over sodium sulfate, filtered and concentrated under reduced pressure to give a residue. The residue was purified by column chromatography by prep-TLC (SiO₂, Petroleum ether/Ethyl acetate 5:1) to give tert-butyl 5-[bis(tert-butoxycarbonyl)amino]-6-bromo-2-iodo-pyrrolo[3,2-b]pyridine-1-carboxylate, Intermediate V (185 mg, 289.84 μmol, 49.50% yield) as a white solid. LCMS (ESI, M+1): m/z=637.9. ¹H NMR (400 MHz, DMSO-d₆) δ=8.55 (s, 1H), 7.31 (s, 1H), 1.68 (s, 9H), 1.34 (s, 18H)

### General Coupling Methods for the Preparation of Examples 1-1 to 1-8

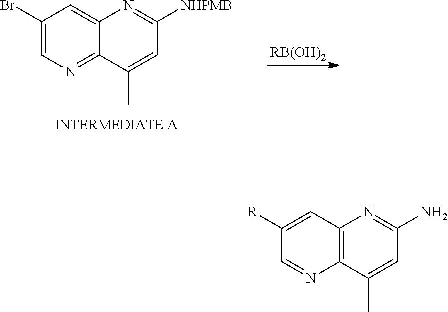

Coupling Method 1-A (CM1-A): A mixture of Intermediate A (279 μmol, 1.00 eq.), corresponding boron ester (558 μmol, 2.00 eq.), Pd(dppf)Cl₂ (27.9 μmol, 0.10 eq.) and cesium carbonate (558 μmol, 2.00 eq.) in dioxane (1.00 mL) and water (0.20 mL) was degassed and stirred at 100° C. for 1 hour under nitrogen atmosphere. The reaction mixture was diluted with water (2.00 mL) and extracted with ethyl acetate (2.00 mL×3). Combined organic phases were washed with brine (2.00 mL), dried over sodium sulfate, filtered and concentrated to give a residue which was used in the deprotection step directly without further purification. The obtained residue was diluted with TFA (2.00 mL) and stirred at 70° C. for 6 hours. The reaction mixture was concentrated under reduced pressure to give a residue. The residue was basified with ammonium hydroxide (0.10 mL) to pH 9. The solution was purified by prep-HPLC (neutral condition) to give the final product.

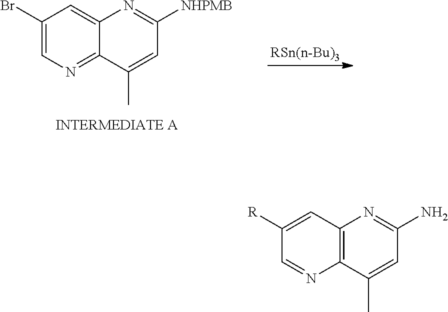

Coupling Method 1-B (CM1-B): A mixture of Intermediate A (279 μmol, 1.00 eq.), corresponding organotin reagent (558 μmol, 2.00 eq.) and Pd(PPh₃)₄ (27.9 μmol, 0.10 eq.) in toluene (1.00 mL) was degassed and stirred at 110° C. for 2 hours. The reaction mixture was concentrated under reduced pressure to give a residue. The residue was purified by prep-TLC (dichloromethane/methyl alcohol 10:1) to give a solid. The obtained solid was diluted with TFA (2.00 mL) and stirred at 70° C. for 6 hours. The reaction mixture was concentrated under reduced pressure to give a residue. The residue was basified with ammonium hydroxide (0.10 mL) to pH 9. The solution was purified by prep-HPLC (neutral condition) to give the final product.

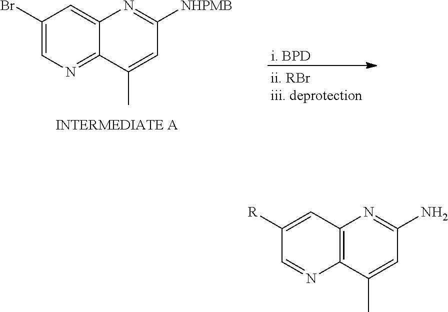

Coupling Method 1-C(CM1-C): A mixture of Intermediate A (1.12 mmol, 1.00 eq.), 4,4,4′,4′,5,5,5′,5′-octamethyl-2,2′-bi(1,3,2-dioxaborolane) (1.34 mmol, 1.20 eq.), potassium acetate (2.23 mmol, 2.00 eq.) and Pd(dppf)Cl₂ (81.7 mg, 112 μmol, 0.10 eq.) in dioxane (5.00 mL) was degassed and stirred at 80° C. for 4 hours under nitrogen atmosphere. The reaction mixture was concentrated under reduced pressure to give a boronic acid (360 mg, crude) as a brown oil, which was used in the next step directly without further purification. A mixture of boronic acid (557 μmol, 1.00 eq.), corresponding bromides (1.11 mmol, 2.00 eq.), Pd(dppf)Cl₂ (55.7 μmol, 0.10 eq.), cesium carbonate (557 μmol, 1.00 eq.) in dioxane (5.00 mL) and water (1.00 mL) was degassed and stirred at 100° C. for 2 hours under nitrogen atmosphere. The reaction mixture was diluted with water (10.0 mL) and extracted with ethyl acetate (10.0 mL×3). Combined organic phases were washed with brine (10.0 mL), dried over sodium sulfate, filtered and concentrated to give a residue. The residue was diluted with trifluoroacetic acid (5.00 mL) and stirred at 70° C. for 5 hours. The reaction mixture was concentrated under reduced pressure to give a residue. The residue was purified by prep-HPLC (neutral condition) to give the final product.

Starting from intermediate A, following the teachings of General Reaction Scheme I and coupling methods CM1-A, CM1-B and CM1-C Examples 1-1 to 1-8 were prepared as shown in Table 1:

TABLE 1

| | | | Yield | |
|---|---|---|---|---|
| | | Coupling | (2-3 | |
| Example | Structure | Method | steps) | Compound Name and Characterization |
| 1-1 | 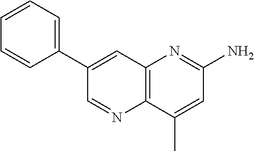 [SMILES: Cc1cc(N)nc2cc(-c3ccccc3)cnc12] | CM1-A | 34% | 4-methyl-7-phenyl-1,5-naphthyridin-2-amine LCMS [M + 1]: 236.3. 1H NMR (400 MHz, MeOD) δ = 8.79 (d, J = 2.4 Hz, 1H), 8.03 (d, J = 2.4 Hz, 1H), 7.81-7.68 (m, 2H), 7.56-7.50 (m, 2H), 7.47- 7.41 (m, 1H), 6.92 (d, J = 0.8 Hz, 1H), 2.65 (d, J = 0.8 Hz, 3H). |
| 1-2 | 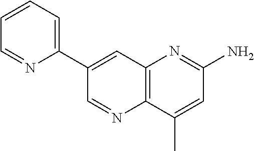 [SMILES: Cc1cc(N)nc2cc(-c3ccccn3)cnc12] | CM1-B | 41% | 4-methyl-7-(pyridin-2-yl)-1,5-naphthyridin-2- amine LCMS [M + 1]: 237.2. H NMR (400 MHz, MeOD) δ = 9.13 (d, J = 2.0 Hz, 1H), 8.73-8.68 (m, 1H), 8.36 (d, J = 2.0 Hz, 1H), 8.03-7.92 (m, 2H), 7.43 (m, 1H), 6.95 (d, J = 1.2 Hz, 1H), 2.65 (d, J = 1.2 Hz, 3H). |
| 1-3 | 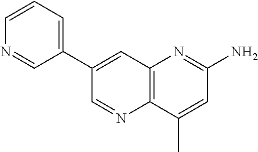 [SMILES: Cc1cc(N)nc2cc(-c3cccnc3)cnc12] | CM1-A | 23% | 4-methyl-7-(pyridin-3-yl)-1,5-paphthyridin-2- amine LCMS [M + 1]: 237.2. ¹H NMR (400 MHz, MeOD) δ = 8.94 (dd, J = 0.8, 2.4 Hz, 1H), 8.81 (d, J = 2.0 Hz, 1H), 8.62 (dd, J = 1.6, 4.8 Hz, 1H), 8.26-8.17 (m, 1H), 8.07 (d, J = 2.0 Hz, 1H), 7.60 (ddd, J = 0.8, 4.8, 8.0 Hz, 1H), 6.95 (d, J = 0.8 Hz, 1H), 2.66 (d, J = 0.8 Hz, 3H) |
| 1-4 | 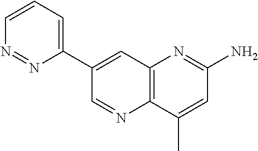 [SMILES: Cc1cc(N)nc2cc(-c3cccnn3)cnc12] | CM1-C | 30% | 4-methyl-7-(pyridazin-3-yl)-1,5-naphthyridin-2- amine LCMS [M + 1]: 238.2. ¹H NMR (400 MHz, MeOD) δ = 9.25 (d, J = 2.2 Hz, 1H), 9.23 (dd, J = 1.6, 5.2 Hz, 1H), 8.47 (d, J = 2.2 Hz, 1H), 8.34 (dd, J = 1.6, 8.8 Hz, 1H), 7.87 (dd, J = 5.2, 8.8 Hz, 1H), 6.99 (d, J = 1.2 Hz, 1H), 2.68 (d, J = 1.2 Hz, 3H). |
| 1-5 | 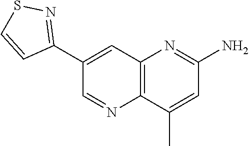 [SMILES: Cc1cc(N)nc2cc(-c3ccsn3)cnc12] | CM1-C | 35% | 7-(isothiazol-3-yl)-4-methyl-1,5-naphtbyridin-2- amine LCMS [M + 1]: 242.8. ¹H NMR (400 MHz, MeOD) δ = 9.19 (d, J = 2.0 Hz, 1H), 9.07 (d, J = 4.8 Hz, 1H), 8.38 (d, J = 2.0 Hz, 1H), 7.97 (d, J = 4.8 Hz, 1H), 6.95 (d, J = 0.8 Hz, 1H), 2.66 (d, J = 0.8 Hz, 3H). |
| 1-6 | 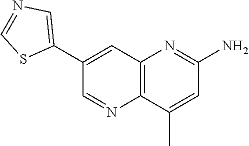 [SMILES: Cc1cc(N)nc2cc(-c3cncs3)cnc12] | CM1-B | 24% | 4-methyl-7-(thiazol-5-yl)-1,5-naphthyridin-2- amine LCMS [M + 1]: 243.2. ¹H NMR (400 MHz, MeOD) δ = 9.09 (s, 1H), 8.83 (d, J = 2.0 Hz, 1H), 8.39 (s, 1H), 8.02 (d, J = 2.0 Hz, 1H), 6.93 (d, J = 1.2 Hz, 1H), 2.63 (d, J = 1.2 Hz, 3H). |
| 1-7 | 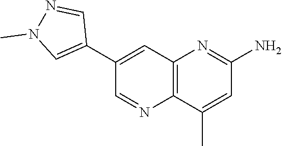 [SMILES: Cc1cc(N)nc2cc(-c3cnn(C)c3)cnc12] | CM1-A | 18% | 4-methyl-7-(1-methyl-1H-pyrazol-4-yl)-1,5- naphthyridin-2-amine LCMS [M + 1]: 240.3. ¹H NMR (400 MHz, MeOD) δ = 8.73 (d, J = 2.0 Hz, 1H), 8.16 (s, 1H), 7.97 (d, J = 0.8 Hz, 1H), 7.91 (d. J = 2.0 Hz, 1H), 6.85 (d, J = 1.2 Hz, 1H), 3.97 (s, 3H), 2.61 (d, J = 1.2 Hz, 3H). |
| 1-8 | 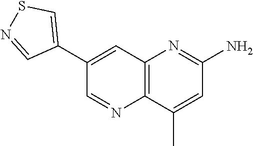 [SMILES: Cc1cc(N)nc2cc(-c3cnsc3)cnc12] | CM1-A | 4% | 7-(isothiazol-4-yl)-4-methyl-1,5-naphthyridin-2- amine LCMS [M + 1]: 243.2. ¹H NMR (400 MHz, MeOD) δ = 9.37 (s, 1H), 9.04 (s, 1H), 8.90 (d, J = 2.0 Hz, 1H), 8.13 (d, J = 2.0 Hz, 1H), 6.94 (d, J = 1.2 Hz, 1H), 2.67-2.64 (d, J = 1.2 Hz, 3H). |

### Example 1-9

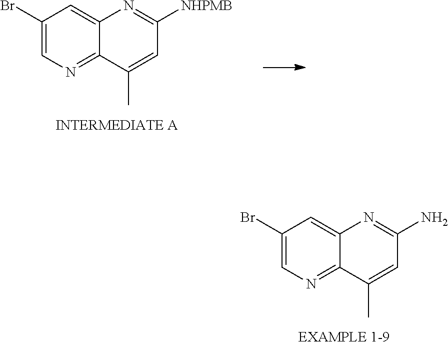

A mixture of Intermediate A (40.0 mg, 112 μmol, 1.00 eq.) and trifluoroacetic acid (770 mg, 6.75 mmol, 0.50 mL, 60.5 eq.) was stirred at 70° C. for 6 hours. The reaction mixture was concentrated under reduced pressure to give a residue. The residue was basified with NH4OH (0.10 mL) to pH 9. The solution was purified by prep-HPLC (neutral condition) to give 7-bromo-4-methyl-1,5-naphthyridin-2-amine, Example 1-9 (4.68 mg, 19.7 μmol, 17.6% yield) as a white solid. LCMS [M+1]: 240.2. ¹H NMR (400 MHz, MeOD-d₄) δ=8.54 (d, J=2.0 Hz, 1H), 8.00 (d, J=2.0 Hz, 1H), 6.93 (d, J=1.2 Hz, 1H), 2.60 (d, J=1.2 Hz, 3H).

### General Coupling Methods for the Preparation of Examples 2-1 to 2-10

Coupling Method 2-A (CM2-A, see General Scheme II): A mixture of a (halomethyl)heterocycle (2.65 mmol, 1.00 eq), 4,4,5,5-tetramethyl-2-(4,4,5,5-tetramethyl-1,3,2-dioxaborolan-2-yl)-1,3,2-dioxaborolane (1.08 g, 4.24 mmol, 1.60 eq), copper(I)iodide (50.5 mg, 265 μmol, 0.10 eq), lithium methoxide (211 mg, 5.57 mmol, 2.10 eq), and triphenylphosphine (90.4 mg, 345 μmol, 0.13 eq) in dimethyl formamide (5.00 mL) was stirred for 12 hours at 25° C. Water (10.0 mL) was added and the resulting mixture was extracted with ethyl acetate (3×15.0 mL). The combined organic layers were concentrated under vacuum to give the residue. The residue was purified by flash silica gel chromatography to give boronate 4b.

To a mixture of Intermediate B (60.0 mg, 111 μmol, 1.00 eq.) and boronate 4 (167 μmol, 1.50 eq.) in either dioxane (0.60 mL) and water (0.20 mL) or DMF (0.8 mL) was added Pd(dppf)Cl₂ (9.10 mg, 11.1 μmol, 0.1 eq.) and potassium carbonate (30.8 mg, 223 μmol, 2.00 eq.). The mixture was degassed and stirred at 100-120° C. for 2 hours. LCMS showed. The reaction mixture was filtered and then the filtrate was concentrated under reduced pressure. The residue was purified by prep-TLC to give Intermediate 5. The latter was diluted with TFA (0.20 mL) and stirred at 80° C. for 0.5 hr. LCMS. The reaction mixture was concentrated under vacuum to give a residue. The residue was added to saturated sodium bicarbonate aqueous solution (20.0 mL) and extracted with ethyl acetate (10.0 mL×3). The combined organic layers were dried over sodium sulfate, filtered and concentrated under reduced pressure to give a residue. The residue was purified by prep-HPLC (basic condition) to give a final product.

Coupling Method 2-B (CM2-B, see General Scheme III): A mixture of (halomethyl)heterocycle (683 μmol, 2.00 eq.), Intermediate C (200 mg, 342 μmol, 1.00 eq.), Pd(dppf)C₂ (27.9 mg, 34.2 μmol, 0.1 eq.), potassium carbonate (94.4 mg, 683 μmol, 2.00 eq.) in dioxane (3.00 mL) and water (0.80 mL) was degassed and stirred at 120° C. for 5 hours under nitrogen atmosphere. The mixture was filtered and concentrated under reduced pressure to give a residue. The residue was purified by column chromatography by prep-TLC to give intermediate 5. Following that a mixture of this intermediate product and TFA (1.00 mL) was stirred at 70° C. for 0.3 hours. The mixture was concentrated under reduced pressure to give a residue. The residue was adjusted to pH 9 with saturated ammonium hydroxide solution. Then the mixture was concentrated under vacuum. The residue was purified by prep-HPLC (basic condition) to give a final product.

Starting from intermediate B, following the teachings of the General Reaction Schemes 11 and III and coupling methods CM2-A, CM2-B Examples 2-1 to 2-10 were prepared as shown in Table 2:

TABLE 2

| | | | Yield | |
|---|---|---|---|---|
| | | Coupling | (2-3 | |
| Example | Structure | Method | steps) | Compound Name and Characterization |
| 2-1 | 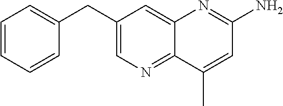 [SMILES: Cc1cc(N)nc2cc(Cc3ccccc3)cnc12] | CM2-A | 35% | 7-benzyl-4-methyl-1,5-naphthyridin-2-amine. LCMS [M + 1]: 250.0. 1H NMR (400 MHz, CDCl₃) δ = 8.57 (d, J = 2.0 Hz, 1H), 7.67 (d, J = 2.0 Hz, 1H), 7.33-7.28 (m, 2H), 7.26-7.19 (m, 3H), 6.75 (d, J = 0.8 Hz, 1H), 4.72 (br s, 2H), 4.13 (s, 2H), 2.68 (d, J = 0.6 Hz, 3H). |
| 2-2 | 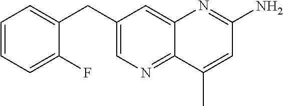 [SMILES: Cc1cc(N)nc2cc(Cc3ccccc3F)cnc12] | CM2-A | 12% | 7-(2-fluorobenzyl)-4-methyl-1,5-naphthyridin-2- amine. LCMS [M + 1]: 268.2. ¹H NMR (400 MHz, CD₃OD) δ = 8.47 (d, J = 2.0 Hz, 1H), 7.64 (d, J = 1.2 Hz, 1H), 7.36 (dt, J = 1.7, 7.6 Hz, 1H), 7.33- 7.27 (m, 1H), 7.19-7.15 (m, 1H), 7.15-7.09 (m, 1H), 6.88 (d, J = 1.0 Hz, 1H), 4.19 (s, 2H), 2.62 (d, J = 1.0 Hz, 3H). |
| 2-3 | 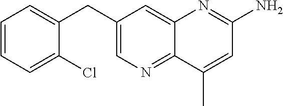 [SMILES: Cc1cc(N)nc2cc(Cc3ccccc3Cl)cnc12] | CM2-A | 2% | 7-(2-chlorobenzyl)-4-methyl-1,5-naphthyridin-2- amine. LCMS [M + 1]: 284.2. ¹H NMR (400 MHz, CD₃OD) δ = 8.47 (d, J = 2.0 Hz, 1H), 7.63-7.56 (m, 1H), 7.43 (ddd, J = 1.6, 7.6, 12.0 Hz, 2H), 7.31 (dquin, J = 1.6, 7.6 Hz, 2H), 6.88 (d, J = 1.2 Hz, 1H), 4.30 (s, 2H), 2.62 (d, J = 1.0 Hz, 3H). |
| 2-4 | 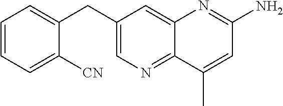 [SMILES: Cc1cc(N)nc2cc(Cc3ccccc3C#N)cnc12] | CM2-B | 24% | 2-((6-amino-8-methyl-1,5-naphthyridin-3- yl)methyl)benzonitrile. LCMS [M + 1]: 275.2. ¹H NMR (400 MHz, MeOD-d₄) δ = 8.51 (d, J = 2.0 Hz, 1H), 7.77 (d, J = 7.6 Hz, 1H), 7.71-7.63 (m, 2H), 7.56 (d, J = 7.8 Hz, 1H), 7.49-7.42 (m, 1H), 6.90 (d, J = 0.8 Hz, 1H), 4.40 (s, 2H), 2.62 (d, J = 1.2 Hz, 3H). |
| 2-5 | 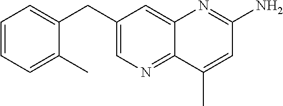 [SMILES: Cc1ccccc1Cc1cnc2c(C)cc(N)nc2c1] | CM2-A | 8% | 4-methyl-7-(2-methylbenzyl)-1,5-naphtbyridin-2- amine LCMS [M + 1]: 264.3. ¹H NMR (400 MHz, MeOD-d₄) δ = 8.42 (d, J = 2.0 Hz, 1H), 7.54- 7.47 (m, 1H), 7.26-7.16 (m, 4H), 6.88 (d, J = 0.9 Hz, 1H), 4.19 (s, 2H), 2.62 (d, J = 0.9 Hz, 3H), 2.26 (s, 3H). |
| 2-6 | 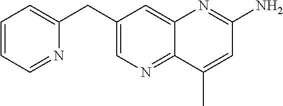 [SMILES: Cc1cc(N)nc2cc(Cc3ccccn3)cnc12] | CM2-B | 8% | 4-methyl-7-(2-pyridylmethyl)-1,5-naphthyridin-2- amine. LCMS [M + 1]: 251.2. ¹H NMR (400 MHz, MeOD-d₄) δ = 8.51 (dd, J = 0.8, 5.2 Hz, 1H), 8.50 (d, J = 2.0 Hz, 1H), 7.82 (dt, J = 2.0, 7.6 Hz, 1H), 7.72-7.68 (m, 1H), 7.42 (d, J = 7.6 Hz, 1H), 7.32 (ddd, J = 0.8, 5.6, 6.8 Hz, 1H), 6.89 (d, J = 1.2 Hz, 1H), 4.33 (s, 2H), 2.62 (d, J = 1.2 Hz, 3H). |
| 2-7 | 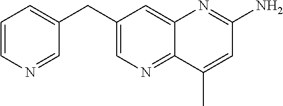 [SMILES: Cc1cc(N)nc2cc(Cc3cccnc3)cnc12] | CM2-B | 19% | 4-methyl-7-(3-pyridylmethyl)-1,5-naphthyridin-2- amine. LCMS [M + 1]: 251.3. ¹H NMR (400 MHz, MeOD) δ = 8.52 (d, J = 2.0 Hz, 1H), 8.45 (d, J = 2.4 Hz, 1H), 8.42 (dd, J = 1.6, 4.8 Hz, 1H), 7.76 (d, J = 8.0 Hz, 1H), 7.64 (d, J = 2.0 Hz, 1H), 7.40 (dd, J = 4.8, 8.0 Hz, 1H), 6.87 (d, J = 0.8 Hz, 1H), 4.20 (s, 2H), 2.60 (s, 3H). |
| 2-8 | 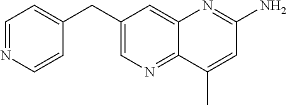 [SMILES: Cc1cc(N)nc2cc(Cc3ccncc3)cnc12] | CM2-B | 10% | 4-methyl-7-(4-pyridylmethyl)-1,5-naphthyridin-2- amine. LCMS [M + 1]: 251.3. ¹H NMR (400 MHz, MeOD) δ = 8.48-8.42 (m, 3H), 7.67 (d, J = 2.0 Hz, 1H), 7.38-7.34 (m, 2H), 6.89 (d, J = 0.8 Hz, 1H), 4.21 (s, 2H), 2.61 (d, J = 0.8 Hz, 3H). |
| 2-9 | 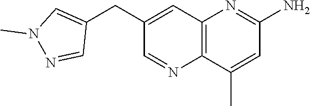 [SMILES: Cc1cc(N)nc2cc(Cc3cnn(C)c3)cnc12] | CM2-B | 5% | 4-methyl-7-[(1-methylpyrazol-4-yl)methyl]-1,5- naphthyridin-2-amine. LCMS [M + 1]: 254.3. ¹H NMR (400 MHz, MeOD) δ = 8.43 (d, J = 2.0 Hz, 1H), 7.68-7.63 (m, 1H), 7.47 (s, 1H), 7.36 (s, 1H), 6.87 (d, J = 0.8 Hz, 1H), 3.99 (s, 2H), 3.85 (s, 3H), 2.60 (d, J = 0.8) Hz, 3H). |
| 2-10 | 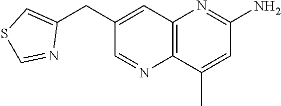 [SMILES: Cc1cc(N)nc2cc(Cc3cscn3)cnc12] | CM2-B | 17% | 4-methyl-7-(thiazol-4-ylmethyl)-1,5-naphthyridin- 2-amine. LCMS [M + 1]: 257.3. ¹H NMR (400 MHz, MeOD) δ = 8.97 (d, J = 2.0 Hz, 1H), 8.47 (d, J = 2.0 Hz, 1H), 7.71-7.65 (m, 1H), 7.40-7.34 (m, 1H), 6.86 (d, J = 0.8 Hz, 1H), 4.33 (s, 2H), 2.60 (d, J = 0.8 Hz, 3H). |

### General Coupling Methods for the Preparation of Examples 3-1 to 3-8

### Coupling Method 3-A (CM3-A):

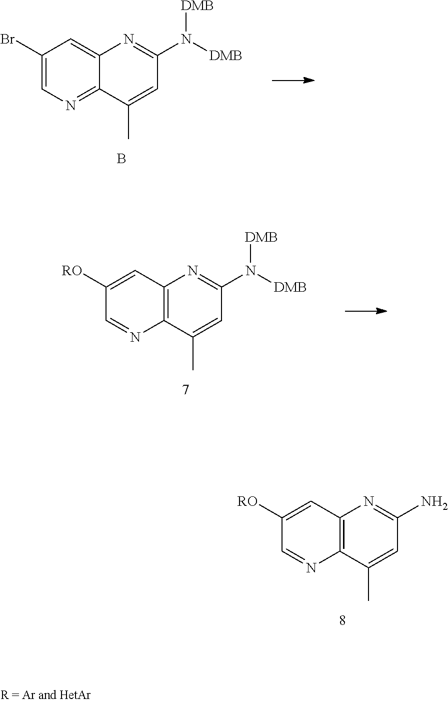

A mixture of Intermediate B (186 μmol, 1.00 eq), phenol ROH (557 μmol, 3.00 eq), copper(I)iodide (37.0 μmol, 0.200 eq), N,N-dimethylglycine (74.0 μmol, 0.400 eq) and cesium carbonate (576 μmol, 3.10 eq) in dioxane (2.00 mL) was degassed and stirred at 100° C. for 12 hours under nitrogen atmosphere. The reaction mixture was filtered and concentrated under reduced pressure to give a residue. The residue was purified by prep-TLC (petroleum ether/ethyl acetate 2:1) to give compound 7. The mixture of compound 7 in TFA (2.00 mL) was stirred at 70° C. for 0.5 hours. The reaction mixture was concentrated under reduced pressure to give a residue. The residue was basified with ammonium hydroxide (2.00 mL) to pH 9. The suspension was filtered, and the filtrate was purified by prep-HPLC (neutral condition) to give final product 8.

### Coupling Method 3-B (CM3-B):

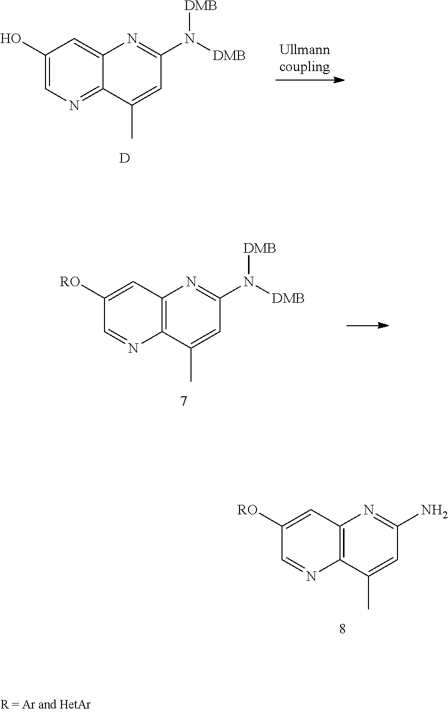

A mixture of Intermediate D (126 μmol, 1.00 eq), bromide RBr (378 μmol, 3.00 eq), copper(I)iodide (25.0 μmol, 0.200 eq), N,N-dimethylglycine (50.0 μmol, 0.400 eq) and cesium carbonate (391 μmol, 3.10 eq) in dioxane (1.00 mL) was degassed and stirred at 100° C. for 12 hours under nitrogen atmosphere. The reaction mixture was filtered and concentrated under reduced pressure to give a residue. The residue was purified by prep-TLC (petroleum ether/ethyl acetate 2:1) to give compound 7. The mixture of compound 7 and TFA (2.00 mL) was stirred at 70° C. for 0.5 hours. The reaction mixture was concentrated under reduced pressure to give a residue. The residue was basified with ammonium hydroxide (2.00 mL) to pH 9. The suspension was filtered, and the filtrate was purified by prep-HPLC (neutral condition) to give final product 8.

### Coupling Method 3-C(CM3-C):

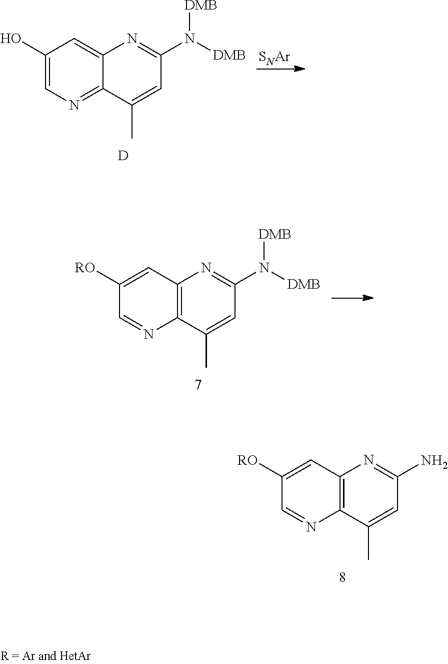

A mixture of Intermediate D (60.0 mg, 126 μmol, 1.00 eq) and arylfluoride RF(379 μmol, 3.00 eg), cesium carbonate (123 mg, 379 μmol, 3.00 eq) in N,N-dimethylformamide (2.00 mL) was heated at 100° C. under nitrogen atmosphere for 3 hours. Water (5.00 mL) was added and the mixture was extracted with ethyl acetate (15.0 mL×3), the combined organic layers were washed with brine (10.0 mL×3), dried over sodium sulfate, filtered and concentrated under vacuum to give a residue which was purified by silica gel chromatography to give intermediate 7. The mixture of compound 7 and TFA (2.00 mL) was stirred at 70° C. for 0.5 hours. The reaction mixture was concentrated under reduced pressure to give a residue. The residue was basified with ammonium hydroxide (2.00 mL) to pH 9. The suspension was filtered, and the filtrate was purified by prep-HPLC (neutral condition) to give final product 8.

Starting from intermediates C or D, following the teachings of General Reaction Scheme IV and coupling methods CM3-A, CM3-B and CM3-C Examples 3-1 to 3-8 were prepared as shown in Table 3:

TABLE 3

| | | Coupling | Yield | |
|---|---|---|---|---|
| Example | Structure | Method | (2 steps) | Compound Name and Characterization |
| 3-1 | 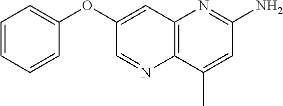 [SMILES: Cc1cc(N)nc2cc(Oc3ccccc3)cnc12] | CM3-A | 53% | 4-methyl-7-phenoxy-1,5-naphthyridin-2-amine. LCMS [M + 1]: 252.2. ¹H NMR (400 MHz, MeOD- d₄) δ = 8.38 (d, J = 2.8 Hz, 1H), 7.49-7.44 (m, 2H), 7.28-7.24 (m, 1H), 7.20 (d, J = 2.4 Hz, 1H), 7.17-7.14 (m, 2H), 6.82 (d, J = 0.8 Hz, 1H), 2.61 (d, J = 0.8 Hz, 3H). |
| 3-2 | 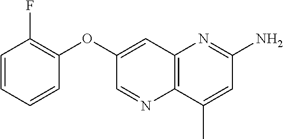 [SMILES: Cc1cc(N)nc2cc(Oc3ccccc3F)cnc12] | CM3-A | 28% | 7-(2-fluorophenoxy)-4-methyl-1,5-naphthyridin-2- amine. LCMS [M + 1]: 270.2. ¹H NMR (400 MHz, MeOD- d₄) δ = 8.40 (d, J = 2.4 Hz, 1H), 7.33-7.28 (m, 4H), 7.09 (m, 1H), 6.80 (d, J = 1.2 Hz, 1H), 2.60 (d, J = 0.8 Hz, 3H). |
| 3-3 | 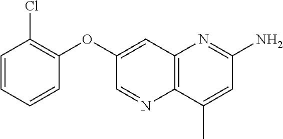 [SMILES: Cc1cc(N)nc2cc(Oc3ccccc3Cl)cnc12] | CM3-A | 24% | 7-(2-chlorophenoxy)-4-methyl-1,5-naphthyridin-2- amine. LCMS [M + 1]: 286.0. ¹H NMR (400 MHz, CDCl₃) δ = 8.52 (d, J = 2.8 Hz, 1H), 7.50 (dd, J = 8.0, 1.6 Hz, 1H), 7.31 (td, J = 7.6, 1.6 Hz, 1H), 7.21 (dd, J = 8.0, 1.6 Hz, 1H), 7.17 (d, J = 8.0, 1.6 Hz, 1H), 7.11 (d, J = 2.8 Hz, 1H), 6.70 (d, J = 1.2 Hz, 1H), 4.73 (br, s, 2H), 2.69 (d, J = 0.8 Hz, 3H). |
| 3-4 | 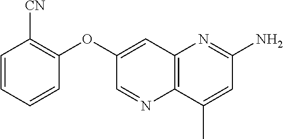 [SMILES: Cc1cc(N)nc2cc(Oc3ccccc3C#N)cnc12] | CM3-C | 41% | 2-((6-amino-8-methyl-1,5-naphthyridin-3- yl)oxy)benzonitrile. LCMS [M + 1]: 277.2. ¹H NMR (400 MHz, MeOD- d₄) δ = 8.46 (d, J = 2.8 Hz, 1H), 7.86 (dd, J = 7.6, 1.6 Hz, 1H), 7.75-7.70 (m, 1H), 7.39 (dt, J = 7.6, 0.8 Hz, 1H), 7.36 (d, J = 2.8 Hz, 1H), 7.21 (d, J = 8.4 Hz, 1H), 6.90 (d, J = 0.8 Hz, 1H), 2.65 (d, J = 0.8 Hz, 3H) |
| 3-5 | 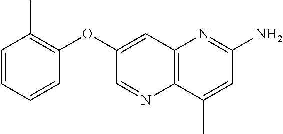 [SMILES: Cc1ccccc1Oc1cnc2c(C)cc(N)nc2c1] | CM3-A | 75% | 4-methyl-7-(2-methylphenoxy)-1,5-naphtbyridin- 2-amine. LCMS [M + 1]: 266.2. ¹H NMR (400 MHz, CDCl₃) δ = 8.58 (d, J = 2.8 Hz, 1H), 7.29 (d, J = 7.2 Hz, 1H), 7.26-7.19 (m, 1H), 7.18-7.12 (m, 1H), 7.07- 7.02 (m, 2H), 6.67 (d, J = 0.8 Hz, 1H), 4.69 (br s, 2H), 2.69 (d, J = 0.8 Hz, 3H), 2.23 (s, 3H). |
| 3-6 | 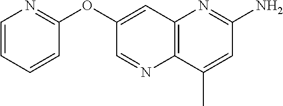 [SMILES: Cc1cc(N)nc2cc(Oc3ccccn3)cnc12] | CM3-B | 52% | 4-methyl-7-(pyridin-2-yloxy)-1,5-naphthyridin-2- amine. LCMS [M + 1]: 253.2. ¹H NMR (400 MHz, MeOD- d₄) δ = 8.42 (d, J = 2.4 Hz, 1H), 8.18 (dd, J = 4.4, 1.6 Hz, 1H), 7.97-7.86 (m, 1H), 7.58 (d, J = 2.4 Hz, 1H), 7.21 (dd, J = 6.4, 4.8 Hz, 1H), 7.16 (d, J = 8.0 Hz, 1H), 6.90 (d, J = 0.8 Hz, 1H), 2.65 (d, J = 0.8 Hz, 3H). |
| 3-7 | 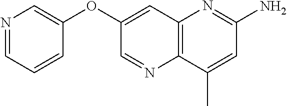 [SMILES: Cc1cc(N)nc2cc(Oc3cccnc3)cnc12] | CM3-B | 42% | 4-methyl-7-(3-pyridyloxy)-1,5-paphthyridin-2- amine. LCMS [M + 1]: 253.2. ¹H NMR (400 MHz, CDCl₃) δ = 8.56 (d, J = 2.8 Hz, 1H), 8.53 (d, J = 2.8 Hz, 1H), 8.49 (dd, J = 4.4, 1.2 Hz, 1H), 7.46-7.43 (m, 1H), 7.36-7.32 (m, 1H), 7.31 (d, J = 2.4 Hz, 1H), 6.73 (d, J = 1.2 Hz, 1H), 4.78 (br, s, 2H), 2.70 (d, J = 0.8 Hz, 3H). |
| 3-8 | 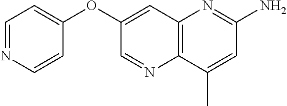 [SMILES: Cc1cc(N)nc2cc(Oc3ccncc3)cnc12] | CM3-B | 40% | 4-methyl-7-(pyridin-4-yloxy)-1,5-naphtbyridin-2- amine. LCMS [M + 1]: 253.2. ¹H NMR (400 MHz, MeOD- d₄) δ = 8.50 (d, J = 1.6 Hz, 1H), 8.49 (d, J = 1.6 Hz, 1H), 8.42 (d, J = 2.4 Hz, 1H), 7.55 (d, J = 2.8 Hz, 1H), 7.11 (d, J = 1.2 Hz, 1H), 7.10 (d, J = 1.6 Hz, 1H), 6.92 (d, J = 0.8 Hz, 1H), 2.65 (d, J = 1.2 Hz, 3H). |

### General Coupling Method for the Preparation of Examples 4-1 to 4-9

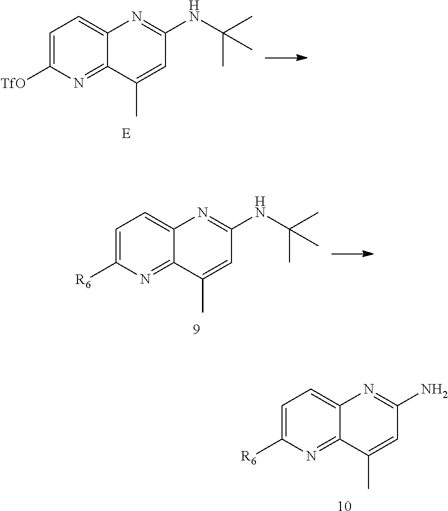

A mixture of Intermediate E (193 μmol, 1.00 eq.), boron esters (289 μmol, 1.50 eq.), Pd(dppf)Cl₂ (19.3 μmol, 0.10 eq.) and cesium carbonate (2.00 eq.) in dioxane (1.00 mL) and water (0.20 mL) was degassed and then stirred at 100° C. for 0.5 hour under nitrogen atmosphere. The reaction mixture was diluted with water (5.00 mL) and extracted with ethyl acetate (5.00 mL×3). The combined organic layers were washed with brine (10.0 mL), dried, filtered and concentrated to give a residue containing compound 9 which was used in the next step without further purification. The residue was treated with trifluoroacetic acid (5.50 mL, 197 eq.) and stirred at 70° C. for 8 hours. The reaction mixture was concentrated under reduced pressure to give a residue. The residue was dissolved in methyl alcohol (3.00 mL) and pH was adjusted to 8 with triethylamine. The solution was purified by prep-HPLC (neutral condition) to give product 10.

Starting from intermediates E, following the teachings of General Reaction Scheme V and the general coupling method above, Examples 4-1 to 4-9 were prepared as shown in Table 4:

TABLE 4

| | | Yield | |
|---|---|---|---|
| Example | Structure | (2 steps) | Compound Name and Characterization |
| 4-1 | 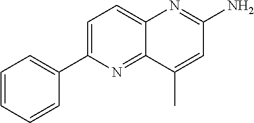 [SMILES: Cc1cc(N)nc2ccc(-c3ccccc3)nc12] | 3% | 4-methyl-6-phenyl-1,5-naphthyridin-2-amine. LCMS [M + 1] = 236.3. ¹H NMR (400 MHz, MeOD) δ = 8.20-8.14 (d, J = 8.4 Hz, 2H), 8.04 (d, J = 8.8 Hz, 1H), 7.89 (d, J = 8.8 Hz, 1H), 7.52- 7.45 (t, J = 7.2 Hz, 2H), 7.43-7.38 (t, J = 7.2 Hz, 1H), 6.94 (s, 1H), 2.73 (d, J = 0.8 Hz, 3H). |
| 4-2 | 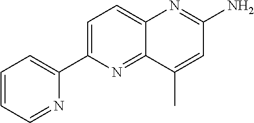 [SMILES: Cc1cc(N)nc2ccc(-c3ccccn3)nc12] | 40% | 4-methyl-6-(2-pyridyl)-1,5-naphthyridin-2-amine. LCMS [M + 1] = 237.3. ¹H NMR (400 MHz, MeOD) δ = 8.63 (br d, J = 4.8 Hz, 1H), 8.55 (d, J = 8.0 Hz, 1H), 8.48 (d, J = 8.8 Hz, 1H), 7.96-7.88 (m, 2H), 7.42-7.36 (m, 1H), 6.95 (s, 1H), 2.74 (s, 3H). |
| 4-3 | 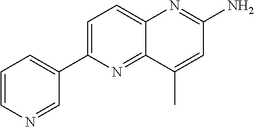 [SMILES: Cc1cc(N)nc2ccc(-c3cccnc3)nc12] | 13% | 4-methyl-6-(3-pyridyl)-1,5-naphthyridin-2-amine. LCMS [M + 1] = 237.3. ¹H NMR (400 MHz, MeOD) δ = 9.34 (d, J = 1.6 Hz, 1H), 8.62-8.58 (dd, J = 2.0 Hz, J = 8.0 Hz, 1H), 8.56 (dd, J = 1.6, 4.8 Hz, 1H), 8.10 (d, J = 8.8 Hz, 1H), 7.93 (d, J = 8.8 Hz, 1H), 7.56 (dd, J = 4.8, 7.6 Hz, 1H), 6.96 (d, J = 0.8 Hz, 1H), 2.72 (d, J = 0.8 Hz, 3H). |
| 4-4 | 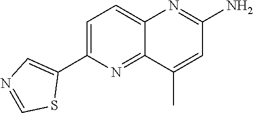 [SMILES: Cc1cc(N)nc2ccc(-c3cncs3)nc12] | 15% | 4-methyl-6-thiazol-5-yl-1,5-naphthyridin-2-amine. LCMS [M + 1] = 243.2. ¹H NMR (400 MHz, MeOD) δ = 9.00 (s, 1H), 8.45 (s, 1H), 8.02 (d, J = 8.8 Hz, 1H), 7.86 (d, J = 8.8 Hz, 1H), 6.93 (s, 1H), 2.65 (s, 3H). |
| 4-5 | 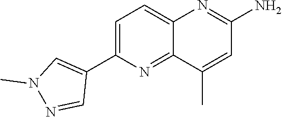 [SMILES: Cc1cc(N)nc2ccc(-c3cnn(C)c3)nc12] | 27% | 4-methyl-6-(1-methylpyrazol-4-yl)-1,5- naphthyridin-2-amine. LCMS [M + 1] = 240.3. ¹H NMR (400 MHz, MeOD) δ = 8.18 (s, 1H), 8.06 (s, 1H), 7.80-7.76 (d, J = 8.8, 1H), 7.75-7.70 (d, J = 8.8, 1H), 6.88 (s, 1H), 3.96 (s, 3H), 2.66 (s, 3H). |
| 4-6 | 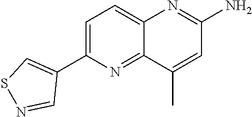 [SMILES: Cc1cc(N)nc2ccc(-c3cnsc3)nc12] | 41% | 6-isothiazol-4-yl-4-methyl-1,5-naphthyridin-2- amine. LCMS [M + 1] = 243.2. ¹H NMR (400 MHz, MeOD-d₄) δ = 9.43 (s, 1H), 9.23 (s, 1H), 7.99 (d, J = 8.6 Hz, 1H), 7.88 (d, J = 8.6 Hz, 1H), 6.94 (d, J = 0.8 Hz, 1H), 2.71 (s, 3H). |
| 4-7 | 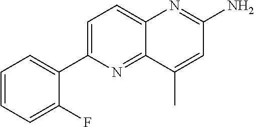 [SMILES: Cc1cc(N)nc2ccc(-c3ccccc3F)nc12] | 11% | 6-(2-fluorophenyl)-4-methyl-1,5-naphthyridin-2- amine. LCMS [M + 1] = 254.3. ¹H NMR (400 MHz, MeOD) δ = 8.10 (dt, J = 1.8, 8.0 Hz, 1H), 7.97- 7.92 (m, 1H), 7.90-7.85 (m, 1H), 7.49-7.40 (m, 1H), 7.34-7.28 (t, J = 7.6, 1H), 7.26-7.19 (m, 1H), 6.95 (d, J = 1.2 Hz, 1H), 2.70 (d, J = 0.8 Hz, 3H). |
| 4-8 | 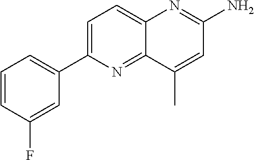 [SMILES: Cc1cc(N)nc2ccc(-c3cccc(F)c3)nc12] | 31% | 6-(3-fluorophenyl)-4-methyl-1,5-naphthyridin-2- amine. LCMS [M + 1] = 254.3. ¹H NMR (400 MHz, MeOD) δ = 8.02 (d, J = 8.8 Hz, 1H), 7.98-7.91 (m, 2H), 7.88 (d, J = 8.8 Hz, 1H), 7.52-7.44 (m, 1H), 7.16-7.08 (m, 1H), 6.93 (s, 1H), 2.71 (s, 3H). |
| 4-9 | 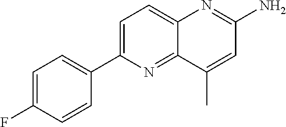 [SMILES: Cc1cc(N)nc2ccc(-c3ccc(F)cc3)nc12] | 33% | 6-(4-fluorophenyl)-4-methyl-1,5-naphthyridin-2- amine. LCMS [M + 1] = 254.2. ¹H NMR (400 MHz, MeOD) δ = 8.20 (br dd, J = 6.0, 8.0 Hz, 2H), 8.00 (br d, J = 8.8 Hz, 1H), 7.87 (br d, J = 8.8 Hz, 1H), 7.20 (br t, J = 8.8 Hz, 2H), 6.93 (s, 1H), 2.71 (s, 3H). |

### Example 4-10

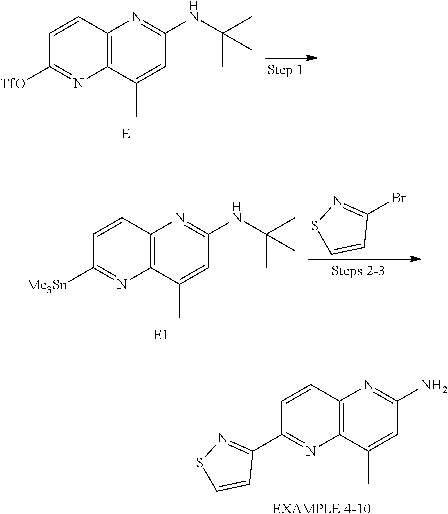

Step 1. A mixture of Intermediate E (120 mg, 330 μmol, 1.00 eq.), trimethyl(trimethylstannyl)stannane (541 mg, 1.65 mmol, 342 μL, 5.00 eq.), Pd(PPh₃)₄ (38.2 mg, 33.3 μmol, 0.10 eq.) and lithium chloride (42.0 mg, 991 μmol, 20.3 μL, 3.00 eq.) in toluene (0.20 mL) and dioxane (1.00 mL) was degassed and stirred at 100° C. for 2 hrs under nitrogen atmosphere. The reaction mixture was concentrated under reduced pressure to give a residue. Crude product compounds E1 (128 mg, crude), a brown oil, was used in the next step without further purification. LCMS [M+1]⁺=380.2

Step 2: A mixture of Intermediate E1 (120 mg, 317 μmol, 1.00 eq.), 3-bromoisothiazole (156 mg, 952 μmol, 3.00 eq.) and Pd(PPh₃)₄ (36.7 mg, 31.7 μmol, 0.10 eq.), in toluene (1.00 mL) was degassed and stirred at 100° C. for 2 hrs under nitrogen atmosphere. The reaction mixture was diluted with water (20.0 mL) and extracted with ethyl acetate (30.0 mL×3). The combined organic layers were washed with brine (20.0 mL), dried, filtered and concentrated under reduced pressure to give a residue. The residue was purified by prep-TLC (SiO₂, Petroleum ether/Ethyl acetate 2:1) to give N-tert-butyl-6-isothiazol-3-yl-4-methyl-1,5-naphthyridin-2-amine (46.0 mg, 71.5 μmol, 22.5% yield, 46.4% purity) as a yellow solid. LCMS [M+1]⁺=299.2.

Step 3: A solution of N-tert-butyl-6-isothiazol-3-yl-4-methyl-1,5-naphthyridin-2-amine (45.0 mg, 70.0 μmol, 1.00 eq.) in TFA (3.91 mL) was stirred at 70° C. for 2 hrs. The reaction mixture was reduced pressure to give a residue. The residue was dissolved in methyl alcohol (3.00 mL) and pH was adjusted to 8 with triethylamine. The residue was purified by prep-HPLC (neutral condition) to give 6-isothiazol-3-yl-4-methyl-1,5-naphthyridin-2-amine, Example 4-10 (8.02 mg, 32.44 μmol, 46.4% yield) as a yellow solid. LCMS [M+1]⁺=243.2. ¹H NMR (400 MHz, MeOD-d₄) δ=8.95 (d, J=4.8 Hz, 1H), 8.35 (d, J=8.8 Hz, 1H), 8.18 (d, J=4.8 Hz, 1H), 7.89 (d, J=8.8 Hz, 1H), 6.94 (d, J=1.2 Hz, 1H), 2.71 (d, J=1.2 Hz, 3H).

### General Coupling Method for the Preparation of Examples 5-1 to 5-3

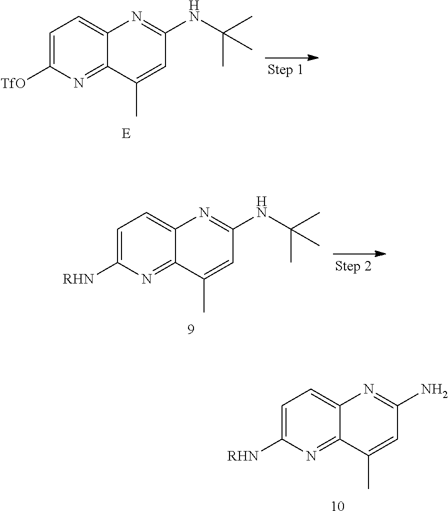

Step 1: To a solution of Intermediate E (86.0 mg, 237 μmol, 1.00 eq.) and a primary amine (2.84 mmol, 12.0 eq.) in dimethylsulfoxide (3.00 mL) was added diisopropylethylamine (91.7 mg, 710 μmol, 124 μL, 3.00 eq.) and 4A molecular sieves (90.0 mg). The mixture was stirred at 100° C. for 12 hours. The reaction mixture was diluted with water (10.0 mL) and extracted with dichloromethane (10.0 mL×2). The combined organic layers were washed with brine (10.0 mL×2), dried over anhydrous sodium sulfate, filtered and concentrated under reduced pressure to give a residue. The residue was purified by prep-TLC (SiO₂, petroleum ether/ethyl acetate 2:1) to give intermediate 9.

Step 2: A solution of Intermediate 9 (58.0 μmol, 1.00 eq.) in trifluoroacetic acid (1.00 mL) was stirred at 70° C. for 5 hours. The reaction mixture was concentrated under reduced pressure to give a residue. The residue was purified by prep-HPLC (neutral condition) to give final compound 10.

Starting from intermediates E, following the teachings of General Reaction Scheme V and the general coupling method above, Examples 5-1 to 5-3 were prepared as shown in Table 5:

TABLE 5

| | | Yield | |
|---|---|---|---|
| Example | Structure | (2 steps) | Compound Name and Characterization |
| 5-1 | 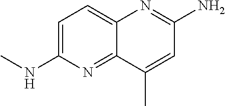 | 18% | N²,8-dimethyl-1,5-naphthyridine-2,6- diamine. LCMS [M + 1]⁺:189.3. ¹H NMR (400 MHz, DMSO-d₆) δ = 7.47 (d, J = 8.8 Hz, 1H), 6.75 (d, J = 9.2 Hz, 1H), 6.65 (s, 1H), 6.50 (q, J = 4.0 Hz, 1H), 5.75 (s, 2H), 2.86 (d, J = 4.8 Hz, 3H), 2.43 (s, 3H). |
| 5-2 | 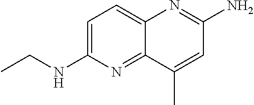 | 13% | N⁶-ethyl-4-methyl-1,5-naphthyridine-2,6- diamine. LCMS [M + 1] = 203.3. ¹H NMR (400 MHz, MeOD) δ = 7.53 (d, J = 9.2 Hz, 1 H), 6.77 (d, J = 8.8 Hz, 1 H), 6.75 (d, J = 1.2 Hz, 1 H), 3.45 (q, J = 7.2 Hz, 2 H), 2.51 (d, J = 1.2 Hz, 3 H), 1.26 (t, J = 7.2 Hz, 3 H). |
| 5-3 | 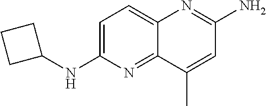 [SMILES: Cc1cc(N)nc2ccc(NC3CCC3)nc12] | 13% | N⁶-cyclobutyl-4-methyl-1,5-naphthyridine-2,6- diamine. LCMS [M + 1] = 229.0. ¹H NMR (400 MHz, CDCl₃) δ = 7.69 (d, J = 8.8 Hz, 1 H), 6.70 (s, 1 H), 6.68 (s, 1 H), 4.74 (br d, J = 4.4 Hz, 1 H), 4.46-4.34 (m, 3 H), 2.57 (d, J = 0.8 Hz, 3 H), 2.53-2.45 (m, 2 H), 1.96-1.88 (m, 2 H), 1.85- 1.77 (m, 2 H). |

### Example 6-1

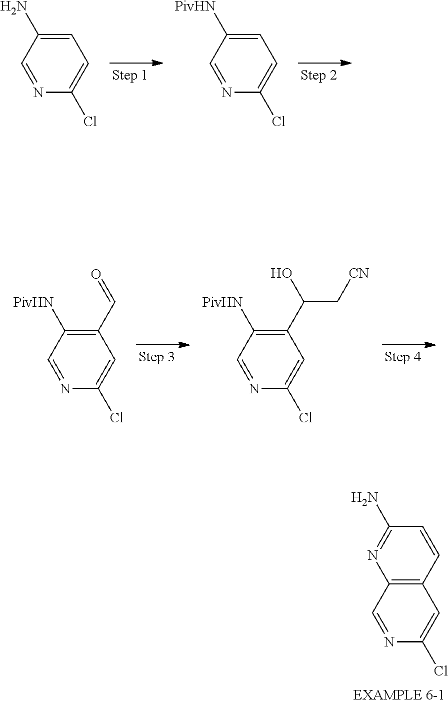

### Example 6-1

Step 1: To an ice-cooled solution of 6-chloropyridin-3-amine (10.0 g, 77.8 mmol, 1.00 eq.) and triethylamine (9.44 g, 93.3 mmol, 13.0 mL, 1.20 eq.) in dichloromethane (70.0 mL) was added a solution of 2,2-dimethylpropanoyl chloride (9.85 g, 81.7 mmol, 10.1 mL, 1.05 eq.) in dichloromethane (30.0 mL) drop wise at 0° C. over a period of 1 hour. The reaction mixture was stirred at 15° C. for 2 hours. The reaction mixture was poured into 5% aqueous solution of sodium hydroxide (2.00 mL) and the dichloromethane layer separated. The organic layer was washed with 5% aqueous solution of sodium hydroxide, dried over sodium sulfate, filtered and concentrated to give a residue. The residue was purified by column chromatography (SiO₂, petroleum ether/ethyl acetate 10-50%) to give N-(6-chloro-3-pyridyl)-2,2-dimethyl-propanamide (15.8 g, 74.3 mmol, 95.5% yield) as a white solid. ¹H NMR (400 MHz, CDCl₃) δ=8.31-8.25 (m, 1H), 8.13-8.00 (m, 1H), 7.42 (br s, 1H), 7.22-7.15 (m, 1H), 1.29-1.14 (s, 9H).

Step 2: To a solution of N-(6-chloro-3-pyridyl)-2,2-dimethyl-propanamide (2.00 g, 9.40 mmol, 1.00 eq.) in tetrahydrofuran (20.0 mL) was added tert-Butyllithium (1.30 M, 15.9 mL, 2.20 eq.) at −78° C. drop wise. After stirring for 1.5 hours, dimethyl formamide (4.81 g, 65.8 mmol, 5.06 mL, 7.00 eq.) was added to the reaction mixture. The reaction mixture was stirred at −78° C. for 1 hour. The reaction mixture was quenched with aq. ammonium chloride (5.00 mL) and extracted with ethyl acetate (10.0 mL-3). Combined organic phase was washed with brine (10.0 mL), dried over sodium sulfate, filtered and concentrated to give a residue. The residue was purified by column chromatography (SiO₂, petroleum ether/ethyl acetate 10-20%) to give N-(6-chloro-4-formyl-3-pyridyl)-2,2-dimethyl-propanamide (430 mg, 1.79 mmol, 9.50% yield) as a yellow solid. ¹H NMR (400 MHz, CDCl₃) δ=10.82 (br s, 1H), 10.00 (s, 1H), 9.94 (s, 1H), 7.61 (s, 1H), 1.37 (s, 9H).

Step 3: To a solution of acetonitrile (251 mg, 6.11 mmol, 321 μL 2.10 eq.) in tetrahydrofuran (5.00 mL) was added LDA (2 M, 3.05 mL, 2.10 eq.) drop wise at −78° C. After stirring for 0.5 hour, a solution of N-(6-chloro-4-formyl-3-pyridyl)-2,2-dimethyl-propanamide (0.70 g, 2.91 mmol, 1.00 eq.) in tetrahydrofuran (5.00 mL) was added to the reaction mixture. The reaction mixture was stirred at −78° C. for 0.5 hour. The reaction mixture was diluted with water (2.00 mL) and extracted with ethyl acetate (2.00 mL×3). Combined organic phase was washed with brine (2.00 mL), dried over sodium sulfate, filtered and concentrated to give a residue. The residue was purified by column chromatography (SiO₂, petroleum ether/ethyl acetate 5-10%) to give N-[6-chloro-4-(2-cyano-1-hydroxy-ethyl)-3-pyridyl]-2,2-dimethyl-propanamide (870 mg, crude) as a yellow solid. LCMS [M+1]:282.1.

Step 3: A mixture of N-[6-chloro-4-(2-cyano-1-hydroxy-ethyl)-3-pyridyl]-2,2-dimethyl-propanamide (870 mg, 3.09 mmol, 1.00 eq.) and hydrochloric acid (3 M, 8.00 mL, 7.77 eq.) was stirred at 160° C. for 5 min in a microwave. The reaction mixture was neutralized with saturated aq. sodium bicarbonate (5.00 mL) to pH 8. The solid was filtered and concentrated under reduced pressure to give 6-chloro-1,7-naphthyridin-2-amine, Example 6-1 (210 mg, 1.15 mmol, 37.3% yield) as a yellow solid. LCMS [M+1]:180.2. ¹H NMR (400 MHz, MeOD-d₄) δ=8.61 (s, 1H), 7.91 (d, J=9.0 Hz, 1H), 7.65 (s, 1H), 7.07 (d, J=9.0 Hz, 1H).

### Example 6-2

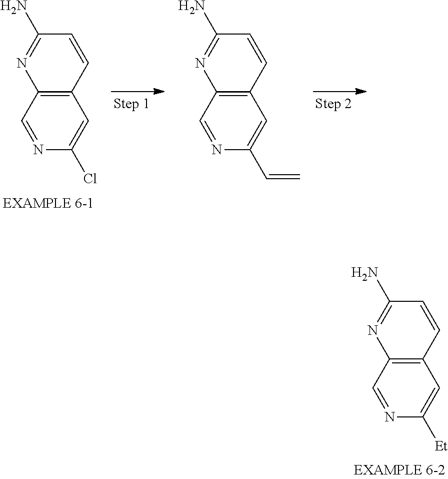

Step 1: A mixture of Example 6-1 (40.0 mg, 223 μmol, 1.00 eq.), Pd(PPh₃)₄ (25.7 mg, 22.3 μmol, 0.10 eq.) and tributyl(vinyl)stannane (141 mg, 445 μmol, 130 μL, 2.00 eq.) in toluene (2.00 mL) was degassed and stirred at 100° C. for 8 hours under nitrogen atmosphere. The reaction mixture was diluted with saturated potassium fluoride solution (10.00 mL) and extracted with ethyl acetate (5.00 mL×2). The combined organic layers were washed with brine (10.0 mL), dried over anhydrous sodium sulfate, filtered and concentrated under reduced pressure to give a residue. The residue was purified by prep-TLC (SiO₂, ethyl acetate) to give 6-vinyl-1,7-naphthyridin-2-amine (17.0 mg, 98.2 μmol, 44.1% yield) as a yellow solid. LCMS [M+1]⁺=172.2.

Step 2: To a solution of 6-vinyl-1,7-naphthyridin-2-amine (10.0 mg, 58.4 μmol, 1.00 eq.) in ethyl acetate (1.00 mL) was added 1-% palladium on activated carbon (10.0 mg, 10.0/o purity) under nitrogen atmosphere. The reaction mixture was purged with hydrogen 3 times. The mixture was stirred at 20° C. for 10 mins under hydrogen (15 psi). The reaction mixture was filtered and concentrated under reduced pressure to give a residue. The residue was purified by prep-HPLC (neutral condition) to give 6-ethyl-1,7-naphthyridin-2-amine, Example 6-2 (1.52 mg, 8.72 μmol, 14.9% yield) as a white solid. LCMS [M+1]⁺=172.2. ¹H NMR (400 MHz, MeOD) δ=8.75 (s, 1H), 7.93 (d, J=9.2 Hz, 1H), 7.47 (s, 1H), 7.05 (d, J=8.8 Hz, 1H), 2.88 (q, J=8.0 Hz, 2H), 1.32-1.35 (m, 3H).

### Example 6-3

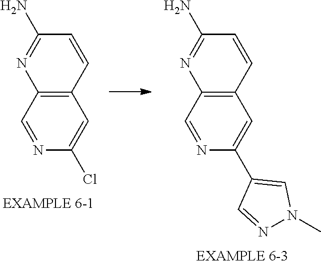 [SMILES: Nc1ccc2cc(Cl)ncc2n1 ; Cn1cc(-c2cc3ccc(N)nc3cn2)cn1]

A mixture of Example 6-1 (20.0 mg, 111 μmol, 1.00 eq.), 1-methyl-4-(4,4,5,5-tetramethyl-1,3,2-dioxaborolan-2-yl)pyrazole (46.3 mg, 223 μmol, 2.00 eq.), Pd(dppf)Cl₂ (8.15 mg, 11.1 μmol, 0.10 eq.), cesium carbonate (72.6 mg, 223 μmol, 2.00 eq.) in dioxane (0.50 mL) and water (0.10 mL) was degassed and stirred at 100° C. for 8 hours under nitrogen atmosphere. The reaction mixture was diluted with water (10.0 mL) and extracted with ethyl acetate (10.0 mL×3). The combined organic layers were washed with brine (20.0 mL×2), dried over sodium sulfate, filtered and concentrated under reduced pressure to give a residue. The residue was purified by prep-HPLC (basic condition) to give 6-(1-methylpyrazol-4-yl)-1,7-naphthyridin-2-amine, Example 6-3 (2.90 mg, 12.9 μmol, 11.6% yield) as a yellow solid. LCMS [M+1]: 226.3. ¹H NMR (400 MHz, CDCl₃) δ=9.05 (s, 1H), 7.98 (d, J=1.2 Hz, 2H), 7.87 (d, J=8.8 Hz, 1H), 7.61 (s, 1H), 6.90 (d, J=8.8 Hz, 1H), 4.92 (br s, 2H), 3.98 (s, 3H).

### Example 6-4

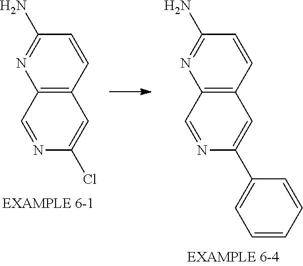 [SMILES: Nc1ccc2cc(Cl)ncc2n1 ; Nc1ccc2cc(-c3ccccc3)ncc2n1]

A mixture of Example 6-1 (30.0 mg, 167 μmol, 1.00 eq.), phenylboronic acid (40.7 mg, 334 μmol, 2.00 eq.), Pd(dppf)Cl₂ (12.2 mg, 16.7 μmol, 0.10 eq.), cesium carbonate (109 mg, 334 μmol, 2.00 eq.) in dioxane (0.50 mL) and water (0.10 mL) was degassed and stirred at 100° C. for 2 hours under nitrogen atmosphere. The reaction mixture was filtered and concentrated under reduced pressure to give a residue. The residue was purified by prep-HPLC (basic condition) to give 6-phenyl-1,7-naphthyridin-2-amine, Example 6-4 (5.44 mg, 24.5 μmol, 14.7% yield) as an off-white solid. LCMS [M+1]: 222.3. ¹H NMR (400 MHz, MeOD) δ=8.90 (s, 1H), 8.05-7.97 (m, 4H), 7.51-7.45 (m, 2H), 7.41-7.36 (m, 1H), 7.08 (d, J=9.0 Hz, 1H).

### General Coupling Methods for the Preparation of Examples 7-1 to 7-4

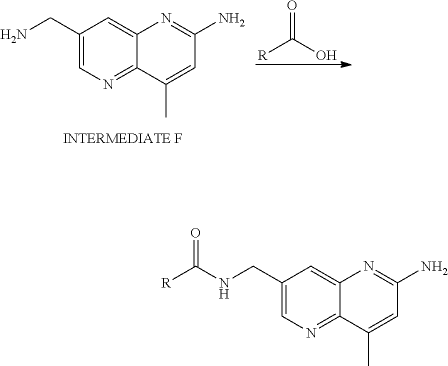

Coupling Method 7-A (CM7-A): To a solution of a carboxylic acid (128 μmol, 1.00 eq.) in dimethyl formamide (1.00 mL) was added HATU (72.7 mg, 191 μmol, 1.50 eq.) and triethylamine (38.7 mg, 383 μmol, 53.2 μL, 3.00 eq.). The mixture was stirred at 20° C. for 0.5 hour and then Intermediate F (31.2 mg, 166 μmol, 1.30 eq.) was added to the mixture and the resulting was stirred at 20° C. for 0.5 hour. The mixture was concentrated under reduced pressure to give a residue. The residue was purified by prep-HPLC (HCl condition) to give a desired amid product.

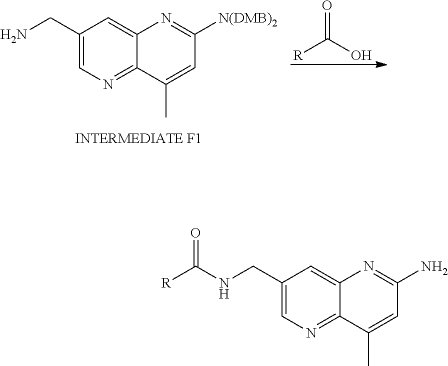

Coupling Method 7-B (CM7-B): To a mixture of Intermediate F1 (217 mg, 443 μmol, 1.10 eq.) and a carboxylic acid (403 μmol, 1.00 eq.) in dichloromethane (1.50 mL) was added T3P (50% in ethyl acetate) (1.28 g, 2.01 mmol, 1.20 mL, 50% purity, 5.00 eq.) and triethylamine (122 mg, 1.21 mmol, 168 μL, 3.00 eq.). The mixture was stirred at 20° C. for 2 hours. The mixture was concentrated under reduced pressure to give a residue. The residue was purified by prep-TLC (SiO₂, dichloromethane/methyl alcohol 10:1) to give an amide intermediate. A solution of the latter in trifluoroacetic acid (1.50 mL) was stirred at 70° C. for 0.5 hour. The mixture was concentrated under reduced pressure to give a residue. The residue was purified by prep-HPLC (HCl condition) to give a desired product.

Starting from intermediates F or F1, following the teachings of General Reaction Scheme VI and coupling methods CM7-A and CM7-B, Examples 7-1 to 7-4 were prepared as shown in Table 6:

TABLE 6

| | | | Yield | |
|---|---|---|---|---|
| | | Coupling | (1-2 | |
| Example | Structare | Method | steps) | Compound Name and Characterization |
| 7-1 | 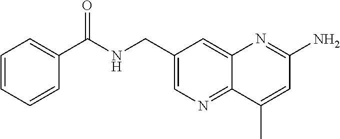 [SMILES: Cc1cc(N)nc2cc(CNC(=O)c3ccccc3)cnc12] | CM7-A | 19% | N-[(6-amino-8-methyl-1,5-naphthyridin-3- yl)methyl]benzamide. LCMS [M + 1]⁺ = 293.2. ¹H NMR (400 MHz, CD₃OD-d₄) δ = 8.82 (d, J = 2.0 Hz, 1H), 7.98 (br d, J = 2.0 Hz, 1H), 7.94- 7.83 (m, 2H), 7.61-7.55 (m, 1H), 7.53- 7.46 (m, 2H), 7.12 (s, 1H), 4.79 (s, 2H), 2.75 (s, 3H). |
| 7-2 | 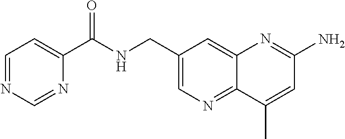 [SMILES: Cc1cc(N)nc2cc(CNC(=O)c3ccncn3)cnc12] | CM7-B | 14% | N-[(6-amino-8-methyl-1,5-naphthyridin-3- yl)methyl]pyrimidine-4-carboxamide. LCMS [M + 1]⁺ = 295.1. ¹H NMR (400 MHz, DMSO-d₆) δ = 14.68-14.08 (m, 1H), 9.92 (br t, J = 6.2 Hz, 1H), 9.48- 9.24 (m, 2H), 9.12 (d, J = 5.0 Hz, 1H), 8.80 (d, J = 2.0 Hz, 1H), 8.48-8.24 (m, 1H), 8.08-8.00 (m, 2H), 7.16 (d, J = 1.0 Hz, 1H), 4.68 (br d, J = 6.2 Hz, 2H), 2.64 (d, J = 0.8 Hz, 3H). |
| 7-3 | 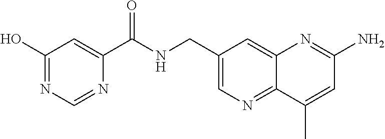 [SMILES: Cc1cc(N)nc2cc(CNC(=O)c3cc(O)ncn3)cnc12] | CM7-B | 7% | N-[(6-amino-8-methyl-1,5-naphthyridin-3- yl)methyl]-6-hydroxy-pyrimidine-4- carboxamide. LCMS [M + 1]⁺ = 311.1. ¹H NMR (400 MHz, CD₃OD) δ = 8.81 (d, J = 1.6 Hz, 1H), 8.32 (s, 1H), 7.99 (d, J = 1.6 Hz, 1H), 7.13 (d, J = 1.2 Hz, 1H), 7.10 (s, 1H), 4.78 (s, 2H), 2.75 (d, J = 1.2 Hz, 3H). |
| 7-4 | 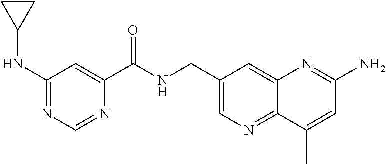 [SMILES: Cc1cc(N)nc2cc(CNC(=O)c3cc(NC4CC4)ncn3)cnc12] | CM7-A | 35% | N-[(6-amino-8-methyl-1,5-naphthyridin-3- yl)methyl]-6- (cyclopropylamino)pyrimidine-4- carboxamide. LCMS [M + 1]⁺ = 350.2. ¹H NMR (400 MHz, CDCl₃) δ ppm 8.67 (d, J = 2.0 Hz, 1 H) 8.36-8.59 (m, 2 H) 7.86 (d, J = 2.0 Hz, 1 H) 7.49 (br s, 1 H) 6.79 (d, J = 0.8 Hz, 1 H) 5.45-5.83 (m, 1 H) 4.86-5.12 (s, 2 H) 4.82 (d, J = 6.0 Hz, 2 H) 2.60-2.71 (m, 4 H) 0.90-1.00 (m, 2 H) 0.61-0.67 (m, 2 H). |

### Example 7-5

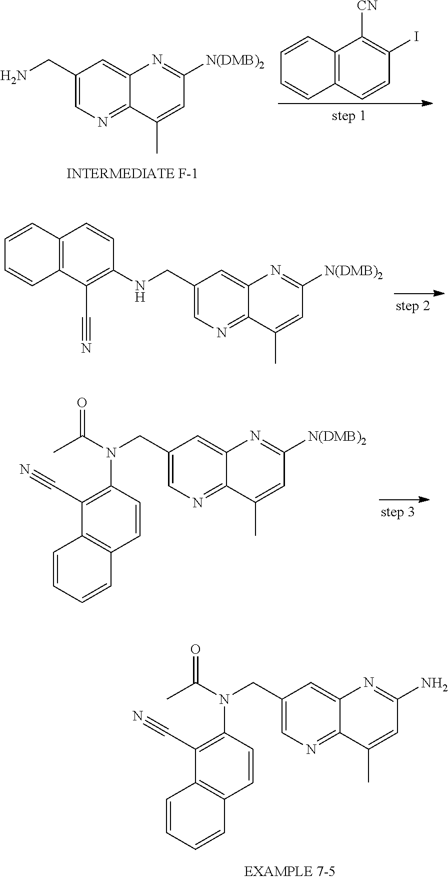

Step 1: A mixture of Intermediate F-1 (200 mg, 409 μmol, 1.00 eq.), 2-iodonaphthalene-1-carbonitrile (120 mg, 430 μmol, 1.00 eq.), sodium tert-butoxide (124 mg, 1.29 mmol, 3.00 eq.), Pd(dppf)Cl₂ (31.5 mg, 43.0 μmol, 0.10 eq.) and DPPF (71.5 mg, 129 μmol, 0.30 eq.) in dioxane (2.00 mL) was degassed and stirred at 100° C. for 2 hours under nitrogen atmosphere. The mixture was concentrated under reduced pressure to give a residue. The residue was purified by flash silica gel chromatography (0-100% Ethyl acetate/Petroleum) to give 2-(((6-(bis(3,4-dimethylbenzyl)amino)-8-methyl-1,5-naphthyridin-3-yl)methyl)amino)-1-naphthonitrile (140 mg, 177 μmol, 41.2% yield) as a white solid. LCMS [M+1]⁺=640.3.

Step 2: To a solution of 2-(((6-(bis(3,4-dimethylbenzyl)amino)-8-methyl-1,5-naphthyridin-3-yl)methyl)amino)-1-naphthonitrile (50.0 mg, 78.2 μmol, 1.00 eq.) in dioxane (2.00 mL) was added pyridine (61.8 mg, 782 μmol, 63.1 μL 10.0 eq.) and acetyl chloride (24.5 mg, 313 μmol, 22.3 μL 4.00 eq.). The mixture was stirred at 100° C. for 12 hours. The mixture was concentrated under reduced pressure to give a residue. The residue was purified by flash silica gel chromatography (0˜100% Ethyl acetate/Petroleum ether to 10% Methanol/Dichloromethane) to give N-[[6-[bis[(2,4-dimethoxyphenyl)methyl]amino]-8-methyl-1,5-naphthyridin-3-yl]methyl]-N-(1-cyano-2-naphthyl)acetamide (25.0 mg, 31.5 μmol, 40.3% yield) as a white solid. LCMS [M+1]⁺=682.5.

Step 3: A solution of N-[[6-[bis[(2,4-dimethoxyphenyl)methyl]amino]-8-methyl-1,5-naphthyridin-3-yl]methyl]-N-(1-cyano-2-naphthyl)acetamide (25.0 mg, 36.7 μmol, 1.00 eq.) in trifluoroacetic acid (1.00 mL) was stirred at 70° C. for 1 hour. The mixture was concentrated under reduced pressure to give a residue. The residue was purified by prep-HPLC (HCl condition) to give N-[(6-amino-8-methyl-1,5-naphthyridin-3-yl)methyl]-N-(1-cyano-2-naphthyl)acetamide, Example 7-5 (6.98 mg, 18.0 μmol, 49.0% yield) as a yellow solid. LCMS [M+1]⁺=382.1. ¹H NMR (400 MHz, MeOD-d₄) δ=8.61 (d, J=2.0 Hz, 1H), 8.37 (d, J=8.8 Hz, 1H), 8.11 (t, J=7.6 Hz, 2H), 8.03 (d, J=2.0 Hz, 1H), 7.84-7.72 (m, 2H), 7.61 (d, J=8.8 Hz, 1H), 7.14 (d, J=1.2 Hz, 1H), 5.50 (d, J=14.8 Hz, 1H), 5.05 (d, J=14.8 Hz, 1H), 2.71 (d, J=0.8 Hz, 3H), 2.01-1.96 (m, 3H)

### Example 7-6

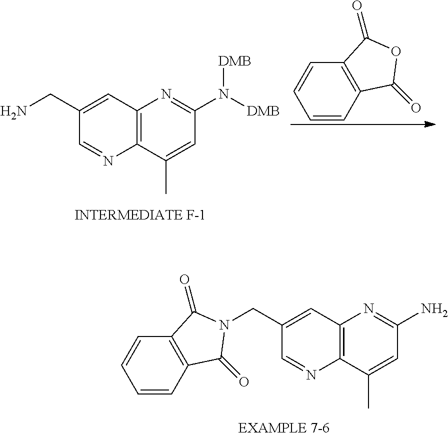

A mixture of Intermediate F1 (240 mg, 492 μmol, 1.00 eq.) and isobenzofuran-1,3-dione (72.9 mg, 492 μmol, 1.00 eq) in acetic acid (2.00 mL) was degassed and stirred at 120° C. for 12 hours under nitrogen atmosphere. The mixture was concentrated under reduced pressure to give a residue. The residue was purified by prep-HPLC (TFA condition), then by prep-TLC (SiO₂, dichloromethane/methyl alcohol 10:1) to give 2-[(6-amino-8-methyl-1,5-naphthyridin-3-yl)methyl]isoindoline-1,3-dione (28.4 mg, 88.2 μmol, 17.9% yield) as a white solid. LCMS [M+1]⁺=319.2. ¹H NMR (400 MHz, DMSO-d₆) δ=8.49 (d, J=2.0 Hz, 1H), 7.92-7.84 (m, 4H), 7.63 (d, J=2.0 Hz, 1H), 6.80 (s, 1H), 6.57-6.49 (m, 2H), 4.91 (s, 2H), 2.54-2.51 (m, 3H).

### Example 7-7

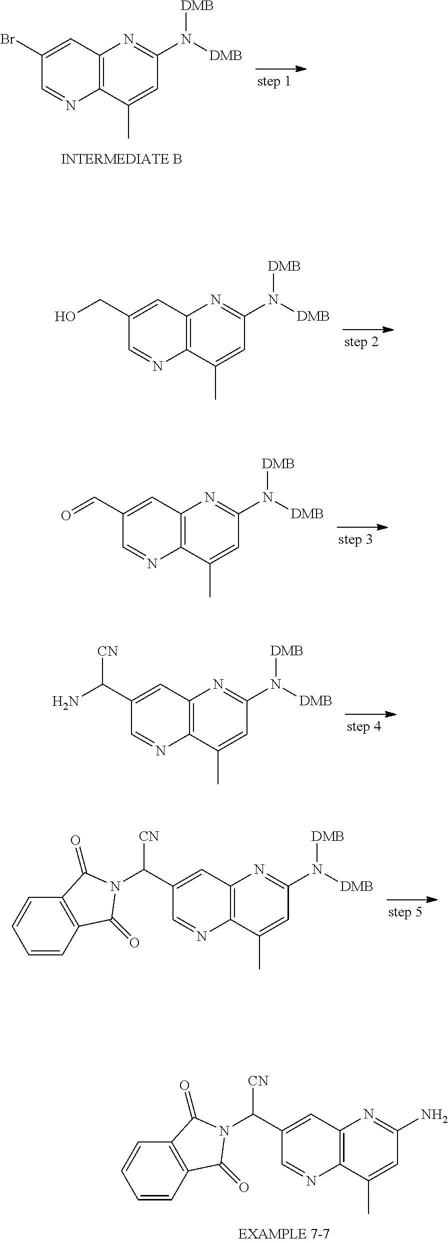

Step 1: A mixture of Intermediate B (500 mg, 929 μmol, 1.00 eq.), tributylstannylmethanol (596 mg, 1.86 mmol, 2.00 eq.) and Pd(PPh₃)₄ (107 mg, 92.9 μmol, 0.10 eq.) in dioxane (10.0 mL) was degassed and stirred at 100° C. for 6 hours under nitrogen atmosphere. The mixture was concentrated under reduced pressure to give a residue. The residue was purified by column chromatography (SiO₂, petroleum ether/ethyl acetate 10:1 to 1:1) to give [6-[bis[(2,4-dimethoxyphenyl)methyl]amino]-8-methyl-1,5-naphthyridin-3-yl]methanol (400 mg, 817 μmol, 88.00% yield) as a white solid. LCMS [ESI, M+1]⁺=490.3.

Step 2: To a solution of [6-[bis[(2,4-dimethoxyphenyl)methyl]amino]-8-methyl-1,5-naphthyridin-3-yl]methanol (400 mg, 817 μmol, 1.00 eq.) in dichloromethane (12.0 mL) was added Dess-Martin periodinane (520 mg, 1.23 mmol, 1.50 eq.). The mixture was stirred at 20° C. for 12 hours. The mixture was filtered and the filter cake was washed with ethyl acetate (50.0 mL×2). The filtrate was diluted with water (50.0 mL) and the aq layer was extracted with ethyl acetate (50.0 mL-3). The combined organic layers were washed with brine (50.0 mL), dried over anhydrous sodium sulfate, filtered and concentrated under reduced pressure to give a residue. The residue was purified by column chromatography (SiO₂, petroleum ether/ethyl acetate 10:1 to 1:1) to give 6-[bis[(2,4-dimethoxyphenyl)methyl]amino]-8-methyl-1,5-naphthyridine-3-carbaldehyde (260 mg, 533 μmol, 65.3% yield) as a yellow solid. LCMS [ESI, M+1]⁺=488.3. ¹H NMR (400 MHz, CDCl₃) δ (ppm)=10.21 (s, 1H), 9.00 (d, J=2.0 Hz, 1H), 8.36 (d, J=2.0 Hz, 1H), 7.07-7.21 (m, 2H) 6.99 (d, J=0.8 Hz, 1H), 6.48 (d, J=2.32 Hz, 2 H), 6.40 (dd, J=8.4, 2.4 Hz, 2H), 4.87 (br s, 4H), 3.80 (d, J=6.8 Hz, 12H) 2.64 (d, J=0.8 Hz, 3H).

Step 3: To a solution of 6-[bis[(2,4-dimethoxyphenyl)methyl]amino]-8-methyl-1,5-naphthyridine-3-carbaldehyde (100 mg, 205 μmol, 1.00 eq.) in methyl alcohol (2.00 mL) was added Ti(i-PrO)₄ (87.4 mg, 308 μmol, 90.8 μL 1.50 eq.), NH (7.0 M in methanol, 1.00 mL, 34.1 eq.). The mixture was stirred at 20° C. for 2 hours. Then TMSCN (50.9 mg, 513 μmol, 64.2 μL 2.50 eq.) was added to the mixture drop-wise and the mixture was stirred for 16 hours at 20° C. The mixture was poured into ice-water (30.0 mL) and exacted with ethyl acetate (30.0 mL×3). The combined organic layers were washed with brine (150 mL), dried over anhydrous sodium sulfate, filtered and concentrated under reduced pressure to give 2-amino-2-(6-(bis(2,4-dimethoxybenzyl)amino)-8-methyl-1,5-naphthyridin-3-yl)acetonitrile (80.0 mg, 160 μmol, 78.1% yield) as a yellow solid. LCMS [ESI, M+1]⁺=514.4.

Step 4: To a solution of 2-amino-2-[6-[bis[(2,4-dimethoxyphenyl)methyl]amino]-8-methyl-1,5-naphthyridin-3-yl]acetonitrile (50.0 mg, 97.4 μmol, 1.00 eq.) in acetic acid (2.00 mL) was added isobenzofuran-1,3-dione (28.8 mg, 195 μmol, 2.00 eq.). The mixture was stirred at 60° C. for 1 hour. The mixture was concentrated under reduced pressure to give a residue. The residue was purified by prep-TLC (SiO₂, petroleum ether/ethyl acetate 10:1) to give 2-[6-[bis[(2,4-dimethoxyphenyl)methyl]amino]-8-methyl-1,5-naphthyridin-3-yl]-2-(1,3-dioxoisoindolin-2-yl)acetonitrile (10.0 mg, 15.5 μmol, 16.0% yield) as a white solid. LCMS [ESI, M+1]⁺=644.4 Step 5: To a solution of 2-[6-[bis[(2,4-dimethoxyphenyl)methyl]amino]-8-methyl-1,5-naphthyridin-3-yl]-2-(1,3-dioxoisoindolin-2-yl)acetonitrile (10.0 mg, 15.5 μmol, 1.00 eq.) in trifluoroacetic acid (1.00 mL) was stirred at 70° C. for 1 hour. The mixture was concentrated under reduced pressure to give a residue. The residue was purified by prep-HPLC (HCl condition) to give 2-(6-amino-8-methyl-1,5-naphthyridin-3-yl)-2-(1,3-dioxoisoindolin-2-yl)acetonitrile, Example 7-7 (4.00 mg, 10.5 μmol, 67.4% yield, HCl) as a yellow gum. LCMS [ESI, M+1]⁺=344.1. ¹H NMR (400 MHz, DMSO-d₆) δ (ppm)=8.88-8.82 (m, 1H), 8.31 (d, J=2.0 Hz, 1H), 7.99-7.88 (m, 4H), 7.30-7.26 (m, 1H), 7.15 (s, 1H), 2.67 (br d, J=1.2 Hz, 1H), 2.66 (d, J=1.2 Hz, 3H), 2.56-2.53 (m, 2H) GENERAL COUPLING METHODS FOR THE PREPARATION OF EXAMPLES 8-1 to 8-2

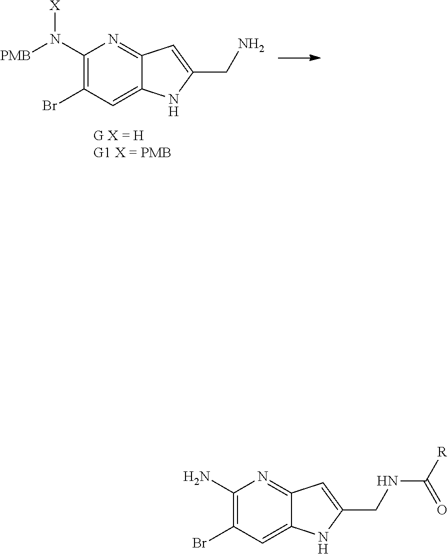

To a solution of Intermediate G (87.2 μmol, 1.00 eq.) and a carboxylic acid (130 μmol, 1.50 eq.) in dichloromethane (2.00 mL) was HATU (66.3 mg, 174 μmol, 2.00 eq.) and diisopropylethylamine (33.8 mg, 261 μmol, 45.5 μL, 3.00 eq.) at 25° C. The mixture was stirred at 25° C. for 1 h. The mixture was concentrated. The residue was purified by silica gel chromatography (Petroleum ether/Ethyl acetate 0-100%) to afford an intermediate product. To a solution of the latter (61.4 μmol, 1.00 eq.) in dichloromethane (2.50 mL) was added trifluoroacetic acid (0.50 mL) at 10° C. The mixture was stirred at 30° C. for 6 hrs. The mixture was basified to pH 8 by addition of sat. sodium bicarbonate, extracted with dichloromethane (100 mL), dried over sodium sulfate, filtered and concentrated. The residue was purified by silica gel chromatography (Dichloromethane/Methanol 0-10%) to afford a final desired product.

Starting from intermediates G, following the teachings of General Reaction Scheme VII and a general coupling method, Examples 8-1 to 8-2 were prepared as shown in Table 7.

TABLE 7

| | | Yield | |
|---|---|---|---|
| | | (2 | |
| Example | Structure | steps) | Compound Name and Characterization |
| 8-1 | 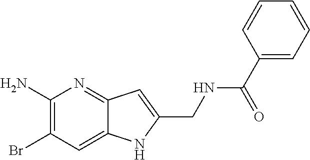 [SMILES: Nc1nc2cc(CNC(=O)c3ccccc3)[nH]c2cc1Br] | 39% | N-[(6-amino-8-methyl-1,5-naphthyridin-3- yl)methyl]benzamide. LCMS [M + 1]⁺ = 293.2. ¹H NMR (400 MHz, CD₃OD- d₄) δ = 8.82 (d, J = 2.0 Hz, 1H), 7.98 (br d, J = 2.0 Hz, 1H), 7.94-7.83 (m, 2H), 7.61-7.55 (m, 1H), 7.53- 7.46 (m, 2H), 7.12 (s, 1H), 4.79 (s, 2H), 2.75 (s, 3H). |
| 8-2 | 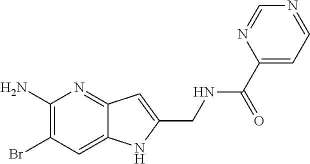 [SMILES: Nc1nc2cc(CNC(=O)c3ccncn3)[nH]c2cc1Br] | 13% | N-[(5-amino-6-bromo-1H-pyrrolo[3,2-b]pyridin-2- yl)methyl]pyrimidine-4-carboxamide. LCMS [ESI, M + 1]⁺ = 346.9. ¹H NMR (400 MHz, MeOD) δ = 9.30 (d, J = 1.2 Hz, 1H), 9.05 (d, J = 5.2 Hz, 1H), 8.32 (s, 1H), 8.12 (dd, J = 1.2, 5.2 Hz, 1H), 6.43 (s, 1H), 4.81-4.77 (m, 2H). |

### Example 9-1

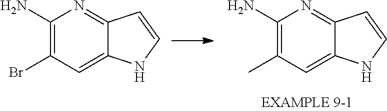

### Example 9-1

To a solution of 6-bromo-1H-pyrrolo[3,2-b]pyridin-5-amine (150 mg, 707 μmol, 1.00 eq.) and methylboronic acid (212 mg, 3.54 mmol, 5.00 eq.) in dioxane (2.50 mL) and water (0.50 mL) was added potassium carbonate (293 mg, 2.12 mmol, 3.00 eq.) and Pd(dppf)Cl₂-DCM (57.8 mg, 70.7 μmol, 0.10 eq.). The reaction mixture was stirred at 80° C. for 16 hours under nitrogen atmosphere before being diluted with water (50.0 mL) and filtered. The filtrate was concentrated. The residue was purified by prep-HPLC (basic condition) and lyophilized to afford 6-methyl-1H-pyrrolo[3,2-b]pyridin-5-amine, Example 9-1 (21.9 mg, 148 μmol, 20.9% yield) as a yellow solid. LCMS [M+1]-: 148.3. ¹H NMR (400 MHz, DMSO-d₆)=10.63 (br s, 1H), 7.30 (s, 1H), 7.19 (t, J=2.8 Hz, 1H), 6.11 (dt, J=0.8, 2.4 Hz, 1H), 5.06 (s, 2H), 2.13 (s, 3H).

### Example 9-2

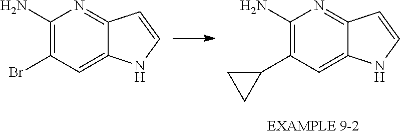 [SMILES: Nc1nc2cc[nH]c2cc1Br ; Nc1nc2cc[nH]c2cc1C1CC1]

To a solution of 6-bromo-1H-pyrrolo[3,2-b]pyridin-5-amine (150 mg, 707 μmol, 1.00 eq.) and cyclopropylboronic acid (304 mg, 3.54 mmol, 5.00 eq.) in dioxane (2.50 mL) and water (0.50 mL) was added potassium carbonate (293 mg, 2.12 mmol, 3.00 eq.) and Pd(dppf)Cl₂-DCM (57.8 mg, 70.7 μmol, 0.10 eq.). The mixture was stirred at 80° C. for 16 hours under nitrogen atmosphere before being diluted with water (50.0 mL) and filtered. The filtrate was concentrated. The residue was purified by prep-HPLC (basic condition) and lyophilized to afford 6-cyclopropyl-1H-pyrrolo[3,2-b]pyridin-5-amine, Example 9-2 (8.30 mg, 47.4 μmol, 6.70% yield) as a white solid. LCMS [M+1]⁺: 174.1. ¹H NMR (400 MHz, DMSO-d₆) δ=10.61 (br s, 1H), 7.21 (t, J=2.8 Hz, 1H), 7.18 (s, 1H), 6.11 (ddd, J=0.8, 2.0, 2.8 Hz, 1H), 5.20 (s, 2H), 1.78-1.69 (m, 1H), 0.92-0.85 (m, 2H), 0.54-0.47 (m, 2H).

### Example 9-3

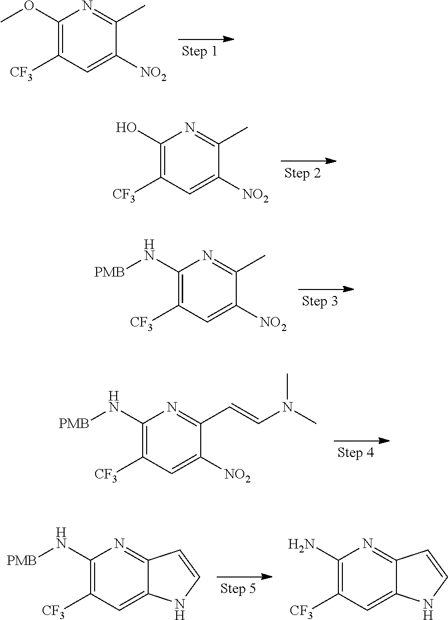

Step 1: To a mixture of 2-methoxy-6-methyl-5-nitro-3-(trifluoromethyl)pyridine (prepared following the method from WO2018215316) (500 mg, 2.12 mmol, 1.00 eq.) in acetonitrile (10.0 mL) was added chlorotrimethylsilane (1.15 g, 10.6 mmol, 1.34 mL, 5.00 eq.) and sodium iodide (1.59 g, 10.6 mmol, 5.00 eq.). The reaction mixture was stirred at 70° C. for 2 hours. The mixture was concentrated and the residue was purified by reversed-phase HPLC (formic acid condition) and lyophilized to afford 6-methyl-5-nitro-3-(trifluoromethyl)pyridin-2-ol (290 mg, 1.26 mmol, 59.7% yield) as a black solid. LCMS [ESI, M+1]: 223.0. ¹H NMR (400 MHz, DMSO-d₆) δ=13.23 (s, 1H), 8.46 (s, 1H), 2.70 (s, 3H).

Step 2: To a mixture of 6-methyl-5-nitro-3-(trifluoromethyl)pyridin-2-ol (250 mg, 1.13 mmol, 1.00 eq.) in acetonitrile (6.00 mL) was added DBU (343 mg, 2.25 mmol, 339 μL, 2.00 eq.), (4-methoxyphenyl)methanamine (463 mg, 3.38 mmol, 437 μL, 3.00 eq.) and BOP (647 mg, 1.46 mmol, 1.30 eq.). The mixture was stirred at 30° C. for 2 hours before being concentrated and purified by flash silica gel chromatography (Ethyl acetate/Petroleum ether 0-20%) to afford N-[(4-methoxyphenyl)methyl]-6-methyl-5-nitro-3-(trifluoromethyl)pyridin-2-amine (280 mg, 818 μmol, 72.7% yield) as a yellow oil. LCMS [ESI, M+1]: 341.9. ¹H NMR (400 MHz, CDCl₃) δ=8.48 (s, 1H), 7.26 (s, 2H), 6.96-6.84 (m, 2H), 5.73 (s, 1

Step 3: To a solution of N-[(4-methoxyphenyl)methyl]-6-methyl-5-nitro-3-(trifluoromethyl)pyridin-2-amine (250 mg, 733 μmol, 1.00 eq.) in dimethyl formamide (4.00 mL) was added N,N-dimethyl formamide dimethyl acetal (436 mg, 3.66 mmol, 486 μL, 5.00 eq.). The mixture was stirred at 90° C. for 3 hours. The reaction mixture was poured into brine (50.0 mL), extracted with ethyl acetate (10.0 mL×3). The combined organic layers were washed with water (30.0 mL), dried over anhydrous sodium sulfate, filtered and concentrated to afford 6-[(E)-2-(dimethylamino)vinyl]-N-[(4-methoxyphenyl)methyl]-5-nitro-3-(trifluoromethyl)pyridin-2-amine (290 mg, 702 μmol, 95.8% yield) as a brown solid. LCMS [ESI, M+1]: 397.2. ¹H NMR (400 MHz, CDCl₃) δ=8.45 (s, 1H), 8.00 (d, J=2.4 Hz, 1H), 7.24 (d, J=8.8 Hz, 2H), 6.93-6.82 (m, 2H), 6.47 (d, J=12.0 Hz, 1H), 5.52 (s, 1H), 4.67 (d, J=5.2 Hz, 2H), 3.79 (s, 3H), 3.03 (d, J=6.4 Hz, 6H).

Step 4: A suspension of Fe (42.3 mg, 757 μmol, 6.00 eq.) in acetic acid (1.00 mL) was stirred at 25° C. for 0.5 hour and then 6-[(E)-2-(dimethylamino)vinyl]-N-[(4-methoxyphenyl)methyl]-5-nitro-3-(trifluoromethyl)pyridin-2-amine (50.0 mg, 126 μmol, 1.00 eq.) was added. The reaction mixture was stirred at 25° C. for 1 hour, filtered and concentrated to afford N-[(4-methoxyphenyl)methyl]-6-(trifluoromethyl)-1H-pyrrolo[3,2-b]pyridin-5-amine (30.0 mg, 93.4 μmol, 74.0% yield) as a brown solid. LCMS [ESI, M+1]: 322.1.

Step 5: To a mixture of N-[(4-methoxyphenyl)methyl]-6-(trifluoromethyl)-1H-pyrrolo[3,2-b]pyridin-5-amine (30.0 mg, 93.4 μmol, 1.00 eq.) in dichloromethane (3.00 mL) was added trifluoroacetic acid (1.00 mL). The mixture was stirred at 25° C. for 16.5 hours. The mixture was basified to pH 7 with ammonium hydroxide and concentrated. The crude material was purified by prep-HPLC (basic condition) and lyophilized to afford 6-(trifluoromethyl)-1H-pyrrolo[3,2-b]pyridin-5-amine, Example 9-3 (2.52 mg, 12.0 μmol, 12.8% yield) as an off-white solid. LCMS [EST, M+1]: 202.1. ¹H NMR (400 MHz, MeOD) δ=7.89 (s, 1H), 7.54 (d, J=3.2 Hz, 1H), 6.32 (dd, J=0.8, 3.2 Hz, 1H).

### General Coupling Methods for the Preparation of Examples 10-1 to 10-10

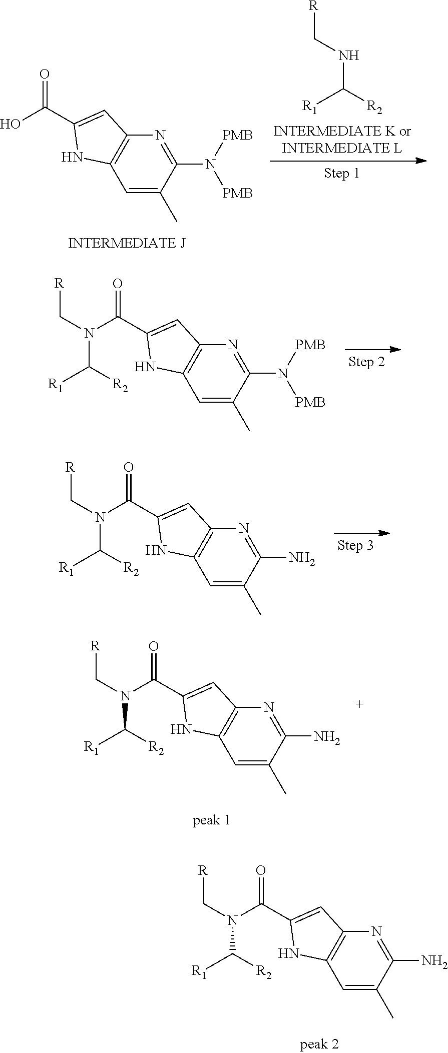

### Coupling Method 10-A (CM10-A):

Step 1: To a solution of acid Intermediate J (100 mg, 231.7 ummol 1 eq.), amine Intermediate K or Intermediate L (278.1 ummol 1.2 eq.) in acetonitrile (5 mL) was added N-(chloro(dimethylamino)methylene)-N-methylmethanaminium hexafluorophosphate(V) (71.5 mg, 254.9 μmmol 1.1 eq.) and 1-methylimidazole (47.6 mg, 579.4 μmmol 2.5 eq.). The mixture was stirred at 25° C. for 2 hours. The reaction mixture was concentrated under reduced pressure to give a residue and purified by prep-TLC (SiO₂, ethyl acetate) to afford di-PMB-protected amide intermediate.

Step 2: To a solution of di-PMB-protected amide intermediate (48.0 mg, 1 eq.) in trifluoroacetic acid (1 mL). The mixture was stirred at 60° C. for 12 hours. The reaction mixture was filtered and concentrated and purified by prep-HPLC to produce racemic amide intermediate.

Step 3: Racemic amide intermediate was separated by prep-SFC (column: DAICEL CHIRALPAK AD(250 mm×30 mm, 10 μm); mobile phase: [0.1% NH₃H₂O IPA]; B %: 60%-60%, 6 min) to afford desired examples peak 1 (S-enantiomer) and peak 2 (R-enantiomer).

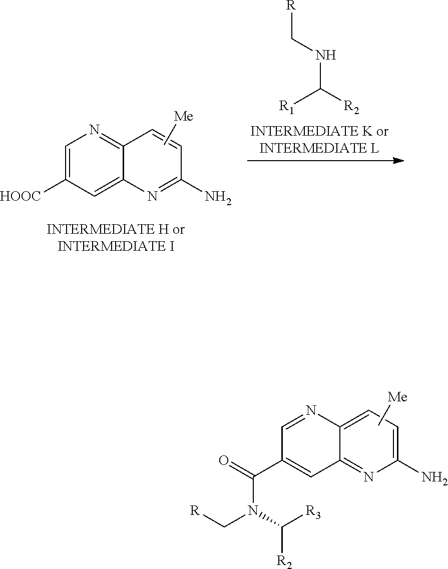

Coupling Method 10-B (CM10-B): To a solution of acid Intermediate H or I (30.0 mg, 1.00 eq.) in N,N-dimethyl acetamide (0.60 mL) was added amine Intermediate K1, K2, L1 or L2 (1.00 eq.), triethylamine (37.3 mg, 2.50 eq.) and PyBOP (92.2 mg, 1.20 eq.). The mixture was stirred at 40° C. for 12 hours. The reaction mixture was diluted with water (10.0 mL) and extracted with ethyl acetate (3×10.0 mL). The combined organic layers were dried over anhydrous sodium sulfate, filtered and concentrated under reduced pressure to give a residue. The residue was purified by prep-HPLC afford desired amide example.

Following general coupling methods CM10-A or CM10-B, Examples 10-1 to 10-10 were prepared as shown in Table 10:

TABLE 10

| | | Coupling | | Compound Name and |
|---|---|---|---|---|
| Example | Structure | Method | Yield | Characterization |
| 10-1 | 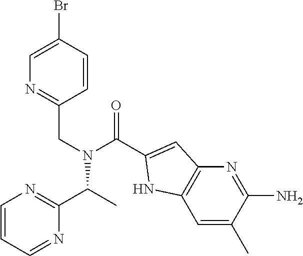 [SMILES: Cc1cc2[nH]c(C(=O)N(Cc3ccc(Br)cn3)[C@H](C)c3ncccn3)cc2nc1N] | CM10- A | 38% | (S)-5-amino-N-((5-bromopyridin- 2-yl)methyl)-6-methyl-N-(1- (pyrimidin-2-yl)ethyl)-1H- pyrrolo[3,2-b]pyridine-2- carboxamide LCMS [M + 1]⁺ = 468.0. ¹H NMR (400 MHz, DMSO-d₆) δ = 11.66- 11.05 (m, 1H), 9.02-8.50 (m, 3H), 8.14-7.86 (m, 1H), 7.68- 7.13 (m, 3H), 6.73-6.00 (m, 1H), 5.34 (br s, 2H), 5.07-4.36 (m, 1H), 2.13 (br s, 3H), 1.72-1.52 (m, 3H) |
| 10-2 | 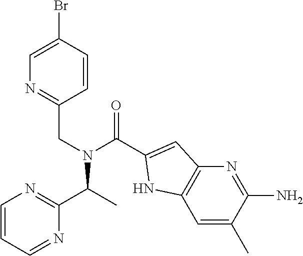 [SMILES: Cc1cc2[nH]c(C(=O)N(Cc3ccc(Br)cn3)[C@@H](C)c3ncccn3)cc2nc1N] | CM10- A | 35% | (R)-5-amino-N-((5-bromopyridin- 2-yl)methyl)-6-methyl-N-(1- (pyrimidin-2-yl)ethyl)-1H- pyrrolo[3,2-b]pyridine-2- carboxamide LCMS [M + 1]⁺ = 468.0. ¹H NMR (400 MHz, DMSO-d₆) δ = 11.31 (br d, J = 3.2 Hz, 1H), 8.80 (br s, 3H), 8.12-7.86 (m, 1H), 7.54- 7.22 (m, 3H), 6.74-5.99 (m, 1H), 5.32 (br s, 2H), 5.12-4.30 (m, 1H), 2.13 (br s, 3H), 1.62 (br s, 2H), 1.23 (s, 3H) |
| 10-3 | 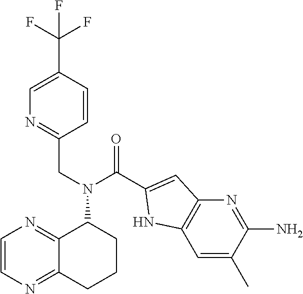 | CM10- A | 7% | (S)-5-amino-6-methyl-N-(5,6,7,8- tetrahydroquinoxalin-5-yl)-N-((5- (trifluoromethyl)pyridin-2- yl)methyl)-1H-pyrrolo[3,2- b]pyridine-2-carboxamide LCMS [M + 1]⁺ = 482.1. ¹H NMR (400 MHz, METHANOL-d₄) δ = 8.96 (br s, 1H), 8.49-8.36 (m, 2H), 8.28-7.99 (m, 2H), 7.79- 7.47 (m, 2H), 6.84-−6.27 (m, 1H), 5.42-5.14 (m, 2H), 3.07- 2.96 (m, 2H), 2.36 (br s, 1H), 2.24 (br d, J = 1.2 Hz, 3H), 2.03 (s, 1H), 1.94-1.86 (m, 1H), 1.29 (br s, 2H) |
| 10-4 | 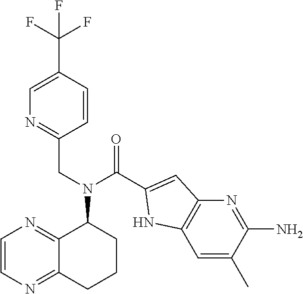 [SMILES: Cc1cc2[nH]c(C(=O)N(Cc3ccc(C(F)(F)F)cn3)[C@H]3CCCc4nccnc43)cc2nc1N] | CM10- A | 7% | (R)-5-amino-6-methyl-N-(5,6,7,8- tetrahydroquinoxalin-5-yl)-N-((5- (trifluoromethyl)pyridin-2- yl)methyl)-1H-pyrrolo[3,2- b]pyridine-2-carboxamide LCMS [M + 1]⁺ = 482.1. ¹H NMR (400 MHz, METHANOL-d4) δ = 9.09-8.60 (m, 1H), 8.48-8.32 (m, 2H), 8.25-7.99 (m, 2H), 7.82- 7.44 (m, 2H), 6.86-6.27 (m, 1H), 5.47-5.04 (m, 2H), 3.06- 2.89 (m, 2H), 2.49-2.34 (m, 1H), 2.25 (br s, 3H), 2.16 (br d, J = 12 Hz, 1H), 2.00-1.86 (m, 1H), 1.29 (br s, 2H) |
| 10-5 | 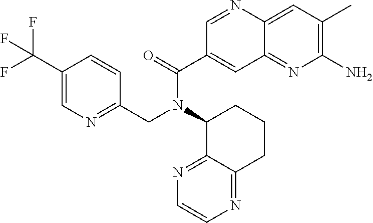 [SMILES: Cc1cc2ncc(C(=O)N(Cc3ccc(C(F)(F)F)cn3)[C@H]3CCCc4nccnc43)cc2nc1N] | CM10- B | 8% | (S)-6-amino-7-methyl-N-(5,6,7,8- tetrahydroquinoxalin-5-yl)-N-((5- (trifluoromethyl)pyridin-2- yl)methyl)-1,5-naphthyridine-3- carboxamide LCMS [M + 1]⁺ = 494.2. ¹H NMR (400 MHz, MeOD-d₄) δ = 9.01- 8.71 (m, 2H), 8.56-8.50 (m, 1H), 8.43-8.36 (m, 1H), 8.31-8.02 (m, 2H), 7.89-7.81 (m, 1H), 7.80- 7.71 (m, 1H), 5.36-5.27 (m, 1H), 5.00 (d, J = 16.5 Hz, 1H), 4.24 (d, J = 16.4 Hz, 1H), 3.07- 2.80 (m, 2H), 2.42-2.24 (m, 4H), 2.22-2.00 (m, 2H), 1.96-1.66 (m, 1H) |
| 10-6 | 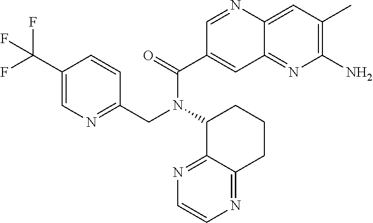 | CM10- B | 49% | (R)-6-amino-7-methyl-N-(5,6,7,8- tetrahydroquinoxalin-5-yl)-N-((5- (trifluoromethyl)pyridin-2- yl)methyl)-1,5-naphthyridine-3- carboxamide LCMS [M + 1]⁺ = 494.1 ¹H NMR (400 MHz, MeOD-d₄) δ = 8.93-8.73 (m, 2H), 8.60-8.51 (m, 1H), 8.45-8.38 (m, 1H), 8.27- 8.05 (m, 2H), 7.92-7.83 (m, 1H), 7.82-7.72 (m, 1H), 5.32 (br dd, J = 6.0, 10.8 Hz, 1H), 5.02 (d, J = 16.4 Hz, 1H), 4.26 (d, J = 16.4 Hz, 1H), 3.09-2.83 (m, 2H), 2.39- |
| | | | | 2.34 (m, 3H), 2.34-2.22 (m, |
| | | | | 1H), 2.21-2.03 (m, 2H), 1.95- |
| | | | | 1.70 (m, 1H) |
| 10-7 | 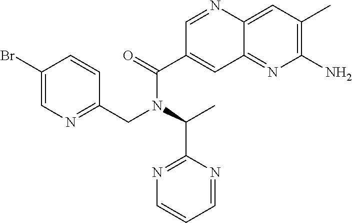 [SMILES: Cc1cc2ncc(C(=O)N(Cc3ccc(Br)cn3)[C@@H](C)c3ncccn3)cc2nc1N] | CM10- B | 22% | (S)-6-amino-N-((5-bromopyridin- 2-yl)methyl)-7-methyl-N-(1- (pyrimidin-2-yl)ethyl)-1,5- naphthyridine-3-carboxamide LCMS [M + 1]⁺ = 480.0. ¹H NMR (400 MHz, MeOD-d₄) δ = 8.82- 8.43 (m, 4H), 8.20-7.98 (m, 1H), 7.95-7.75 (m, 2H), 7.45-7.16 (m, 2H), 5.92-5.25 (m, 1H), 4.96 (br d, J = 16.4 Hz, 1H), 4.61 (br d, J = 16.4 Hz, 1H), 2.42-2.27 (m, 3H), 1.68 (br d, J = 7.2 Hz, 3H) |
| 10-8 | 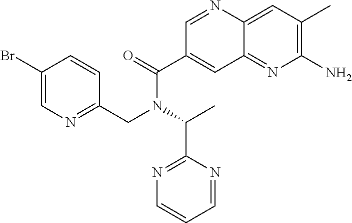 [SMILES: Cc1cc2ncc(C(=O)N(Cc3ccc(Br)cn3)[C@H](C)c3ncccn3)cc2nc1N] | CM10- B | 11% | (R)-6-amino-N-((5-bromopyridin- 2-yl)methyl)-7-methyl-N-(1- (pyrimidin-2-yl)ethyl)-1,5- naphthyridine-3-carboxamide LCMS [M + 1]⁺ = 480.0. ¹H NMR (400 MHz, MeOD-d₄) δ = 8.85- 8.45 (m, 4H), 8.22-8.00 (m, 1H), 7.98-7.76 (m, 2H), 7.46-7.20 (m, 2H), 5.95-5.23 (m, 1H), 4.98 (br d, J = 16.4 Hz, 1H), 4.63 (br d, J = 16.4 Hz, 1H), 2.38 (br s, 3H), 1.70 (d, J = 7.2 Hz, 3H) |
| 10-9 | 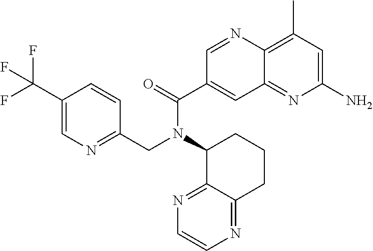 [SMILES: Cc1cc(N)nc2cc(C(=O)N(Cc3ccc(C(F)(F)F)cn3)[C@H]3CCCc4nccnc43)cnc12] | CM10- B | 38% | (S)-6-amino-8-methyl-N-(5,6,7,8- tetrahydroquinoxalin-5-yl)-N-((5- (trifluoromethyl)pyridin-2- yl)methyl)-1,5-naphthyridine-3- carboxamide LCMS [M + 1]⁺ = 494.2. ¹H NMR (400 MHz, MeOD-d₄) δ = 8.89- 8.78 (m, 1H), 8.78-8.57 (m, 1H), 8.55-8.51 (m, 1H), 8.42-8.38 (m, 1H), 8.16-7.97 (m, 2H), 7.81- 7.71 (m, 1H), 6.99-6.94 (m, 1H), 5.36-5.24 (m, 1H), 5.04- 4.97 (m, 1H), 4.24 (d, J = 16.6 Hz, 1H), 3.06-2.83 (m, 2H), 2.64- 2.57 (m, 3H), 2.40-2.24 (m, 1H), 2.19-2.01 (m, 2H), 1.96-1.68 (m, 1H) |
| 10-10 | 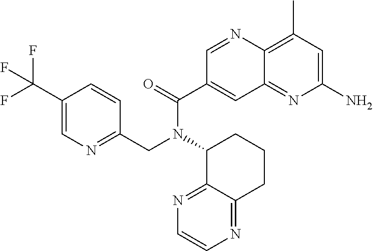 [SMILES: Cc1cc(N)nc2cc(C(=O)N(Cc3ccc(C(F)(F)F)cn3)[C@@H]3CCCc4nccnc43)cnc12] | CM10- B | 25% | (R)-6-amino-8-methyl-N-(5,6,7,8- tetrahydroquinoxalin-5-yl)-N-((5- (trifluoromethyl)pyridin-2- yl)methyl)-1,5-naphthyridine-3- carboxamide LCMS [M + 1]⁺ = 494.3. ¹H NMR (400 MHz, MeOD-d₄) δ = 8.89- 8.56 (m, 2H), 8.55-8.51 (m, 1H), 8.42-8.38 (m, 1H), 8.16-7.96 (m, 2H), 7.81-7.72 (m, 1H), 6.98- 6.93 (m, 1H), 5.35-5.24 (m, 1H), 5.01 (d, J = 16.5 Hz, 1H), 4.24 (d, J = 16.5 Hz, 1H), 3.07- 2.82 (m, 2H), 2.64-2.56 (m, 3H), 2.41-2.22 (m, 1H), 2.21-2.01 (m, 2H), 1.92-1.71 (m, 1H) |

### Examples 10-11 and 10-12

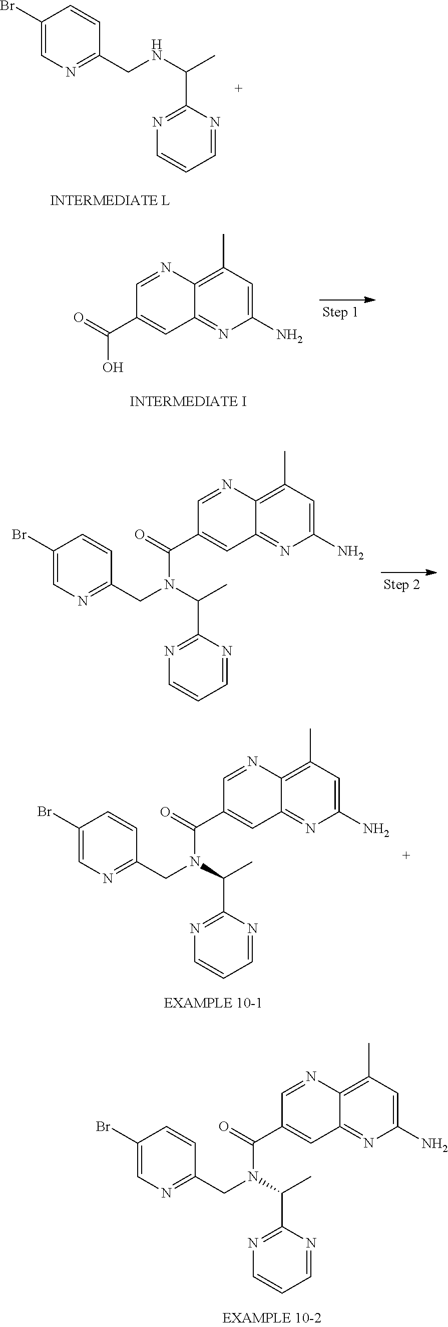 [SMILES: CC(NCc1ccc(Br)cn1)c1ncccn1 ; Cc1cc(N)nc2cc(C(=O)O)cnc12 ; Cc1cc(N)nc2cc(C(=O)N(Cc3ccc(Br)cn3)C(C)c3ncccn3)cnc12 ; Cc1cc(N)nc2cc(C(=O)N(Cc3ccc(Br)cn3)[C@@H](C)c3ncccn3)cnc12 ; Cc1cc(N)nc2cc(C(=O)N(Cc3ccc(Br)cn3)[C@H](C)c3ncccn3)cnc12]

Step 1: To a solution of Intermediate I (200 mg, 984 μmol, 1.0 eq.) and Intermediate L (231 mg, 787 μmol, 0.8 eq.) in pyridine (3.0 mL) was added EDCI (283 mg, 1.48 mmol, 1.5 eq.). The mixture was stirred at 40° C. for 2 hours. The mixture was concentrated under reduced pressure to give a residue. The residue was purified by prep-HPLC to give 6-amino-N-[(5-bromo-2-pyridyl)methyl]-8-methyl-N-(1-pyrimidin-2-ylethyl)-1,5-naphthyridine-3-carboxamide (60.0 mg, 125 μmol, 12.7% yield) as a white solid. LCMS [M+1]-=478.1

Step 2: Racemic 6-amino-N-[(5-bromo-2-pyridyl)methyl]-8-methyl-N—(I-pyrimidin-2-ylethyl)-1,5-naphthyridine-3-carboxamide (30.0 mg, 62.5 μmol) was purified by prep-SFC (column: daicel chiralcel OX (250 mm×30 mm, 10 um);mobile phase: [MeOH-ACN]; B %: 70%-70%, 5.1 min) to give 6-amino-N-[(5-bromo-2-pyridyl)methyl]-8-methyl-N-[(1S)-1-pyrimidin-2-ylethyl]-1,5-naphthyridine-3-carboxamide, Example 10-1 (5.0 mg, 10.5 μmol, 16.7% yield) as a white solid, LCMS [M+1]⁺=478.2, ¹H NMR (400 MHz, DMSO-d₆) δ=8.79 (d, J=5.2 Hz, 2H), 8.65-8.54 (m, 2H), 7.97-7.88 (m, 2H), 7.43-7.39 (m, 1H), 7.36 (br d, 0.1=8.4 Hz, 1H), 6.90-6.82 (m, 1H), 6.73-6.65 (m, 2H), 5.29 (br d, J=6.4 Hz, 1H), 4.83 (br d, J=16.4 Hz, 1H), 4.38 (br d, J=16.4 Hz, 1H), 2.55 (s, 3H), 1.58 (br d, J=7.2 Hz, 3H) and 6-amino-N-[(5-bromo-2-pyridyl)methyl]-8-methyl-N-[(1R)-1-pyrimidin-2-ylethyl]-1,5-naphthyridine-3-carboxamide, Example 10-12 (5.0 mg, 10.5 μmol, 16.7% yield) as a white solid, LCMS [M+1]⁺=478.2. ¹H NMR (400 MHz, DMSO-d₆) 5=8.80 (d, J=5.2 Hz, 2H), 8.68-8.53 (m, 2H), 7.96-7.87 (m, 2H), 7.43-7.39 (m, 1H), 7.36 (br d, J=8.4 Hz, 1H), 6.91-6.82 (m, 1H), 6.74-6.65 (m, 2H), 5.37-5.22 (m, 1H), 4.83 (br d, J=16.4 Hz, 1H), 4.38 (br d, J=16.4 Hz, 1H), 2.55 (s, 3H), 1.58 (br d, J=6.4 Hz, 3H).

### Example 11-1

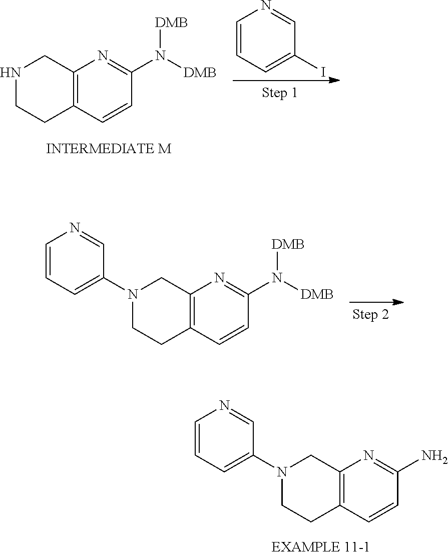

Step 1: A mixture of Intermediate M (60.0 mg, 293 μmol, 1.00 eq.), 3-iodopyridine (66.0 mg, 322 μmol, 1.10 eq.), sodium tert-butoxide (84.4 mg, 878 μmol, 3.00 eq.) and methanesulfonato(2-dicyclohexylphosphino-2,6-bis(dimethylamino)-1,1-biphenyl)(2-amino-1,1-biphenyl-2-yl)palladium(II) (11.8 mg, 14.6 μmol, 0.05 eq.) in toluene (2.00 mL) was degassed and stirred at 100° C. for 12 hours under nitrogen atmosphere. The mixture was concentrated in vacuum to give a residue. The residue was purified by prep-TLC to give N, N-bis(3,4-dimethoxybenzyl)-7-(pyridin-3-yl)-5,6,7,8-tetrahydro-1,7-naphthyridin-2-amine (50.0 mg, 177 μmol, 60.6% yield) as a yellow oil. LCMS [M+1]⁺=527.4.

Step 2: A solution of N,N-bis(3,4-dimethoxybenzyl)-7-(pyridin-3-yl)-5,6,7,8-tetrahydro-1,7-naphthyridin-2-amine (25.0 mg, 54.0 μmol, 1.00 eq.) in trifluoroacetic acid (1.00 mL) was stirred at 20° C. for 10 min. The mixture was concentrated in vacuum to give a residue. The residue was purified by prep-HPLC (HCl condition) to give 7-(3-pyridyl)-6,8-dihydro-5H-1,7-naphthyridin-2-amine, Example 11-1 (6.01 mg, 22.1 μmol, 40.9% yield, HCl salt) as a white solid. LCMS [M+1]⁺=−227.2. ¹H NMR (400 MHz, DMSO-d₆) δ=8.45 (d, J=2.4 Hz, 1H), 8.21 (d, J=5.2 Hz, 1H), 8.08-8.03 (m, 1H), 8.02-7.92 (m, 1H), 7.92-7.80 (m, 1H), 7.80-7.66 (m, 1H), 6.87 (d, J=9.2 Hz, 1H), 4.61 (s, 2H), 3.73 (br t, J=5.6 Hz, 2H), 2.75 (br s, 2H), 2.51 (s, 2H).

### Example 11-2

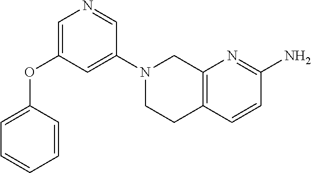 [SMILES: Nc1ccc2c(n1)CN(c1cncc(Oc3ccccc3)c1)CC2]

7-(5-phenoxy-3-pyridyl)-6,8-dihydro-5H-1,7-naphthyridin-2-amine, Example 11-2 was synthesized from Intermediate M and 3-bromo-5-phenoxy-pyridine using the same 2-step procedure as described for Example 11-1 to afford the title compound as (6.87 mg, 19.4 μmol, 19% yield over 2 steps) as a white oil. LCMS [M+1]⁺=319.2. ¹H NMR (400 MHz, DMSO-d₆) δ=8.30 (d, J=2.0 Hz, 1H), 8.19-7.99 (m, 1H), 7.84 (d, J=1.6 Hz, 1H), 7.81-7.71 (m, 1H), 7.71-7.60 (m, 1H), 7.54-7.36 (m, 2H), 7.29-7.22 (m, 1H), 7.18 (d, J=7.6 Hz, 2H), 6.89 (d, J=9.2 Hz, 1H), 4.60 (s, 2H), 3.71 (br t, J=5.6 Hz, 2H), 2.73 (br s, 2H), 2.51 (s, 2H).

### Example 11-3

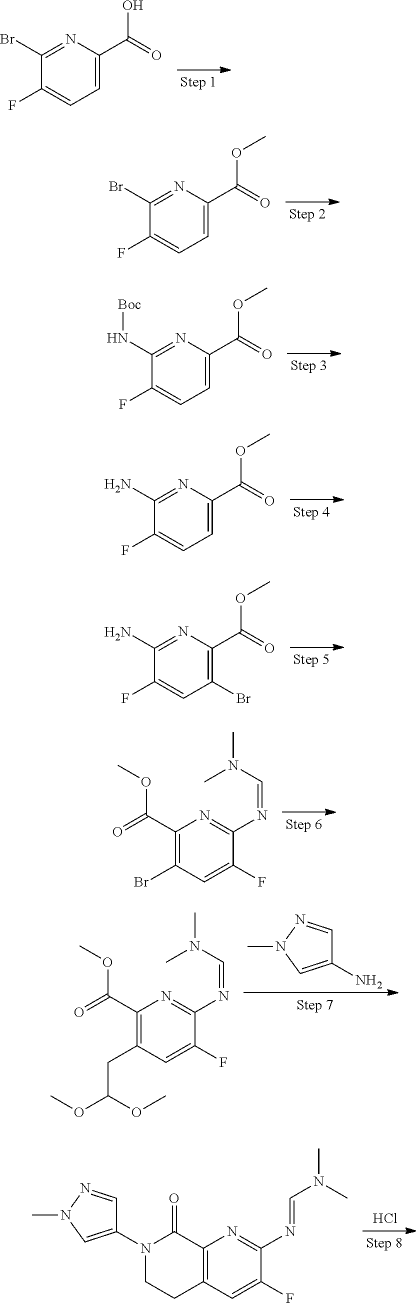

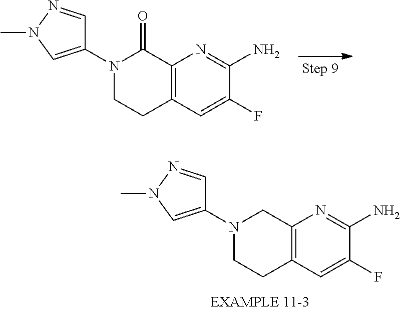 [SMILES: Cn1cc(N2CCc3cc(F)c(N)nc3C2=O)cn1 ; Cn1cc(N2CCc3cc(F)c(N)nc3C2)cn1]

Step 1: To a solution of 6-bromo-5-fluoro-pyridine-2-carboxylic acid (12.5 g, 56.8 mmol, 1.00 eq.) in methanol (150 mL) was added con. Sulfuric acid (1.11 g, 11.4 mmol, 606 μL 0.20 eq.). The mixture was stirred at 90° C. for 1 hour. The mixture was basified neutralized with saturated aqueous sodium carbonate (300 mL), and extracted with ethyl acetate (200 mL-2). The combined organic layers were washed with brine (250 mL), dried over anhydrous sodium sulfate, filtered and concentrated under reduced pressure to give methyl 6-bromo-5-fluoropicolinate (12.5 g, crude) as a white solid. ¹H NMR (400 MHz, DMSO-d₆) δ=8.16 (dd, J=3.6, 8.4 Hz, 1H), 8.07-8.01 (m, 1H), 3.89 (s, 3H)

Step 2: A mixture of methyl 6-bromo-5-fluoro-pyridine-2-carboxylate (24.5 g, 105 mmol, 1.0 eq.), tert-butyl carbamate (14.7 g, 126 mmol, 1.2 eq.), Pd(OAc)₂ (1.18 g, 5.23 mmol, 0.05 eq.), Xantphos (3.03 g, 5.23 mmol, 0.05 eq.) and cesium carbonate (102 g, 314 mmol, 3.00 eq.) in dioxane (200 mL) was degassed and stirred at 90° C. for 2 hours under nitrogen atmosphere. The reaction mixture was diluted with water (300 mL) and extracted with ethyl acetate (300 mL×3). The combined organic layers were washed with brine (1000 mL), dried over anhydrous sodium sulfate, filtered and concentrated under reduced pressure to give a residue. The residue was purified by column chromatography (SiO₂, Petroleum ether/Ethyl acetate 1:0 to 3:1) to give methyl 6-amino-5-fluoropicolinate and methyl 6-((tert-butoxycarbonyl)amino)-5-fluoropicolinate (6.00 g, crude) as a yellow solid. ¹H NMR (400 MHz, DMSO-d₆) δ=7.98-7.88 (m, 2H), 7.46 (d, J=8.0, 10.8 Hz, 1H), 7.26 (d, J=2.8, 8.0 Hz, 1H), 6.62 (s, 2H), 3.86 (s, 3H), 3.79 (s, 3H), 1.43 (s, 9H).

Step 3: To a solution of methyl 6-(tert-butoxycarbonylamino)-5-fluoro-pyridine-2-carboxylate (6.00 g, 22.2 mmol, 1.00 eq.) in DCM (20.0 mL) was added HCl/dioxane (4M, 40.0 mL). The mixture was stirred at 25° C. for 1 hour. The mixture was basified to pH 7 with sodium bicarbonate (aq. 1.0 M). The resulting mixture was filtered and dried under reduced pressure to give methyl 6-amino-5-fluoropicolinate (6.00 g, crude) as a yellow solid

Step 4: To a solution of methyl 6-amino-5-fluoro-pyridine-2-carboxylate (3.00 g, 17.6 mmol, 1.00 eq.) in acetonitrile (50.0 mL) were added NBS (3.45 g, 19.4 mmol, 1.10 eq.) and acetic acid (106 mg, 1.76 mmol, 101 μL 0.10 eq.). The mixture was stirred at 30° C. for 2 hours. The residue was diluted with water (150 mL) and extracted with ethyl acetate (100 mL×3). The combined organic layers were washed with brine 150 mL, dried over anhydrous sodium sulfate, filtered and concentrated under reduced pressure to give a residue. The residue was purified by column chromatography (SiO₂, Petroleum ether/Ethyl acetate 1:0 to 3:1) to give methyl 6-amino-3-bromo-5-fluoropicolinate (2.50 g, 8.30 mmol, 47.1% yield) as a yellow solid. LCMS [M-14]=234.7. ¹H NMR (400 MHz, DMSO-d₆) δ=7.82 (d, J=10.4 Hz, 1H), 7.03-6.65 (m, 2H), 3.82 (s, 3H).

Step 5: To a solution of methyl 6-amino-3-bromo-5-fluoro-pyridine-2-carboxylate (2.00 g, 8.03 mmol, 1.00 eq.) in DMF-DMA (10.0 mL). The mixture was stirred at 100° C. for 1 hour. The residue was diluted with water (15 mL) and extracted with ethyl acetate (30 mL×2). The combined organic layers were washed with brine (15 mL), dried over anhydrous sodium sulfate. The residue was filtered and concentrated under reduced pressure to give (Z)-methyl 3-bromo-6-(((dimethylamino)methylene)amino)-5-fluoropicolinate (2.20 g, 7.23 mmol, 90.1% yield) as a red solid. ¹H NMR (400 MHz, DMSO-<6) δ=8.40 (s, 1H), 7.99 (d, J=9.6 Hz, 1H), 3.86 (s, 3H), 3.14 (s, 3H), 3.03 (s, 3H).

Step 6: To a solution of methyl 3-bromo-6-[(Z)-dimethylaminomethyleneamino]-5-fluoro-pyridine-2-carboxylate (2.20 g, 7.23 mmol, 1.00 eq.) and 2-bromo-1,1-dimethoxy-ethane (1.59 g, 9.40 mmol, 1.10 mL, 1.30 eq.) in DME (2.00 mL) were added Ir[dF(CF₃)ppy]₂(dtbpy)(PF₆) (81.2 mg, 72.3 μmol, 0.01 eq.), NiCl₂-dtbbpy (43.2 mg, 109 μmol, 0.015 eq.), TTMSS (1.80 g, 7.23 mmol, 2.23 mL, 1.00 equiv) and sodium carbonate (1.53 g, 14.5 mmol, 2.00 eq.). The vial was sealed and placed under nitrogen atmosphere. The reaction was stirred and irradiated with a 10 W blue LED lamp (3 cm away), with cooling to maintain the reaction temperature at 25° C. for 12 hours. The residue was diluted with water (150 mL) and extracted with ethyl acetate (100 mL×3). The combined organic layers were washed with brine 150 mL, dried over anhydrous sodium sulfate, filtered and concentrated under reduced pressure to give a residue. The residue was purified by column chromatography (SiO₂, Petroleum ether/Ethyl acetate 1:0 to 1:1) to give (Z)-methyl 3-(2,2-dimethoxyethyl)-6-(((dimethylamino)methylene)amino)-5-fluoropicolinate (1.40 g, 1.88 mmol, 25.9% yield, 42.0% purity) as a yellow oil. LCMS [M+1]⁺=313.7. ¹H NMR (400 MHz, DMSO-d₆) δ=8.43 (s, 1H), 7.62 (d, J=2.4 Hz, 1H), 4.46 (t, J=5.2 Hz, 1H), 3.81 (s, 3H), 3.25 (s, 2H), 3.22 (s, 6H), 3.11 (s, 3H), 3.01 (s, 3H).

Step 7: To a solution of (Z)-methyl 3-(2,2-dimethoxyethyl)-6-(((dimethylamino)methylene)amino)-5-fluoropicolinate (300 mg, 402.14 μmol, 42% purity, 1.00 eq.) and 1-methylpyrazol-4-amine (117 mg, 1.21 mmol, 3.00 eq.) in dichloromethane (1.00 mL) was added TFA (6.93 g, 60.8 mmol, 4.50 mL, 151 eq.) and triethylsilane (187 mg, 1.61 mmol, 257 μL 4.00 eq.). The mixture was stirred at 25° C. for 2 hours. The mixture was filtered and concentrated under reduced pressure to give a residue. The residue was purified by prep-HPLC to give (E)-N-(3-fluoro-7-(1-methyl-1H-pyrazol-4-yl)-8-oxo-5,6,7,8-tetrahydro-1,7-naphthyridin-2-yl)-N,N-dimethylformimidamide (40.0 mg, 124 μmol, 30.8% yield) as a yellow solid. LCMS [M+1]⁺=317.1.

Step 8: To a solution of (E)-N-(3-fluoro-7-(1-methyl-1H-pyrazol-4-yl)-8-oxo-5,6,7,8-tetrahydro-1,7-naphthyridin-2-yl)-N,N-dimethylformimidamide (15.0 mg, 46.5 μmol, 1.00 eq.) in methanol (0.50 mL) was added HCl (1.00 M, 46.5 μL 1.00 eq.). The mixture was stirred at 80° C. for 1 hour. The mixture was filtered and concentrated under reduced pressure to give a residue which was purified by prep-HPLC to give 2-amino-3-fluoro-7-(1-methyl-1H-pyrazol-4-yl)-6,7-dihydro-1,7-naphthyridin-8(5H)-one (12.0 mg, 45.5 μmol, 97.8% yield) as a white solid. LCMS [M+1]⁺=262.2. ¹H NMR (400 MHz, DMSO-d₆) δ=8.07 (s, 1H), 7.66 (s, 1H), 7.40 (d, J=10.8 Hz, 1H), 6.39 (s, 2H), 3.89 (br t, J=6.4 Hz, 2H), 3.83 (s, 3H), 2.93 (br t, J=6.4 Hz, 2H).

Step 9: To a solution of 2-amino-3-fluoro-7-(1-methylpyrazol-4-yl)-5,6-dihydro-1,7-naphthyridin-8-one (8.00 mg, 30.3 μmol, 1.00 eq.) in THF (1.00 mL) was added BH₃-Me₂S (10.0 M, 6.06 μL 2.00 eq.) at 0° C. The mixture was stirred at 20° C. for 1 hour. The reaction was warmed to room temperature. Under a nitrogen purge, the reaction was quenched with HCl (1.0 M, 5.0 ml) carefully, and then was added sodium hypochlorite (1.0 M, 5.0 ml). The mixture was filtered and concentrated under reduced pressure to give a residue, which was purified by prep-HPLC to give 3-fluoro-7-(1-methyl-1H-pyrazol-4-yl)-5,6,7,8-tetrahydro-1,7-naphthyridin-2-amine, Example 11-3 (1.60 mg, 6.47 μmol, 21.3% yield) as a white solid. LCMS [M+1]⁺=248.1. ¹H NMR (400 MHz, MeOD) δ=7.33 (s, 1H), 7.30 (s, 1H), 7.12 (d, J=11.2 Hz, 1H), 3.91 (s, 2H), 3.82 (s, 3H), 3.23 (t, J=6.0 Hz, 2H), 2.81 (t, J=5.6 Hz, 2H).

### Example 11-4

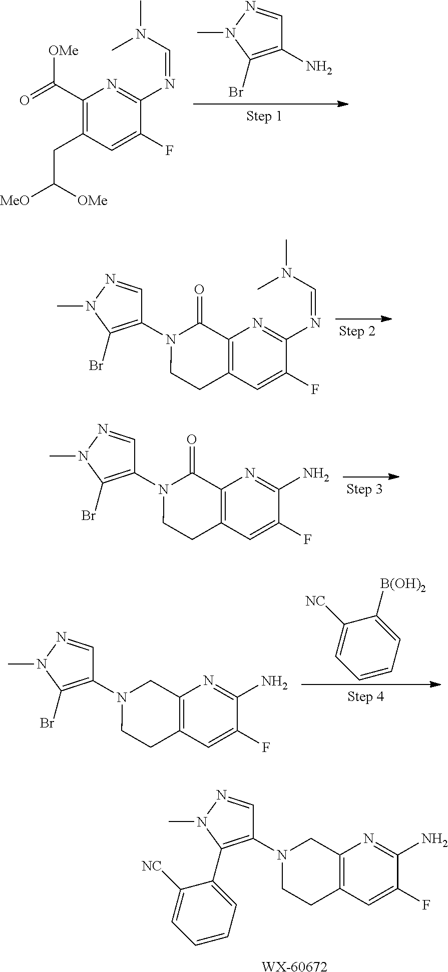

Step 1: To a solution of (Z)-methyl 3-(2,2-dimethoxyethyl)-6-(((dimethylamino)methylene)amino)-5-fluoropicolinate (See step 6 of Example 11-3), (500 mg, 1.60 mmol, 1.00 eq.) and 5-bromo-1-methyl-1H-pyrazol-4-amine (845 mg, 4.80 mmol, 3.00 eq.) in dichloromethane (2.00 mL) were added trifluoroacetic acid (11.5 g, 101 mmol, 7.50 mL, 63.3 eq.) and Et₃SiH (744 mg, 6.40 mmol, 1.02 mL, 4.00 eq.). The mixture was stirred at 25° C. for 2 hours. The reaction mixture was filtered and concentrated under reduced pressure to give a residue. The residue was purified by prep-HPLC. The desired fractions were collected and the aqueous layers was lyophilized to give (Z)—N-(7-(5-bromo-1-methyl-1H-pyrazol-4-yl)-3-fluoro-8-oxo-5,6,7,8-tetrahydro-1,7-naphthyridin-2-yl)-N,N-dimethylformimidamide (290 mg, crude) as a yellow solid.

Step 2: To a solution of (Z)—N-(7-(5-bromo-1-methyl-1H-pyrazol-4-yl)-3-fluoro-8-oxo-5,6,7,8-tetrahydro-1,7-naphthyridin-2-yl)-N,N-dimethylformimidamide (160 mg, 404 μmol, 1.00 eq.) in methanol (2.00 mL) was added hydrochloric acid (1.0 M, 405 μL 1.00 eq.). The mixture was stirred at 80° C. for 1 hour. The mixture was concentrated in vacuum. The residue was purified by prep-HPLC to give 2-amino-7-(5-bromo-1-methyl-1H-pyrazol-4-yl)-3-fluoro-6,7-dihydro-1,7-naphthyridin-8(5H)-one (74.0 mg, 213 μmol, 52.7% yield) as a white solid. LCMS [M+3]=342.1

Step 3: To a solution of 2-amino-7-(5-bromo-1-methyl-1H-pyrazol-4-yl)-3-fluoro-6,7-dihydro-1,7-naphthyridin-8(5H)-one (110 mg, 323 μmol, 1.00 eq.) in THF (4.00 mL) was added BH₃-Me₂S (10.0 M, 323 μL 10.0 eq.). The mixture was stirred at 60° C. for 2 hours. It was quenched by methanol (3.00 mL), then stirred for 1 hour. The mixture was concentrated in vacuum. The residue was purified by reversed phase flash [water (0.1% FA)/acetonitrile]. The desired fractions were collected and neutralized with solid sodium bicarbonate, concentrated under vacuum to remove acetonitrile. The aqueous phase was extracted with ethyl acetate (10.0 mL×2). The combined organic phase was dried with anhydrous sodium sulfate, filtered and concentrated in vacuum to give 7-(5-bromo-1-methyl-1H-pyrazol-4-yl)-3-fluoro-5,6,7,8-tetrahydro-1,7-naphthyridin-2-amine (60.0 mg, 182 μmol, 56.3% yield) as a white solid. LCMS [M+1]⁺=325.8.

Step 4: A mixture of 7-(5-bromo-1-methyl-1H-pyrazol-4-yl)-3-fluoro-5,6,7,8-tetrahydro-1,7-naphthyridin-2-amine (45.0 mg, 138 μmol, 1.00 eq.), (2-cyanophenyl)boronic acid (24.3 mg, 165 μmol, 1.20 eq.), potassium phosphate (1.50 M, 275.9 μL 3.00 eq.), cataCXium® A Pd G₃ (10.0 mg, 13.8 μmol, 0.10 eq.) in toluene (1.00 mL) was degassed and stirred at 90° C. for 1 hour under nitrogen atmosphere. The mixture was diluted with water (3.0 mL) and extracted with ethyl acetate (3.0 mL-5). The combined organic phase was dried over anhydrous sodium sulfate, filtered and concentrated in vacuum. The residue was purified by prep-HPLC to give 2-(4-(2-amino-3-fluoro-5,6-dihydro-1,7-naphthyridin-7(8H)-yl)-1-methyl-1H-pyrazol-5-yl)benzonitrile, Example 11-4 (3.50 mg, 10.0 μmol, 7.28% yield) as a yellow solid. LCMS [M+1]⁺=349.2. ¹H NMR (400 MHz, METHANOL-d₄) δ=7.90 (d, J=7.6 Hz, 1H), 7.84-7.78 (m, 1H), 7.69-7.60 (m, 2H), 7.49 (s, 1H), 7.05 (d, J=11.2 Hz, 1H), 3.78 (br d, J=4.0 Hz, 2H), 3.69 (s, 3H), 3.06 (q, J=6.0 Hz, 2H), 2.70-2.55 (m, 2H).

### Example 12-1

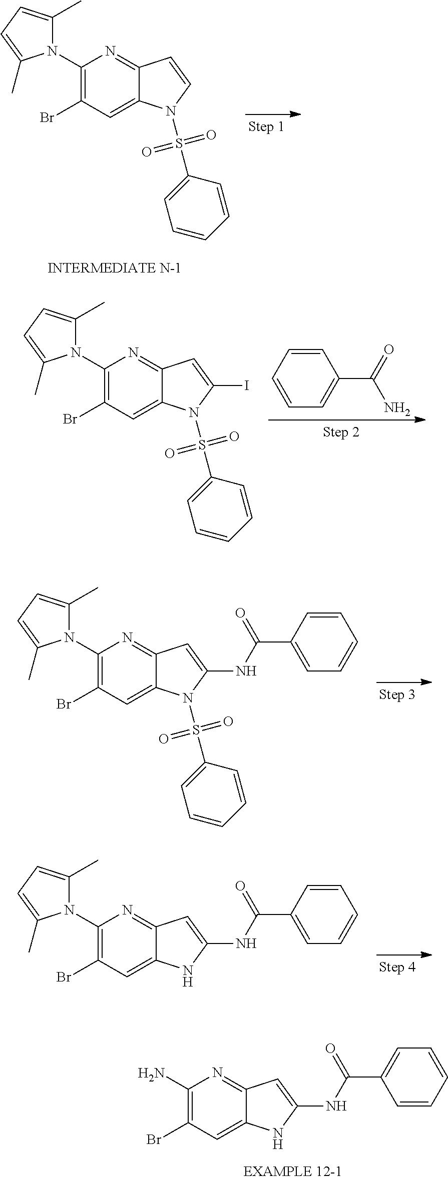

### Example 12-1

Step 1: To a solution of Intermediate N-1 (1.20 g, 2.79 mmol, 1.00 eq.) in THF (15.0 mL) was added LDA (2 M, 2.79 mL, 2.00 eq.) and TMEDA (486 mg, 4.18 mmol, 631 μL 1.50 eq.) drop-wise at −78° C. The mixture was stirred for 0.5 hr and then iodine (1.42 g, 5.58 mmol, 1.12 mL, 2.00 eq.) in THF (3.00 mL) was added drop-wise and the mixture was stirred at −78° C. for 0.25 hr. The reaction mixture was quenched with saturated ammonium chloride aqueous solution (20. mL) and extracted with ethyl acetate (20.0 mL×3), the combined organic phase was dried and concentrated in vacuum. The residue was purified by column chromatography (SiO₂, Petroleum ether/Ethyl acetate 50:1 to 30:1) to give 1-(benzenesulfonyl)-6-bromo-5-(2,5-dimethylpyrrol-1-yl)-2-iodo-pyrrolo[3,2-b]pyridine (1.00 g, 1.80 mmol, 64.5% yield) as a yellow solid. LCMS [M+1]: 555.9

Step 2: A mixture of 1-(benzenesulfonyl)-6-bromo-5-(2,5-dimethylpyrrol-1-yl)-2-iodo-pyrrolo[3,2-b]pyridine (400 mg, 719 μmol, 1.00 eq.), benzamide (105 mg, 863 μmol, 1.20 eq.), Pd₂(dba)₃ (65.9 mg, 71.9 μmol, 0.10 eq.), Xantphos (83.2 mg, 144 μmol, 0.20 eq.) and cesium carbonate (469 mg, 1.44 mmol, 2.00eq.) in dioxane (4.00 mL) was degassed and stirred at 100° C. for 1 hr under nitrogen atmosphere. The reaction mixture was filtered and concentrated in vacuum. The residue was purified by prep-TLC (SiO₂, Petroleum ether/Ethyl acetate 3:1) to give N-[1-(benzenesulfonyl)-6-bromo-5-(2,5-dimethylpyrrol-1-yl)pyrrolo[3,2-b]pyridin-2-yl]benzamide (120 mg, 218 μmol, 30.4% yield) as a yellow solid. ¹H NMR (400 MHz, DMSO) δ=10.84 (s, 1H), 8.67 (d, J=0.8 Hz, 1H), 8.07-8.00 (m, 2H), 7.93-7.87 (m, 2H), 7.77-7.68 (m, 2H), 7.67-7.56 (m, 4H), 7.03 (s, 1H), 5.79 (s, 2H), 1.85 (s, 6H).

Step 3: To a solution of N-[1-(benzenesulfonyl)-6-bromo-5-(2,5-dimethylpyrrol-1-yl)pyrrolo[3,2-b]pyridin-2-yl]benzamide (110 mg, 200 μmol, 1.00 eq.) in methyl alcohol (3.00 mL) was added sodium methoxide (54.1 mg, 1.00 mmol, 5.00 eq.), the mixture was stirred at 20° C. for 20 hrs. The pH of the reaction mixture was adjusted to 7 with acetic acid and the resulting was concentrated in vacuum to give a residue. The residue was purified by column chromatography (SiO₂, Petroleum ether/Ethyl acetate 4:1) to give N-[6-bromo-5-(2,5-dimethylpyrrol-1-yl)-1H-pyrrolo[3,2-b]pyridin-2-yl]benzamide (60.0 mg, 147 μmol, 73.2% yield) as a yellow solid. LCMS [M+1]: 409.1.

Step 4: A mixture of N-[6-bromo-5-(2,5-dimethylpyrrol-1-yl)-1H-pyrrolo[3,2-b]pyridin-2-yl]benzamide (50.0 mg, 122 μmol, 1.00 eq.), triethylamine (61.8 mg, 611 μmol, 85.0 μL 5.00 eq.) and hydroxylamine hydrochloride (170 mg, 2.44 mmol, 20.0 eq.) in ethyl alcohol (1.50 mL) in a sealed tube was heated to 110° C. and stirred for 3 hrs. The reaction mixture was filtered and concentrated in vacuum. The residue was purified by prep-HPLC (HCl condition) to give N-(5-amino-6-bromo-1H-pyrrolo [3, 2-b] pyridin-2-yl)benzamide, Example 12-1 (13.6 mg, 40.3 μmol, 33.0% yield) as a yellow solid. LCMS [M+1]: 331.1. ¹H NMR (400 MHz, DMSO) δ=12.02 (br s, 1H), 11.76 (s, 1H), 8.25 (s, 1H), 8.12-7.99 (m, 2H), 7.72-7.65 (m, 1H), 7.64-7.56 (m, 2H), 7.47-7.28 (m, 2H), 6.33 (d, J=1.2 Hz, 1H).

### Example 12-2

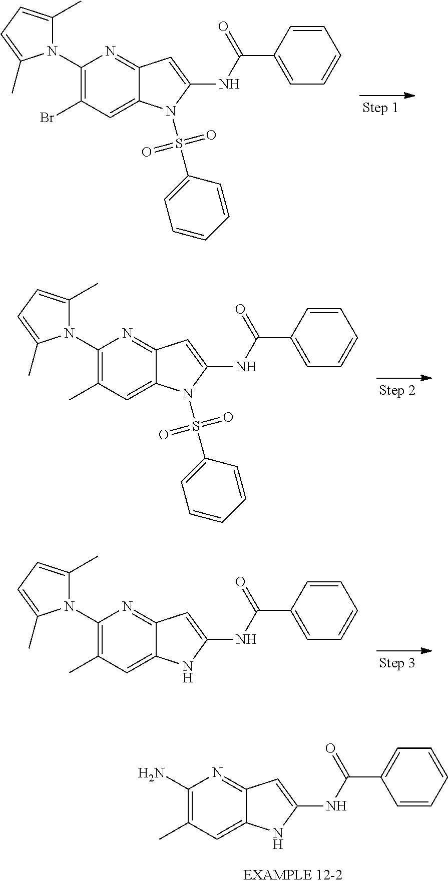 [SMILES: Cc1ccc(C)n1-c1nc2cc(NC(=O)c3ccccc3)n(S(=O)(=O)c3ccccc3)c2cc1Br ; Cc1cc2c(cc(NC(=O)c3ccccc3)n2S(=O)(=O)c2ccccc2)nc1-n1c(C)ccc1C ; Cc1cc2[nH]c(NC(=O)c3ccccc3)cc2nc1-n1c(C)ccc1C ; Cc1cc2[nH]c(NC(=O)c3ccccc3)cc2nc1N]

### Example 12-2

Step 1: A mixture of methylboronic acid (65.4 mg, 1.09 mmol, 3.00 eq.), N-[1-(benzenesulfonyl)-6-bromo-5-(2,5-dimethylpyrrol-1-yl)pyrrolo[3,2-b]pyridin-2-yl]benzamide (See Step 2 example 12-1) (200 mg, 364 μmol, 1.00 eq.), potassium carbonate (101 mg, 728 μmol, 2.00 eq.), Pd(dppf)Cl₂ (23.7 mg, 36.4 μmol, 0.10 eq.) in dioxane (2.00 mL) was degassed and stirred at 100° C. for 2 hrs under nitrogen atmosphere. The reaction mixture was concentrated under reduced pressure to give a residue. The residue was purified by prep-TLC (petroleum ether/ethyl acetate 2:1) to give N-[1-(benzenesulfonyl)-5-(2,5-dimethylpyrrol-1-yl)-6-methyl-pyrrolo[3,2-b]pyridin-2-yl]benzamide (120 mg, 248 μmol, 68.0% yield) as a yellow oil. LCMS [M+1]⁺=485.2. ¹H NMR (400 MHz, CDCl₃) δ=10.36 (s, 1H), 8.21 (s, 1H), 8.07-8.04 (m, 2H), 7.82-7.78 (m, 2H), 7.71-7.58 (m, 6H), 7.52-7.48 (m, 2H), 7.44 (s, 1H), 2.10 (s, 3H), 2.05 (s, 3H), 1.91 (s, 3H)

Step 2: To a solution of N-[1-(benzenesulfonyl)-5-(2,5-dimethylpyrrol-1-yl)-6-methyl-pyrrolo[3,2-b]pyridin-2-yl]benzamide (110 mg, 227 μmol, 1.00 eq.) in methyl alcohol (10.0 mL) was added sodium methoxide (123 mg, 2.27 mmol, 10.0 eq.). The mixture was stirred at 25° C. for 12 hrs. The reaction mixture was concentrated under reduced pressure to give a residue. The residue was diluted with water (10.0 mL) and pH was adjusted to 6 with 1N HCl (5.00 ML). The mixture was extracted with ethyl acetate (20.0 mL×3). Combined organic phase was washed with brine (20.0 mL), dried over anhydrous sodium sulfate, filtered and concentrated to give a residue. The residue was purified by reversed prep-HPLC (0.1% FA condition) to give N-[5-(2,5-dimethylpyrrol-1-yl)-6-methyl-1H-pyrrolo[3,2-b]pyridin-2-yl]benzamide (48.0 mg, 127 μmol, 56.0% yield) as a yellow oil. LCMS [M+1]⁺=345.2.

Step 3: To a solution of N-[5-(2,5-dimethylpyrrol-1-yl)-6-methyl-1H-pyrrolo[3,2-b]pyridin-2-yl]benzamide (40.0 mg, 116 μmol, 1.00 eq.) in ethyl alcohol (0.50 mL) was added hydroxylamine hydrochloride (807 mg, 11.6 mmol, 100 eq.) and TEA (235 mg, 2.32 mmol, 323 μL 20.0 eq.). The mixture was stirred at 110° C. for 12 hrs in a sealed tube. The reaction mixture was concentrated under reduced pressure to give a residue. The residue was purified by prep-HPLC (HCl condition) then by prep-TLC (DCM/methyl alcohol 10:1) and finally by another prep-HPLC (HCl condition) to give N-(5-amino-6-methyl-1H-pyrrolo[3,2-b]pyridin-2-yl)benzamide, Example 12-2 (0.88 mg, 2.88 μmol, 2.48% yield, HCl) as a yellow solid. LCMS [M+1]⁺=267.1. ¹H NMR (400 MHz, DMSO) δ=13.55-13.40 (m, 1H), 11.86 (s, 1H), 11.55 (s, 1H), 8.09-8.00 (m, 2H), 7.88 (s, 1H), 7.70-7.63 (m, 1H), 7.62-7.55 (m, 2H), 7.13 (br s, 2H), 6.28 (d, J=1.6 Hz, 1H), 2.20 (s, 3H).

### Example 12-3

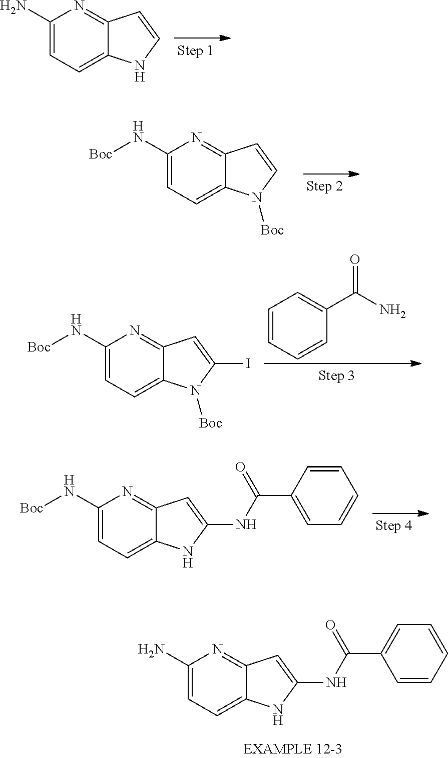

Step 1: To a solution of 1H-pyrrolo[3,2-b]pyridin-5-amine (500 mg, 2.95 mmol, 1.00 eq., HCl) in THF (20.0 mL) was added di-tert-butyl dicarbonate (1.29 g, 5.90 mmol, 1.35 mL, 2.00 eq.), DMAP (7.20 mg, 56.0 μmol, 0.02 eq.) and TEA (895 mg, 8.84 mmol, 1.23 mL, 3.00 eq.). The mixture was stirred at 25° C. for 3 hrs. The mixture was concentrated under reduced pressure to give a residue. The residue was purified by column chromatography (SiO₂, Petroleum ether/Ethyl acetate 50:1 to 15:1) to give tert-butyl 5-(tert-butoxycarbonylamino)pyrrolo[3,2-b]pyridine-1-carboxylate (220 mg, 641 μmol, 21.7% yield) as a yellow oil. LCMS [M+1]⁺=334.3. ¹H NMR (400 MHz, CDCl₃) δ=8.33 (br dd, J=2.4, 4.4 Hz, 1H), 7.93 (d, J=9.2 Hz, 1H), 7.77 (br d, J=2.4 Hz, 1H), 6.57 (d, J=3.6 Hz, 1H), 1.68 (s, 9H), 1.54 (s, 9H).

Step 2: To a solution of tert-butyl 5-(tert-butoxycarbonylamino)pyrrolo[3,2-b]pyridine-1-carboxylate (100 mg, 300 μmol, 1.00 eq.) and N,N,N′,N′-tetramethylethane-1,2-diamine (52.3 mg, 450 μmol, 67.9 μL 1.50 eq.) in THF (10.0 mL) was added LDA (2 M, 300 μL 2.00 eq.) at −78° C. under nitrogen atmosphere. After stirring at −78° for 10 min, iodine (91.4 mg, 360 μmol, 72.5 μL 1.20 eq.) was added to the reaction mixture. The mixture was stirred at −78° C. for 0.5 hrs. The mixture was quenched with water (2.00 mL) and extracted with ethyl acetate (10.0 mL×3). The combined organic phase was washed with brine, dried over sodium sulfate, filtered and concentrated under reduced pressure to give a residue. The residue was purified by prep-TLC (petroleum ether/ethyl acetate 3:1) to give tert-butyl 5-(tert-butoxycarbonylamino)-2-iodo-pyrrolo[3,2-h]pyridine-1-carboxylate (100 mg, 205 μmol, 68.2% yield) as a white solid. LCMS [M+1]⁺=460.1. ¹H NMR (400 MHz, CDCl₃) 5=8.28 (d, J=9.2 Hz, 1H), 7.86 (d, J=9.2 Hz, 1H), 7.43 (s, 1H), 7.01 (s, 1H), 1.72 (s, 9H), 1.54 (s, 9H).

Step 3: A mixture of benzamide (26.4 mg, 218 μmol, 2.00 eq.), tert-butyl 5-(tert-butoxycarbonylamino)-2-iodo-pyrrolo[3,2-b]pyridine-1-carboxylate (50.0 mg, 109 μmol, 1.00 eq.), Xantphos (12.6 mg, 21.8 μmol, 0.20 eq.), Pd₂(dba)₃ (9.97 mg, 10.9 μmol, 0.10 eq.) and cesium carbonate (71.0 mg, 218 μmol, 2.00 eq.) in dioxane (1.00 mL) was degassed and stirred at 100° C. for 1 hr under nitrogen atmosphere. The mixture was concentrated under reduced pressure to give a residue. The residue was purified by prep-TLC (petroleum ether/ethyl acetate 2:1) to give a residue to give tert-butyl N-(2-benzamido-1H-pyrrolo[3,2-b]pyridin-5-yl)carbamate (30.0 mg, 26.7 μmol, 24.5% yield, 31.3% purity) as a yellow solid. LCMS [M+1]⁺=353.2.

Step 4: A mixture of tert-butyl N-(2-benzamido-1H-pyrrolo[3,2-b]pyridin-5-yl)carbamate (23.4 mg, 66.3 μmol, 1.00 eq.) in TFA (1.00 mL) and DCM (3.00 mL) was degassed and stirred at 25° C. for 1 hr under nitrogen atmosphere. The mixture was concentrated under reduced pressure to give a residue. The residue was purified by prep-HPLC (HCl condition) to give N-(5-amino-1H-pyrrolo[3,2-b]pyridin-2-yl)benzamide, Example 12-3 (3.19 mg, 10.9 μmol, 16.5% yield, HCl) as a yellow solid. LCMS [M+1]⁺=253.1. ¹H NMR (400 MHz, DMSO-d₆) δ=13.69-13.26 (m, 1H), 11.99 (d, J=1.6 Hz, 1H), 11.68 (s, 1H), 8.10-8.04 (m, 2H), 7.99 (d, J=8.8 Hz, 1H), 7.70-7.64 (m, 1H), 7.63-7.56 (m, 2H), 7.36 (s, 2H), 6.47 (d, J=8.8 Hz, 1H), 6.32 (d, J=1.6 Hz, 1H).

### Example 12-4

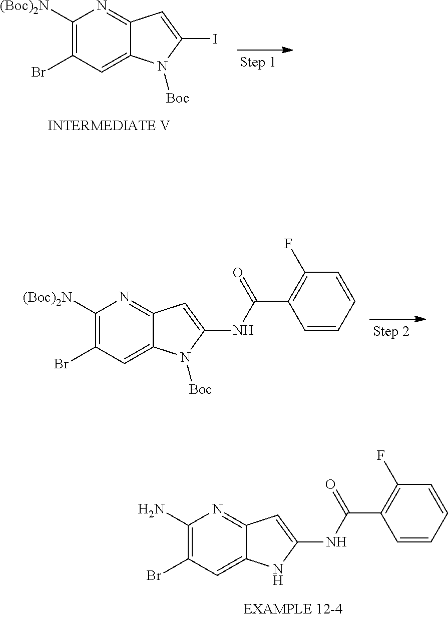

Step 1: A mixture of Intermediate V (200 mg, 313 μmol, 1.00 eq.), 2-fluorobenzamide (52.3 mg, 376 μmol, 1.20 eq.), Pd₂(dba)₃ (28.7 mg, 31.3 μmol, 0.10 eq.), Xantphos (36.3 mg, 62.7 μmol, 0.20 eq.) and cesium carbonate (204 mg, 627 μmol, 2.00 eq.) in dioxane (2.00 mL) was degassed and stirred at 100° C. for 1 hour under nitrogen atmosphere. The reaction mixture was diluted with water (40.0 mL) and extracted with ethyl acetate (30.0 mL×2). The combined organic layers were washed with brine (40.0 mL), dried over sodium sulfate, filtered and concentrated under reduced pressure to give a residue. The residue was purified by prep-TLC (SiO₂, Petroleum ether/Ethyl acetate 3:1) to give tert-butyl 5-[bis(tert-butoxycarbonyl)amino]-6-bromo-2-[(2-fluorobenzoyl)amino]pyrrolo[3,2-b]pyridine-1-carboxylate (22.0 mg, 33.9 μmol, 10.8% yield) as a white solid. LCMS [M+1]⁺=651.2

Step 2: To a solution of tert-butyl 5-[bis(tert-butoxycarbonyl)amino]-6-bromo-2-[(2-fluorobenzoyl)amino]pyrrolo[3,2-b]pyridine-1-carboxylate (20.0 mg, 30.8 μmol, 1.00 eq.) in methylene chloride (1.00 mL) was added TFA (462 mg, 4.05 mmol, 0.30 mL, 132 eq.). The mixture was stirred at 25° C. for 0.5 hour. The reaction mixture was concentrated under reduced pressure to give a residue. The residue was purified by prep-HPLC (HCl condition) to give N-(5-amino-6-bromo-1H-pyrrolo[3,2-b]pyridin-2-yl)-2-fluoro-benzamide, Example 12-4 (11.4 mg, 29.4 μmol, 95.6% yield, hydrochloric acid) as a green solid. LCMS [M+1]⁺=349.0. ¹H NMR (400 MHz, DMSO-d₆) δ=12.01 (s, 1H) 11.82 (s, 1H) 8.24 (d, J=0.8 Hz, 1H) 7.78 (td, J=7.5, 2.0 Hz, 1H) 7.63-7.69 (m, 1H) 7.24-7.49 (m, 4H) 6.27 (d, J=1.2 Hz, 1H).

### Example 13-1

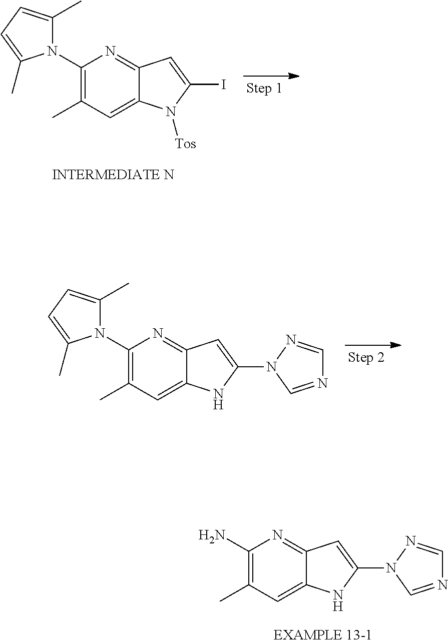

Step 1: To a solution of Intermediate N (190 mg, 375 μmol, 1.00 eq.) and 1H-1,2,4-triazole (38.9 mg, 563 μmol, 1.50 eq.) in dimethylsulfoxide (2.00 mL) was added cesium carbonate (367 mg, 1.13 mmol, 3.00 eq.) and Cu(acac)₂ (19.6 mg, 75.1 μmol, 0.20 eq.). The mixture was stirred at 120° C. for 16 hours. The mixture was purified by reverse phase chromatography with (0-85% of acetonitrile in water, 1‰ formic acid) to afford 5-(2,5-dimethylpyrrol-1-yl)-6-methyl-2-(1,2,4-triazol-1-yl)-1H-pyrrolo[3,2-b]pyridine (38.0 mg, 124 μmol, 33.1% yield) as a gray solid. LCMS [ESI, M+1]⁺=293.2. ¹HNMR (400 MHz, DMSO-d₆) δ=9.35 (s, 1H), 8.38 (s, 1H), 7.82 (s, 1H), 6.92 (s, 1H), 5.80 (s, 2H), 1.97 (s, 3H), 1.84 (s, 6H).

Step 2: To a solution of 5-(2,5-dimethylpyrrol-1-yl)-6-methyl-2-(1,2,4-triazol-1-yl)-1H-pyrrolo[3,2-b]pyridine (33.0 mg, 112 μmol, 1.00 eq.) in ethyl alcohol (2.00 mL) was added conc.HCl (408 mg, 4.03 mmol, 0.40 mL, 36.0% purity, 35.69 eq.). The mixture was stirred at 120° C. for 2 hours under microwave irradiation. The mixture was concentrated. The residue was purified by Prep-HPLC (HCl condition) to afford 6-methyl-2-(1,2,4-triazol-1-yl)-1H-pyrrolo[3,2-b]pyridin-5-amine, Example 13-1 (10.5 mg, 47.1 μmol, 41.8% yield) as a pink solid. LCMS [ESI, M+1]⁺=215.1. ¹HNMR (400 MHz, MeOD) δ=9.19 (s, 1H), 8.27 (s, 1H), 7.95 (s, 1H), 6.78 (s, 1H), 2.34 (s, 3H).

### Example 13-2

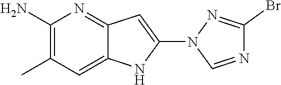

2-(3-bromo-1,2,4-triazol-1-yl)-6-methyl-1H-pyrrolo[3,2-b]pyridin-5-amine, Example 13-2 was prepared from Intermediate N and 3-bromo-1H-1,2,4-triazole following the two step procedure described for Example 13-1 to afford the title compound as (2.25 mg, 6.51 μmol, 6% yield over two steps, Formic acid salt) as off-white solid; LCMS [ESI, M+1]⁺: 293.1/295.1; ¹H NMR (400 MHz, MeOD-d₄) δ=9.01 (s, 1H), 8.38 (br s, 1H), 7.71 (s, 1H), 6.63 (s, 1H), 2.29 (s, 3H);

### Example 13-3

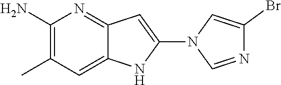

2-(4-bromoimidazol-1-yl)-6-methyl-1H-pyrrolo[3,2-b]pyridin-5-amine, Example 13-3 was prepared from Intermediate N and following the two step procedure described for Example 13-1 to afford the title compound (8.13 mg, 22.6 μmol, 14% yield over 2 steps, hydrochloride) as a white solid. LCMS [ESI, M+1]: 294.0. ¹H NMR (400 MHz, DMSO-d₆) δ=14.34-14.13 (m, 1H), 13.35-13.10 (m, 1H), 8.44 (s, 1H), 8.10 (s, 1H), 7.96 (s, 1H), 7.50 (s, 2H), 6.72 (d, J=1.2 Hz, 1H), 2.25 (s, 3H)

### Example 13-4

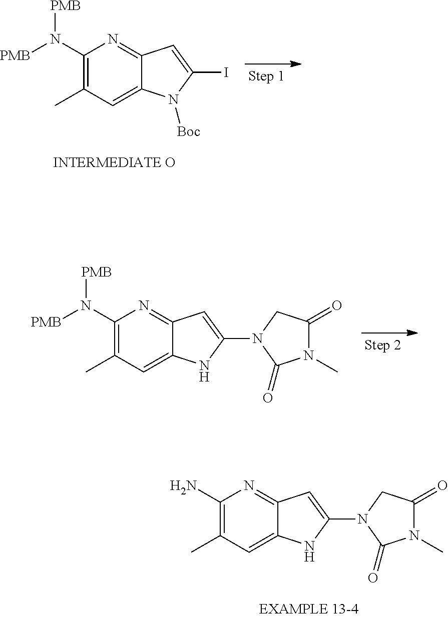

Step 1: A mixture of Intermediate O (280 mg, 536 μmol, 1 equiv), 3-methylimidazolidine-2,4-dione (91.80 mg, 804.55 μmol, 1.5 equiv), BrettPhos Pd G₃ (48.62 mg, 53.64 μmol, 0.1 equiv), RuPhos (25.03 mg, 53.64 μmol, 0.1 equiv) and potassium phosphate (341 mg, 1.61 mmol, 3.00 equiv) in ter-amyl alcohol (4 mL) was degassed and stirred at 130° C. for 18 hours under nitrogen atmosphere. The reaction mixture was concentrated under reduced pressure to give a residue. The residue was purified by prep-HPLC to give 1-[5-[bis[(4-methoxyphenyl)methyl]amino]-6-methyl-1H-pyrrolo[3,2-b]pyridin-2-yl]-3-methyl-imidazolidine-2,4-dione (51.0 mg, 96.8 μmol, 18.0% yield) as a yellow solid. LCMS (EST, M+1): m/z=500.2.

Step 2: A solution of 1-[5-[bis[(4-methoxyphenyl)methyl]amino]-6-methyl-1H-pyrrolo[3,2-b]pyridin-2-yl]-3-methyl-imidazolidine-2,4-dione (51.0 mg, 102 μmol, 1.00 equiv) in trifluoroacetic acid (1.00 mL) was stirred at 40° C. for 2 hours. The reaction mixture was concentrated under reduced pressure to give a residue. The residue was purified by prep-HPLC (HCl condition) to give 1-(5-amino-6-methyl-1H-pyrrolo[3,2-b]pyridin-2-yl)-3-methyl-imidazolidine-2,4-dione, Example 13-4 (9.57 mg, 31.9 μmol, 31.3% yield, HCl) as an off-white solid. LCMS [EST, M+1]: 260.0. ¹H NMR (400 MHz, DMSO-d₆) δ=14.03-13.74 (m, 1H), 12.10-11.89 (m, 1H), 7.83 (s, 1H), 7.20 (s, 2H), 6.24-6.09 (m, 1H), 4.51 (s, 2H), 2.96 (s, 3H), 2.20 (s, 3H).

### Example 13-5

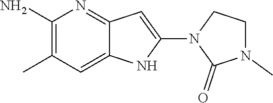

1-(5-amino-6-methyl-1H-pyrrolo[3,2-b]pyridin-2-yl)-3-methyl-imidazolidin-2-one, Example 13-5 was prepared from Intermediate O and 1-methylimidazolidin-2-one following the two step procedure described for Example 13-4 to afford the title compound (6.27 mg, 24.7 μmol, 24.0% yield over 2 steps) as a yellow solid. LCMS [M+1]⁺=246.1. ¹H NMR (400 MHz, MeOD-d₄) δ=7.37 (s, 1H), 5.62 (s, 1H), 3.88-3.81 (m, 2H), 3.63-3.57 (m, 2H), 2.89 (s, 3H), 2.21 (s, 3H)

### Example 13-6

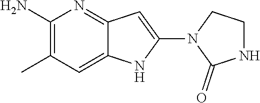

1-(5-amino-6-methyl-1H-pyrrolo[3,2-b]pyridin-2-yl)imidazolidin-2-one, Example 13-5 was prepared from Intermediate O and 2-methylbutan-2-ol following the two step procedure described for Example 13-4 to afford the title compound (12.0 mg, 50.2 μmol, 21% yield over two steps, formic acid salt) as a yellow solid. LCMS [M+1]⁺=232.1. 1H NMR (400 MHz, MeOD-d₄) δ=7.70 (s, 1H), 5.79 (s, 1H), 3.97-3.88 (m, 2H), 3.64-3.56 (m, 2H), 2.18 (s, 3H).

### Example 13-7

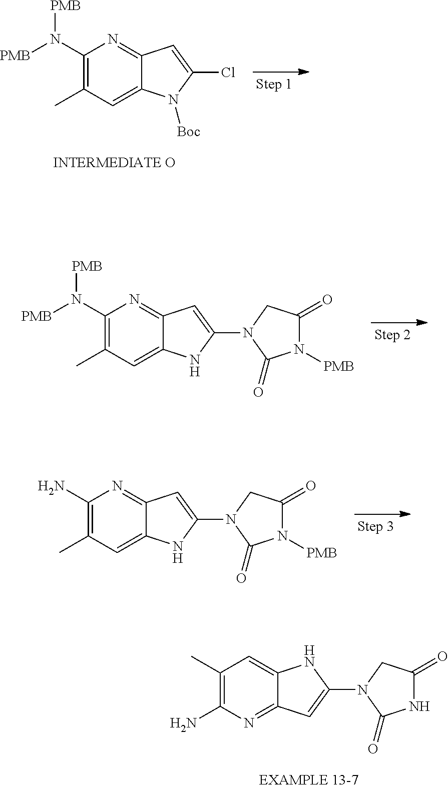

Step 1. To a mixture of Intermediate O (500 mg, 958 μmol, 1.00 eq.) and 3-[(4-methoxyphenyl)methyl]imidazolidine-2,4-dione (211 mg, 958 μmol, 1.00 eq.) in 2-methyl-2-butanol (5.00 mL) were added BrettPhos Pd G₃ (86.8 mg, 95.8 μmol, 0.10 eq.), RuPhos (89.4 mg, 192 μmol, 0.20 eq.) and potassium phosphate (610 mg, 2.87 mmol, 3.00 eq.) in one portion at 20° C. under nitrogen atmosphere. The mixture was stirred at 120° C. for 18 hours. The reaction mixture was diluted with water (20.0 mL) and extracted with ethyl acetate (3×30.0 mL). The combined organic layers were washed with brine (30.0 mL), dried over anhydrous sodium sulfate, filtered and concentrated under reduced pressure. The residue was purified by reversed phase flash column chromatography [water (0.1% formic acid)/acetonitrile)] and then re-purified by column chromatography (SiO₂, petroleum ether/ethyl acetate 1:0 to 20:1) to give 1-(5-(bis(4-methoxybenzyl)amino)-6-methyl-1H-pyrrolo[3,2-b]pyridin-2-yl)-3-(4-methoxybenzyl)imidazolidine-2,4-dione (84.0 mg, 128 μmol, 13.0% yield) as a white solid. LCMS [M+1]⁺=606.3. ¹H NMR (400 MHz, CDCl₃) δ=10.29-9.40 (m, 1H), 7.40 (d, J=8.6 Hz, 3H), 7.22 (br d, J=8.4 Hz, 4H), 6.88 (d, J=8.8 Hz, 2H), 6.78 (d, J=8.4 Hz, 4H), 6.32-5.64 (m, 1H), 4.71 (s, 3H), 4.43-4.22 (m, 6H), 3.76 (s, 6H), 2.44 (s, 3H).

Step 2: To mixture of 1-(5-(bis(4-methoxybenzyl)amino)-6-methyl-1H-pyrrolo[3,2-b]pyridin-2-yl)-3-(4-methoxybenzyl)imidazolidine-2,4-dione (80.0 mg, 132 μmol, 1.00 eq.) in DCM (1.00 mL) was stirred at 60° C. for 5 hours. The reaction mixture was filtered and concentrated under reduced pressure. Crude 1-(5-amino-6-methyl-1H-pyrrolo[3,2-b]pyridin-2-yl)-3-(4-methoxybenzyl)imidazolidine-2,4-dione was used directly in the next step without further purification (48.0 mg, 131 μmol, 99.00% yield) as a dark brown solid. LCMS [M+1]⁺=366.0.

Step 3: To a mixture of 1-(5-amino-6-methyl-1H-pyrrolo[3,2-b]pyridin-2-yl)-3-(4-methoxybenzyl)imidazolidine-2,4-dione (48.0 mg, 131 μmol, 1.00 eq.) in trifluoroacetic acid (0.50 mL) was added trifluoromethanesulfonic acid (85.0 mg, 566 μmol, 0.05 mL, 4.31 eq.) in one portion under nitrogen atmosphere. The mixture was stirred at 80° C. for 2 hours. The reaction mixture was filtered and concentrated under reduced pressure. The residue was purified by prep-HPLC to give 1-(5-amino-6-methyl-1H-pyrrolo[3,2-b]pyridin-2-yl)imidazolidine-2,4-dione, Example 13-7 (7.72 mg, 26.5 μmol, 20.2% yield, FA salt) as a white solid. LCMS [M+1]=246.1. ¹H NMR (400 MHz, MeOD-d₄) δ=8.43 (s, 1H), 7.81 (s, 1H), 6.06 (s, 1H), 4.48 (s, 2H), 2.29 (s, 3H).

### Example 14-1

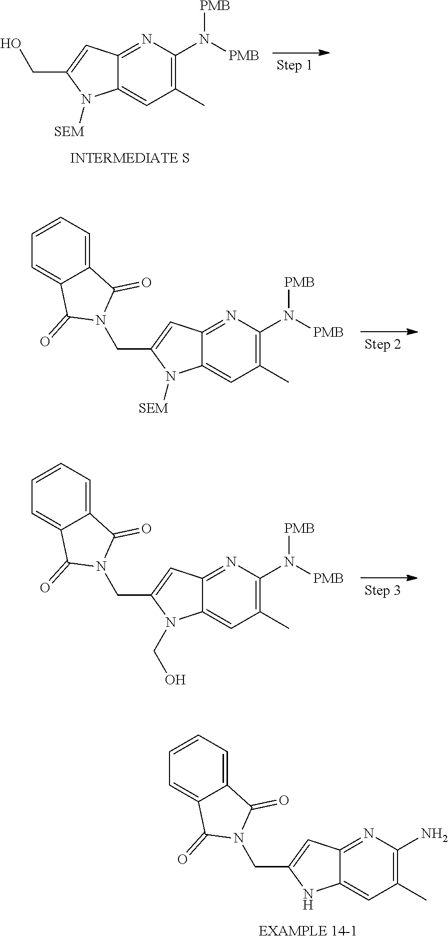

Step 1: To a solution of Intermediate S (200 mg, 365 μmol, 1.00 eq.), isoindoline-1,3-dione (59.1 mg, 402 μmol, 1.10 eq.) and triphenylphosphine (144 mg, 548 μmol, 1.50 eq.) in tetrahydrofuran (2.00 mL) was added diisopropylazodicarboxylate (111 mg, 548 μmol, 106 μL 1.50 eq.) at 0° C. The mixture was stirred at 20° C. for 3 hours. Then the mixture was heated to 40° C. and stirred for 3 hours. The mixture was diluted with water (30.0 mL) and extracted with ethyl acetate (15.0 mL×2). The organic layer was washed with brine (20.0 ML), dried over sodium sulfate, filtered and concentrated. The residue was purified by flash silica gel chromatography (10-30% Ethyl acetate/Petroleum) to afford 2-((5-(bis(4-methoxybenzyl)amino)-6-methyl-1-((2-(trimethylsilyl)ethoxy)methyl)-1H-pyrrolo[3,2-b]pyridin-2-yl)methyl)isoindoline-1,3-dione (200 mg, 295 μmol, 80.9% yield) as a light yellow oil. ¹H NMR (400 MHz, CDCl₃) δ=7.86 (br dd, J=3.2, 5.2 Hz, 2H), 7.73 (br dd, J=2.8, 4.8 Hz, 2H), 7.20 (br d, J=8.2 Hz, 3H), 6.77 (br d, J=8.2 Hz, 4H), 6.33 (br s, 4H), 5.59 (s, 1H), 4.99 (td, J=6.0, 12.4 Hz, 5H), 4.20-4.14 (m, 4H), 3.79-3.72 (m, 8H), 2.49-2.41 (m, 3H), 1.86 (td, J=3.2, 6.4 Hz, 1H), -0.03-0.11 (m, 9H).

Step 2: To a solution of 2-((5-(bis(4-methoxybenzyl)amino)-6-methyl-1-((2-(trimethylsilyl)ethoxy)methyl)-1H-pyrrolo[3,2-b]pyridin-2-yl)methyl)isoindoline-1,3-dione (200 mg, 295 μmol, 1.00 eq.) in dichloromethane (8.00 mL) was added trifluoroacetic acid (2.46 g, 21.6 mmol, 1.60 mL, 73.1 eq) and the mixture was stirred at 20° C. for 3 h. The mixture was concentrated to afford 2-((5-amino-1-(hydroxymethyl)-6-methyl-1H-pyrrolo[3,2-b]pyridin-2-yl)methyl)isoindoline-1,3-dione (99.0 mg, crude) as a yellow oil.

Step 3: To a solution of 2-((5-amino-1-(hydroxymethyl)-6-methyl-1H-pyrrolo[3,2-b]pyridin-2-yl)methyl)isoindoline-1,3-dione (99.0 mg, 294 μmol, 1.00 eq.) in dioxane (5.00 mL) was added ammonium hydroxide (413 mg, 2.94 mmol, 453 μL 25.0% purity, 10.0 eq.). Then the mixture was stirred at 60° C. for 2 hours. The mixture was concentrated. The residue was purified by prep-HPLC (HCl condition) and lyophilized to afford 2-((5-amino-6-methyl-1H-pyrrolo[3,2-b]pyridin-2-yl)methyl)isoindoline-1,3-dione, Example 14-1 (39.4 mg, 112 μmol, 38.2% yield, hydrochloric acid salt) as an off-white solid. LCMS [ESI, M+1]: 307.1. ¹H NMR (400 MHz, DMSO-d₆) δ=13.61 (br s, 1H), 11.95 (s, 1H), 7.97-7.84 (m, 5H), 7.34 (s, 2H), 6.33 (s, 1H), 4.93 (s, 2H), 2.21 (s, 3H).

### Example 14-2

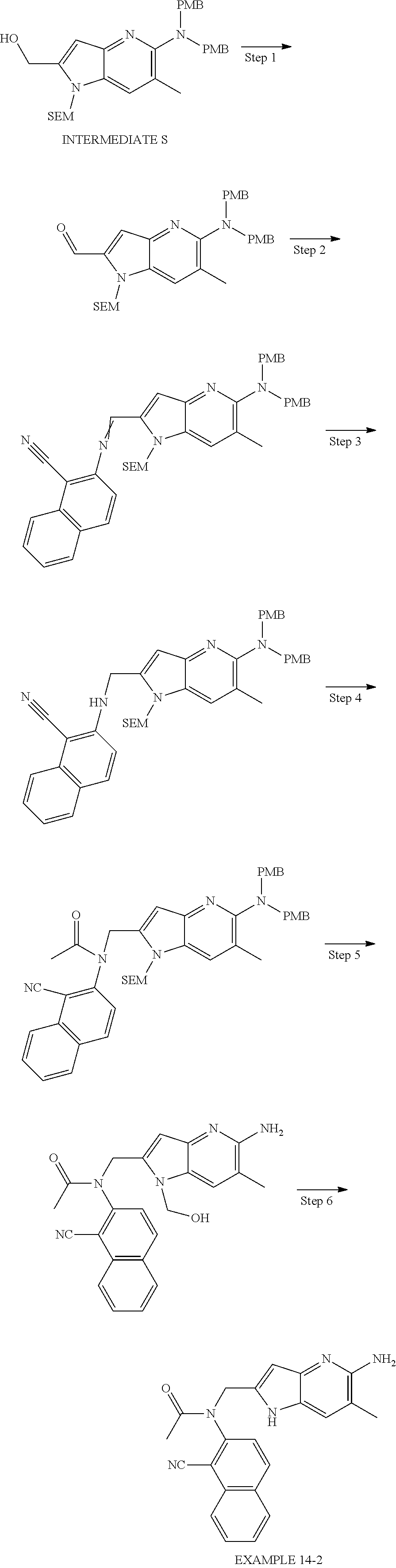

Step 1: To a solution of Intermediate S (1.50 g, 2.74 mmol, 1.00 eq.) in dichloromethane (15.0 mL) was added manganese(IV) oxide (2.38 g, 27.4 mmol, 10.0 eq.) at 25° C. The mixture was stirred at 35° C. for 16 hours. The mixture was filtered. The filter cake was washed with dichloromethane (10.0 mL×3). The combined filtrate was concentrated in vacuo. The residue was purified by flash silica gel chromatography (14% ethyl acetate/Petroleum ether) to afford 5-[bis[(4-methoxyphenyl)methyl]amino]-6-methyl-1-(2-trimethylsilylethoxymethyl)pyrrolo[3,2-b]pyridine-2-carbaldehyde (1.19 g, 2.01 mmol, 73.3% yield) as a yellow oil: LCMS [ESI, M+1]⁺: 546.4. ¹H NMR (400 MHz, CDCl₃) δ=9.89 (s, 1H), 7.68 (br s, 1H), 7.21 (d, J=8.4 Hz, 4H), 6.81 (d, J=8.8 Hz, 5H), 5.95 (s, 2H), 4.25 (br s, 4H), 3.78 (s, 7H), 3.58-3.51 (m, 2H), 2.57 (s, 3H), 0.95-0.84 (m, 2H), -0.07 (s, 9H).

Step 2: To a solution of 5-[bis[(4-methoxyphenyl)methyl]amino]-6-methyl-1-(2-trimethylsilylethoxymethyl)pyrrolo[3,2-b]pyridine-2-carbaldehyde (300 mg, 550 μmol, 1.00 eq.) and 2-aminonaphthalene-1-carbonitrile (92.5 mg, 550 μmol, 1.00 eq.) in toluene (6.00 mL) was added tetraisopropoxytitanium (312 mg, 1.10 mmol, 324 μL 2.00 eq.). The mixture was stirred at 110° C. for 14 hours and then at 125° C. for another 3 hours. The mixture was concentrated in vacuo to afford 2-[[5-[bis[(4-methoxyphenyl)methyl]amino]-6-methyl-1-(2-trimethylsilylethoxymethyl)pyrrolo[3,2-b]pyridin-2-yl]methyleneamino]naphthalene-1-carbonitrile (380 mg, crude) as a yellow solid;

Step 3: To a solution of 2-[[5-[bis[(4-methoxyphenyl)methyl]amino]-6-methyl-1-(2-trimethylsilylethoxymethyl)pyrrolo[3,2-b]pyridin-2-yl]methyleneamino]naphthalene-1-carbonitrile (380 mg, 546 μmol, 1.00 eq.) in tetrahydrofuran (6.00 mL) was added sodium cyanoborohydride (51.5 mg, 819 μmol, 1.50 eq.) at 0° C. under nitrogen atmosphere. The mixture was stirred at 25° C. for 16 hours. The mixture was diluted with ethyl acetate (10.0 mL) and then filtered. The filter cake was washed with ethyl acetate (5.00 mL-3). The combined filtrate was concentrated in vacuo. The residue was purified by flash silica gel chromatography (21% ethyl acetate/Petroleum ether). The collected mixture was concentrated in vacuo to afford 2-[[5-[bis[(4-methoxyphenyl)methyl]amino]-6-methyl-1-(2-trimethylsilylethoxymethyl)pyrrolo[3,2-b]pyridin-2-yl]methylamino]naphthalene-1-carbonitrile (215 mg, 280 μmol, 51.3% yield) as a yellow oil; LCMS [ESI, M+1]⁺: 698.4.

Step 4: To a solution of 2-[[5-[bis[(4-methoxyphenyl)methyl]amino]-6-methyl-1-(2-trimethylsilylethoxymethyl)pyrrolo[3,2-b]pyridin-2-yl]methylamino]naphthalene-1-carbonitrile (215 mg, 308 μmol, 1.00 eq.) in N,N-dimethylformamide (2.50 mL) was added sodium hydride (24.6 mg, 616 μmol, 60% purity, 2.00 eq.) at 0° C. under nitrogen atmosphere. The mixture was stirred at 0° C. for 0.5 hour. Acetyl chloride (48.4 mg, 616 μmol, 44.0 μL 2.00 eq.) was added to the mixture at 0° C. under nitrogen atmosphere. The resulting mixture was stirred at 25° C. for 16 hours. The mixture was cooled to 0° C. Another batch sodium hydride (24.6 mg, 616 μmol, 60% purity, 2.00 eq.) was added to the mixture at 0° C. under nitrogen atmosphere. The mixture was stirred at 0° C. for 0.5 hour. Then another batch acetyl chloride (48.4 mg, 616 μmol, 44.0 μL 2.00 eq.) was added to the mixture at 0° C. Then the resulting mixture was warmed up to 25° C. slowly and stirred at 25° C. for 16 hours. The mixture was poured into water (15.0 mL) and extracted with ethyl acetate (10.0 mL×3). The combined organic layers were washed with water (10.0 mL), dried over anhydrous sodium sulfate, filtered and concentrated in vacuo. The residue was purified by flash silica gel chromatography (28% ethyl acetate/Petroleum ether). The collected mixture was concentrated in vacuo to afford N-[[5-[bis[(4-methoxyphenyl)methyl]amino]-6-methyl-1-(2-trimethylsilylethoxymethyl)pyrrolo[3,2-b]pyridin-2-yl]methyl]-N-(1-cyano-2-naphthyl)acetamide (136 mg, 178 μmol, 57.8% yield) as a yellow oil. LCMS [ESI, M+1]⁺: 740.4.

Step 5: To a solution of N-[[5-[bis[(4-methoxyphenyl)methyl]amino]-6-methyl-1-(2-trimethylsilylethoxymethyl)pyrrolo[3,2-b]pyridin-2-yl]methyl]-N-(1-cyano-2-naphthyl)acetamide (136 mg, 184 μmol, 1.00 eq.) in dichloromethane (2.00 mL) was added trifluoroacetic acid (616 mg, 5.40 mmol, 0.40 mL, 29.4 eq.). The mixture was stirred at 25° C. for 2 hours. The mixture was concentrated in vacuo to afford N-[[5-amino-1-(hydroxymethyl)-6-methyl-pyrrolo[3,2-b]pyridin-2-yl]methyl]-N-(1-cyano-2-naphthyl)acetamide (70.0 mg, 175 μmol, 95.4% yield) as a yellow oil. LCMS [EST, M+1]⁺: 400.2.

Step 6: A solution of N-[[5-amino-1-(hydroxymethyl)-6-methyl-pyrrolo[3,2-b]pyridin-2-yl]methyl]-N-(1-cyano-2-naphthyl)acetamide (70.0 mg, 175 μmol, 1.00 eq.) and ammonium hydroxide (1.82 g, 13.0 mmol, 2.00 mL, 25% purity, 74.1 eq) was stirred at 25° C. for 16 hours. The resulting mixture was concentrated in vacuo. The residue was purified by Prep-HPLC (HCl condition) to afford N-[(5-amino-6-methyl-1H-pyrrolo[3,2-b]pyridin-2-yl)methyl]-N—(I-cyano-2-naphthyl)acetamide, Example 14-2 (14.1 mg, 33.5 μmol, 16.8% yield, hydrochloride) as a yellow solid; LCMS [EST, M+1]⁺: 370.2. ¹H NMR (400 MHz, MeOD-d₄) δ=8.31 (d, J=8.8 Hz, 1H), 8.12 (br t, J=8.0 Hz, 2H), 7.86 (s, 1H), 7.83-7.71 (m, 2H), 7.49 (d, J=8.8 Hz, 1H), 6.18 (s, 11H), 5.32 (br d, J=15.2 Hz, 1H), 5.03 (br d, J=15.2 Hz, 1H), 2.28 (s, 3H), 1.95 (s, 3H).

### Example 14-3

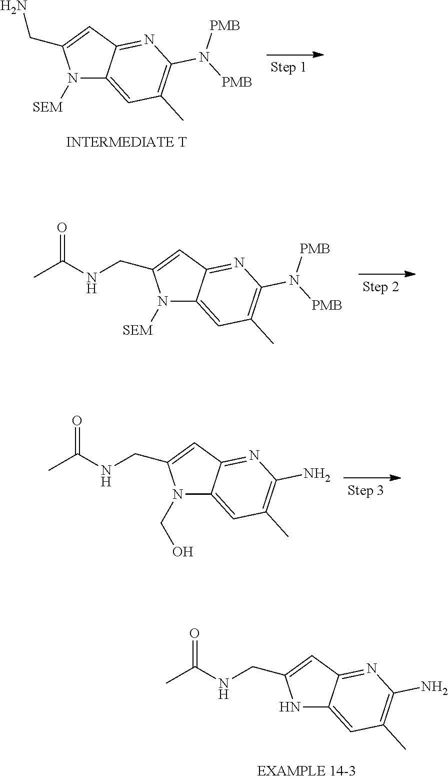

Step 1: To a solution of Intermediate T (450 mg, 823 μmol, 1.00 eq.) in tetrahydrofuran (9.00 mL) was added triethylamine (125 mg, 1.23 mmol, 172 μL 1.50 eq.) and acetic anhydride (126 mg, 1.23 mmol, 116 μL 1.50 eq.). The mixture was stirred at 20° C. for 2 hours. The mixture was diluted with water (30.0 mL) and extracted with ethyl acetate (10.0 mL×3). The organic layer was washed with brine (20.0 mL), dried over sodium sulfate, filtered and concentrated. The residue was purified by flash silica gel chromatography (10-50% Ethyl acetate/Petroleum ether) to afford N-((5-(bis(4-methoxybenzyl)amino)-6-methyl-1-((2-(trimethylsilyl)ethoxy)methyl)-1H-pyrrolo[3,2-b]pyridin-2-yl)methyl)acetamide (320 mg, 369 μmol, 44.9% yield, 67.9% purity) as a light yellow oil. LCMS [ESI, M+1]⁺=589.3. ¹HNMR (400 MHz, CDCl₃) δ=7.71-7.63 (m, 4H), 7.58-7.52 (m, 2H), 7.51-7.43 (m, 4H), 7.24 (br d, J=8.4 Hz, 4H), 6.80 (br d, J=8.4 Hz, 4H), 5.47 (s, 2H), 4.66 (br d, J=5.6 Hz, 2H), 4.37 (s, 3H), 3.76 (s, 6H), 3.53 (br t, J=8.0 Hz, 2H), 2.50 (s, 3H), 2.05 (s, 4H), 0.96-0.86 (m, 3H), -0.04 (s, 9H).

Step 2: To a solution of N-((5-(bis(4-methoxybenzyl)amino)-6-methyl-1-((2-(trimethylsilyl)ethoxy)methyl)-1H-pyrrolo[3,2-b]pyridin-2-yl)methyl)acetamide (50.0 mg, 84.9 μmol, 1.00 eq) in dichloromethane (1.50 mL) was added trifluoroacetic acid (462 mg, 4.05 mmol, 300 μL 47.7 eq.). The mixture was stirred at 20° C. for 3 hours. The mixture was concentrated to afford N-((5-amino-1-(hydroxymethyl)-6-methyl-1H-pyrrolo[3,2-b]pyridin-2-yl)methyl)acetamide (21.0 mg, crude) as a light yellow oil.

Step 3: To a solution of N-((5-amino-1-(hydroxymethyl)-6-methyl-1H-pyrrolo[3,2-b]pyridin-2-yl)methyl)acetamide (21.0 mg, 84.6 μmol, 1.00 eq.) in dioxane (1.50 mL) was added ammonium hydroxide (119 mg, 846 μmol, 130 μL 25.0% purity, 10.0 eq.). The mixture was stirred at 20° C. for 2 hours. The mixture was concentrated. The residue was purified by prep-HPLC (neutral condition) and lyophilized to afford N-((5-amino-6-methyl-1H-pyrrolo[3,2-b]pyridin-2-yl)methyl)acetamide, Example 14-3 (5.94 mg, 21.4 μmol, 25.3% yield, formic acid salt) as yellow solid. LCMS [ESI, M+1]⁺: 219.1. ¹H NMR (400 MHz, MeOD-d₄) δ=7.77 (s, 1H), 6.27 (s, 1H), 4.96 (s, 2H), 2.27 (1, 3H), 2.01 (s, 3H).

### Example 14-4

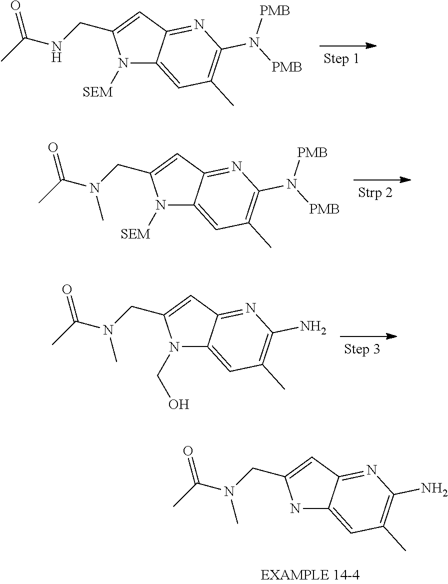

Step 1: To a solution of N-((5-(bis(4-methoxybenzyl)amino)-6-methyl-1-((2-(trimethylsilyl)ethoxy)methyl)-1H-pyrrolo[3,2-b]pyridin-2-yl)methyl)acetamide (see step 1 of Example 14-3) (100 mg, 170 μmol, 1.00 eq.) in dimethyl formamide (2.00 mL) was added sodium hydride (10.2 mg, 255 μmol, 60% purity, 1.50 eq.) at 0° C. under nitrogen atmosphere. The mixture was stirred at 0° C. for 0.5 hours. Then a solution of methyl iodide (60.3 mg, 425 μmol, 26.4 μL 2.50 eq.) in dimethyl formamide (1.00 mL) was added to the reaction mixture. The resulting mixture was stirred at 20° C. for 2 hours. The mixture was quenched with saturated ammonium chloride (30.0 mL) and extracted with ethyl acetate (15.0 mL). The organic layer was dried over sodium sulfate, filtered and concentrated. The residue was purified by flash silica gel chromatography (10-50% Ethyl acetate/Petroleum ether) to afford N-((5-(bis(4-methoxybenzyl)amino)-6-methyl-1-((2-(trimethylsilyl)ethoxy) methyl)-1H-pyrrolo[3,2-b]pyridin-2-yl)methyl)-N-methylacetamide (75.0 mg, 90.3 μmol, 53.2% yield, 72.6% purity) as a light yellow oil. LCMS [ESI, M+1]⁺=603.3. ¹HNMR (400 MHz, CDCl₃) δ=7.71-7.65 (m, 1H), 7.59-7.53 (m, 1H), 7.24 (br d, J=8.0 Hz, 4H), 6.79 (d, J=8.4 Hz, 4H), 5.52-5.30 (m, 2H), 4.85-4.78 (m, 2H), 4.28-4.18 (m, 2H), 3.77 (s, 6H), 2.99-2.94 (m, 2H), 2.49 (s, 3H), 2.14 (s, 2H), 0.95-0.79 (m, 3H), 0.04-0.13 (m, 9H).

Step 2: To a solution of N-((5-(bis(4-methoxybenzyl)amino)-6-methyl-1-((2-(trimethylsilyl)ethoxy)methyl)-1H-pyrrolo[3,2-b]pyridin-2-yl)methyl)-N-methylacetamide (50.0 mg, 82.9 μmol, 1.00 eq.) in dichloromethane (1.50 mL) was added trifluoroacetic acid (462 mg, 4.05 mmol, 0.30 mL, 48.9 eq.). The mixture was stirred at 20° C. for 2 hours. The mixture was concentrated to afford N-((5-amino-1-(hydroxymethyl)-6-methyl-1H-pyrrolo[3,2-b]pyridin-2-yl)methyl)-N-methylacetamide (21.0 mg, crude) as a light yellow oil. LCMS [ESI, M+1]⁺=263.1.

Step 3: To a solution of N-((5-amino-1-(hydroxymethyl)-6-methyl-1H-pyrrolo[3,2-b]pyridin-2-yl)methyl)-N-methylacetamide (21.0 mg, 80.1 μmol, 1.00 eq.) in dioxane (1.00 mL) was added ammonium hydroxide (112 mg, 801 μmol, 123 μL 25.0% purity, 10.0 eq). The mixture was stirred at 20° C. for 3 hours. The residue was purified by prep-HPLC (HCl condition) followed by re-purification by prep-HPLC (neutral condition) to afford N-((5-amino-6-methyl-1H-pyrrolo[3,2-b]pyridin-2-yl)methyl)-N-methylacetamide, Example 14-4 (8.55 mg, 36.4 μmol, 45.5% yield) as a yellow gum. LCMS [ESI, M+1]⁺=233.1. ¹HNMR (400 MHz, DMSO-d₆) δ=10.60-10.04 (m, 1H), 7.26 (s, 1H), 6.02 (s, 1H), 5.06-4.67 (m, 2H), 4.56 (s, 2H), 2.97-2.79 (m, 3H), 2.15 (s, 3H), 2.07 (br s, 3H).

### Example 14-5

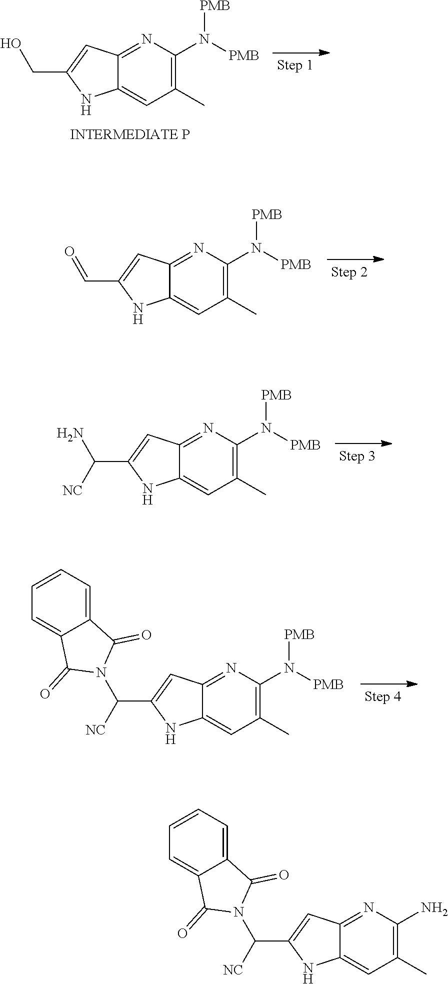

Step 1: To a solution of Intermediate P (645 mg, 1.55 mmol, 1.00 eq.) in dichloromethane (16.0 mL) and dimethylsulfoxide (3.00 mL) was added manganese dioxide (2.69 g, 30.9 mmol, 20.0 eq.). The mixture was stirred at 25° C. for 16 h. The mixture was filtered and concentrated. The residue was purified by silica gel chromatography (Petroleum ether/Ethyl acetate, 0-70% of Ethyl acetate) to afford 5-[bis[(4-methoxyphenyl)methyl]amino]-6-methyl-1H-pyrrolo[3,2-b]pyridine-2-carbaldehyde (630 mg, 1.44 mmol, 93.2% yield) as a yellow solid. LCMS [ESI, M+1]: 416.3. ¹H NMR (400 MHz, CDCl₃) (=9.84 (s, 1H), 9.07 (br s, 1H), 7.53 (s, 1H), 7.28 (d, J=1.2 Hz, 1H), 7.21 (d, J=8.4 Hz, 4H), 6.85-6.76 (m, 4H), 4.24 (s, 4H), 3.77 (s, 6H), 2.53 (s, 3H).

Step 2: To a solution of 5-[bis[(4-methoxyphenyl)methyl]amino]-6-methyl-1H-pyrrolo[3,2-b]pyridine-2-carbaldehyde (300 mg, 722 μmol, 1.00 eq.) in dichloromethane (6.00 mL) was added titanium(IV) isopropoxide (410 mg, 1.44 mmol, 426 μL 2.00 eq.) and ammonia in methanol (7.00 M, 3.00 mL, 29.0 eq.). The mixture was stirred at 25° C. for 2h. Then trimethylsilyl cyanide (179 mg, 1.81 mmol, 225 μL 2.50 eq.) was added dropwise and stirred for 16 h. The mixture was poured to ice-water (30 mL), extracted with dichloromethane (30 mL), dried over sodium sulfate, filtered and concentrated to afford 2-amino-2-[5-[bis[(4-methoxyphenyl)methyl]amino]-6-methyl-1H-pyrrolo[3,2-b]pyridin-2-yl]acetonitrile (330 mg, crude) as a blue gum. LCMS [ESI, M+1]: 442.2

Step 3: To a solution of 2-amino-2-[5-[bis[(4-methoxyphenyl)methyl]amino]-6-methyl-1H-pyrrolo[3,2-b]pyridin-2-yl]acetonitrile (290 mg, 656 μmol, 1.00 eq) in acetic acid (12.0 mL) was added isobenzofuran-1,3-dione (194 mg, 1.31 mmol, 2.00 eq.). The mixture was stirred at 60° C. for 1 h. The mixture was diluted with water (100 mL), extracted with ethyl acetate (100 mL), washed with sat. sodium bicarbonate (100 mL), dried over sodium sulfate, filtered and concentrated. The residue was purified by silica gel chromatography (Petroleum ether/Ethyl acetate, 0-50% of Ethyl acetate) to afford 2-[5-[bis[(4-methoxyphenyl)methyl]amino]-6-methyl-1H-pyrrolo[3,2-b]pyridin-2-yl]-2-(1,3-dioxoisoindolin-2-yl)acetonitrile (35.0 mg, 58.1 μmol, 8.86% yield) as a red gum.

Step 4: To a solution of 2-[5-[bis[(4-methoxyphenyl)methyl]amino]-6-methyl-1H-pyrrolo[3,2-b]pyridin-2-yl]-2-(1,3-dioxoisoindolin-2-yl)acetonitrile (33.0 mg, 57.7 μmol, 1.00 eq.) in dichloromethane (2.00 mL) was added trifluoroacetic acid (616 mg, 5.40 mmol, 0.40 mL, 93.5 eq.). The mixture was stirred at 25° C. for 16 h. The mixture was concentrated. The residue was purified by Prep-HPLC (formic acid condition) to afford 2-(5-amino-6-methyl-1H-pyrrolo[3,2-b]pyridin-2-yl)-2-(1,3-dioxoisoindolin-2-yl)acetonitrile, Example 14-5 (16.9 mg, 38.9 μmol, 67.4% yield, HOAc salt) as a yellow solid. LCMS [ESI, M+1]: 332.1. 1H NMR (400 MHz, MeOD) δ=8.63-8.24 (m, 1H), 8.01-7.86 (m, 3H), 7.85-7.74 (m, 1H), 7.71-7.46 (m, 2H), 6.64 (d, J=6.4 Hz, 1H), 2.28 (d, J=11.6 Hz, 3H).

### Example 14-6

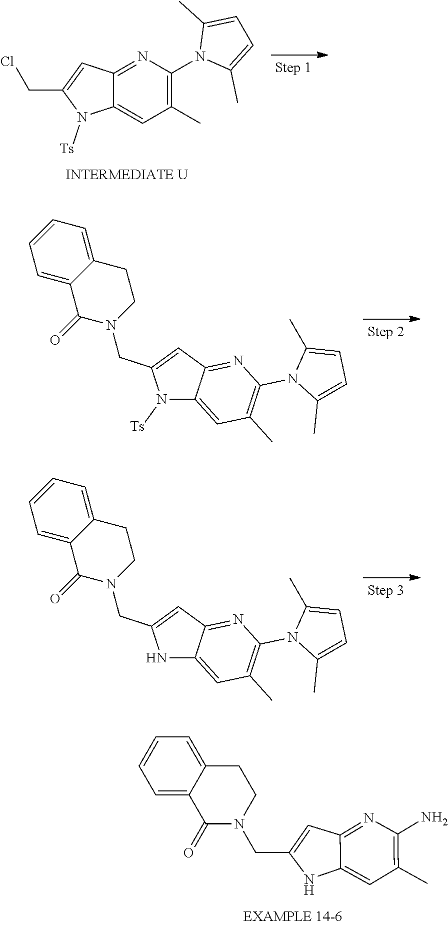 [SMILES: Cc1ccc(S(=O)(=O)n2c(CCl)cc3nc(-n4c(C)ccc4C)c(C)cc32)cc1 ; Cc1ccc(S(=O)(=O)n2c(CN3CCc4ccccc4C3=O)cc3nc(-n4c(C)ccc4C)c(C)cc32)cc1 ; Cc1cc2[nH]c(CN3CCc4ccccc4C3=O)cc2nc1-n1c(C)ccc1C ; Cc1cc2[nH]c(CN3CCc4ccccc4C3=O)cc2nc1N]

Step 1: To a solution of 3,4-dihydro-2H-isoquinolin-1-one (51.6 mg, 351 μmol, 1.50 eq.) in dimethylformamide (5.00 mL) was added sodium hydride (14.0 mg, 351 μmol, 60% purity, 1.50 eq.) at 0° C. The reaction mixture was stirred at 0° C. for 15 min, Intermediate U (100 mg, 234 μmol, 1.00 eq.) was added and the resulting was stirred at 0° C. for 1 hour. The reaction was quenched with saturated ammonium chloride (10.0 mL). The mixture was extracted with ethyl acetate (3×30.0 mL). The combined organic extracts were washed with brine (20.0 mL) and then dried over anhydrous sodium sulfate and concentrated under vacuum to give a residue. The residue was purified by prep-TLC (petroleum ether/ethyl acetate 1:1) to give 2-[[5-(2,5-dimethylpyrrol-1-yl)-6-methyl-1-(p-tolylsulfonyl)pyrrolo[3,2-b]pyridin-2-yl]methyl]-3,4-dihydroisoquinolin-1-one (90 mg, 156 μmol, 66.7% yield) as a yellow solid. LCMS1 [M+1]⁺=539.1

Step 2: To a solution of 2-[[5-(2,5-dimethylpyrrol-1-yl)-6-methyl-1-(p-tolylsulfonyl)pyrrolo[3,2-b]pyridin-2-yl]methyl]-3,4-dihydroisoquinolin-1-one (90.0 mg, 167 μmol, 1.00 eq.) in methanol (3.00 mL) was added sodium methoxide (4.00 M, 209 μL 5.00 eq.), the mixture was stirred at 20° C. for 2.5 hours. The pH of the mixture was neutralized to 7 with 1M hydrochloric acid. The mixture was extracted with dichloromethane (3×10.0 mL). The combined organic extracts were dried over anhydrous sodium sulfate, and concentrated under vacuum to get residue. The residue was purified by prep-TLC (SiO2, petroleum ether/ethyl acetate 1:1) to afford 2-[[5-(2,5-dimethylpyrrol-1-yl)-6-methyl-1H-pyrrolo[3,2-b]pyridin-2-yl]methyl]-3,4-dihydroisoquinolin-1-one (40.0 mg, 104 μmol, 62.2% yield) as a yellow solid.

Step 3: To a solution of 2-[[5-(2, 5-dimethylpyrrol-1-yl)-6-methyl-1H-pyrrolo[3,2-b]pyridin-2-yl]methyl]-3,4-dihydroisoquinolin-1-one (20.0 mg, 52.0 μmol, 1.00 eq.) in ethanol (1.50 mL) was added hydrochloric acid (12 M, 0.15 mL, 34.6 eq.). The sealed tube was heated at 120° C. for 1 hour under microwave irradiation. The mixture was concentrated under vacuum to produce residue. The residue was purified by prep-HPLC (HCl condition) to give 2-[(5-amino-6-methyl-1H-pyrrolo[3,2-b]pyridin-2-yl)methyl]-3,4-dihydroisoquinolin-1-one, Example 14-6 (8.44 mg, 24.2 μmol, 46.6% yield, HCl) as an orange solid. LCMS [M+1]⁺=307.1. ¹H NMR (400 MHz, MeOD-d₄) δ=8.01 (dd, J=0.8, 7.6 Hz, 1H), 7.88 (s, 1H), 7.53-7.47 (m, 1H), 7.42-7.35 (m, 1H), 7.29 (d, J=7.6 Hz, 1H), 6.40 (s, 1H), 4.93 (s, 2H), 3.66 (t, J=6.8 Hz, 2H), 3.05 (t, J=6.4 Hz, 2H), 2.29 (s, 3H).

### Example 14-7

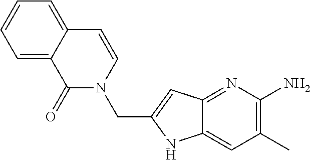 [SMILES: Cc1cc2[nH]c(Cn3ccc4ccccc4c3=O)cc2nc1N]

2-[(5-amino-6-methyl-1H-pyrrolo[3,2-b]pyridin-2-yl)methyl]isoquinolin-1-one, Example 14-7 was prepared from Intermediate U and 2H-isoquinolin-1-one following the three step procedure described for Example 14-6 to afford the title compound (12.6 mg, 35.87 μmol, 17% yield over 3 steps, HCl salt) as an orange solid. LCMS: [M+1]⁺=305.1. ¹H NMR (400 MHz, MeOD-d₄) δ=8.35 (d, J=8.0 Hz, 1H), 7.88 (s, 1H), 7.78-7.70 (m, 1H), 7.68-7.64 (m, 1H), 7.59-7.53 (m, 1H), 7.47 (d, J=7.6 Hz, 1H), 6.74 (d, J=7.2 Hz, 1H), 6.38 (s, 1H), 5.38 (s, 2H), 2.28 (s, 3H).

### Example 15-1

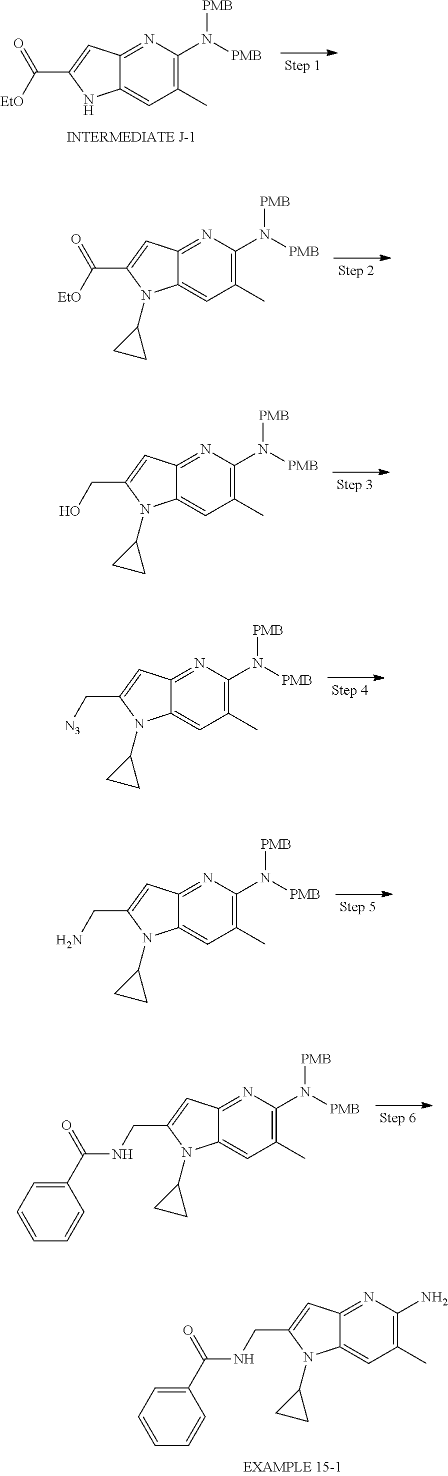

Step 1: A mixture of Intermediate J-1 (1.66 g, 3.60 mmol, 1.00 eq.), cyclopropylboronic acid (1.86 g, 21.6 mmol, 6.00 eq.), 2-(2-pyridyl)pyridine (1.13 g, 7.21 mmol, 2.00 eq.), copper acetate (1.31 g, 7.21 mmol, 2.00 eq.) and sodium carbonate (2.29 g, 21.6 mmol, 6.00 eq.) in dichloromethane (25.0 mL) was degassed and purged with oxygen (15 Psi) for 3 times, and then the mixture was stirred at 120° C. for 5 hours under oxygen atmosphere. The mixture was concentrated under reduced pressure. The residue was purified by flash silica gel chromatography (0˜60% Ethyl acetate/Petroleum ether) to give ethyl 5-[bis[(4-methoxyphenyl)methyl]amino]-1-cyclopropyl-6-methyl-pyrrolo[3,2-b]pyridine-2-carboxylate (1.40 g, 2.79 mmol, 77.5% yield) as a yellow solid. LCMS [M+1]⁺=500.3. 1H NMR (400 MHz, CDCl₃-d) δ=8.70 (dd, J=0.8, 4.8 Hz, 1H), 7.67 (s, 1H), 7.24-7.21 (m, 4H), 6.82-6.76 (m, 4H), 4.21 (s, 4H), 3.77 (s, 6H), 3.47 (tt, J=3.6, 7.2 Hz, 1H), 2.53 (s, 3H), 1.41 (t, J=7.2 Hz, 3H), 1.23-1.16 (m, 2H), 0.97-0.89 (m, 2H).

Step 2: A mixture of ethyl 5-[bis[(4-methoxyphenyl)methyl]amino]-1-cyclopropyl-6-methyl-pyrrolo[3,2-h]pyridine-2-carboxylate (1.30 g, 2.60 mmol, 1.00 eq.), Lithium aluminum hydride (198 mg, 5.20 mmol, 2.00 eq.) in tetrahydrofuran (1.00 mL) was degassed and stirred at 0° C. for 3 hours under nitrogen atmosphere. The mixture was quenched by water (0.40 mL), stirred for 5 min, then treated with aqueous sodium hydroxide solution (15%) (0.40 mL), stirred for 5 min, then treated with water (1.20 mL) stirred for 5 min at 0° C., filtered and concentrated under reduced pressure to give a residue. The residue was purified by flash silica gel chromatography (0-˜100% Ethyl acetate/Petroleum ether) to give [5-[bis[(4-methoxyphenyl)methyl]amino]-1-cyclopropyl-6-methyl-pyrrolo[3,2-b]pyridin-2-yl]methanol (1.00 g, 2.18 mmol, 83.8% yield) as a white solid. LCMS [M+1]⁺=458.4. ¹H NMR (400 MHz, CDCl₃-d) 5=7.59 (s, 1H), 7.23 (d, J=8.8 Hz, 4H), 6.78 (d, J=8.8 Hz, 4H), 6.52 (s, 1H), 4.90 (br s, 2H), 4.18 (s, 4H), 3.77 (s, 6H), 3.26-3.14 (m, 1H), 2.49 (s, 3H), 1.17-1.08 (m, 4H).

Step 3: To a solution of [5-[bis[(4-methoxyphenyl)methyl]amino]-1-cyclopropyl-6-methyl-pyrrolo[3,2-b]pyridin-2-yl]methanol (1.00 g, 2.00 mmol, 1.00 eq.) in dichloromethane (15.0 mL) was added DPPA (1.80 g, 6.56 mmol, 1.42 mL, 3 eq) at 0° C., then DBU (998 mg, 6.56 mmol, 988 μL 3.00 eq.) at 0° C., The mixture was stirred at 0-20° C. for 2 hours. The mixture was concentrated under reduced pressure to give a residue. The residue was purified by column chromatography (SiO₂, Petroleum ether/Ethyl acetate 10:1 to dichloromethane/methyl alcohol=10:1) to give 2-(azidomethyl)-1-cyclopropyl-N,N-bis[(4-methoxyphenyl)methyl]-6-methyl-pyrrolo[3,2-b]pyridin-5-amine (0.90 g, 1.52 mmol, 69.5% yield) as a white solid. LCMS [M+1]=483.3.

Step 4: A mixture of 2-(azidomethyl)-1-cyclopropyl-N,N-bis[(4-methoxyphenyl)methyl]-6-methyl-pyrrolo[3,2-b]pyridin-5-amine (0.90 g, 1.86 mmol, 1.00 eq.), triphenylphosphine (2.45 g, 9.32 mmol, 5.00 eq.) in tetrahydrofuran (12.0 mL) and water (4.0 mL) was degassed and stirred at 50° C. for 3 hours under nitrogen atmosphere. The mixture was concentrated under reduced pressure to give a residue. The crude product was purified by reversed-phase HPLC (0.1% FA condition) to give 2-(aminomethyl)-1-cyclopropyl-N,N-bis[(4-methoxyphenyl)methyl]-6-methyl-pyrrolo[3,2-b]pyridin-5-amine (0.60 g, 1.27 mmol, 67.9% yield) as a white solid. LCMS [M+1]⁺=457.2. ¹H NMR (400 MHz, CDC-d) δ=7.54 (s, 1H), 7.23 (d, J=8.8 Hz, 4H), 6.78 (d, J=8.8 Hz, 4H), 6.44 (s, 1H), 4.18 (s, 4H), 4.12 (s, 2H), 3.76 (s, 6H), 3.16-3.10 (m, 1H), 2.47 (s, 3H), 1.15-1.10 (m, 2H), 1.06-1.01 (m, 2H).

Step 5: To a solution of benzoic acid (241 mg, 1.97 mmol, 301 μL 1.50 eq.) and triethylamine (266 mg, 2.63 mmol, 366 μL 2.00 eq.) in dimethyl formamide (2.00 mL) was added HATU (750 mg, 1.97 mmol, 1.50 eq.). The mixture was stirred at 20° C. for 20 minutes. Then 2-(aminomethyl)-1-cyclopropyl-N,N-bis[(4-methoxyphenyl)methyl]-6-methyl-pyrrolo[3,2-b]pyridin-5-amine (0.60 g, 1.31 mmol, 1.00 eq) was added and the mixture was stirred for 2 hours. The mixture was concentrated under reduced pressure to give a residue. The crude product was purified by reversed-phase HPLC (0.1% FA condition) to give N-[[5-[bis[(4-methoxyphenyl)methyl]amino]-1-cyclopropyl-6-methyl-pyrrolo[3,2-b]pyridin-2-yl]methyl]benzamide (0.40 g, 522 μmol, 39.7% yield) as a white solid. LCMS [M+1]⁺=561.5. ¹H NMR (400 MHz, CDCl₃-d) δ=7.90 (br s, 1H), 7.80 (br d, J=7.2 Hz, 2H), 7.53-7.41 (m, 3H), 7.14 (br d, J=8.1 Hz, 4H), 6.79 (br d, J=8.4 Hz, 4H), 6.69-6.61 (m, 1H), 4.93 (br d, J=5.6 Hz, 2H), 4.31 (br s, 4H), 3.75 (s, 6H), 3.32-3.25 (m, 1H), 2.55 (br s, 3H), 1.28 (br d, J=7.1 Hz, 2H), 1.09 (br s, 2H).

Step 6. A mixture of N-[[5-[bis[(4-methoxyphenyl)methyl]amino]-1-cyclopropyl-6-methyl-pyrrolo[3,2-b]pyridin-2-yl]methyl]benzamide (70.0 mg, 125 μmol, 1.00 eq.) in trifluoroacetic acid (3.00 mL) and trifluoromethanesulfonic acid (1.00 mL) was stirred at 50° C. for 2 hours under nitrogen atmosphere. The mixture was concentrated under reduced pressure to give a residue. The residue was purified by prep-HPLC (HCl condition) to give N-[(5-amino-1-cyclopropyl-6-methyl-pyrrolo[3,2-b]pyridin-2-yl)methyl]benzamide, Example 15-1 (27.2 mg, 74.4 μmol, 59.60/yield, HCl) as a yellow solid. LCMS [M+1]⁺=321.1. ¹H NMR (400 MHz, MeOD-d₄) δ=8.14 (s, 1H), 7.91-7.85 (m, 2H), 7.61-7.55 (m, 1H), 7.53-7.47 (m, 2H), 6.30 (s, 1H), 4.88 (br s, 2H), 3.40-3.34 (m, 1H), 2.33 (s, 3H), 1.33-1.28 (m, 2H), 1.17-1.11 (m, 2H).

### Example 15-2

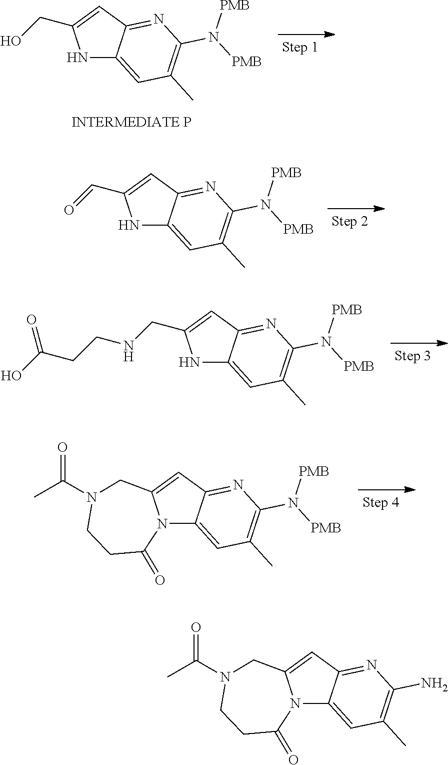

Step 1: A mixture of Intermediate P (560 mg, 1.34 mmol, 1.00 eq.) and manganese dioxide (2.33 g, 26.8 mmol, 20.0 eq.) in dichloromethane (10.0 mL) and dimethyl sulfoxide (1.00 mL) was stirred at 25° C. for 12 hours. The mixture was filtered and the filter cake was washed with dichloromethane (10.0 mL×3). The combined filtrate was concentrated. The residue was purified by flash silica gel chromatography (30-50% Ethyl acetate/Petroleum ether) to obtain 5-[bis[(4-methoxyphenyl)methyl]amino]-6-methyl-1H-pyrrolo[3,2-b]pyridine-2-carbaldehyde (468 mg, 1.09 mmol, 81.5% yield) as a yellow solid. LCMS [ESI, M+1]: 416.2. ¹H NMR (400 MHz, CD₃OD-d₆) δ=9.82 (s, 1H), 7.68 (s, 1H), 7.22 (s, 1H), 7.15 (d, J=8.4 Hz, 4H), 6.80 (d, J=8.4 Hz, 4H), 4.20 (s, 4H), 3.74 (s, 6H), 2.53 (s, 3H).

Step 2: A mixture of 5-[bis[(4-methoxyphenyl)methyl]amino]-6-methyl-1H-pyrrolo[3,2-b]pyridine-2-carbaldehyde (468 mg, 1.13 mmol, 1.00 eq.) and 3-aminopropanoic acid (201 mg, 2.25 mmol, 2.00 eq.), acetic acid (6.76 mg, 113 μmol, 6.44 μL 0.10 eq.) in methanol (5.00 mL) and dichloromethane (5.00 mL) was stirred at 25° C. for 0.5 hour. Then sodium cyanoborohydride (142 mg, 2.25 mmol, 2.00 eq.) was added to above mixture and the resulting mixture was stirred at 25° C. for 12 hours. The mixture was concentrated. The residue was purified by flash silica gel chromatography (30% methanol/dichloromethane) to obtain 3-[[5-[bis[(4-methoxyphenyl)methyl]amino]-6-methyl-1H-pyrrolo[3,2-b]pyridin-2-yl]methylamino]propanoic acid (300 mg, 559 μmol, 49.6% yield) as a white solid. LCMS [ESI, M+1]: 489.3. ¹H NMR (400 MHz, CD₃OD-d₆) δ=7.59 (s, 1H), 7.14 (d, J=8.4 Hz, 4H), 6.77 (d, J=8.8 Hz, 4H), 6.62 (s, 1H), 4.36 (s, 2H), 4.15 (s, 4H), 3.72 (s, 6H), 3.20 (t, J=6.4 Hz, 2H), 2.51 (t, J=6.4 Hz, 2H), 2.46 (s, 3H).

Step 3: To a solution of 3-[[5-[bis[(4-methoxyphenyl)methyl]amino]-6-methyl-1H-pyrrolo[3,2-b]pyridin-2-yl]methylamino]propanoic acid (45.0 mg, 92.1 μmol, 1.00 eq.) and acetic anhydride (20.7 mg, 203 μmol, 19.0 μL 2.20 eq.) in dichloromethane (2.00 mL) was added triethylamine (32.6 mg, 322 μmol, 44.9 μL 3.50 eq.). The resulting mixture was stirred at 25-30° C. for 12 hours. The mixture was concentrated. The residue was purified by prep-TLC (silica gel, 100% ethyl acetate) to obtain 11-acetyl-5-[bis [(4-methoxyphenyl)methyl]amino]-4-methyl-1,6,11-triazatricyclo[7.5.0.0^{2,7}]tetradeca-2,4,6,8-tetraen-14-one (23.0 mg, 44.4 μmol, 48.2% yield) as a colorless oil. LCMS [EST, M+1]: 513.3

Step 4: A mixture of 11-acetyl-5-[bis[(4-methoxyphenyl)methyl]amino]-4-methyl-1,6,11-triazatricyclo[7.5.0.0,]tetradeca-2,4,6,8-tetraen-14-one (23.0 mg, 44.9 μmol, 1.00 eq.) in dichloromethane (0.50 mL) and trifluoroacetic acid (0.10 mL) was stirred at 25-30° C. for 2 hours. The mixture was concentrated. The residue was purified by Prep-HPLC (HCl condition), followed by lyophilization to obtain 11-acetyl-5-amino-4-methyl-1,6,11-triazatricyclo[7.5.0.0^{2,7}]tetradeca-2,4,6,8-tetraen-14-one (6.81 mg, 21.6 μmol, 48.1% yield, hydrochloric salt) as a white solid. LCMS [ESI, M+1]: 273.2. ¹H NMR (400 MHz, DMSO-d₆, T=25° C.) δ=8.56 (d, J=5.2 Hz, 1H), 7.84 (br s, 2H), 6.94-6.66 (m, 1H), 5.02 (br d, J=2.8 Hz, 2H), 3.84-3.63 (m, 2H), 3.33 (br d, J=6.4 Hz, 2H), 2.25 (s, 3H), 2.09-1.97 (m, 3H). ¹H NMR (400 MHz, DMSO-d₆, T=80° C.) δ=8.54 (s, 1H), 6.92-6.60 (m, 1H), 5.01 (s, 2H), 3.81-3.72 (m, 2H), 3.33 (br s, 2H), 2.28 (s, 3H), 2.04 (br s, 3H).

### Example 15-3

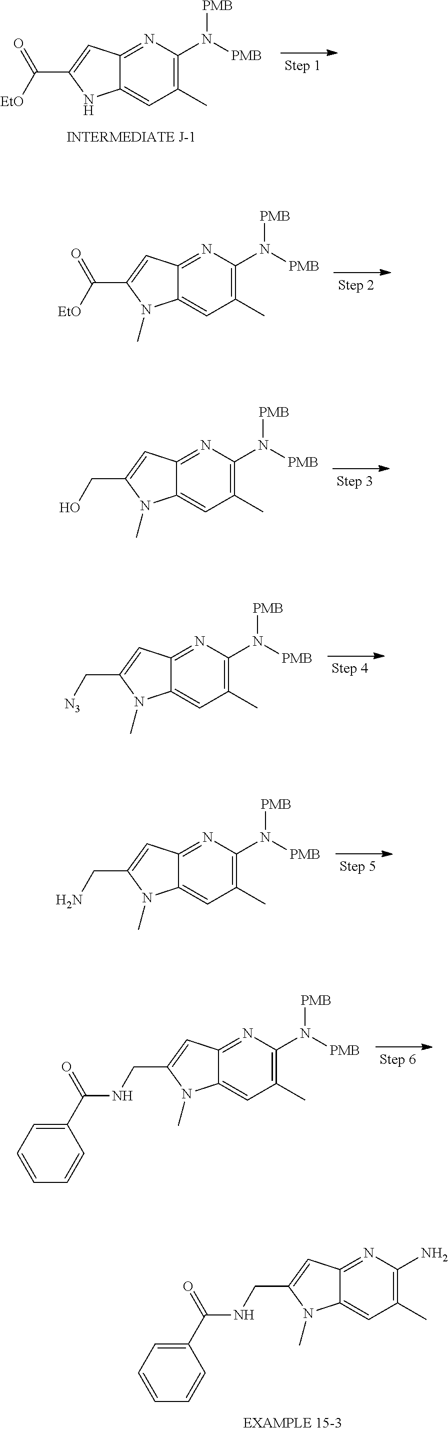

Step 1: To a solution of Intermediate J-1 (1.50 g, 3.26 mmol, 1.00 eq.) in dimethyl formamide (15.0 mL) was added sodium hydride (196 mg, 4.90 mmol, 60% purity, 1.50 eq.) at 0° C. and the mixture was stirred at 20° C. for 1 hour. Then iodomethane (927 mg, 6.53 mmol, 406 μL 2.00 eq.) was added to the mixture and the mixture was stirred at 20° C. for 2 hours. The mixture was quenched with water (15.0 mL) and exacted with ethyl acetate (15 mL×2). The combined organic phase was washed with brine (30 mL×2), dried over anhydrous sodium sulfate, filtered and concentrated under reduced pressure to give ethyl 5-[bis[(4-methoxyphenyl)methyl]amino]-1,6-dimethyl-pyrrolo[3,2-b]pyridine-2-carboxylate (1.40 g, 2.96 mmol, 90.6% yield) as a white solid. LCMS [EST, M+1]⁺=474.2. ¹H NMR (400 MHz, DMSO-d₆) δ (ppm)=7.82 (s, 1H), 7.26-7.16 (m, 4H), 7.09-7.03 (m, 1H), 6.81 (d, J=8.8 Hz, 4H), 4.30 (q, J=7.2 Hz, 2H), 4.20-4.11 (m, 4H), 3.95 (s, 3H), 3.68 (s, 6H), 2.51 (s, 3H), 1.32 (t, J=7.2 Hz, 3H).

Steps 2-6: The subsequent 5-step synthesis utilized the same procedure as described in Steps 2-6 of Example 15-1 to produce N-[(5-amino-1,6-dimethyl-pyrrolo[3,2-b]pyridin-2-yl)methyl]benzamide (2.00 mg, 6.70 μmol, -1% yield over 5 steps) as a white solid. LCMS [ESI, M+1]⁺=295.2. ¹H NMR (400 MHz, CD₃OD) δ (ppm)=9.08 (br d, J=2.8 Hz, 1H), 8.06 (s, 1H), 7.86 (br d, J=7.2 Hz, 2H), 7.63-7.53 (m, 1H), 7.52-7.39 (m, 2H), 6.38 (s, 1H), 4.77 (br d, J=3.9 Hz, 2H), 3.86 (s, 3H), 2.32 (s, 3H).

### Example 15-4

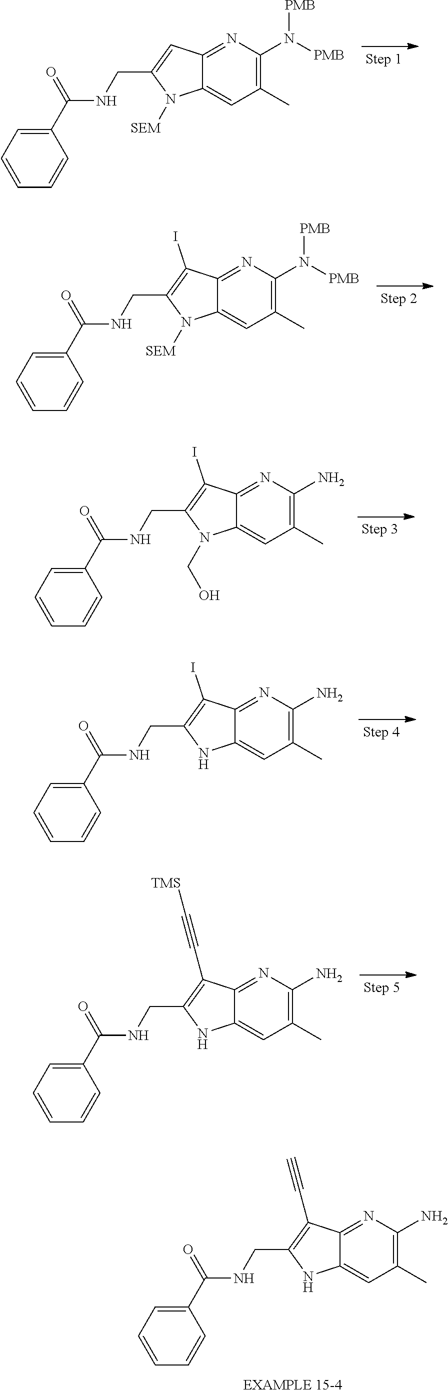

### Example 15-4

Step 1: To a solution of N-((5-(bis(4-methoxybenzyl)amino)-6-methyl-1-((2-(trimethylsilyl)ethoxy)methyl)-1H-pyrrolo[3,2-b]pyridin-2-yl)methyl)benzamide (see Step 1 of Example 15-6) (300 mg, 461 μmol, 1.00 eq.) in dimethyl formamide (8.00 mL) was added NIS (156 mg, 691 μmol, 1.50 eq.). Then the mixture was stirred at 25° C. for 3 hours. The mixture was diluted with water (50.0 mL) and extracted with ethyl acetate (20 mL×3). The organic layer was washed with brine (50.0 mL), dried over sodium sulfate, filtered and concentrated. The residue was purified by flash silica gel chromatography (5-33% Ethyl acetate/Petroleum ether) to afford N-((5-(bis(4-methoxybenzyl)amino)-3-iodo-6-methyl-1-((2-(trimethylsilyl)ethoxy)methyl)-1H-pyrrolo[3,2-b]pyridin-2-yl)methyl)benzamide (250 mg, 292 μmol, 63.3% yield) as a light yellow oil. LCMS [ESI, M+1]⁺=777.3.

Step 2: To a solution of N-((5-(bis(4-methoxybenzyl)amino)-3-iodo-6-methyl-1-((2-(trimethylsilyl)ethoxy)methyl)-1H-pyrrolo[3,2-b]pyridin-2-yl)methyl)benzamide (250 mg, 322 μmol, 1.00 eq.) in dichloromethane (8.00 mL) was added trifluoroacetic acid (2.46 g, 21.6 mmol, 1.60 mL, 67.2 eq.). The mixture was stirred at 20° C. for 12 hours. The mixture was concentrated to afford N-((5-amino-1-(hydroxymethyl)-3-iodo-6-methyl-1H-pyrrolo[3,2-b]pyridin-2-yl)methyl)benzamide (140 mg, crude) as a light yellow oil.

Step 3: To a solution of N-((5-amino-1-(hydroxymethyl)-3-iodo-6-methyl-1H-pyrrolo[3,2-b]pyridin-2-yl)methyl)benzamide (140 mg, 321 μmol, 1.00 eq.) in dioxane (5.00 mL) was added ammonium hydroxide (450 mg, 3.21 mmol, 494 μL 25% purity, 10.0 eq.). The mixture was stirred at 20° C. for 2 hours. The mixture was concentrated to afford N-((5-amino-3-iodo-6-methyl-1H-pyrrolo[3,2-b]pyridin-2-yl)methyl)benzamide (130 mg, crude) as a light yellow oil.

Step 4: To a solution of N-((5-amino-3-iodo-6-methyl-1H-pyrrolo[3,2-b]pyridin-2-yl)methyl)benzamide (130 mg, 320 μmol, 1.00 eq.) and triethylamine (324 mg, 3.20 mmol, 445 μL 10.0 eq.) in tetrahydrofuran (5.00 mL) was added copper(I) iodide (6.09 mg, 32.0 μmol, 0.10 eq.), bis(triphenylphosphine)palladium(II)dichloride (67.4 mg, 96.0 μmol, 0.30 eq.) and ethynyltrimethylsilane (314 mg, 3.20 mmol, 443 μL 10.0 eq.). The mixture was stirred at 50° C. for 3 hours under nitrogen atmosphere. The mixture was concentrated. The residue was purified by flash silica gel chromatography (10˜100% Ethyl acetate/Petroleum ether) to afford N-((5-amino-6-methyl-3-((trimethylsilyl)ethynyl)-1H-pyrrolo[3,2-b]pyridin-2-yl)methyl)benzamide (60.0 mg, 123 μmol, 38.5% yield) as a light yellow oil. LCMS [ESI, M+1]⁺=377.3.

Step 5: To a solution of N-((5-amino-6-methyl-3-((trimethylsilyl)ethynyl)-1H-pyrrolo[3,2-b]pyridin-2-yl)methyl)benzamide (60.0 mg, 159 μmol, 1.00 eq.) in tetrahydrofuran (1.00 mL) was added tetrabutylammonium fluoride (1M in tetrahydrofuran, 478 μL 3.00 eq.). The mixture was stirred at 20° C. for 1 hour. The mixture was diluted with water (20.0 mL) and extracted with ethyl acetate (10.0 mL×2). The organic layer was washed with brine (10.0 mL), dried over sodium sulfate, filtered and concentrated. The residue was purified by prep-HPLC (neutral condition) and lyophilized to afford N-((5-amino-3-ethynyl-6-methyl-1H-pyrrolo[3,2-b]pyridin-2-yl)methyl)benzamide, Example 15-4 (2.12 mg, 6.81 μmol, 4.27% yield) as a yellow solid. LCMS [ESI, M+1]=305.2. ¹HNMR (400 MHz, CDCl₃) δ=9.30 (br s, 1H), 7.86-7.74 (m, 2H), 7.58-7.51 (m, 1H), 7.49-7.42 (m, 2H), 7.29 (d, J=0.4 Hz, 1H), 7.01-6.93 (m, 1H), 4.80 (d, J=6.0 Hz, 2H), 4.42 (s, 2H), 3.40 (s, 1H), 2.23 (d, J=0.4 Hz, 3H).

### Example 15-5

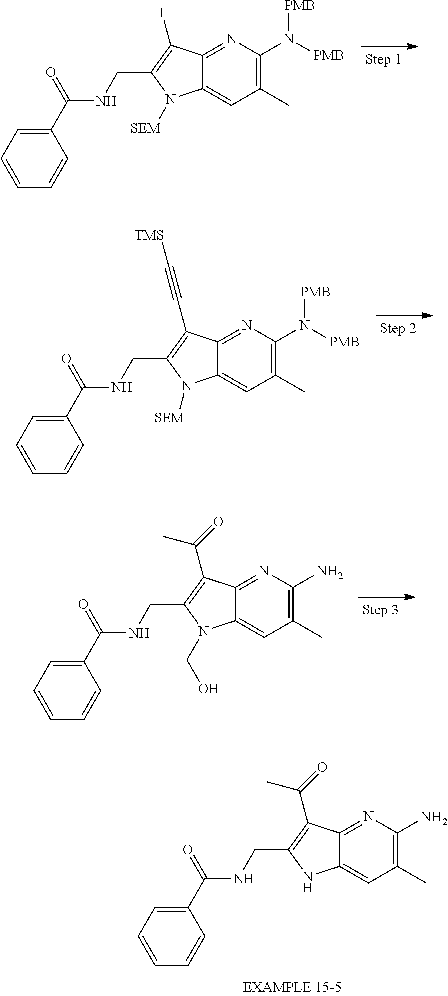

Step 1: To a solution of N-((5-(bis(4-methoxybenzyl)amino)-3-iodo-6-methyl-1-((2-(trimethylsilyl)ethoxy)methyl)-1H-pyrrolo[3,2-b]pyridin-2-yl)methyl)benzamide (see Step 1 of Example 15-4) (90.0 mg, 116 μmol, 1.00 eq.) and triethylamine (117 mg, 1.16 mmol, 161 μL 10.0 eq.) in tetrahydrofuran (2.50 mL) was added copper(I) iodide (2.21 mg, 11.6 μmol, 0.10 eq.), bis(triphenylphosphine)palladium(II)dichloride (24.4 mg, 34.8 μmol, 0.30 eq.) and ethynyltrimethylsilane (114 mg, 1.16 mmol, 161 μL 10.0 eq). The mixture was stirred at 50° C. for 3 hours under nitrogen atmosphere. The mixture was diluted with water (30.0 mL) and extracted with ethyl acetate (10.0 mL×3). The organic layer was washed with brine (30.0 mL), dried over sodium sulfate, filtered and concentrated. The residue was purified by flash silica gel chromatography (5˜20% Ethyl acetate/Petroleum ether) to afford N-((5-(bis(4-methoxybenzyl)amino)-6-methyl-1-((2-(trimethylsilyl)ethoxy)methyl)-3-((trimethylsilyl)ethynyl)-1H-pyrrolo[3,2-b]pyridin-2-yl)methyl)benzamide (70.0 mg, 86.7 μmol, 74.9% yield) as a light yellow oil. LCMS [EST, M+1]⁺=747.4.

Compound 61042A-3: To a solution of N-((5-(bis(4-methoxybenzyl)amino)-6-methyl-1-((2-(trimethylsilyl)ethoxy)methyl)-3-((trimethylsilyl)ethynyl)-1H-pyrrolo[3,2-b]pyridin-2-yl)methyl)benzamide) (40.0 mg, 53.5 μmol, 1.00 eq.) in dichloromethane (1.50 mL) was added trifluoroacetic acid (528 mg, 4.63 mmol, 343 μL 86.5 eq.). The mixture was stirred at 20° C. for 3 hours. The mixture was concentrated. to afford N-((3-acetyl-5-amino-1-(hydroxymethyl)-6-methyl-1H-pyrrolo[3,2-b]pyridin-2-yl)methyl)benzamide (18.0 mg, crude) as a light yellow oil.

Compound WX-61042A: To a solution of N-((3-acetyl-5-amino-1-(hydroxymethyl)-6-methyl-1H-pyrrolo[3,2-b]pyridin-2-yl)methyl)benzamide (18.0 mg, 51.1 μmol, 1.00 eq.) in dioxane (1.00 mL) was added ammonium hydroxide (35.8 mg, 255 μmol, 39.3 μL 25% purity, 5.00 eq.). The mixture was stirred at 20° C. for 0.5 hours. The mixture was concentrated. The residue was purified by prep-HPLC (basic condition) and lyophilized to give N-((3-acetyl-5-amino-6-methyl-1H-pyrrolo[3,2-b]pyridin-2-yl)methyl)benzamide, Example 15-5 (6.34 mg, 19.2 μmol, 37.5% yield) as a white solid. LCMS [ESI, M+1]⁺=323.2. ¹HNMR (400 MHz, Methanol-d₄) δ=7.96-7.88 (m, 2H), 7.63-7.52 (m, 2H), 7.52-7.44 (m, 2H), 5.01 (s, 2H), 2.79 (s, 3H), 2.25 (s, 3H).

### Example 15-6

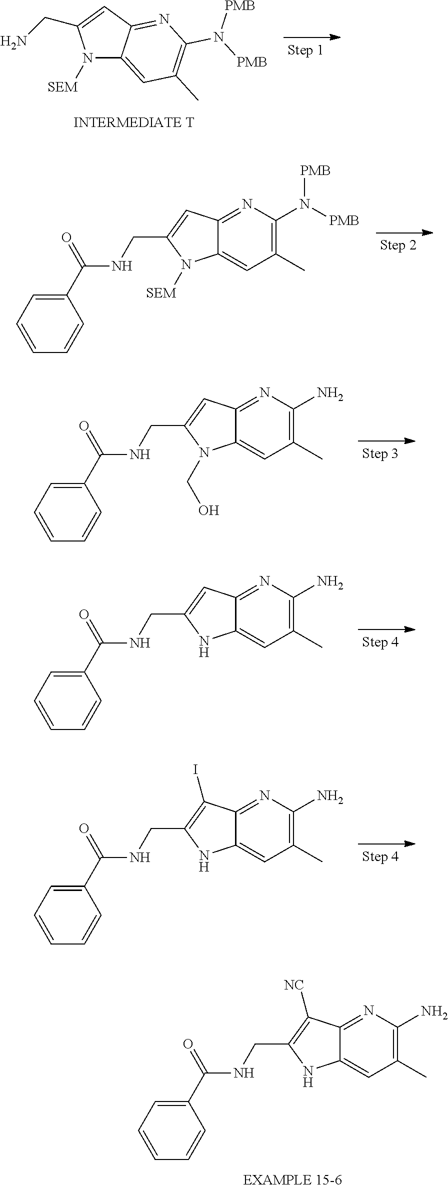

Step 1: To a solution of Intermediate T (600 mg, 1.10 mmol, 1.00 eq.) and benzoic acid (161 mg, 1.32 mmol, 201 μL 1.20 eq.) in dichloromethane (15.0 mL) was added HATU (626 mg, 1.65 mmol, 1.50 eq.) and N,N-diisopropylethylamine (284 mg, 2.19 mmol, 382 μL 2.00 eq.). The mixture was stirred at 20° C. for 12 hours. The mixture was diluted with water (100 mL) and extracted with ethyl acetate (50.0 mL×2). The organic layer was washed with brine (100 mL), dried over sodium sulfate, filtered and concentrated. The residue was purified by flash silica gel chromatography (10-33% Ethyl acetate/Petroleum ether) to afford N-((5-(bis(4-methoxybenzyl)amino)-6-methyl-1-((2-(trimethylsilyl)ethoxy)methyl)-1H-pyrrolo[3,2-b]pyridin-2-yl)methyl)benzamide (500 mg, 754 μmol, 68.7% yield) as a light yellow oil. LCMS [ESI, M+1]⁺=651.4. ¹HNMR (400 MHz, CDCl₃) δ=7.80 (d, J=7.2 Hz, 2H), 7.53-7.38 (m, 4H), 7.22 (d, J=8.4 Hz, 4H), 6.85-6.73 (m, 5H), 6.63 (s, 1H), 5.45 (s, 2H), 4.87 (d, J=5.2 Hz, 2H), 4.19 (s, 4H), 3.77 (s, 6H), 3.55-3.47 (m, 2H), 2.49 (s, 3H), 0.87-0.77 (m, 2H), -0.09 (s, 9H).

Step 2: To a solution of N-((5-(bis(4-methoxybenzyl)amino)-6-methyl-1-((2-(trimethylsilyl)ethoxy)methyl)-1H-pyrrolo[3,2-b]pyridin-2-yl)methyl)benzamide (500 mg, 768 μmol, 1.00 eq.) in dichloromethane (15.0 mL) was added trifluoroacetic acid (4.62 g, 40.5 mmol, 3.00 mL, 52.8 eq.). The mixture was stirred at 20° C. for 16 hours. The mixture was concentrated to afford N-((5-amino-1-(hydroxymethyl)-6-methyl-1H-pyrrolo[3,2-b]pyridin-2-yl)methyl)benzamide (238 mg, crude) as a light yellow oil. LCMS [ESI, M+1]⁺=311.2.

Step 3: To a solution of N-((5-amino-1-(hydroxymethyl)-6-methyl-1H-pyrrolo[3,2-b]pyridin-2-yl)methyl)benzamide (238 mg, 767 μmol, 1.00 eq.) in dioxane (6.00 mL) was added ammonium hydroxide (1.08 g, 7.67 mmol, 1.18 mL, 25% purity, 10.0 eq.). The mixture was stirred at 20° C. for 3 hours. The mixture was concentrated to afford N-((5-amino-6-methyl-1H-pyrrolo[3,2-b]pyridin-2-yl)methyl)benzamide (214 mg, crude) as a light yellow oil. LCMS [ESI, M+1]⁺=281.2.

Step 4: To a solution of N-((5-amino-6-methyl-1H-pyrrolo[3,2-b]pyridin-2-yl)methyl)benzamide (214 mg, 763 μmol, 1.00 eq.) in dimethyl formamide (6.00 mL) was added NIS (258 mg, 1.15 mmol, 1.50 eq.). The mixture was stirred at 20° C. for 3 hours. The mixture was diluted with water (50.0 mL) and extracted with ethyl acetate (20.0 mL×3). The organic layer was washed with brine (50.0 mL), dried over sodium sulfate, filtered and concentrated. The residue was purified by prep-TLC (SiO₂, dichloromethane/methanol 10:1, R_{f}=0.5) to afford N-((5-amino-3-iodo-6-methyl-1H-pyrrolo[3,2-b]pyridin-2-yl)methyl)benzamide (100 mg, 206 μmol, 27.0% yield) as a light yellow oil. LCMS [ESI, M+1]⁺=406.9.

Step 5: To a solution of N-((5-amino-3-iodo-6-methyl-1H-pyrrolo[3,2-b]pyridin-2-yl)methyl)benzamide (25.0 mg, 61.5 μmol, 1.00 eq.) in dimethyl formamide (1.00 mL) was added zinc cyanide (21.7 mg, 185 μmol, 11.7 μL 3.00 eq), zinc powder (1.21 mg, 18.5 μmol, 0.30 eq.) tris(dibenzylideneacetone)dipalladium(0) (5.64 mg, 6.15 μmol, 0.10 eq.) and 1,1′-bis(diphenylphosphino)ferrocene (6.82 mg, 12.3 μmol, 0.20 eq.). The mixture was stirred at 100° C. for 6 hours under nitrogen atmosphere. The mixture was filtered. The filtrate was diluted with water (20.0 mL) and extracted with ethyl acetate (10.0 mL). The organic layer was dried over sodium sulfate, filtered and concentrated. The residue was purified by prep-HPLC (basic condition) and SFC ([0.1% ammonium hydroxide IPA to afford N-((5-amino-3-cyano-6-methyl-1H-pyrrolo[3,2-b]pyridin-2-yl)methyl)benzamide, Example 15-6 (3.84 mg, 12.2 μmol, 19.8% yield) as a white solid. LCMS [ESI, M+1]⁺=306.1. ¹HNMR (400 MHz, DMSO-d₆) δ=11.87-11.62 (m, 1H), 9.17 (br t, J=5.2 Hz, 1H), 8.04-7.83 (m, 2H), 7.59-7.53 (m, 1H), 7.52-7.46 (m, 2H), 7.45-7.38 (m, 1H), 6.07-5.43 (m, 2H), 4.68 (d, J=5.6 Hz, 2H), 2.13 (s, 3H).

### Example 15-7

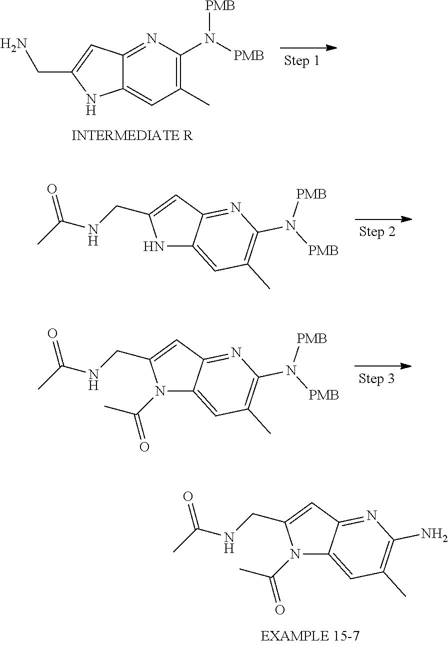

Step 1: To a solution of Intermediate R (40.0 mg, 96.0 μmol, 1.00 eq.) in tetrahydrofuran (1.00 mL) was added triethylamine (38.9 mg, 384 μmol, 53.5 μL 4.00 eq.) and acetic anhydride (29.4 mg, 288 μmol, 27.0 μL 3.00 eq.). The mixture was stirred at 20° C. for 3 hours. The mixture was diluted with water (20.0 mL) and extracted with ethyl acetate (10.0 mL×2). The organic layer was washed with brine (20.0 mL), dried over sodium sulfate, filtered and concentrated. The residue was purified by flash silica gel chromatography (10-100/o Ethyl acetate/Petroleum ether) to afford N-((5-(bis(4-methoxybenzyl)amino)-6-methyl-1H-pyrrolo[3,2-b]pyridin-2-yl)methyl)acetamide (30.0 mg, 64.8 μmol, 67.5% yield) as a light yellow oil. LCMS [ESI, M+1]⁺=459.3

Step 2: To a solution of N-((5-(bis(4-methoxybenzyl)amino)-6-methyl-1H-pyrrolo[3,2-b]pyridin-2-yl)methyl)acetamide (30.0 mg, 65.4 μmol, 1.00 eq) and acetic acid (4.71 mg, 78.5 μmol, 4.49 μL 1.20 eq.) in dichloromethane (1 mL) was added N,N′-methanediylidenedicyclohexanamine (20.3 mg, 98.1 μmol, 19.9 μL 1.50 eq.) and 4-dimethylaminopyridine (799 ug, 6.54 μmol, 0.10 eq.). The mixture was stirred at 25° C. for 16 hours. The mixture was concentrated. The residue was purified by flash silica gel chromatography (10˜50% Ethyl acetate/Petroleum ether) to afford N-((1-acetyl-5-(bis(4-methoxybenzyl)amino)-6-methyl-1H-pyrrolo[3,2-b]pyridin-2-yl)methyl)acetamide (15.0 mg, 30.0 μmol, 45.8% yield) as a light yellow oil. ¹H NMR (400 MHz, CDCl₃) δ=7.68 (s, 1H), 7.20 (d, J=8.4 Hz, 4H), 6.82-6.77 (m, 4H), 6.73 (s, 1H), 6.48 (br t, J=6.4 Hz, 1H), 4.72 (d, J=6.4 Hz, 2H), 4.22 (s, 4H), 3.77 (s, 6H), 2.77 (s, 3H), 2.50 (s, 3H), 1.99 (s, 3H).

Step 3: To a solution of N-((1-acetyl-5-(bis(4-methoxybenzyl)amino)-6-methyl-1H-pyrrolo[3,2-b]pyridin-2-yl)methyl)acetamide (15.0 mg, 30.0 μmol, 1.00 eq) in dichloromethane (0.60 mL) was added trifluoroacetic acid (185 mg, 1.62 mmol, 120 μL 54.1 eq.). The mixture was stirred at 20° C. for 12 hours. The mixture was concentrated. The residue was purified by prep-HPLC (neutral condition) and lyophilized to afford N-((1-acetyl-5-amino-6-methyl-1H-pyrrolo[3,2-b]pyridin-2-yl)methyl)acetamide, Example 15-7 (5.94 mg, 21.4 μmol, 25.3% yield) as a yellow solid. LCMS [ESI, M+1]⁺=261.2. ¹H NMR (400 MHz, DMSO-d₆) δ=8.30 (t, J=5.2 Hz, 1H), 7.84 (s, 1H), 6.33 (s, 1H), 5.58 (s, 2H), 4.57 (d, J=5.6 Hz, 2H), 2.70 (s, 3H), 2.16 (s, 3H), 1.92 (s, 3H).

### Example 15-8

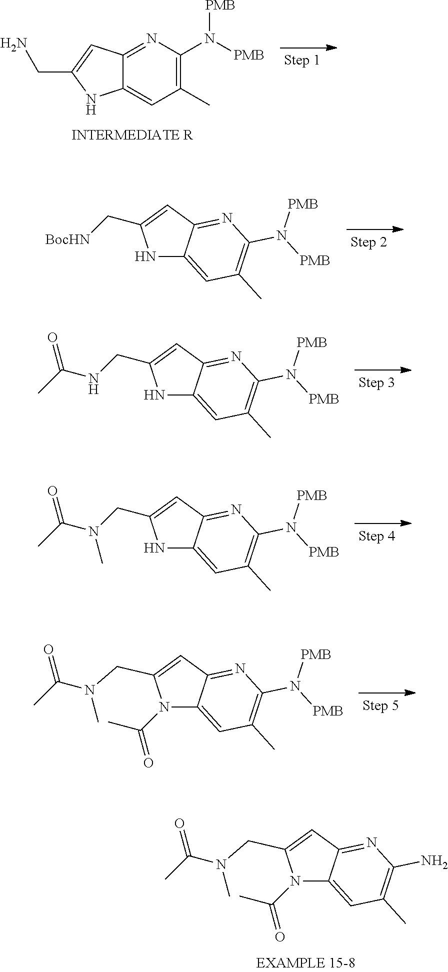

Step 1: To a solution of Intermediate R (100 mg, 240 μmol, 1.00 eq.) in tetrahydrofuran (1.00 mL) was added triethylamine (97.2 mg, 960 μmol, 134 μL 4.00 eq.) and di-tert-butyl dicarbonate (157 mg, 720 μmol, 165 μL 3.00 eq.). The mixture was stirred at 20° C. for 3 hours. The mixture was diluted with water (30.0 mL) and extracted with ethyl acetate (10.0 mL-2). The organic layer was washed with brine (20.0 mL), dried over sodium sulfate, filtered and concentrated. The residue was purified by flash silica gel chromatography (10-50% Ethyl acetate/Petroleum ether) to afford tert-butyl ((5-(bis(4-methoxybenzyl)amino)-6-methyl-1H-pyrrolo[3,2-b]pyridin-2-yl)methyl)carbamate (100 mg, 189 μmol, 78.7% yield) as a light yellow oil. LCMS [EST, M+1]=517.2

Step 2: To a solution of tert-butyl ((5-(bis(4-methoxybenzyl)amino)-6-methyl-1H-pyrrolo[3,2-b]pyridin-2-yl)methyl)carbamate (100 mg, 194 μmol, 1.00 eq.) in tetrahydrofuran (3.00 mL) was added Lithium Aluminum Hydride (36.7 mg, 968 μmol, 5.00 eq.) at 0° C. The mixture was stirred at 60° C. for 16 hours. The mixture was quenched with sodium sulfate at 0-5° C. under nitrogen. The mixture was filtered and the filter cake was washed with tetrahydrofuran (10.0 mL×3). The combined organic layers were concentrated to afford N,N-bis(4-methoxybenzyl)-6-methyl-2-((methylamino)methyl)-1H-pyrrolo[3,2-b]pyridin-5-amine (80.0 mg, crude) as a light yellow oil. LCMS [ESI, M+1]⁺=431.2

Step 3: To a solution of N,N-bis(4-methoxybenzyl)-6-methyl-2-((methylamino)methyl)-1H-pyrrolo[3,2-b]pyridin-5-amine (100 mg, 232 μmol, 1.00 eq.) in tetrahydrofuran (3.00 mL) was added triethylamine (118 mg, 1.16 mmol, 162 μL 5.00 eq.) and acetic anhydride (71.1 mg, 697 μmol, 65.3 μL 3.00 eq.). The mixture was stirred at 20° C. for 3 hours. The mixture was concentrated. The residue was purified by flash silica gel chromatography (10-100/o Ethyl acetate/Petroleum ether) to afford N-((5-(bis(4-methoxybenzyl)amino)-6-methyl-1H-pyrrolo[3,2-b]pyridin-2-yl)methyl)-N-methylacetamide (70.0 mg, 119 μmol, 51.3% yield) as a light yellow oil. LCMS [ESI, M+1]⁺=473.4

Step 4: To a solution of N-((5-(bis(4-methoxybenzyl)amino)-6-methyl-1H-pyrrolo[3,2-b]pyridin-2-yl)methyl)-N-methylacetamide (50.0 mg, 106 μmol, 1.00 eq.) in tetrahydrofuran (2.50 mL) was added acetic anhydride (21.6 mg, 212 μmol, 19.8 μL 2.00 eq.), triethylamine (21.4 mg, 212 μmol, 29.5 μL 2.00 eq.) and 4-dimethylaminopyridin (12.9 mg, 106 μmol, 1.00 eq.). The mixture was stirred at 60° C. for 24 hours. The mixture was concentrated. The residue was purified by flash silica gel chromatography (5˜-50% Ethyl acetate/Petroleum ether) to afford N-((1-acetyl-5-(bis(4-methoxybenzyl)amino)-6-methyl-1H-pyrrolo[3,2-b]pyridin-2-yl)methyl)-N-methylacetamide (40.0 mg, 67.6 μmol, 63.9% yield) as s light yellow oil. LCMS [ESI, M+1]⁺=515.4

Step 5: To a solution of N-((1-acetyl-5-(bis(4-methoxybenzyl)amino)-6-methyl-1H-pyrrolo[3,2-b]pyridin-2-yl)methyl)-N-methylacetamide (40.0 mg, 77.7 μmol, 1.00 eq.) in dichloromethane (1.00 mL) was added trifluoroacetic acid (308 mg, 2.70 mmol, 0.20 mL, 34.8 eq.). The mixture was stirred at 20° C. for 16 hours. The mixture was concentrated. The residue was purified by prep-HPLC (HCl condition) and lyophilized to afford N-((1-acetyl-5-amino-6-methyl-1H-pyrrolo[3,2-b]pyridin-2-yl)methyl)-N-methylacetamide, Example 5-8 (4.50 mg, 14.48 μmol, 18.6% yield, hydrochloric acid salt) as a yellow solid. LCMS [ESI, M+1]⁺=275.2. ¹H NMR (400 MHz, DMSO-d₆) δ=8.35 (s, 1H), 7.91-6.74 (m, 1H), 6.44-6.27 (m, 1H), 5.00-4.77 (m, 2H), 2.93 (br d, J=4.4 Hz, 3H), 2.78 (s, 3H), 2.29 (s, 3H), 2.17-1.97 (m, 3H).

### Example 16-1

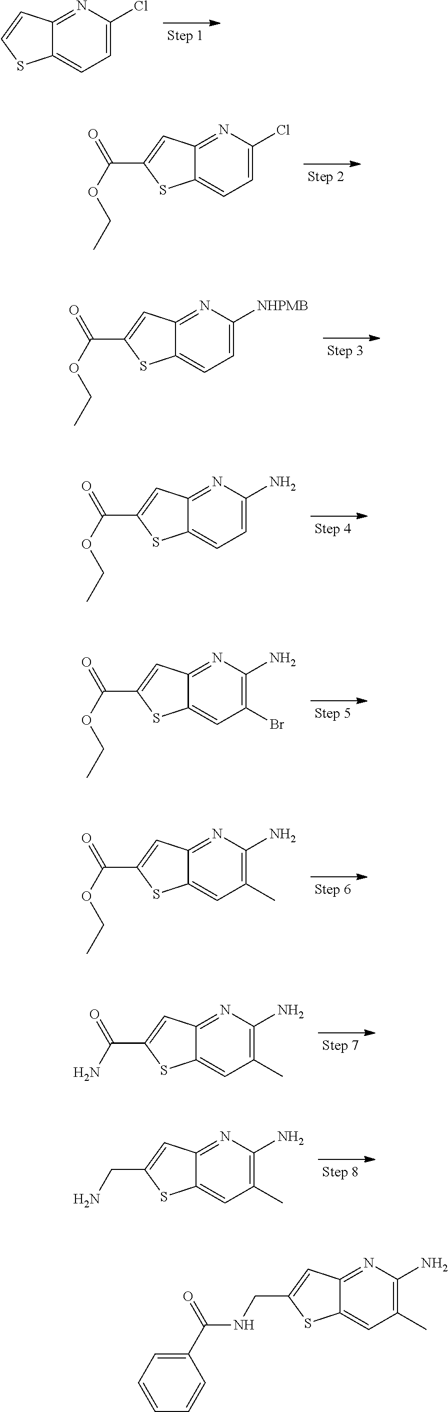

Step 1: A mixture of 5-chlorothieno[3,2-b]pyridine (3.30 g, 19.5 mmol, 1.00 eq.) in tetrahydrofuran (50.0 mL) was added n-BuLi (2.50 M, 11.7 mL, 1.50 eq.) at −78° C. under nitrogen atmosphere, and the mixture was stirred at −78° C. for 2 hours under nitrogen atmosphere. Ethyl carbonochloridate (3.17 g, 29.2 mmol, 2.78 mL, 1.50 eq.) was added at −78° C. The mixture was warmed to 25° C. and stirred at 25° C. for 2 hours under nitrogen atmosphere. The reaction mixture was quenched by addition of water (50.0 mL) at 25° C., and then extracted with ethyl acetate (100 mL×3). The combined organic layers were washed with brine (100 mL), Extracted with ethyl acetate (100.0 mL). The organic layer was dried over sodium sulfate, filtered and concentrated under reduced pressure to give a residue. The residue was purified by column chromatography (SiO₂, Petroleum ether/Ethyl acetate 20:1) to give ethyl 5-chlorothieno[3,2-b]pyridine-2-carboxylate (0.80 g, 3.31 mmol, 20.1% yield) as a yellow solid. LCMS [M+1]⁺=242.1

Step 2: To a solution of ethyl 5-chlorothieno[3,2-b]pyridine-2-carboxylate (800 mg, 3.23 mmol, 1.00 eq.) in toluene (10.0 mL) was added (4-methoxyphenyl)methanamine (885 mg, 6.45 mmol, 835 μL 2.00 eq.), BrettPhos Pd Cn; (293 mg, 323 μmol, 0.10 eq.) and cesium carbonate (3.15 g, 9.68 mmol, 3.00 eq.). The mixture was stirred at 100° C. for 2 hours. The reaction mixture was diluted with water (50.0 mL) and extracted with ethyl acetate (50.0 mL-3). The combined organic layers were washed with brine (50.0 mL), dried over sodium sulfate, filtered and concentrated under reduced pressure to give a residue. The residue was purified by prep-TLC (SiO₂, petroleum ether/ethyl acetate 5:1) to give ethyl 5-[(4-methoxyphenyl)methylamino]thieno[3,2-b]pyridine-2-carboxylate (700 mg, 2.04 mmol, 63.4% yield) as a yellow solid. LCMS [M+1]⁺=343.3

Step 3: To a solution of ethyl 5-[(4-methoxyphenyl)methylamino]thieno[3,2-b]pyridine-2-carboxylate (800 mg, 2.34 mmol, 1.00 eq.) was added TFA (15.4 g, 135 mmol, 10.0 mL, 57.8 eq.). The mixture was stirred at 80° C. for 12 hours. The reaction mixture was concentrated under reduced pressure to give ethyl 5-aminothieno[3,2-b]pyridine-2-carboxylate (350 mg, crude) as a yellow solid. LCMS [M+1]⁺=223.2.

Step 4: To a solution of ethyl 5-aminothieno[3,2-b]pyridine-2-carboxylate (350 mg, 1.57 mmol, 1.00 eq.) in tetrahydrofuran (5.00 mL) was added NBS (336 mg, 1.89 mmol, 1.20 eq.) at 0° C. The mixture was stirred at 0-25° C. for 1 hour. The reaction mixture was quenched by addition of aq. sodium bicarbonate (10.0 mL) at 25° C., and then extracted with ethyl acetate (20.0 mL×3). The combined organic layers were washed with brine (20.0 mL), dried over sodium sulfate, filtered and concentrated under reduced pressure to give a residue. The residue was purified by prep-TLC (SiO₂, petroleum ether/ethyl acetate 3:1) to give ethyl 5-amino-6-bromo-thieno[3,2-b]pyridine-2-carboxylate (300 mg, 996 μmol, 63.4% yield) as a yellow solid. LCMS [M+1]⁺=302.7

Step 5: A mixture of ethyl 5-amino-6-bromo-thieno[3,2-b]pyridine-2-carboxylate (260 mg, 863 μmol, 1.00 eq.), methylboronic acid (258 mg, 4.32 mmol, 5.00 eq.), Ad₂nBuP Pd G₃(cataCXium® A Pd G₃) (62.9 mg, 86.3 μmol, 0.10 eq.) and potassium phosphate (550 mg, 2.59 mmol, 3.00 eq.) in dioxane (5.00 mL) and water (1.00 mL) was degassed and stirred at 80° C. for 2 hours under nitrogen atmosphere. The reaction mixture was diluted with water (30.0 mL) and extracted with ethyl acetate (30.0 mL×3). The combined organic layers were washed with brine (30.0 mL), dried over sodium sulfate, filtered and concentrated under reduced pressure to give a residue. The residue was purified by column chromatography (SiO₂, petroleum ether/ethyl acetate 5:1 to 1:1) to give ethyl 5-amino-6-methyl-thieno[3,2-b]pyridine-2-carboxylate (130 mg, 550 μmol, 63.7% yield) as a yellow solid. LCMS [M+1]⁺=237.2.

Step 6: A mixture of ethyl 5-amino-6-methyl-thieno[3,2-b]pyridine-2-carboxylate (130 mg, 762 μmol, 1.00 eq.) and ammonium hydroxide (9.00 g, 260 mmol, 10.00 mL, 10.0% purity, 34.0 eq.) was stirred at 60° C. for 2 hours. The reaction mixture was filtered and concentrated under reduced pressure to give 5-amino-6-methyl-thieno[3,2-b]pyridine-2-carboxamide (100 mg, 483 μmol, 63.4% yield) as a yellow solid. LCMS [M+1]⁺=208.

Step 7: To a solution of 5-amino-6-methyl-thieno[3,2-b]pyridine-2-carboxamide (100 mg, 483 μmol, 1.00 eq.) in tetrahydrofuran (4.00 mL) was added lithium aluminium hydride (54.9 mg, 1.45 mmol, 3.00 eq.). The mixture was stirred at 60° C. for 2 hours. The reaction mixture was quenched by addition of aq. sodium sulfate (5.00 mL at 0° C.), and then extracted with ethyl acetate (10.0 mL×3). The combined organic layers were washed with brine (5.00 mL), dried over sodium sulfate, filtered and concentrated under reduced pressure to give 2-(aminomethyl)-6-methyl-thieno[3,2-b]pyridin-5-amine (80.0 mg, 414 μmol, 85.8% yield) as a yellow solid. LCMS [M+1]⁺=194.0.

Step 8: To a solution of 2-(aminomethyl)-6-methyl-thieno[3,2-b]pyridin-5-amine (60.0 mg, 310 μmol, 1.00 eq.) in DMF (1.00 mL) was added HATU (153 mg, 404 μmol, 1.30 eq.), triethyl amine (40.8 mg, 404 μmol, 56.2 μL 1.30 eq.) and benzoic acid (41.7 mg, 341 μmol, 52.1 μL 1.10 eq.). The mixture was stirred at 0-25° C. for 1 hour. The reaction mixture was filtered and concentrated under reduced pressure to give a residue. The residue was purified by prep-HPLC (neutral conditions) to give N-[(5-amino-6-methyl-thieno[3,2-b]pyridin-2-yl)methyl]benzamide, Example 16-1 (15.0 mg, 49.1 μmol, 15.8% yield) as an off-white solid. LCMS [M+1]⁺=298.0. ¹H NMR (400 MHz, METHANOL-d₄) δ=7.87-7.84 (m, 2H), 7.75 (s, 1H), 7.57-7.52 (m, 1H), 7.50-7.45 (m, 2H), 7.08 (s, 1H), 4.80 (s, 2H), 2.22 (s, 3H).

### Example A

This Example illustrates that exemplary compounds of Formula (I) in the present invention are capable of binding to PRMT5 in presence of MTA.

The PRMT5 binding activity of compounds of the present invention was determined using an HTRF binding assay or SPR assay.

### HTRF Binding Assay

A recombinant human dual expressed Avi PRMT5/His-MEP50) protein (corresponding to amino acids for PRMT5 2-637, and 2-342 for MEP50 expressed in baculovirus) was incubated with target fragments in final buffer (25 mM ADA pH 7.2, 30 μM MTA, 1 mM TCEP, 50 mM NaCl, 0.002% Tween, 5 nM proprietary Tracer binding compound prepared in-house), overnight at 2-8° C. After overnight incubation the binding is monitored after the addition of 0.5 nM Anti-His-Tb (Cisbio) after 1 hr incubation at RT (˜20-24 hrs total binding time). The HTRF signal was measured using a Clariostar reader (BMG) excitation filter (Ex Tr), dichroic filter (LP TP) and emission filters (F 665-10 and F 620-10) manufacturer's instructions. The HTRF ratio was calculated using the formula: [emission 665/emission 620]*10000. IC50's were fit using Xlfit software (IDBS) with the Hill equation fixed to 1 (fit Background+Bmax/(1+((x/IC50)\hat{ }Hill))) and the results for exemplary compounds of Formula (I) is shown in Table A1

TABLE A1
IC₅₀ Values for PRMT5 Binding Activity by Exemplary Compounds
of Formula (I) in the Presence of MTA in the HTRF Assay

| Example | IC₅₀ (μM) |
|---|---|
| 1-1 | 17.191 |
| 1-2 | 81.989 |
| 1-3 | 2.085 |
| 1-4 | 7.458 |
| 1-5 | 55.487 |
| 1-6 | 51.007 |
| 1-7 | 7.915 |
| 1-8 | 11.352 |
| 2-1 | 6.641 |
| 2-10 | 2.135 |
| 2-2 | 0.354 |
| 2-3 | 1.309 |
| 2-4 | 0.992 |
| 2-5 | 8.826 |
| 2-6 | 2.849 |
| 2-7 | 0.581 |
| 2-8 | 35.529 |
| 2-9 | 0.204 |
| 3-1 | 8.988 |
| 3-2 | 5.110 |
| 3-3 | 4.804 |
| 3-4 | 33.852 |
| 3-5 | 39.625 |
| 3-6 | 40.603 |
| 3-7 | 0.546 |
| 3-8 | 84.049 |
| 4-10 | 13.510 |
| 4-2 | 13.918 |
| 4-3 | 23.894 |
| 4-4 | 29.593 |
| 4-5 | 64.093 |
| 4-6 | 21.770 |
| 4-7 | 10.148 |
| 4-9 | 14.015 |
| 5-1 | 100.000 |
| 5-3 | 87.914 |
| 6-1 | 100.000 |
| 7-1 | 0.158 |
| 7-2 | 0.313 |
| 7-3 | 1.056 |
| 7-4 | 0.029 |
| 7-5 | 0.003 |
| 7-6 | 0.021 |
| 7-7 | 0.024 |
| 8-1 | 0.009 |
| 8-2 | 0.013 |
| 9-1 | 2.196 |
| 9-2 | 24.723 |
| 9-3 | 20.330 |
| 11-1 | 3.676 |
| 11-2 | 0.073 |
| 11-3 | 100.000 |
| 11-4 | 1.427 |
| 12-1 | 1.409 |
| 12-2 | 5.461 |
| 12-3 | 18.283 |
| 12-4 | 1.621 |
| 13-1 | 1.283 |
| 13-2 | 4.265 |
| 13-3 | 2.358 |
| 13-4 | 50.624 |
| 13-5 | 31.796 |
| 13-6 | 6.688 |
| 13-7 | 2.290 |
| 14-1 | 0.007 |
| 14-2 | 0.002 |
| 14-3 | 0.102 |
| 14-4 | 0.124 |
| 14-5 | 0.037 |
| 14-6 | 0.007 |
| 14-7 | 0.008 |
| 15-1 | 0.362 |
| 15-2 | 2.465 |
| 15-3 | 0.138 |
| 15-4 | 17.031 |
| 15-5 | 100.000 |
| 15-6 | 2.642 |
| 15-7 | 0.581 |
| 15-8 | 1.010 |
| 16-1 | 0.163 |

### SPR Binding Assay

In vivo biotinylated PRMT5-MEP50 was diluted to 4.5 μM in 25 mM Bicine pH 7.6, 100 mM NaCl, 1 mM TCEP, and 0.05% Tween-20 and injected at 5 μl/min flow rate into flow cell 2 (FC2) of a Series S Sensor Chip SA (Cytiva) in a Biacore T200 or in a Biacore 8K plus (Cytiva). SPR screening was performed in MTA running buffer (25 mM Bicine pH 7.6, 100 mM NaCl, 1 mM TCEP, 20 μM MTA, 0.05% Tween-20 and 2% DMSO). The biotinylated PRMT5-MEP50 surface was equilibrated with MTA running buffer for 12 hours prior to the start. The test compound affinity was determined using multi-cycle injection of each fragment from 0.001 to 500 μM over the PRMT5•MTA at a flow rate of 30 μl/min and with association and dissociation times of 20 and 60 seconds respectively. PRMT5•MTA surface activity was confirmed at the initiation, and the end of the run by titration of EPZ015666 (K_{D}=11 and 13 μM respectively). Subsequently, compound titration was repeated in SAM-running buffer (25 mM Bicine pH 7.6, 100 mM NaCl, 1 mM TCEP, 20 μM SAM, 0.05% Tween-20, and 2% DMSO). The PRMT5•SAM surface was equilibrated for at least 5 hours prior to compound titration and the PRMT5•SAM surface activity was confirmed at the end of the fragment titration run by titration of EPZ015666 (K_{D}<1 nM). After double referencing, the steady-state response was extracted for each fragment concentration and was fit to the Langmuir isotherm equation to determine the equilibrium dissociation constant (K_{D}).

The data in Table A2 was generated using a surface plasmon resonance (“SPR”) binding assay.

TABLE A2
IC₅₀ Values for PRMT5 Binding Activity by
Exemplary Compounds of Formula (I) in the
Presence of MTA and SAM in the SPR Assay

K_{D} μM

| Example | MTA | SAM |
|---|---|---|
| 1-8 | 7.499 | 19.1 |
| 2-2 | 5.285 | 39.1 |
| 2-3 | 4.676 | 31.5 |
| 2-4 | 0.874 | 3.09 |
| 2-7 | 0.232 | 1.22 |
| 2-9 | 0.137 | 0.748 |
| 3-7 | 2.460 | |
| 4-7 | 31.971 | 81.38 |
| 7-1 | 0.094 | 0.396 |
| 7-2 | 0.151 | 2.220 |
| 7-3 | 0.682 | 12.5 |
| 7-4 | 0.022 | 0.081 |
| 7-5 | 0.002 | 0.002 |
| 7-6 | 0.012 | 0.105 |
| 8-1 | 0.005 | 0.033 |
| 8-2 | 0.009 | 0.137 |
| 9-1 | 0.456 | 3.36 |
| 9-2 | 3.260 | 19.6 |
| 9-3 | 11.7 | 29.2 |
| 12-1 | 2.54 | 6.29 |
| 12-4 | 1.89 | 4.95 |
| 13-1 | 0.51 | 1.18 |
| 13-3 | 0.68 | 2.01 |
| 13-4 | 10.7 | 30.9 |
| 14-1 | 0.001 | 0.006 |
| 14-2 | 0.001 | 0.001 |
| 14-3 | 0.012 | 0.038 |
| 14-4 | 0.017 | 0.080 |
| 14-5 | 0.203 | 0.727 |
| 14-6 | 0.001 | 0.006 |
| 14-7 | 0.002 | 0.006 |
| 15-1 | 0.066 | |
| 15-5 | 774 | 631 |
| 15-8 | 0.043 | 0.341 |
| 16-1 | 0.072 | 1.351 |

This Example illustrates that exemplary compounds of Formula (I) of the present invention cooperatively inhibit PRMT5 enzymatic activity in the presence of MTA.

The PRMT5 inhibitory activity of compounds of the present invention was determined using a PRMT5:MEP50 FlashPlate Assay (Reaction Biology Corporation).

### PRMT5:MEP50 FlashPlate Assay

The assay uses purified human, PRMT5 enzyme to convert S-adenosyl-L-[methyl-³H]methionine plus histone H4 L-arginine to S-adenosyl-L-homocysteine plus histone H4 [methyl-³H]-L-arginine. The assay was carried out using Streptavidin-coated FlashPlates (Perkin Elmer), which contained a scintillant embedded in the plastic of the plate. The histone H4 peptide substrate was conjugated with biotin, which binds to the streptavidin-coated well of the plate, placing the H4 peptide in close proximity to the side well and the scintillant. The transfer of the tritiated methyl group from S-adenosyl-L-[methyl-³H]methionine to the bound histone H4 peptide generated a radiolabeled histone H4, which was quantitated by measuring in a scintillation counter to determine the activity of PRMT5 enzyme in the presence and absence of compound. The assay reactions also were conducted in the presence and absence of MTA to determine whether the compounds exhibit MTA-cooperative activity. Briefly, compounds of the present invention were solubilized in 100% DMSO at a highest concentration of 10 mM. For IC, determinations, the initial starting concentration for the serial dilutions of each compound was 50 μM. Control samples lacking compound, PRMT5/MEP50 complex or various reaction components also were prepared and processed in parallel with compound test samples. SAH was used as a positive control for assay validation. To measure PRMT5 inhibitory activity, 3 nM PRMT5/MEP50 complex (Reaction Biology Corporation) was preincubated with test compound in assay buffer containing 40 nM histone H4 peptide (amino acids 1-15)-Biotin conjugate for 20 min at room temperature. The enzymatic reaction was initiated by adding 1 μM tritiated S-adenosyl methionine (final concentration) and the reaction is allowed to proceed for 20 min. The reaction was stopped and the amount of bound, tritiated H4 peptide in each sample was determined using a scintillation counter. The IC₅₀ value for each compound was calculated from each 10-point dose-response curve for samples plus and minus MTA using GraphPad Prism software and the results for exemplary compounds of Formula (I) is shown in Table 9a.

TABLE B1
IC₅₀ Values for PRMT5-mediated Enzymatic Activity
by Exemplary Compounds of Formula (I) in the Presence
and Absence of MTA in the FlashPlate Assay

| | IC₅₀ + 2 μM | IC₅₀ |
|---|---|---|
| Example | MTA (μM) | (μM) |
| 1-8 | >300 | >300 |
| 2-3 | >300 | >300 |
| 2-4 | >300 | >300 |
| 2-9 | 17.1 | 243 |
| 3-7 | 26.1 | 351 |
| 4-7 | >300 | >300 |
| 7-1 | 5.57 | 62.6 |
| 7-2 | 22 | >100 |
| 7-3 | 60 | >100 |
| 7-4 | 1.66 | 18.2 |
| 7-5 | 0.0203 | 6.46 |
| 7-6 | 0.745 | >300 |
| 7-7 | 2.39 | >300 |
| 8-1 | 0.419 | 4.29 |
| 8-2 | 0.362 | 5.79 |
| 9-1 | 27.9 | 188 |
| 9-2 | 97.8 | 232 |
| 9-3 | 210 | 431 |
| 11-2 | 5.8 | |
| 11-4 | 79.8 | >300 |
| 12-1 | >100 | >100 |
| 12-2 | 44.7 | 60.2 |
| 12-3 | 97.7 | 95.9 |
| 12-4 | 48.86 | 100 |
| 13-1 | 176 | >300 |
| 13-2 | 72.8 | 83.8 |
| 13-3 | 131 | >300 |
| 13-4 | >300 | >300 |
| 13-5 | 70 | >300 |
| 13-6 | 183 | 60.3 |
| 13-7 | >300 | >300 |
| 14-1 | 0.0121 | 10.1 |
| 14-2 | 0.00172 | 0.798 |
| 14-3 | 0.138 | 271 |
| 14-4 | 1.24 | 170 |
| 14-5 | 4.26 | 245 |
| 14-6 | 0.504 | 43.2 |
| 14-7 | 0.474 | 36.7 |
| 15-1 | 44.9 | >300 |
| 15-2 | 57.9 | >300 |
| 15-6 | 76.7 | >300 |
| 16-1 | 18.2 | >300 |

### PRMT5:MEP50 HotSpot Assay

The assay uses recombinant full-length histone H2A as the PRMT5 substrate. Enzymatic transfer of the tritiated methyl group from S-adenosyl-L-[methyl-3H]methionine to the histone H2A protein generated a radiolabeled histone H2A4 by measuring in a scintillation counter to determine the activity of PRMT5 enzyme in the presence and absence of compound. The assay reactions also were conducted in the presence of MTA to determine whether the compounds exhibit MTA-cooperative activity. Briefly, compounds of the present invention were solubilized in 100% DMSO at a highest concentration of 10 mM. For IC₅₀ determinations, the initial starting concentration for the serial dilutions of each compound was 50 μM. Control samples lacking compound, PRMT5/MEP50 complex or various reaction components also were prepared and processed in parallel with compound test samples. SAH was used as a positive control for assay validation. To measure PRMT5 inhibitory activity. 1 nM PRMT5/MEP50 complex (Reaction Biology Corporation) was preincubated with test compound in assay buffer containing 5 μM full-length histone H2A for 20 min at room temperature. The enzymatic reaction was initiated by adding 1 μM tritiated S-adenosyl methionine (final concentration) and the reaction was allowed to proceed for 60 min. The reaction was stopped and transferred to filter paper for detection. The amount of tritiated H2A in each sample was determined using a scintillation counter. The IC₅₀ value for each compound was calculated from each 10-point dose-response curve using GraphPad Prism software and the results for exemplary compounds of Formula (I) is shown in Table B2.

TABLE B2
IC₅₀ Values for PRMT5-mediated Enzymatic Activity
by Exemplary Compounds of Formula (I) in the
Presence of MTA in the HotSpot Assay

| | IC₅₀ + 2 μM |
|---|---|
| Example | MTA (μM) |
| 7-5 | 1.34 |
| 10-1 | 9.07 |
| 10-1 | 9.07 |
| 10-11 | 10.4 |
| 10-2 | 0.123 |
| 10-3 | 10.3 |
| 10-4 | 0.379 |
| 10-5 | 14.7 |
| 10-6 | 0.925 |
| 10-7 | 16.2 |
| 10-8 | 0.0602 |
| 10-9 | 2.53 |
| 10-9 | 33.1 |
| 14-1 | 0.626 |
| 14-2 | 0.553 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula I: 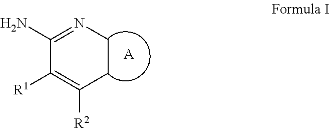
wherein:
A is selected from 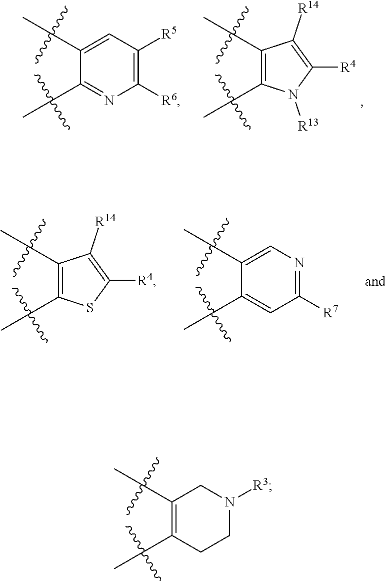
R¹ is hydrogen, Br, F, —C1-C2 alkyl, —C3-C4 cycloalkyl or —CF₃;
R² is hydrogen or C1-C2 alkyl;
R³ is hydrogen, pyrazolyl optionally substituted with C1-C3 alkyl or phenyl optionally substituted with cyano, or pyridine optionally substituted with —O-phenyl;
R⁴ is
hydrogen,
—C(O)—O—(C1-C2 alkyl),
-L⁴-NH—C(O)-phenyl where phenyl is optionally substituted with one or more fluoro,
-L⁴-NH—C(O)-pyrimidine, imidazole or triazole where the imidazole and triazole are optionally substituted with bromo,
-L⁴-(CO)N(R¹⁰)(R¹¹) where R¹⁰ is pyridyl(C₁-C₆ alkyl) where the pyridyl is optionally substituted with halogen or trifluoromethyl and R¹¹ is pyridyl(C₁-C₆ alkyl), pyrimidinyl(C₁-C₆ alkyl) or 5,6,7,8-tetrahydroquinoxalinyl,
-L⁴-1,3-dioxoisoindolin-2-yl(C₀-C₂ alkyl) where the alkyl is optionally substituted with cyano,
-L⁴-1-oxo-3,4-dihydroisoquinolin-2-yl(C₀-C₂ alkyl) where the alkyl is optionally substituted with cyano,
-L⁴-1-oxoisoquinolin-2-yl(C₀-C₂ alkyl) where the alkyl is optionally substituted with cyano,
-L⁴-2,4-dioxoimidazolidin-1-yl,
-L⁴-NH—C(O)(C₁-C₂ alkyl)R¹²) where R¹² is hydrogen, C₁-C₂ alkyl, or naphthyl optionally substituted with cyano, or
-L⁴-3-(C₁-C₃ alkyl)-2,4-dioxoimidazolidin-1-yl,
where L⁴ is absent or C₁-C₂-alkyl, and provided that when R⁴ is hydrogen, at least one of R¹ and R² is not hydrogen;
R⁵ is
hydrogen,
-L⁵-phenyl optionally substituted with one or more substituents selected from fluoro, chloro, cyano and C1-C2 alkyl,
-L⁵-pyrimidine optionally substituted with one or more substituents selected from hydroxy and —NH-cyclopropyl,
-L⁵-pyridine,
-L⁵-pyradazine,
-L⁵-isoxazole,
-L⁵-thiazole,
-L⁵-1,3-dioxoisoindolin-2-yl,
-L⁵-(CO)N(R¹⁶)(R¹⁷) where R¹⁶ is pyridyl(C₁-C₆ alkyl) where the pyridyl is optionally substituted with halogen or trifluoromethyl and R¹⁷ is pyridyl(C₁-C₆ alkyl), pyrimidinyl(C₁-C₆ alkyl) or 5,6,7,8-tetrahydroquinoxalinyl,
-L⁵-NH—C(O)(C1-C2 alkyl)(R¹⁸) where R¹⁸ is hydrogen, C₁-C₂ alkyl, or naphthyl optionally substituted with cyano, or
-L⁵-1-methyl-pyrazole or bromo,
where L⁵ is absent, —CH₂—NH—C(O)—, C1-C2 alkylene optionally substituted with cyano, —O— or —CH₂OCH₂—;
R⁶ is
hydrogen,
-L⁶-phenyl optionally substituted with fluoro,
-L⁶-pyridine,
-L⁶-isothiazole,
-L⁶-thiazole,
-L⁶-1-methyl-pyrazole, —NH—(C1-C2 alkyl) or —NH—(C3-C4 cycloalkyl),
where L⁶ is absent or C1-C1-alkylene, and provided that at least one of R⁵ and R⁶ is not hydrogen;
R⁷ is C1-C2 alkyl, chloro, 1-methyl-pyrazole or phenyl optionally substituted with one or more substituents selected from fluoro, chloro, cyano and C1-C2 alkyl;
R¹³ is hydrogen, C₂-C₃ acyl, C₁-C₂ alkyl or C₃-C₆ cycloalkyl; or
R¹³ and R⁴ together with the atoms to which they are attached form a 5-7 membered ring containing one nitrogen atom, and wherein the ring is optionally substituted with one or more of oxo and C₂-C₃ acyl; and
R¹⁴ is hydrogen, cyano or C₂-C₃ acyl;
or a pharmaceutically acceptable salt thereof.

2. The compound according to claim 1, wherein R¹ is hydrogen or R¹ is Br, methyl, ethyl or cyclopropyl.

3. (canceled)

4. The compound of claim 1, wherein R² is methyl.

5. The compound of claim 1, wherein A is

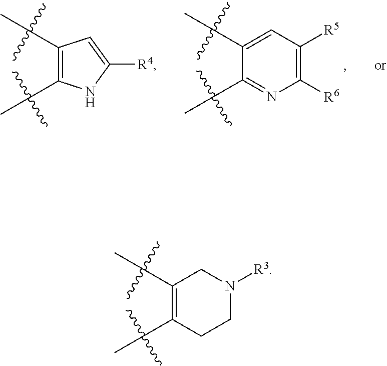

6. The compound of claim 1, wherein R⁴ is hydrogen.

7. (canceled)

8. The compound of claim 1, wherein L⁵ is absent.

9. The compound of claim 1, wherein L⁵ is methylene or wherein L⁵ is —O—.

10. The compound of claim 1, wherein L⁵ is —O—.

11. The compound of claim 8, wherein R⁵ is -phenyl optionally substituted with one or more substituents selected from fluoro, chloro, cyano and C1-C2 alkyl, or R⁵ is
pyrimidine optionally substituted with one or more substituents selected from hydroxy and —NH-cyclopropyl.

12. The compound of claim 1, wherein L⁵ —CH₂—NH—C(O)—.

13. The compound of claim 1, wherein R⁵ is hydrogen and R⁶ is not hydrogen.

14. The compound of claim 1, wherein R⁵ is not hydrogen and R⁶ is hydrogen.

15. The compound of claim 1, wherein A is

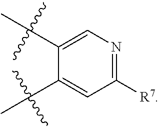

16. The compound of claim 15, wherein R⁷ is C1-C2 alkyl, chloro, 1-methyl-pyrazole or phenyl.

17. The compound of claim 15, wherein R⁷ is phenyl optionally substituted with one or more substituents selected from fluoro, chloro, cyano and C1-C2 alkyl.

18. (canceled)

19. The compound of claim 1, wherein the compound is: 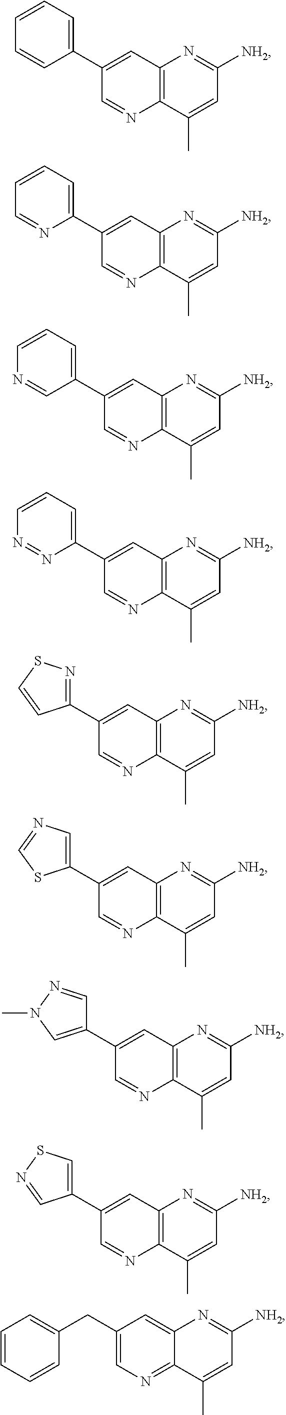 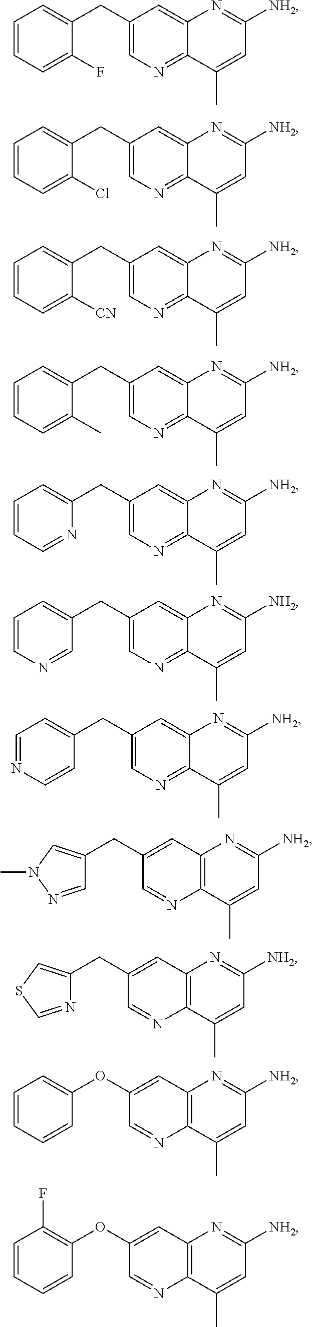 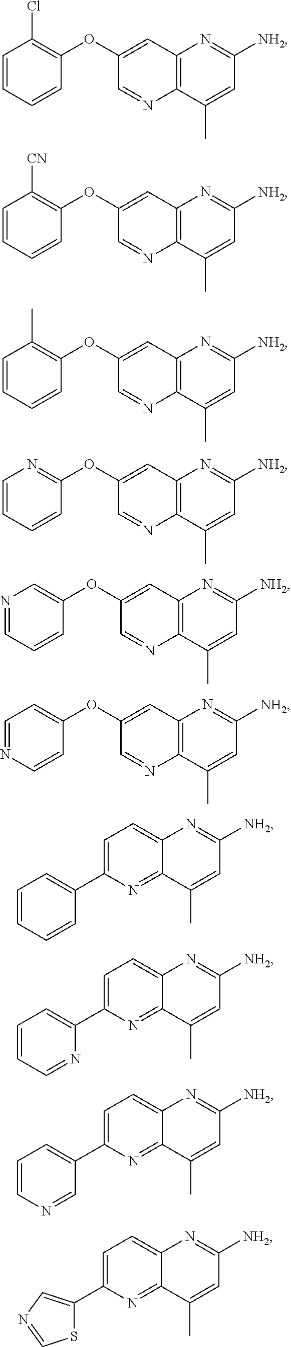 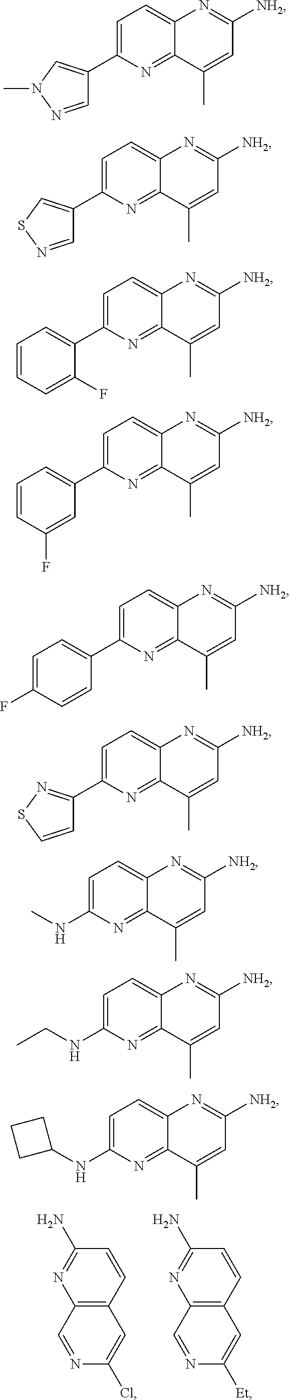 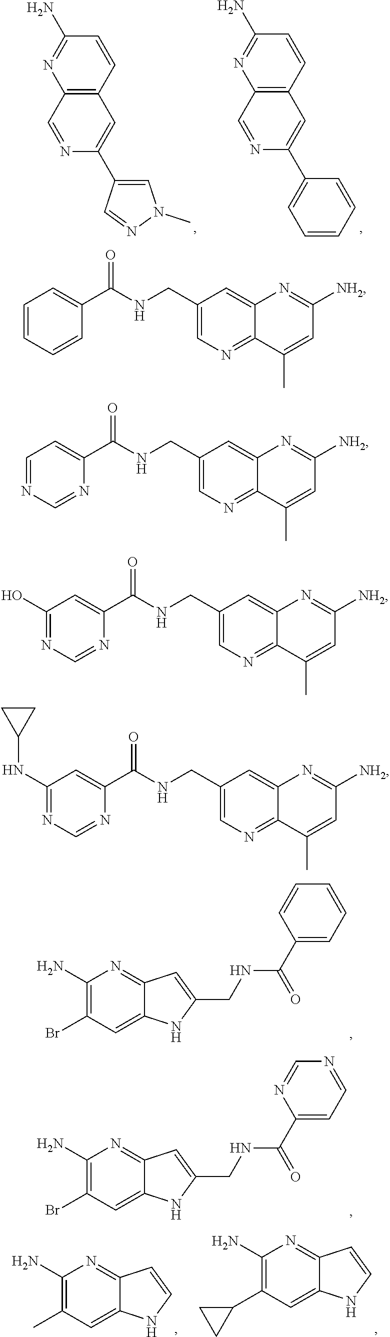 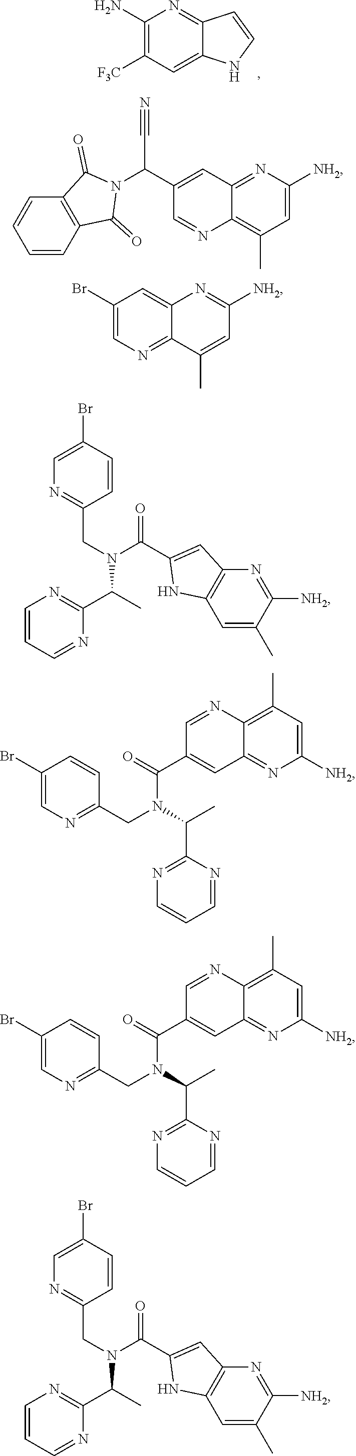 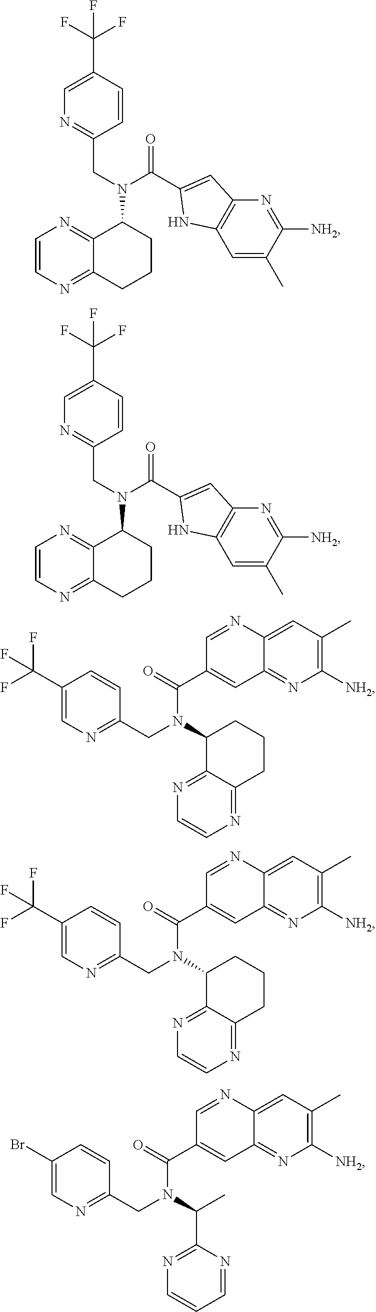 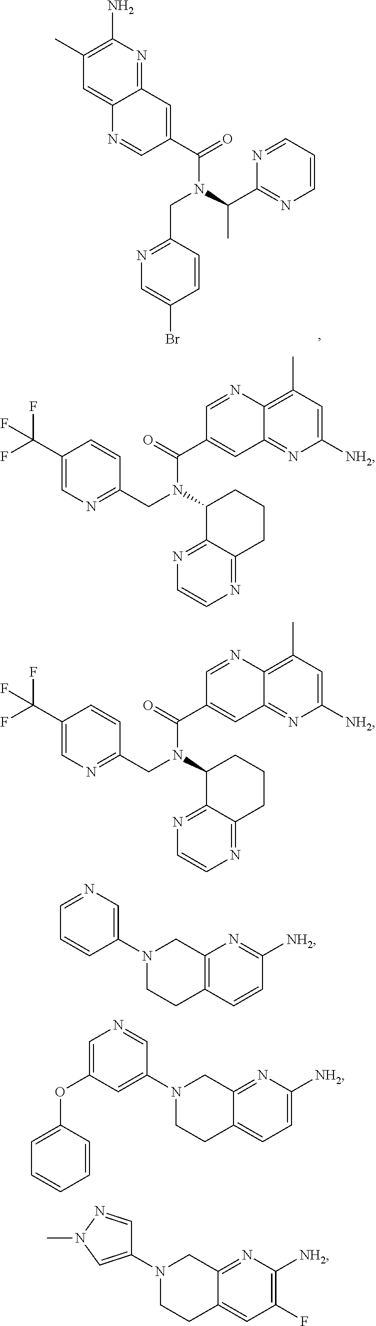 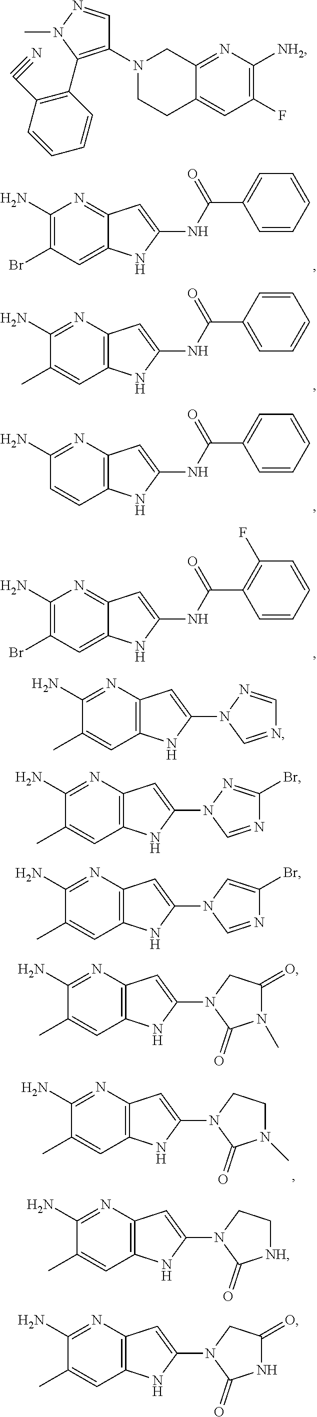 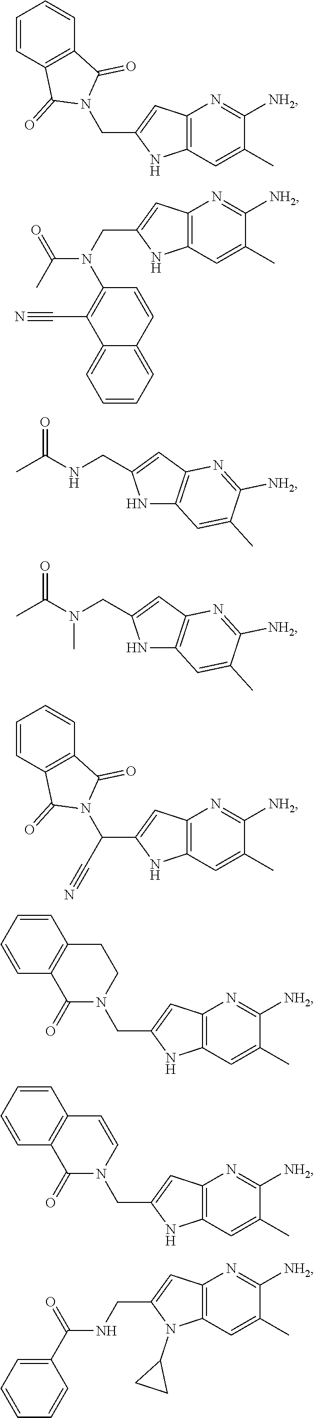 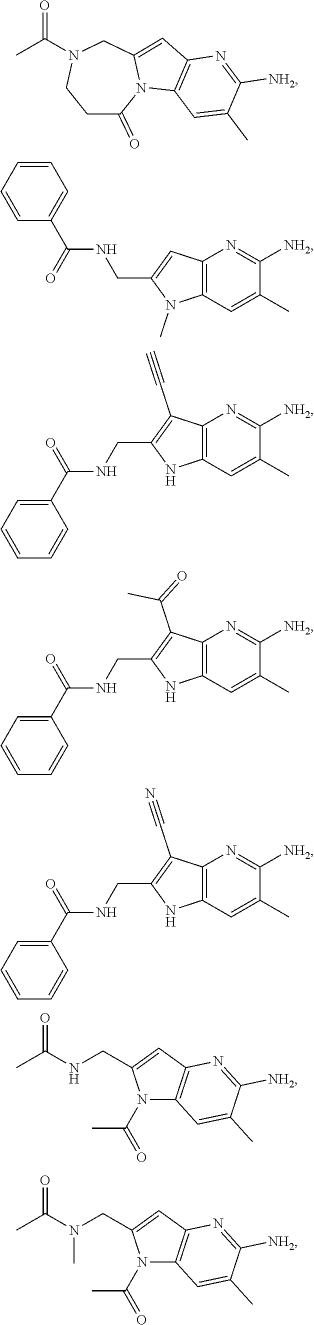 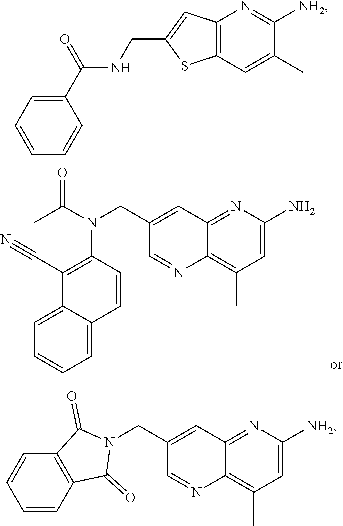
or a pharmaceutically acceptable salt thereof.

20. A pharmaceutical composition, comprising a therapeutically effective amount of a compound of Formula I according to claim 1 or a pharmaceutically acceptable salt thereof, and a pharmaceutically acceptable excipient.

21. A method for inhibiting PRMT5 activity in a cell, comprising contacting the cell in which inhibition of PRMT5 activity is desired with an effective amount of a compound of Formula I according to claim 1 or a pharmaceutically acceptable salt thereof, or a pharmaceutical composition comprising a therapeutically effective amount of a compound of Formula I according to claim 1 or a pharmaceutically acceptable salt thereof, and a pharmaceutically acceptable excipient.

22. A method for treating cancer comprising administering to a patient having cancer a therapeutically effective amount of a compound of Formula I according to claim 1 or a pharmaceutically acceptable salt or solvate thereof, alone or combined with a pharmaceutically acceptable carrier, excipient or diluents.

23. (canceled)

24. (canceled)

25. The method according to claim 22, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

26. (canceled)

27. The method according to claim 22, wherein the cancer is hepatocellular carcinoma, breast cancer, skin cancer, bladder cancer, liver cancer, pancreatic cancer, or head and neck cancer.
